(19) United States
(12) Patent Application Publication
(10) Pub. No.: US 2018/0127786 A1
(43) Pub. Date: May 10, 2018

(54) COMPOSITIONS AND METHODS FOR GENE EDITING

(71) Applicant: CASEBIA THERAPEUTICS LIMITED LIABILITY PARTNERSHIP, San Francisco, CA (US)

(72) Inventors: Axel Bouchon, Leverkusen (DE); Ante S. Lundberg, Cambridge, MA (US); Lawrence Klein, Cambridge, MA (US); Peter G. Nell, San Francisco, CA (US); Basha Stankovich, Alameda, CA (US)

(21) Appl. No.: 15/715,068

(22) Filed: Sep. 25, 2017

Related U.S. Application Data
(60) Provisional application No. 62/398,555, filed on Sep. 23, 2016.

(51) Int. Cl. C12N 15/90; C12N 15/11; C12N 9/22

(52) U.S. Cl. CPC C12N 15/907; C12N 15/11; A61K 48/00; C12N 2800/80; C12N 9/22

(57) ABSTRACT

Provided include materials and methods for treating a subject with one or more conditions associated with WAS gene whether ex vivo or in vivo. Also provided include materials and methods for editing and/or modulating the expression of WAS gene in a cell by genome editing.

### CROSS-REFERENCES TO RELATED APPLICATIONS

This application claims the benefit of U.S. Provisional Application No. 62/398,555, filed Sep. 23, 2016, which is incorporated herein by reference in entirety and for all purposes.

### REFERENCE TO A SEQUENCE LISTING SUBMITTED AS AN ASCII FILE

The present application is being filed along with a Sequence Listing in electronic format. The Sequence Listing file, entitled SEQ_LIST.txt, was created on Sep. 21, 2017, and is 9.58 Mega Bytes in size. The information in electronic format of the Sequence Listing is incorporated herein by reference in its entirety.

### FIELD

The disclosures provided herewith relates to the field of gene editing and specifically to the alteration of the WAS gene.

### BACKGROUND

Genome engineering refers to the strategies and techniques for the targeted, specific modification of the genetic information (genome) of living organisms. Genome engineering is a very active field of research because of the wide range of possible applications, particularly in the areas of human health; the correction of a gene carrying a harmful mutation, for example, or to explore the function of a gene. Early technologies developed to insert a transgene into a living cell were often limited by the random nature of the insertion of the new sequence into the genome. Random insertions into the genome may result in disrupting normal regulation of neighboring genes leading to severe unwanted effects. Furthermore, random integration technologies offer little reproducibility, as there is no guarantee that the sequence would be inserted at the same place in two different cells. Recent genome engineering strategies, such as ZFNs, TALENs, HEs and MegaTALs, enable a specific area of the DNA to be modified, thereby increasing the precision of the correction or insertion compared to early technologies. These newer platforms offer a much larger degree of reproducibility, but still have their limitations.

Despite efforts from researchers and medical professionals worldwide who have been trying to address genetic disorders, and despite the promise of genome engineering approaches, there still remains a critical need for developing safe and effective treatments involving WAS gene related indications.

By using genome engineering tools to create permanent changes to the genome that can address the WAS gene related disorders or conditions with a single treatment, the resulting therapy may completely remedy certain WAS gene related indications and/or diseases.

### SUMMARY OF THE INVENTION

Provided herein are cellular, ex vivo and in vivo methods for creating permanent changes to the genome by inserting or deleting one or more nucleotides from a gene or genes in the genome, e.g., in WAS gene. In other cases, WAS gene may be defective such that replacing all or a part of WAS gene would be therapeutic. Such methods are also contemplated herein. The methods are useful for correcting, eliminating or modulating the expression or function of one or more gene products encoded at, within, or near the WAS gene (neighboring genes) or other DNA sequences that encode regulatory elements of the WAS gene. Also provided herein are components, kits, and compositions for performing such methods. Also provided are cells produced by such methods which may be useful in treating any WAS gene related disorder or disorder of a gene in the genomic neighborhood (upstream or downstream) of WAS gene.

Accordingly, provided here is a method of editing a genome in a cell. The method can have inserting a nucleic acid sequence of a Wiskott-Aldrich syndrome gene (WAS gene) or functional derivative thereof into a genomic sequence of the cell, wherein the cell has one or more mutation(s) in the genome which results in reduction of the expression of endogenous WAS gene as compared to the expression in a normal cell that does not have such mutation(s).

In embodiments, the method can further have providing the following to the cell: (a) a deoxyribonucleic acid (DNA) endonuclease or an oligonucleotide encoding said DNA endonuclease and (b) a targeting oligonucleotide having a first region of at least 15 bases complementary to the genomic sequence. In some embodiments, the WAS gene or functional derivative thereof is inserted using a donor template having the nucleic acid sequence of the WAS gene or functional derivative thereof.

In embodiments, the DNA endonuclease is an enzyme selected from the group consisting of any of those in Table 1, Table 2, and variants having at least 70% homology to any of those listed in Table 1 or Table 2.

In embodiments, the DNA endonuclease is Cas 9.

In embodiments, the oligonucleotide encoding the DNA endonuclease is codon optimized.

In embodiments, the oligonucleotide encoding said DNA endonuclease is a deoxyribonucleic acid (DNA) sequence.

In embodiments, the oligonucleotide encoding the DNA endonuclease is a ribonucleic acid (RNA) sequence.

In embodiments, the RNA sequence encoding the DNA endonuclease is linked to the targeting oligonucleotide via a covalent bond.

In embodiments, the targeting oligonucleotide is a guide RNA (gRNA).

In embodiments, the first region of the gRNA is selected from those listed in Table 4 and variants thereof having at least 85% homology to any of those listed in Table 4.

In embodiments, the genomic sequence is at, within, or near the WAS gene or WAS gene regulatory elements.

In embodiments, the genomic sequence is in an intergenic region that is upstream of the promoter of the endogenous WAS gene in the genome.

In embodiments, the intergenic region is at least 500 bp upstream of the first exon of the endogenous WAS gene in the genome.

In embodiments, the inserting is at, within, or near a safe harbor locus or a safe harbor site.

In embodiments, the safe-harbor locus is selected from the group consisting of albumin gene, AAVS 1 gene, HRPT gene, CCR5 gene, globin gene, TTR gene. TF gene, F9 gene, Alb gene, Gys2 gene and PCSK9 gene.

In embodiments, the safe harbor site is selected from the group consisting of the following regions: AAVS1 19q13.4-qter, HRPT 1q31.2, CCR5 3p21.31, Globin 11p15.4, TTR 18q12.1, TF 3q22.1, F9 Xq27.1, Alb 4q13.3, Gys2 12p12.1, and PCSK9 1p32.3.

In embodiments, the genomic sequence is at, within, or near the AAVS1 gene.

In embodiments, the genomic sequence is in an intergenic region that is upstream of the promoter of the AAVS1 gene in the genome.

In embodiments, the intergenic region is at least 2.5 kb upstream of the first exon of the AAVS1 gene in the genome.

In embodiments, the intergenic region is about 2.5 kb to about 5 kb upstream of the first exon of the AAVS1 gene in the genome.

In embodiments, one or more of the foregoing-mentioned oligonucleotides are encoded in an Adeno Associated Virus (AAV) vector.

In embodiments, the DNA endonuclease and/or one or more of the foregoing-mentioned oligonucleotide are formulated in a liposome or lipid nanoparticle.

In embodiments, the DNA endonuclease is formulated in a liposome or lipid nanoparticle.

In embodiments, the liposome or lipid nanoparticle further has the targeting oligonucleotide.

In embodiments, one or more of the foregoing-mentioned (a), (b) and (c) are provided to the cell via electroporation.

In embodiments, one or more of the foregoing-mentioned (a), (b) and (c) are provided to the cell via chemical transfection.

In embodiments, the DNA endonuclease is precomplexed with the targeting oligonucleotide, forming a Ribonucleoprotein (RNP) complex, prior to the provision to the cell.

In embodiments, the RNP is provided to the cell via electroporation.

In embodiments, the foregoing-mentioned one or more mutation(s) are present at, within, or near the endogenous WAS gene in the genome.

In embodiments, the expression of endogenous WAS gene in the cell is about 10% about 20%, about 30%, about 40%, about 50%, about 60%, about 70%, about 80%, about 90% or about 100% reduced as compared to the expression of endogenous WAS gene expression in the normal cell.

In embodiments, the expression of the introduced WAS gene or functional derivative thereof in the cell is at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous WAS gene of the cell.

In embodiments, the expression of the introduced WAS gene or functional derivative thereof in the cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the cell.

In embodiments, the cell is a stem cell.

In embodiments, the stem cell is a CD34⁺ hematopoietic stem and progenitor cell (HSPC).

Also provided herein is a method of treating a subject for a Wiskott-Aldrich syndrome (WAS) gene related condition or disorder. The method has providing a genetically modified cell to the subject, wherein a genome of the genetically modified cell is edited such that an exogenous nucleic acid sequence of a WAS gene or functional derivative thereof is inserted in the genome.

In embodiments, the subject is a patient having or is suspected of having Wiskott-Aldrich syndrome (WAS).

In embodiments, the subject is diagnosed with a risk of the Wiskott-Aldrich syndrome (WAS) gene related condition or disorder.

In embodiments, the genetically modified cell is autologous.

In embodiments, the autologous cell has one or more mutation(s) in the genome which results in reduction of the expression of endogenous WAS gene as compared to the expression of endogenous WAS gene in a normal cell that does not have such mutation(s).

In embodiments, the foregoing-mentioned one or more mutation(s) are present at, within, or near the endogenous WAS gene in the genome.

In embodiments, the expression of endogenous WAS gene in the genetically modified cell is about 10%, about 20%, about 30%, about 40%, about 50%, about 60%, about 70%, about 80%, about 90% or about 100% reduced as compared to the expression of endogenous WAS gene expression in a normal cell that does not have such mutation(s).

In embodiments, the expression of the introduced WAS gene or functional derivative thereof in the genetically modified cell is at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous WAS gene of the genetically modified cell.

In embodiments, the expression of the introduced WAS gene or functional derivative thereof in the genetically modified cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the genetically modified cell.

In embodiments, the cell is a stem cell.

In embodiments, the stem cell is a CD34⁺ hematopoietic stem and progenitor cell (HSPC).

In embodiments, the method further has obtaining a biological sample from the subject, wherein the biological sample has a CD34⁺ cell, and editing the genome of at least one cell by inserting the exogenous nucleic acid sequence of a WAS gene or functional derivative thereof into a genomic sequence of the cell, thereby producing the genetically modified cell.

In embodiments, the exogenous nucleic acid sequence is inserted at, within, or near the WAS gene or WAS gene regulatory elements.

In embodiments, the genomic sequence is in an intergenic region that is upstream of the promoter of the endogenous WAS gene in the genome.

In embodiments, the intergenic region is at least 500 bp upstream of the first exon of the endogenous WAS gene in the genome.

In embodiments, the exogenous nucleic acid sequence is inserted at, within, or near a safe harbor locus or a safe harbor site.

In embodiments, the safe-harbor locus is selected from the group consisting of albumin gene, AAVS1 gene, HRPT gene, CCR5 gene, globin gene, TTR gene, TF gene, F9 gene, Alb gene, Gys2 gene and PCSK9 gene.

In embodiments, the safe harbor site is selected from the group consisting of the following regions: AAVS1 19q13.4-qter, HRPT 1q31.2, CCR5 3p21.31, Globin 11p15.4, TTR 18q12.1, TF 3q22.1, F9 Xq27.1, Alb 4q13.3, Gys2 12p12.1, and PCSK9 1p32.3.

In embodiments, the exogenous nucleic acid sequence is inserted at, within, or near the AAVS1 gene.

In embodiments, the genomic sequence is in an intergenic region that is upstream of the promoter of the AAVS1 gene in the genome.

In embodiments, the intergenic region is at least 2.5 kb upstream of the first exon of the AAVS1 gene in the genome.

In embodiments, the intergenic region is about 2.5 kb to about 5 kb upstream of the first exon of the AAVS1 gene in the genome.

Also provided herein is a composition having a guide RNA (gRNA) sequence having a sequence selected from those listed in Table 4 and/or variants thereof having at least 85% homology to any of those listed in Table 4.

In embodiments, the composition further has a DNA endonuclease or an oligonucleotide encoding said DNA endonuclease.

In embodiments, the composition further has a donor template having a nucleic acid sequence of a WAS gene or functional derivative thereof.

In embodiments, the DNA endonuclease is an enzyme selected from the group consisting of any of those in Table 1, Table 2, and variants having at least 70% homology to any of those listed in Table 1 or Table 2.

In embodiments, the DNA endonuclease is Cas 9.

In embodiments, the oligonucleotide encoding the DNA endonuclease is codon optimized.

In embodiments, the oligonucleotide encoding said DNA endonuclease is a deoxyribonucleic acid (DNA) sequence.

In embodiments, the oligonucleotide encoding the DNA endonuclease is a ribonucleic acid (RNA) sequence.

In embodiments, the RNA sequence encoding said DNA endonuclease is linked to the gRNA via a covalent bond.

In embodiments, the composition further has a liposome or lipid nanoparticle.

In embodiments, the DNA endonuclease is precomplexed with the gRNA, forming a Ribonucleoprotein (RNP) complex.

Also provided herein is a composition having a guide RNA (gRNA) sequence that has a spacer sequence complementary to (i) a genomic sequence at, within, or near Wiskott-Aldrich syndrome (WAS) gene or (ii) a genomic sequence at, within, or near a safe harbor locus or a safe harbor site.

In embodiments, the safe harbor locus is selected from the group consisting of albumin gene, AAVS1 gene, HRPT gene, CCR5 gene, globin gene, TTR gene, TF gene, F9 gene, Alb gene, Gys2 gene and PCSK9 gene.

In embodiments, the safe harbor site is selected from the group consisting of the following regions: AAVS1 19q13.4-qter. HRPT 1q31.2, CCR5 3p21.31, Globin 11p15.4, TTR 18q12.1, TF 3q22.1, F9 Xq27.1, Alb 4q13.3, Gys2 12p12.1, and PCSK9 1p32.3.

In embodiments, the spacer sequence is 15 bases to 20 bases in length.

In embodiments, the complementarity between the spacer sequence to the genomic sequence is at least 80%, at least 85%, at least 90%, at least 95%, at least 96%, at least 97%, at least 98%, at least 99% or at least 100%.

In embodiments, the composition further has one or more of the following: a deoxyribonucleic acid (DNA) endonuclease or an oligonucleotide encoding the DNA endonuclease and a donor template having a nucleic acid sequence of a WAS gene or functional derivative thereof.

In embodiments, the DNA endonuclease is an enzyme selected from the group consisting of any of those in Table 1, Table 2, and variants having at least 70% homology to any of those listed in Table 1 or Table 2.

In embodiments, the DNA endonuclease is Cas 9.

In embodiments, the oligonucleotide encoding said DNA endonuclease is codon optimized.

In embodiments, the oligonucleotide encoding said DNA endonuclease is a ribonucleic acid (RNA) sequence.

In embodiments, the RNA sequence encoding the DNA endonuclease is linked to the gRNA via a covalent bond.

In embodiments, the composition further has a liposome or lipid nanoparticle.

In embodiments, the DNA endonuclease is precomplexed with the gRNA, forming a Ribonucleoprotein (RNP) complex.

Also provided herein is a kit having any of the foregoing-mentioned composition and further having instructions for use.

Also provided herein is a method for editing a Wiskott-Aldrich syndrome gene (WAS gene) in a human cell by genome editing comprising the step of introducing into the human cell one or more deoxyribonucleic acid (DNA) endonucleases to effect one or more single-strand breaks (SSBs) or double-strand breaks (DSBs) within or near the WAS gene or WAS gene regulatory elements that results in a permanent insertion or deletion of at least one nucleotide thereby affecting the expression or function of WAS gene products.

Also provided herein is a method of altering the contiguous genomic sequence of WAS gene in a cell, tissue or organism comprising contacting said contiguous WAS gene genomic sequence with: (a) an enzyme selected from the group consisting of any of those in Table 1, Table 2, and variants having at least 70% homology to any of those listed in Table 1 or Table 2, (b) at least one targeting oligonucleotide (sgRNA or gRNA) capable of hybridizing to said genomic sequence and (c) optionally a donor oligonucleotide.

In some embodiments, one or more targeting oligonucleotide (sgRNA or gRNA) independently comprises a first region of at least 15 linked nucleosides complementary to the genomic sequence and a second region of between 5 and 15 linked nucleosides operably connected to said first region via a covalent bond or hybridization forces.

Also provided herein is an ex vivo method for treating a patient have a WAS gene related condition or disorder comprising the steps of: i) creating a patient specific induced pluripotent stem cell (iPSC), ii) editing within or near an a Wiskott-Aldrich syndrome gene (WAS gene) or other DNA sequences that encode regulatory elements of the WAS gene of the iPSC; iii) differentiating the genome-edited iPSC into a selected cell type, and iv) implanting the cell type into the patient.

In some embodiments, the editing step comprises introducing into the iPSC one or more deoxyribonucleic acid (DNA) endonucleases to effect one or more single-strand breaks (SSBs) or double-strand breaks (DSBs) and is selected from any of those in Table 1, Table 2, and variants having at least 70% homology to any of those listed in Table 1 or Table 2.

Also provided herein is an ex vivo method for treating a patient with Wiskott-Aldrich Syndrome (WAS) comprising the steps of: i) isolating a mesenchymal stem cell from the patient, ii) editing within or near a Wiskott-Aldrich syndrome gene (WAS gene) or other DNA sequences that encode regulatory elements of the WAS gene of the mesenchymal stem cell, iii) differentiating the genome-edited mesenchymal stem cell into a differentiated cell, and iv) implanting the differentiated cell into the patient.

Also provided herein is an in vivo method for treating a patient with a WAS gene related disorder comprising the step of editing the Wiskott-Aldrich syndrome gene (WAS gene) in a cell of the patient.

In some embodiments, the editing step comprises introducing into the cell one or more deoxyribonucleic acid (DNA) endonucleases to effect one or more single-strand breaks (SSBs) or double-strand breaks (DSBs) selected from any of those in Table 1, Table 2, and variants having at least 70% homology to any of those listed in Table 1 or Table 2.

Also provided herein is a method of altering the contiguous genomic sequence of WAS gene in a cell comprising contacting said cell with a gene editing nuclease, wherein said nuclease is encoded as a chemically modified mRNA.

In some embodiments, the modified mRNA is chemically modified in the coding region.

In some embodiments, the gene editing nuclease is selected from any of those in Table 1. Table 2, and variants having at least 70% homology to any of those listed in Table 1 or Table 2.

In some embodiments, the chemically modified mRNA is codon optimized.

In some embodiments, the gene editing nuclease is formulated in a liposome or lipid nanoparticle.

In some embodiments, the gene editing nuclease is formulated in a lipid nanoparticle which also comprises one or more gRNAs or one or more sgRNAs.

In some embodiments, the method further comprises introducing into the cell a donor template comprising at least a portion of the wild-type WAS gene.

In some embodiments, the at least a portion of the wild-type WAS gene comprises one or more sequences selected from the group consisting of a WAS gene exon, a WAS gene intron, a sequence comprising an exon:intron junction of WAS gene.

In some embodiments, the method further has introducing into the cell a donor template comprising at least a portion of a wild-type neighboring gene of WAS gene.

In some embodiments, the donor template is either a single or double stranded polynucleotide.

In some embodiments, the method further has introducing one or more gRNAs or one or more sgRNAs.

In some embodiments, one or more gRNAs or one or more sgRNAs are chemically modified.

In some embodiments, one or more gRNAs or one or more sgRNAs is precomplexed with the gene editing nuclease.

In some embodiments, the pre-complexing involves a covalent attachment of said one or more gRNAs or one or more sgRNAs to said gene editing complex.

In some embodiments, the alteration of the contiguous genomic sequence occurs 5′, 3′ or at the site of one or more SNPs of WAS gene.

In some embodiments, the gene editing enzyme is encoded in an AAV vector particle, where the AAV vector serotype is selected from the group consisting of AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3.bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2. AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1. AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22. AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63. AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02. AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC 12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24. AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73. AAVrh.74, AAVrh.8, AAVrh.8R, AAVrh8R, AAVrh8R A586R mutant, AAVrh8R R533A mutant, BAAV, BNP61 AAV, BNP62 AAV. BNP63 AAV, bovine AAV, caprine AAV, Japanese AAV10, true type AAV (ttAAV), UPENN AAV10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8 and those listed in Tables 4 and 5.

In some embodiments, the donor template is encoded in an AAV vector particle, where the AAV vector serotype is selected from the group consisting of AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu 15, AAV33.8/hu.16, AAV3-9/rh.52, AAV3a, AAV3b, AAV4, AAV4-19/rh.55, AAV42.12, AAV42-10, AAV42-1 1, AAV42-12, AAV42-13, AAV42-15, AAV42-1b, AAV42-2, AAV42-3a, AAV42-3b, AAV42-4, AAV42-5a, AAV42-5b, AAV42-6b. AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu 19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22. AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54. AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC 12, AAV-PAEC2, AAV-PAEC4, AAV-PAEC6, AAV-PAEC7, AAV-PAEC8, AAVpi.1. AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV. BNP63 AAV, bovine AAV, caprine AAV, Japanese AAV10, true type AAV (ttAAV), UPENN AAV10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1. AAV SM 10-2, AAV SM 10-8 and those listed in Tables 4 and 5.

In some embodiments, the one or more gRNAs or one or more sgRNAs is encoded in an AAV vector particle, where the AAV vector serotype is selected from the group consisting of AAV1, AAV10, AAV106.1/hu.37, AAV1, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-01, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54. I/hu.21, AAV54.2/hu.22, AAV54.4R/hu.27, AAV54.5/hu.23, AAV54.7/hu 24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV10, true type AAV (ttAAV), UPENN AAV10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8 and those listed in Tables 4 and 5. The method of any one of claims 1, 6, 11, 16, or 20-45, wherein the Cas9 or Cpf1 mRNA is formulated into a lipid nanoparticle, and the gRNA is delivered to the cell by electroporation and donor template is delivered to the cell by an adeno-associated virus (AAV) vector.

Provided herein is a method for editing a Wiskott-Aldrich syndrome gene (WAS gene) in a mammalian or other type cell by genome editing including the step of introducing into the cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene (neighboring genes) that results in a permanent insertion and/or deletion, to effect correction, or modulation of expression or function of the WAS gene and results in restoration of WAS protein activity, function or levels. As used herein, a WAS gene related disorder includes any disorder that is causally related to WAS gene or a gene that is the upstream or downstream or opposing strand gene of WAS gene in the chromosome.

Also provided herein is an ex vivo method for treating a patient (e.g., a human) with Wiskott-Aldrich Syndrome (WAS) or WAS gene related disorder, the method having the steps of: creating a patient specific induced pluripotent stem cell (iPSC); editing at, within, or near a Wiskott-Aldrich syndrome gene (WAS gene) or other DNA sequences that encode regulatory elements of the WAS gene of the iPSC; differentiating the genome-edited iPSC into a cell of choice, such as a hepatocyte; and implanting the differentiated and edited stem cell into the patient.

The step of creating a patient specific induced pluripotent stem cell (iPSC) can have: a) isolating a somatic cell from the patient; and b) introducing a set of pluripotency-associated genes into the somatic cell to induce the somatic cell to become a pluripotent stem cell. The somatic cell can be a fibroblast. The set of pluripotency-associated genes can be one or more of the genes selected from the group consisting of OCT4, SOX1, SOX2. SOX3, SOX15, SOX18, NANOG, KLF1, KLF2, KLF4, KLF5, c-MYC, n-MYC, REM2, TERT and LIN28.

The step of editing at, within, or near a Wiskott-Aldrich syndrome gene (WAS gene) or other DNA sequences that encode regulatory elements of the WAS gene of the iPSC can include introducing into the iPSC one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and results in restoration of WAS protein activity.

The step of differentiating the genome-edited iPSC into another cell, e.g., a hepatocyte may have one or more of the following: contacting the genome-edited iPSC with one or more of activin, B27 supplement, FGF4, HGF, BMP2, BMP4, Oncostatin M, dexamethasone.

The step of implanting the differentiated cells into the patient can include implanting the cell into the patient by local injection, systemic infusion, or combinations thereof.

Also provided herein is an ex vivo method for treating a patient (e.g., a human) with Wiskott-Aldrich Syndrome (WAS) or WAS gene related disorder, the method having the steps of: performing a biopsy of the patient's tissue; isolating a tissue specific progenitor cell or primary cell; editing the WAS gene or other DNA sequences that encode regulatory elements of the progenitor cell or primary cell; and implanting the progenitor cell or primary cell into the patient.

The step of isolating a tissue specific progenitor cell or primary cell can include: perfusion of fresh tissues with digestion enzymes, cell differential centrifugation, cell culturing, or combinations thereof.

The step of editing at, within, or near a Wiskott-Aldrich syndrome gene (WAS gene) or other DNA sequences that encode regulatory elements of the WAS gene of the progenitor cell or primary cell may include introducing into the progenitor cell or primary cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity.

Also provided herein is an ex vivo method for treating a patient (e.g., a human) with Wiskott-Aldrich Syndrome (WAS), the method having the steps of: isolating a mesenchymal stem cell from the patient; editing at, within, or near a Wiskott-Aldrich syndrome gene (WAS gene) or other DNA sequences that encode regulatory elements of the WAS gene of the mesenchymal stem cell; differentiating the genome-edited mesenchymal stem cell into another cell; and implanting the differentiated cell into the patient.

The mesenchymal stem cell can be isolated from the patient's bone marrow or peripheral blood. The step of isolating a mesenchymal stem cell from the patient can include aspiration of bone marrow and isolation of mesenchymal cells by density centrifugation using Percoll™.

The step of editing at, within, or near the Wiskott-Aldrich syndrome gene (WAS gene) or other DNA sequences that encode regulatory elements of the WAS gene of the mesenchymal stem cell can include introducing into the mesenchymal stem cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity.

Also provided herein is an in vivo method for treating a patient (e.g., a human) with Wiskott-Aldrich Syndrome (WAS) having the step of editing a Wiskott-Aldrich syndrome gene (WAS gene) in a cell of the patient.

The step of editing a Wiskott-Aldrich syndrome gene (WAS gene) in a cell of the patient can include introducing into the cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity. This method may also include editing one or more neighboring genes to WAS gene. As used herein, a neighboring gene is a gene found immediately upstream, downstream or on the opposing strand of the genomic DNA relative to WAS gene.

The one or more DNA endonucleases can be a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog, recombination of the naturally occurring molecule, codon-optimized, or modified version thereof, and combinations of any of the foregoing. Any of the endonucleases disclosed herein may be used.

The methods of the present disclosure may include introducing into the cell one or more polynucleotides encoding the one or more DNA endonucleases. The method can include introducing into the cell one or more ribonucleic acids (RNAs) encoding the one or more DNA endonucleases. The one or more polynucleotides or one or more RNAs can be one or more modified polynucleotides or one or more modified RNAs.

The method can include introducing into the cell one or more DNA endonucleases wherein the one or more DNA endonucleases is a protein or polypeptide. Such proteins or polypeptides may include other elements such as cell penetrating proteins, signals for localization such as nuclear localization signals, stabilization domains, additional conjugates and/or cleavage sites or signals.

The method can further include introducing into the cell one or more guide ribonucleic acids (gRNAs). The one or more gRNAs can be single-molecule guide RNA (sgRNAs). The one or more gRNAs or one or more sgRNAs can be one or more modified gRNAs, one or more modified sgRNAs, or combinations thereof. The one or more DNA endonucleases can be pre-complexed with one or more gRNAs, one or more sgRNAs, or combinations thereof.

The method can further include introducing into the cell a polynucleotide donor template having at least a portion of the wild-type WAS gene. DNA sequences that encode wild-type regulatory elements of the WAS gene, and/or cDNA. The reference sequences of WAS gene, e.g, a genomic sequence containing the WAS locus and WAS mRNA sequence can be obtained via NCBI database using the ID No. NG_007877.1 and NM_000377.2, respectively. The sequence of WAS gene or any regulatory and neighboring sequences can be obtained from such resources and used to generate a suitable donor template. The at least a portion of the wild-type WAS gene or cDNA can be any of the exons or introns as defined herein. Such portions may include more than one intron or exon as well as sequence regions bridging exons and introns, e.g., intron:exon junctions, intronic regions, fragments or combinations thereof, or the entire WAS gene or cDNA. In some embodiments, such portions can also include upstream and/or downstream sequences of the wild-type WAS gene. Therefore, in some embodiments a polynucleotide donor template can have a WAS gene or cDNA and a sequence neighboring (or surrounding) the endogenous WAS gene. In some embodiments, a polynucleotide donor template can have a WAS gene or cDNA and an upstream sequence of the endogenous WAS gene (e.g, at least or about 500 bp upstream sequence including the proximal promoter of the endogenous WAS gene). In some embodiments, a polynucleotide donor template can have a WAS gene or cDNA and a downstream sequence the endogenous WAS gene. The donor template can be either a single or double stranded polynucleotide. The donor template can have homologous arms to the pq34.11 region.

The method can further have introducing into the cell one guide ribonucleic acid (gRNA) and a polynucleotide donor template having at least a portion of the wild-type WAS gene. The method can further have introducing into the cell one guide ribonucleic acid (gRNA) and a polynucleotide donor template having at least a portion of a codon optimized or modified WAS gene. The one or more DNA endonucleases can be one or more Cas9 or Cpf1 endonucleases that effect one single-strand break (SSB) or double-strand break (DSB) at a locus at, within, or near the WAS gene (or codon optimized or modified WAS gene) or other DNA sequences that encode regulatory elements of the WAS gene that facilitates insertion of a new sequence from the polynucleotide donor template into the chromosomal DNA at the locus that results in a permanent insertion or correction of a part of the chromosomal DNA of the WAS gene or other DNA sequences that encode regulatory elements of the WAS gene proximal to the locus. The gRNA can have a spacer sequence that is complementary to a segment of the locus. Proximal can mean nucleotides both upstream and downstream of the locus.

The method can further have introducing into the cell two guide ribonucleic acid (gRNAs) and a polynucleotide donor template having at least a portion of the wild-type WAS gene. The one or more DNA endonucleases can be one or more Cas9 or Cpf1 endonucleases that effect or create at least two (e.g., a pair) single-strand breaks (SSBs) and/or double-strand breaks (DSBs), the first at a 5′ locus and the second at a 3′ locus, at, within, or near the WAS gene or other DNA sequences that encode regulatory elements of the WAS gene that facilitates insertion of a new sequence from the polynucleotide donor template into the chromosomal DNA between the 5′ locus and the 3′ locus that results in a permanent insertion or correction of the chromosomal DNA between the 5′ locus and the 3′ locus at, within, or near the WAS gene or other DNA sequences that encode regulatory elements of the WAS gene. The first guide RNA can have a spacer sequence that is complementary to a segment of the 5′ locus and the second guide RNA can have a spacer sequence that is complementary to a segment of the 3′ locus.

The one or two gRNAs can be one or two single-molecule guide RNA (sgRNAs). The one or two gRNAs or one or two sgRNAs can be one or two modified gRNAs or one or two modified sgRNAs. The one or more DNA endonucleases can be pre-complexed with one or two gRNAs or one or two sgRNAs.

The at least a portion of the wild-type WAS gene or cDNA can be any of the exons or introns as defined herein. Such portions may include more than one intron or exon as well as sequence regions bridging exons and introns, e.g., intron:exon junctions, intronic regions, fragments or combinations thereof, or the entire WAS gene or cDNA.

The donor template can be either a single or double stranded polynucleotide. The donor template can have homologous arms to the chromosomal region encoding the gene target.

The SSB, DSB, 5′ DSB, and/or 3′ DSB can be in any intron, exon, or junction thereof.

The gRNA or sgRNA can be directed to one or more of the following pathological variants, such as any of the single nucleotide polymorphisms associated with WAS gene disclosed herein.

The insertion or correction can be by homology directed repair (HDR).

Cas9 or Cpf1 mRNA, gRNA, and donor template can be formulated into separate lipid nanoparticles or co-formulated into a lipid nanoparticle.

The Cas9 or Cpf1 mRNA can be formulated into a lipid nanoparticle, and the gRNA and donor template can be delivered to the cell by an adeno-associated virus (AAV) vector.

The Cas9 or Cpf1 mRNA can be formulated into a lipid nanoparticle, and the gRNA can be delivered to the cell by electroporation and donor template can be delivered to the cell by an adeno-associated virus (AAV) vector.

The restoration of WAS protein activity can be compared to wild-type or normal WAS protein activity.

Also provided herein is one or more guide ribonucleic acids (gRNAs) for editing a WAS gene in a cell from a patient with Wiskott-Aldrich Syndrome (WAS). The one or more gRNAs and/or sgRNAs can have a spacer sequence selected from the group consisting of any nucleic acid 12-200 nucleotides in length which act to guide the endonuclease to the gene. The one or more gRNAs can be one or more single-molecule guide RNAs (sgRNAs). The one or more gRNAs or one or more sgRNAs can be one or more modified gRNAs or one or more modified sgRNAs.

### BRIEF DESCRIPTION OF THE DRAWINGS

Various aspects of materials and methods disclosed and described in this specification can be better understood by reference to the accompanying figures, in which:

FIG. 1A is a plasmid (CTx-1) having a codon optimized gene for S. pyogenes Cas9 endonuclease. The CTx-1 plasmid also has a gRNA scaffold sequence, which includes a 15-200 bp spacer sequence.

FIG. 1B is a plasmid (CTx-2) having a different codon optimized gene for S. pyogenes Cas9 endonuclease. The CTx-2 plasmid also has a gRNA scaffold sequence, which includes a 15-200 bp spacer sequence.

FIG. 1C is a plasmid (CTx-3) having yet another different codon optimized gene for S. pyogenes Cas9 endonuclease. The CTx-3 plasmid also has a gRNA scaffold sequence, which includes a 15-200 bp spacer sequence.

FIG. 2A is a depiction of the type II CRISPR/Cas system.

FIG. 2B is another depiction of the type II CRISPR/Cas system.

FIG. 3 is a viral vector (pAAV_WAS_mCherry-HA) having WAS cDNA for insertion and mCherry marker gene.

FIG. 4 is a viral vector (pAAV_MND_WAS_mCherry AAV HA) having WAS cDNA for insertion and mCherry marker gene.

### DETAILED DESCRIPTION

### I. Introduction

### Genome Editing

Genome editing generally refers to the process of modifying the nucleotide sequence of a genome, preferably in a precise or pre-determined manner. Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and NHEJ, as recently reviewed in Cox et al., Nature Medicine 21(2), 121-31 (2015). These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature 518, 174-76 (2015); Kent et al., Nature Structural and Molecular Biology, Adv. Online doi:10.1038/nsmb.2961(2015); Mateos-Gomez et al., Nature 518, 254-57 (2015); Ceccaldi et al., Nature 528, 258-62 (2015). In some instances, it may be possible to predict likely repair outcomes based on analysis of potential microhomologies at the site of the DNA break.

Each of these genome editing mechanisms can be used to create desired genomic alterations. A step in the genome editing process can be to create one or two DNA breaks, the latter as double-strand breaks or as two single-stranded breaks, in the target locus as near the site of intended mutation. This can be achieved via the use of site-directed polypeptides, as described and illustrated herein.

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair or non-homologous end joining or alternative non-homologous end joining (A-NHEJ) or microhomology-mediated end joining). NHEJ can repair cleaved target nucleic acid without the need for a homologous template. This can sometimes result in small deletions or insertions (InDels) in the target nucleic acid at the site of cleavage, and can lead to disruption or alteration of gene expression. HDR can occur when a homologous repair template, or donor, is available. The homologous donor template can have sequences that can be homologous to sequences flanking the target nucleic acid cleavage site. The sister chromatid can be used by the cell as the repair template. However, for the purposes of genome editing, the repair template can be supplied as an exogenous nucleic acid, such as a plasmid, duplex oligonucleotide, single-strand oligonucleotide, double-stranded oligonucleotide, or viral nucleic acid. With exogenous donor templates, an additional nucleic acid sequence (such as a transgene) or modification (such as a single or multiple base change or a deletion) can be introduced between the flanking regions of homology so that the additional or altered nucleic acid sequence also becomes incorporated into the target locus. MMEJ can result in a genetic outcome that is similar to NHEJ in that small deletions and insertions can occur at the cleavage site. MMEJ can make use of homologous sequences of a few base pairs flanking the cleavage site to drive a favored end-joining DNA repair outcome. In some instances, it may be possible to predict likely repair outcomes based on analysis of potential microhomologies in the nuclease target regions.

Thus, in some cases, homologous recombination can be used to insert an exogenous polynucleotide sequence into the target nucleic acid cleavage site. An exogenous polynucleotide sequence is termed a donor polynucleotide (or donor or donor sequence or polynucleotide donor template) herein. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

### CRISPR Endonuclease System

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary structures (e.g., hairpins) and/or have unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

### Type II CRISPR Systems

crRNA biogenesis in a Type II CRISPR system in nature requires a trans-activating CRISPR RNA (tracrRNA). The tracrRNA can be modified by endogenous RNaseIII, and then hybridizes to a crRNA repeat in the pre-crRNA array. Endogenous RNaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA, and the tracrRNA and the crRNA associate with a site-directed polypeptide (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and international patent application publication number WO2013/176772 provides numerous examples and applications of the CRISPR/Cas endonuclease system for site-specific gene editing.

### Type V CRISPR Systems

Type V CRISPR systems have several important differences from Type II systems. For example, Cpf1 is a single RNA-guided endonuclease that, in contrast to Type II systems, lacks tracrRNA. In fact, Cpf1-associated CRISPR arrays can be processed into mature crRNAs without the requirement of an additional trans-activating tracrRNA. The Type V CRISPR array can be processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems can start with 20-24 nucleotides of spacer sequence followed by about 22 nucleotides of direct repeat. Also. Cpf1 can utilize a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

### Cas Genes/Polypeptides and Protospacer Adjacent Motifs

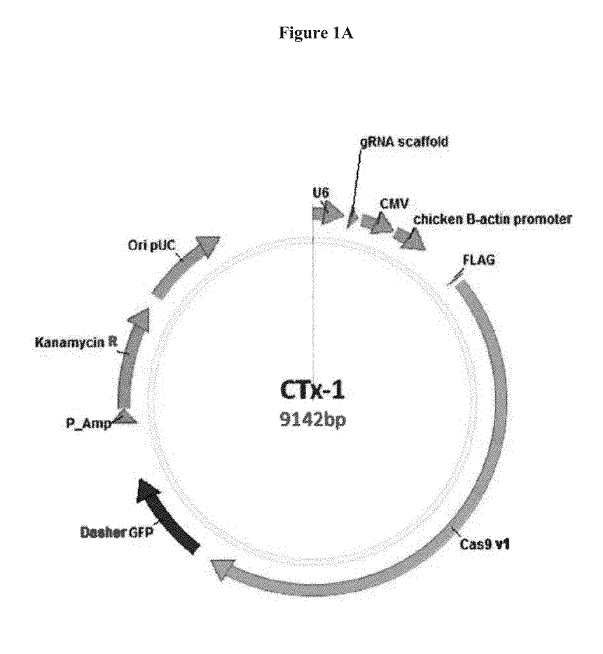

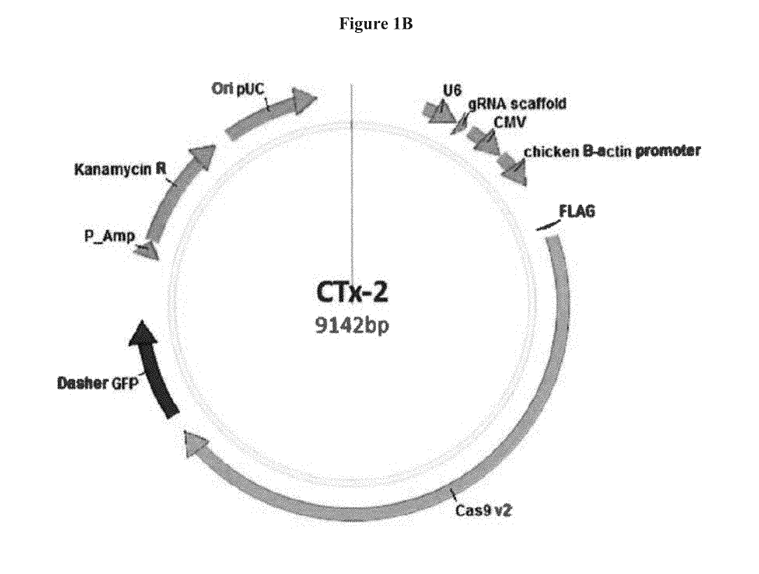

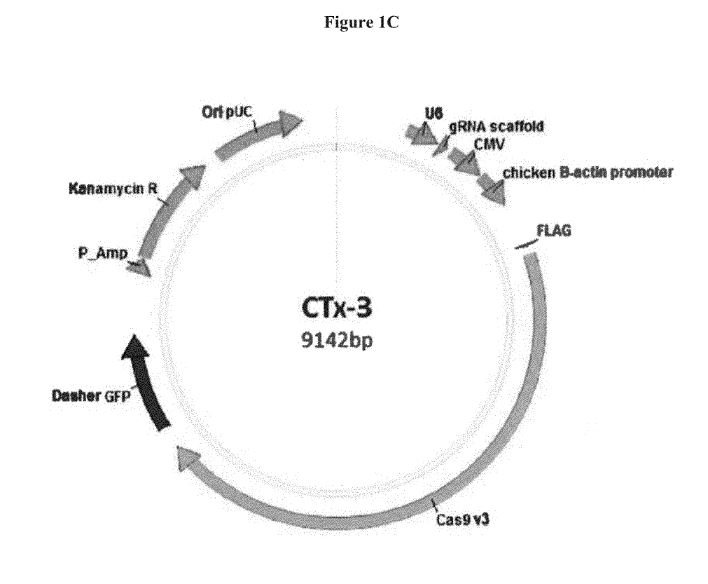

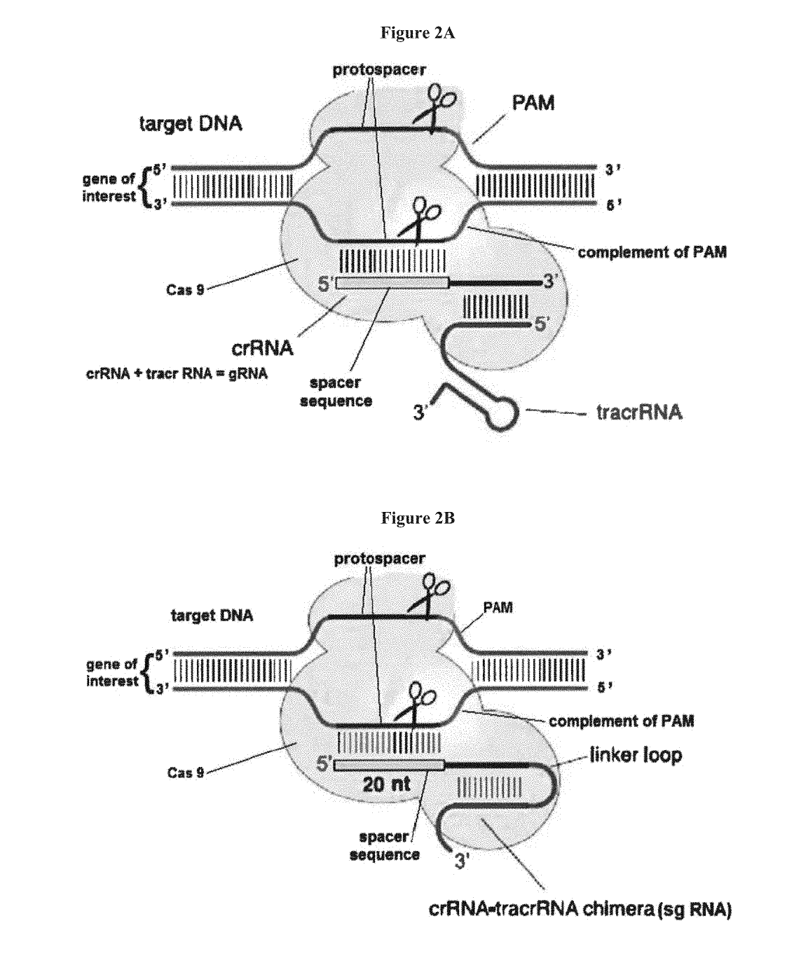

Exemplary CRISPR/Cas polypeptides include the Cas9 polypeptides in FIG. 1 of Fonfara et al., Nucleic Acids Research. 42: 2577-2590 (2014). The CRISPR/Cas gene naming system has undergone extensive rewriting since the Cas genes were discovered. FIG. 5 of Fonfara, supra, provides PAM sequences for the Cas9 polypeptides from various species.

For monogenic disorders with recessive inheritance, it is likely that correcting one of the mutant alleles per cell will be sufficient for correction. The correction of one allele can coincide with one copy that remains with the original mutation, or a copy that was cleaved and repaired by non-homologous end joining (NHEJ) and therefore was not properly corrected. Bi-allelic correction can also occur. Various editing strategies that can be employed for specific mutations are discussed below.

Correction of one or possibly both of the mutant alleles provides an important improvement over existing therapies, such as introduction of WAS gene expression cassettes through lentivirus delivery and integration. Gene editing to correct the mutation has the advantage of restoration of correct expression levels and temporal control. Sequencing the patient's Wiskott-Aldrich syndrome gene alleles allows for design of the gene editing strategy to best correct the identified mutation(s).

For example, the mutation can be corrected by the insertions or deletions that arise due to the imprecise NHEJ repair pathway. If the patient's WAS gene has an inserted or deleted base, a targeted cleavage can result in a NHEJ-mediated insertion or deletion that restores the frame. Missense mutations can also be corrected through NHEJ-mediated correction using one or more guide RNA. The ability or likelihood of the cut(s) to correct the mutation can be designed or evaluated based on the local sequence and micro-homologies. NHEJ can also be used to delete segments of the gene, either directly or by altering splice donor or acceptor sites through cleavage by one gRNA targeting several locations, or several gRNAs. This may be useful if an amino acid, domain or exon contains the mutations and can be removed or inverted, or if the deletion otherwise restored function to the protein. Pairs of guide strands have been used for deletions and corrections of inversions.

Alternatively, the donor for correction by homology directed repair (HDR) contains the corrected sequence with small or large flanking homology arms to allow for annealing. HDR is essentially an error-free mechanism that uses a supplied homologous DNA sequence as a template during DSB repair. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

In addition to correcting mutations by NHEJ or HDR, a range of other options are possible. If there are small or large deletions or multiple mutations, a cDNA can be knocked in that contains the exons affected. A full length cDNA can be knocked into any “safe harbor”—i.e., non-deleterious insertion point that is not the WAS gene itself—with or without suitable regulatory sequences. If this construct is knocked-in near the WAS gene regulatory elements, it should have physiological control, similar to the normal gene. Two or more (e.g., a pair) nucleases can be used to delete mutated gene regions, though a donor would usually have to be provided to restore function. In this case two gRNA and one donor sequence would be supplied.

### II. Compositions and Methods

Provided herein are cellular, ex vivo and in vivo methods for using genome engineering tools to create permanent changes to the genome by: 1) correcting, by insertions or deletions that arise due to the imprecise NHEJ pathway, one or more mutations at, within, or near the WAS gene, 2) correcting, by HDR, one or more mutations at, within, or near the WAS gene, or 3) knocking-in WAS gene cDNA or a minigene (which may have one or more exons or introns or natural or synthetic introns) into the gene locus or at a heterologous location in the genome (such as a safe harbor locus, such as, e.g., targeting an AAVS1 (PPP1R12C), an ALB gene, an Angpt13 gene, an ApoC3 gene, an ASGR2 gene, a CCR5 gene, a FIX (F9) gene, a G6PC gene, a Gys2 gene, an HGD gene, a Lp(a) gene, a Pcsk9 gene, a Serpinal gene, a TF gene, and a TTR gene). Assessment of efficiency of HDR mediated knock-in of cDNA into the first exon can utilize cDNA knock-in into “safe harbor” sites such as: single-stranded or double-stranded DNA having homologous arms to one of the following regions, for example: ApoC3 (chr11:116829908-116833071), Angpt13 (chr1:62,597,487-62,606,305), Serpinal (chr14:94376747-94390692), Lp(a) (chr6:160531483-160664259), Pcsk9 (chr1:55,039,475-55,064,852), FIX (chrX: 139,530,736-139,563,458), ALB (chr4:73,404,254-73,421,411), TTR (chr18:31,591,766-31,599,023), TF (chr3:133,661,997-133,779,005), G6PC (chr17:42,900,796-42,914,432), Gys2 (chr12:21,536,188-21,604.857), AAVS1(PPP1R12C) (chr19:55,090,912-55,117,599), HGD (chr3:120,628,167-120,682,570), CCR5 (chr3:46,370,854-46,376,206), ASGR2 (chr17:7,101,322-7,114,310). Such methods use endonucleases, such as CRISPR-associated (Cas9, Cpf1 and the like) nucleases, to permanently insert, edit or correct one or more mutations at, within, or near the genomic locus of the WAS gene or other DNA sequences that encode regulatory elements of the WAS gene. In this way, examples set forth in the present disclosure can help to restore the reading frame or the wild-type sequence of, or otherwise correct, the gene with a single treatment (rather than deliver potential therapies for the lifetime of the patient).

Non-limiting examples of Cas9 orthologs from other bacterial strains including but not limited to, Cas proteins identified in Acaryochloris marina MBIC 11017; Acetohalobium arabaticum DSM 5501; Acidithiobacillus caldus; Acidithiobacillus ferrooxidans ATCC 23270; Alicyclobacillus acidocaldarius LAA; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS 10; Burkholderiales bacterium 1_1_47; Caldicelulosiruptor becscii DSM 6725; Candidatus Desulforudis audaxviator MP104C; Caldicellulosiruptor hydrothermalis_108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcistis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscllatoria sp. PCC 6506; Pelotomaculum_thermopropionicum_SI; Petrotoga mobilis SJ95; Polaromonas naphihalenivorans CJ2; Polaromonas sp. JS666; Pseudoalteromonas haloplanktis TAC 125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococus sp. PCC 7335; and Thermosipho africanus TCF52B (Chylinski et al., RNA Biol., 2013; 10(5): 726-737, the contents of which are incorporated herein by reference in their entirety).

In addition to Cas9 orthologs, other Cas9 variants such as fusion proteins of inactive dCas9 and effector domains with different functions may be served as a platform for genetic modulation. Any of the foregoing enzymes may be useful in the present disclosure.

Further examples of endonucleases which may be utilized in embodiments of the present disclosures are given in Table 1 and 2. These proteins may be modified before use or may be encoded in a nucleic acid sequence such as a DNA, RNA or mRNA or within a vector construct such as the plasmids or AAV vectors taught herein. Further, they may be codon optimized.

Table 1 is a non-exhaustive listing of endonucleases and protospacer adjacent motifs (PAMs). In Table 1, VP64 is an activator, m4 is a mutant endonuclease sequence, NLS is a nuclear localization signal on the C terminus (e.g., SV40 NLS). The identification number from Uniprot and the European Nucleotide Archive (ENA) databases are also provided for some endonucleases.

TABLE 1
Endonuclease orthologs and Protospacer adjacent motifs (PAMs)

| | | Protein or | | |
|---|---|---|---|---|
| | | DNA SEQ | | |
| Species | Other Name | ID NO | Source | PAM |
| Streptococcus pyogenes | SpCas9; | 1 | Uniprot ID: Q99ZW2 | NGG |
| | SpyCas9 | | | |
| Streptococcus pyogenes | SP-cas; | 2 | Esvelt; Nature Methods, vol 10, | NGG |
| | SpCas9; | | No 11, November 2013; ENA | |
| | SpyCas9 | | ID: AAK33936 | |
| Streptococcus pyogenes | SP-casm4 | 3 | Esvelt; Nature Methods, vol 10, | NGG |
| | | | No 11, November 2013 | |
| Streptococcus pyogenes | cas9-SP- | 4 | Esvelt; Nature Methods, vol 10, | NGG |
| | NLS | | No 11, November 2013 | |
| Streptococcus pyogenes | cas9- | 5 | Esvelt; Nature Methods, vol 10, | NGG |
| | SP3xNLS | | No 11, November 2013 | |
| Streptococcus pyogenes | cas9- | 6 | Esvelt; Nature Methods, vol 10, | NGG |
| | SPm4VP64 | | No 11, November 2013 | |
| Streptococcus pyogenes | cas9- | 7 | Esvelt; Nature Methods, vol 10, | NGG |
| | SPm4VP64 | | No 11, November 2013 | |
| | N | | | |
| Streptococcus | St-Cas9 | 8 | UniProt ID: G3ECR1 | NNAGAAW |
| thermophiles | | | | |
| Streptococcus | St-Cas9 | 9 | ENA ID: AEM62887 | NNAGAAW |
| thermophiles | | | | |
| Streptococcus | ST1-cas | 10 | Esvelt; Nature Methods, vol 10, | NNAGAAW |
| thermophiles | | | No 11, November 2013 | |
| Streptococcus | ST-casm4 | 11 | Esvelt; Nature Methods, vol 10, | NNAGAAW |
| thermophiles | | | No 11, November 2013 | |
| Streptococcus | cas9-ST1 | 12 | Esvelt; Nature Methods, vol 10, | NNAGAAW |
| thermophiles | | | No 11, November 2013 | |
| Streptococcus | cas9- | 13 | Esvelt; Nature Methods, vol 10, | NNAGAAW |
| thermophiles | ST13xNLS | | No 11, November 2013 | |
| Streptococcus | cas9- | 14 | Esvelt; Nature Methods, vol 10, | NNAGAAW |
| thermophiles | ST1m4VP64 | | No 11, November 2013 | |
| Streptococcus | cas9- | 15 | Esvelt; Nature Methods, vol 10, | NNAGAAW |
| thermophiles | ST1m4VP64 | | No 11, November 2013 | |
| | N | | | |
| Neisseria meningitidis | NM-cas | 16 | Esvelt; Nature Methods, vol 10, | NNNNGATT |
| | | | No 11, November 2013 | |
| Neisseria meningitidis | NM-casm4 | 17 | Esvelt; Nature Methods, vol 10, | NNNNGATT |
| | | | No 11, November 2013 | |
| Neisseria meningitidis | cas9-NM | 18 | Esvelt; Nature Methods, vol 10, | NNNNGATT |
| | | | No 11, November 2013 | |
| Neisseria meningitidis | cas9- | 19 | Esvelt; Nature Methods, vol 10, | NNNNGATT |
| | NM3xNLS | | No 11, November 2013 | |
| Neisseria meningitidis | cas9- | 20 | Esvelt; Nature Methods, vol 10, | NNNNGATT |
| | NMm4VP64 | | No 11, November 2013 | |
| Neisseria meningitidis | cas9- | 21 | Esvelt; Nature Methods, vol 10, | NNNNGATT |
| | NMm4VP64 | | No 11, November 2013 | |
| | N | | | |
| Treponema denticola | TD-cas | 22 | Esvelt; Nature Methods, vol 10, | NAAAAC |
| | | | No 11, November 2013 | |
| Treponema denticola | TD-casm4 | 23 | Esvelt; Nature Methods, vol 10, | NAAAAC |
| | | | No 11, November 2013 | |
| Streptococcus aureas | SaCas9 | 24 | UniProt ID: J7RUA5 | NNGRRT |
| Streptococcus aureas | SaCas9 | 25 | ENA ID: CCK7413 | NNGRRT |
| Francisella tularensis | cas9 | 26 | Uniprot ID: A0Q5Y3 | NGG |
| Francisella tularensis | cas9 | 27 | ENA ID: ABK89648 | NGG |
| Francisella tularensis | FnCpf1 | 28 | UniProt ID: A0Q7Q2 | TTN or YTN |
| subsp. novicida (strain | | | | |
| U112) | | | | |
| Acidaminococcus sp. | AsCpf1 | 29 | UniProt ID: U2UMQ6 | TTN or YTN |
| (strain BV3L6) | | | | |

Table 2 is a non-exhaustive listing of endonucleases. Provided in Table 2 are the strain, GI number, NCBI Reference number and the sequence identifier for the amino acid sequence.

TABLE 2
Endonuclease orthologs

| Strain | GI No. | NCBI Ref. No. | SEQ ID NO |
|---|---|---|---|
| 72 Bradyrhizobium sp. BTAil | 500990533 | WP_012044026.1 | 30 |
| 79 Candidatus Puniceispirillum marinum IMCC1322 | 502812437 | WP_013047413.1 | 31 |
| Acidaminococcus intestini RyC-MR95 | 496307041 | WP_009016219.1 | 32 |
| Acidaminococcus sp. D21 | 227824983 | ZP_03989815.1 | 33 |
| Acidothermus cellulolyticus 11B | 500040068 | WP_011720786.1 | 34 |
| Acidovorax avenae subsp. avenae ATCC 19860 | 503358116 | WP_013592777.1 | 35 |
| Acidovorax ebreus TPSY | 501844634 | WP_012655176.1 | 36 |
| Actinobacillus minor NM305 | 240949037 | ZP_04753391.1 | 37 |
| Actinobacillus pleuropneumoniae serovar 10 str. | 307256472 | ZP_07538254.1 | 38 |
| D13039 | | | |
| Actinobacillus succinogenes 130Z | 500711346 | WP_011979028.1 | 39 |
| Actinobacillus suis H91-0380 | 504804175 | WP_014991277.1 | 40 |
| Actinomyces coleocanis DSM 15436 | 227494853 | ZP_03925169.1 | 41 |
| Actinomyces georgiae F0490 | 420151340 | ZP_14658459.1 | 42 |
| Actinomyces naeslundii str. Howell 279 | 400293272 | ZP_10795148.1 | 43 |
| Actinomyces sp. ICM47 | 396585058 | ZP_10485490.1 | 44 |
| Actinomyces sp. oral taxon 175 str. F0384 | 343523232 | ZP_08760194.1 | 45 |
| Actinomyces sp. oral taxon 180 str. F0310 | 315605738 | ZP_07880770.1 | 46 |
| Actinomyces sp. oral taxon 181 str. F0379 | 429758968 | ZP_19291474.1 | 47 |
| Actinomyces sp. oral taxon 848 str. F0332 | 269219760 | ZP_06163614.1 | 48 |
| Actinomyces turicensis ACS-279-V-Col4 | 405979650 | ZP_11037993.1 | 49 |
| Akkermansia muciniphila ATCC BAA-835 | 501389468 | WP_012421034.1 | 50 |
| Alcanivorax sp. W11-5 | 407803669 | ZP_11150502.1 | 51 |
| Alicycliphilus denitrificans BC | 319760940 | YP_004124877.1 | 52 |
| Alicycliphilus denitrificans K601 | 503282466 | WP_013517127.1 | 53 |
| Alicyclobacillus hesperidum URH17-3-68 | 403744858 | ZP_10953934.1 | 54 |
| Aminomonas paucivorans DSM 12260 | 312879015 | ZP_07738815.1 | 55 |
| Anaerococcus tetradius ATCC 35098 | 227501312 | ZP_03931361.1 | 56 |
| Anaerophaga sp. HS1 | 371776944 | ZP_09483266.1 | 57 |
| Anaerophaga thermohalophila DSM 12881 | 346224232 | ZP_08845374.1 | 58 |
| Azospirillum sp. B510 | 502738540 | WP_012973524.1 | 59 |
| Bacillus cereus BAG4X12-1 | 423439645 | ZP_17416574.1 | 60 |
| Bacillus cereus BAG4X2-1 | 423445130 | ZP_17422033.1 | 61 |
| Bacillus cereus Rock1-15 | 229113166 | ZP_04242662.1 | 62 |
| Bacillus smithii 7_3_47FAA | 365156657 | ZP_09352959.1 | 63 |
| Bacillus thuringiensis serovar finitimus YBT-020 | 447027827 | WP_001105083.1 | 64 |
| Bacteroides coprophilus DSM 18228 | 224026357 | ZP_03644723.1 | 65 |
| Bacteroides coprosuis DSM 18011 | 333031006 | ZP_08459067.1 | 66 |
| Bacteroides dorei DSM 17855 | 212694363 | ZP_03302491.1 | 67 |
| Bacteroides eggerthii 1_2_48FAA | 317474201 | ZP_07933477.1 | 68 |
| Bacteroides faecis 27-5 | 380696107 | ZP_09860966.1 | 69 |
| Bacteroides fluxus YTT 12057 | 329965125 | ZP_08302094.1 | 70 |
| Bacteroides fragilis 638R | 492255239 | WP_005791619.1 | 71 |
| Bacteroides fragilis NCTC 9343 | 496648031 | WP_009293010.1 | 72 |
| Bacteroides nordii CL02T12C05 | 393788929 | ZP_10377053.1 | 73 |
| Bacteroides sp. 2_1_16 | 265767599 | ZP_06095265.1 | 74 |
| Bacteroides sp. 20_3 | 301311869 | ZP_07217791.1 | 75 |
| Bacteroides sp. 3_1_19 | 298377533 | ZP_06987485.1 | 76 |
| Bacteroides sp. D2 | 383115507 | ZP_09936263.1 | 77 |
| Bacteroides sp. D2 | 383110723 | ZP_09931542.1 | 78 |
| Bacteroides uniformis CL03T00C23 | 423303159 | ZP_17281158.1 | 79 |
| Bacteroides vulgatus CL09T03C04 | 423312075 | ZP_17290012.1 | 80 |
| Bacteroidetes oral taxon 274 str. F0058 | 298373376 | ZP_06983365.1 | 81 |
| Barnesiella intestinihominis YIT 11860 | 404487228 | ZP_11022414.1 | 82 |
| Belliella baltica DSM 15883 | 504586551 | WP_014773653.1 | 83 |
| Bergeyella zoohelcum ATCC 43767 | 423317190 | ZP_17295095.1 | 84 |
| Bergeyella zoohelcum CCUG 30536 | 406673990 | ZP_11081206.1 | 85 |
| Bifidobacterium bifidum S17 | 503128334 | WP_013362995.1 | 86 |
| Bifidobacterium dentium Bdl | 502666262 | WP_012902199.1 | 87 |
| Bifidobacterium longum DJO10A | 501448754 | WP_012472203.1 | 88 |
| Bifidobacterium longum subsp. longum 2-2B | 419852381 | ZP_14375259.1 | 89 |
| Bifidobacterium longum subsp. longum KACC 91563 | 494117278 | WP_007057059.1 | 90 |
| Bifidobacterium sp. 12_1_47BFAA | 317482066 | ZP_07941090.1 | 91 |
| Brevibacillus laterosporus GI-9 | 421874297 | ZP_16305903.1 | 92 |
| Burkholderiales bacterium 1_1_47 | 303257695 | ZP_07343707.1 | 93 |
| Caenispirillum salinarum AK4 | 427429481 | ZP_18919511.1 | 94 |
| Campylobacter coli 1098 | 419564797 | ZP_14102166.1 | 95 |
| Campylobacter coli 111-3 | 419536531 | ZP_14076011.1 | 96 |
| Campylobacter coli 132-6 | 419572019 | ZP_14108954.1 | 97 |
| Campylobacter coli 151-9 | 419603415 | ZP_14137966.1 | 98 |
| Campylobacter coli 1909 | 419576091 | ZP_14112759.1 | 99 |
| Campylobacter coli 1957 | 419581876 | ZP_14118158.1 | 100 |
| Campylobacter coli 2692 | 419553162 | ZP_14091426.1 | 101 |
| Campylobacter coli 59-2 | 419578074 | ZP_14114609.1 | 102 |
| Campylobacter coli 67-8 | 419587721 | ZP_14123627.1 | 103 |
| Campylobacter coli 80352 | 419558307 | ZP_14096178.1 | 104 |
| Campylobacter coli 80352 | 419559505 | ZP_14097234.1 | 105 |
| Campylobacter jejuni subsp. doylei 269.97 | 500764549 | WP_011990840.1 | 106 |
| Campylobacter jejuni subsp. jejuni 110-21 | 419676124 | ZP_14205367.1 | 107 |
| Campylobacter jejuni subsp. jejuni 129-258 | 419619138 | ZP_14152637.1 | 108 |
| Campylobacter jejuni subsp. jejuni 1336 | 283956897 | ZP_06374370.1 | 109 |
| Campylobacter jejuni subsp. jejuni 140-16 | 419681578 | ZP_14210407.1 | 110 |
| Campylobacter jejuni subsp. jejuni 1577 | 419685099 | ZP_14213672.1 | 111 |
| Campylobacter jejuni subsp. jejuni 1854 | 419689467 | ZP_14217734.1 | 112 |
| Campylobacter jejuni subsp. jejuni 1997-10 | 419666522 | ZP_14196520.1 | 113 |
| Campylobacter jejuni subsp. jejuni 2008-1025 | 419650041 | ZP_14181271.1 | 114 |
| Campylobacter jejuni subsp. jejuni 2008-872 | 419654778 | ZP_14185684.1 | 115 |
| Campylobacter jejuni subsp. jejuni 2008-979 | 419660762 | ZP_14191198.1 | 116 |
| Campylobacter jejuni subsp. jejuni 2008-988 | 419656328 | ZP_14187138.1 | 117 |
| Campylobacter jejuni subsp. jejuni 2008-988 | 419655317 | ZP_14186170.1 | 118 |
| Campylobacter jejuni subsp. jejuni 260.94 | 86152042 | ZP_01070255.1 | 119 |
| Campylobacter jejuni subsp. jejuni 414 | 283953849 | ZP_06371379.1 | 120 |
| Campylobacter jejuni subsp. jejuni 51037 | 419674189 | ZP_14203604.1 | 121 |
| Campylobacter jejuni subsp. jejuni 51494 | 419619463 | ZP_14152929.1 | 122 |
| Campylobacter jejuni subsp. jejuni 53161 | 419647275 | ZP_14178699.1 | 123 |
| Campylobacter jejuni subsp. jejuni 60004 | 419629136 | ZP_14161873.1 | 124 |
| Campylobacter jejuni subsp. jejuni 81116 | 500850126 | WP_012006786.1 | 125 |
| Campylobacter jejuni subsp. jejuni 84-25 | 88596565 | ZP_01099802.1 | 126 |
| Campylobacter jejuni subsp. jejuni 87459 | 419680124 | ZP_14209037.1 | 127 |
| Campylobacter jejuni subsp. jejuni ATCC 33560 | 419643715 | ZP_14175404.1 | 128 |
| Campylobacter jejuni subsp. jejuni CF93-6 | 86149266 | ZP_01067497.1 | 129 |
| Campylobacter jejuni subsp. jejuni CG8486 | 148925683 | ZP_01809371.1 | 130 |
| Campylobacter jejuni subsp. jejuni HB93-13 | 86152450 | ZP_01070655.1 | 131 |
| Campylobacter jejuni subsp. jejuni LMG 23210 | 419696801 | ZP_14224621.1 | 132 |
| Campylobacter jejuni subsp. jejuni LMG 23211 | 419697443 | ZP_14225176.1 | 133 |
| Campylobacter jejuni subsp. jejuni LMG 23263 | 419628620 | ZP_14161448.1 | 134 |
| Campylobacter jejuni subsp. jejuni LMG 23264 | 419632476 | ZP_14165011.1 | 135 |
| Campylobacter jejuni subsp. jejuni LMG 23269 | 419634246 | ZP_14166646.1 | 136 |
| Campylobacter jejuni subsp. jejuni LMG 23357 | 419641132 | ZP_14173041.1 | 137 |
| Campylobacter jejuni subsp. jejuni NCTC 11168 | 218563121 | YP_002344900.1 | 138 |
| Campylobacter jejuni subsp. jejuni NW | 424845990 | ZP_18270589.1 | 139 |
| Campylobacter jejuni subsp. jejuni PT14 | 504829395 | WP_015016497.1 | 140 |
| Campylobacter lari | 345468028 | BAK69486.1 | 141 |
| Capnocytophaga canimorsus Cc5 | 503763492 | WP_013997568.1 | 142 |
| Capnocytophaga gingivalis ATCC 33624 | 228473057 | ZP_04057814.1 | 143 |
| Capnocytophaga ochracea DSM 7271 | 506262077 | WP_015781852.1 | 144 |
| Capnocytophaga sp. CM59 | 402830627 | ZP_10879324.1 | 145 |
| Capnocytophaga sp. oral taxon 324 str. F0483 | 429756885 | ZP_19289459.1 | 146 |
| Capnocytophaga sp. oral taxon 326 str. F0382 | 429752492 | ZP_19285347.1 | 147 |
| Capnocytophaga sp. oral taxon 329 str. F0087 | 332882466 | ZP_08450086.1 | 148 |
| Capnocytophaga sp. oral taxon 335 str. F0486 | 420149252 | ZP_14656431.1 | 149 |
| Capnocytophaga sp. oral taxon 380 str. F0488 | 429748017 | ZP_19281243.1 | 150 |
| Capnocytophaga sp. oral taxon 412 str. F0487 | 393778597 | ZP_10366863.1 | 151 |
| Capnocytophaga sputigena Capno | 213962376 | ZP_03390639.1 | 152 |
| Catellicoccus marimammalium M35/04/3 | 424780480 | ZP_18207353.1 | 153 |
| Catenibacterium mitsuokai DSM 15897 | 224543312 | ZP_03683851.1 | 154 |
| Chryseobacterium sp. CF314 | 399023756 | ZP_10725810.1 | 155 |
| Clostridium cellulolyticum H10 | 506406750 | WP_015926469.1 | 156 |
| Clostridium perfringens C str. JGS1495 | 169343975 | ZP_02864966.1 | 157 |
| Clostridium perfringens D str. JGS1721 | 182624245 | ZP_02952031.1 | 158 |
| Clostridium spiroforme DSM 1552 | 169349750 | ZP_02866688.1 | 159 |
| Coprococcus catus GD/7 | 291520705 | CBK78998.1 | 160 |
| Coriobacterium glomerans PW2 | 503474914 | WP_013709575.1 | 161 |
| Corynebacterium accolens ATCC 49725 | 227502575 | ZP_03932624.1 | 162 |
| Corynebacterium accolens ATCC 49726 | 306835141 | ZP_07468181.1 | 163 |
| Corynebacterium diphtheriae 241 | 504067105 | WP_014301099.1 | 164 |
| Corynebacterium diphtheriae 31A | 504082104 | WP_014316098.1 | 165 |
| Corynebacterium diphtheriae BH8 | 504083379 | WP_014317373.1 | 166 |
| Corynebacterium diphtheriae bv. intermedius str. | 419861895 | ZP_14384519.1 | 167 |
| NCTC 5011 | | | |
| Corynebacterium diphtheriae C7 (beta) | 504084437 | WP_014318431.1 | 168 |
| Corynebacterium diphtheriae HC02 | 504085624 | WP_014319618.1 | 169 |
| Corynebacterium diphtheriae NCTC 13129 | 499236428 | WP_010933968.1 | 170 |
| Corynebacterium diphtheriae VA01 | 504077139 | WP_014311133.1 | 171 |
| Corynebacterium matruchotii ATCC 14266 | 305681510 | ZP_07404317.1 | 172 |
| Corynebacterium matruchotii ATCC 33806 | 225021644 | ZP_03710836.1 | 173 |
| Dinoroseobacter shibae DFL 12 | 501128074 | WP_012177079.1 | 174 |
| Dolosigranulum pigrum ATCC 51524 | 375088882 | ZP_09735219.1 | 175 |
| Dorea longicatena DSM 13814 | 153855454 | ZP_01996585.1 | 176 |
| Eggerthella sp. YY7918 | 503746753 | WP_013980829.1 | 177 |
| Elusimicrobium minutum Pei191 | 501382854 | WP_012414420.1 | 178 |
| Enterococcus faecalis ATCC 29200 | 229548613 | ZP_04437338.1 | 179 |
| Enterococcus faecalis ATCC 4200 | 256617555 | ZP_05474401.1 | 180 |
| Enterococcus faecalis D6 | 257086028 | ZP_05580389.1 | 181 |
| Enterococcus faecalis E1Sol | 257080914 | ZP_05575275.1 | 182 |
| Enterococcus faecalis Fly1 | 257084992 | ZP_05579353.1 | 183 |
| Enterococcus faecalis OG1RF | 384512368 | WP_002413717.1 | 184 |
| Enterococcus faecalis R508 | 424761124 | ZP_18188706.1 | 185 |
| Enterococcus faecalis T11 | 257419486 | ZP_05596480.1 | 186 |
| Enterococcus faecalis TX0012 | 315149830 | EFT93846.1 | 187 |
| Enterococcus faecalis TX0012 | 422729710 | ZP_16786108.1 | 188 |
| Enterococcus faecalis TX0470 | 312900261 | ZP_07759573.1 | 189 |
| Enterococcus faecalis TX1342 | 422701955 | ZP_16759795.1 | 190 |
| Enterococcus faecalis TX4244 | 422695652 | ZP_16753631.1 | 191 |
| Enterococcus faecium 1,141,733 | 257888853 | ZP_05668506.1 | 192 |
| Enterococcus faecium 1,231,408 | 257893735 | ZP_05673388.1 | 193 |
| Enterococcus faecium E1133 | 430847551 | ZP_19465387.1 | 194 |
| Enterococcus faecium E3083 | 431757680 | ZP_19546309.1 | 195 |
| Enterococcus faecium PC4.1 | 293379700 | ZP_06625836.1 | 196 |
| Enterococcus faecium TX1330 | 227550972 | ZP_03981021.1 | 197 |
| Enterococcus faecium TX1337RF | 424765774 | ZP_18193145.1 | 198 |
| Enterococcus hirae ATCC 9790 | 392988474 | WP_010737004.1 | 199 |
| Enterococcus italicus DSM 15952 | 315641599 | ZP_07896667.1 | 200 |
| Eubacterium dolichum DSM 3991 | 160915782 | ZP_02077990.1 | 201 |
| Eubacterium rectale ATCC 33656 | 502252724 | WP_012742555.1 | 202 |
| Eubacterium sp. AS15 | 402309258 | ZP_10828253.1 | 203 |
| Eubacterium ventriosum ATCC 27560 | 154482474 | ZP_02024922.1 | 204 |
| Eubacterium yurii subsp. margaretiae ATCC 43715 | 306821691 | ZP_07455288.1 | 205 |
| Facklamia hominis CCUG 36813 | 406671118 | ZP_11078357.1 | 206 |
| Fibrobacter succinogenes subsp. succinogenes S85 | 502574305 | WP_012819984.1 | 207 |
| Filifactor alocis ATCC 35896 | 504028100 | WP_014262094.1 | 208 |
| Finegoldia magna ACS-171-V-Col3 | 302380288 | ZP_07268759.1 | 209 |
| Finegoldia magna ATCC 29328 | 501247123 | WP_012290141.1 | 210 |
| Finegoldia magna SY403409CC001050417 | 417926052 | ZP_12569464.1 | 211 |
| Flavobacteriaceae bacterium S85 | 372210605 | ZP_09498407.1 | 212 |
| Flavobacterium branchiophilum FL-15 | 503850157 | WP_014084151.1 | 213 |
| Flavobacterium columnare ATCC 49512 | 503931814 | WP_014165808.1 | 214 |
| Flavobacterium columnare ATCC 49512 | 503930464 | WP_014164458.1 | 215 |
| Flavobacterium psychrophilum JIP02/86 | 150025575 | YP_001296401.1 | 216 |
| Fluviicola taffensis DSM 16823 | 503453227 | WP_013687888.1 | 217 |
| Francisella cf. novicida 3523 | 504361318 | WP_014548420.1 | 218 |
| Francisella cf. novicida Fx1 | 504362543 | WP_014549645.1 | 219 |
| Francisella novicida FTG | 208779141 | ZP_03246487.1 | 220 |
| Francisella novicida GA99-3548 | 254374175 | ZP_04989657.1 | 221 |
| Francisella novicida U112 | 489129153 | WP_003038941.1 | 222 |
| Francisella tularensis subsp. novicida GA99-3549 | 254372717 | ZP_04988206.1 | 223 |
| Fructobacillus fructosusKCTC 3544 | 339625081 | ZP_08660870.1 | 224 |
| Fusobacterium nucleatum subsp. vincentii ATCC | 34762592 | ZP_00143587.1 | 225 |
| 49256 | | | |
| Fusobacterium sp. 1_1_41FAA | 294782278 | ZP_06747604.1 | 226 |
| Fusobacterium sp. 3_1_27 | 294785695 | ZP_06750983.1 | 227 |
| Fusobacterium sp. 3_1_36A2 | 256845019 | ZP_05550477.1 | 228 |
| Galbibacter sp. ck-I2-15 | 408370397 | ZP_11168174.1 | 229 |
| gamma proteobacterium HdN1 | 503028472 | WP_013263448.1 | 230 |
| gamma proteobacterium HTCC5015 | 254447899 | ZP_05061364.1 | 231 |
| Gardnerella vaginalis 1500E | 415717744 | ZP_11466979.1 | 232 |
| Gardnerella vaginalis 284V | 415703177 | ZP_11459085.1 | 233 |
| Gardnerella vaginalis 5-1 | 298252606 | ZP_06976400.1 | 234 |
| Gemella haemolysans ATCC 10379 | 241889924 | ZP_04777222.1 | 235 |
| Gemella morbillorum M424 | 317495358 | ZP_07953728.1 | 236 |
| Gluconacetobacter diazotrophicus PA1 5 | 209542524 | WP_012553074.1 | 237 |
| Gluconacetobacter diazotrophicus PA1 5 | 501182811 | WP_012225906.1 | 238 |
| Gordonibacter pamelaeae 7-10-1-b | 295106015 | CBL03558.1 | 239 |
| Haemophilus parainfluenzae ATCC 33392 | 325578067 | ZP_08148261.1 | 240 |
| Haemophilus parainfluenzae CCUG 13788 | 359298684 | ZP_09184523.1 | 241 |
| Haemophilus parainfluenzae T3T1 | 503831578 | WP_014065572.1 | 242 |
| Haemophilus sputorum HK 2154 | 402304649 | ZP_10823715.1 | 243 |
| Helcococcus kunzii ATCC 51366 | 375092427 | ZP_09738707.1 | 244 |
| Helicobacter canadensis MIT 98-5491 | 253828136 | ZP_04871021.1 | 245 |
| Helicobacter cinaedi ATCC BAA-847 | 396079277 | BAM32653.1 | 246 |
| Helicobacter cinaedi CCUG 18818 | 313144862 | ZP_07807055.1 | 247 |
| Helicobacter cinaedi PAGU611 | 504479905 | WP_014667007.1 | 248 |
| Helicobacter mustelae 12198 | 502787413 | WP_013022389.1 | 249 |
| Ignavibacterium album JCM 16511 | 504374771 | WP_014561873.1 | 250 |
| Ilyobacter polytropus DSM 2926 | 503154365 | WP_013389026.1 | 251 |
| Indibacter alkaliphilus LW1 | 404451234 | ZP_11016204.1 | 252 |
| Joostella marina DSM 19592 | 386818981 | ZP_10106197.1 | 253 |
| Kingella kingae PYKK081 | 381401699 | ZP_09926592.1 | 254 |
| Kordia algicida OT-1 | 163754820 | ZP_02161941.1 | 255 |
| Lactobacillus animalis KCTC 3501 | 335357451 | ZP_08549321.1 | 256 |
| Lactobacillus brevis subsp. gravesensis ATCC 27305 | 227509761 | ZP_03939810.1 | 257 |
| Lactobacillus buchneri CD034 | 504753359 | WP_014940461.1 | 258 |
| Lactobacillus buchneri NRRL B-30929 | 503493991 | WP_013728652.1 | 259 |
| Lactobacillus casei BL23 | 191639137 | YP_001988303.1 | 260 |
| Lactobacillus casei Lc-10 | 418010298 | ZP_12650076.1 | 261 |
| Lactobacillus casei M36 | 417996992 | ZP_12637259.1 | 262 |
| Lactobacillus casei str. Zhang | 501468426 | WP_012491871.1 | 263 |
| Lactobacillus casei T71499 | 417999832 | ZP_12640037.1 | 264 |
| Lactobacillus casei UCD174 | 418002962 | ZP_12643066.1 | 265 |
| Lactobacillus casei W56 | 504765121 | WP_014952223.1 | 266 |
| Lactobacillus coryniformis subsp. coryniformis | 333394446 | ZP_08476265.1 | 267 |
| KCTC 3167 | | | |
| Lactobacillus coryniformis subsp. torquens KCTC | 336393381 | ZP_08574780.1 | 268 |
| 3535 | | | |
| Lactobacillus curvatus CRL 705 | 354808135 | ZP_09041575.1 | 269 |
| Lactobacillus farciminis KCTC 3681 | 336394701 | ZP_08576100.1 | 270 |
| Lactobacillus farciminis KCTC 3681 | 336394882 | ZP_08576281.1 | 271 |
| Lactobacillus fermentum 28-3-CHN | 260662220 | ZP_05863116.1 | 272 |
| Lactobacillus fermentum ATCC 14931 | 227514633 | ZP_03944682.1 | 273 |
| Lactobacillus florum 2F | 408790128 | ZP_11201760.1 | 274 |
| Lactobacillus gasseri JV-V03 | 300361537 | ZP_07057714.1 | 275 |
| Lactobacillus hominis CRBIP 24.179 | 395244248 | ZP_10421219.1 | 276 |
| Lactobacillus iners LactinV 11V1-d | 309803917 | ZP_07698001.1 | 277 |
| Lactobacillus jensenii 269-3 | 238854567 | ZP_04644902.1 | 278 |
| Lactobacillus jensenii 27-2-CHN | 256852176 | ZP_05557562.1 | 279 |
| Lactobacillus johnsonii DPC 6026 | 504380459 | WP_014567561.1 | 280 |
| Lactobacillus mucosae LM1 | 377831443 | ZP_09814419.1 | 281 |
| Lactobacillus paracasei subsp. paracasei 8700:2 | 239630053 | ZP_04673084.1 | 282 |
| Lactobacillus pentosus IG1 | 339637353 | CCC16263.1 | 283 |
| Lactobacillus pentosus KCA1 | 392947436 | ZP_10313071.1 | 284 |
| Lactobacillus pentosus MP-10 | 334881121 | CCB81940.1 | 285 |
| Lactobacillus plantarum ZJ316 | 505192536 | WP_015379638.1 | 286 |
| Lactobacillus rhamnosus GG | 504382875 | WP_014569977.1 | 287 |
| Lactobacillus rhamnosus HN001 | 199597394 | ZP_03210824.1 | 288 |
| Lactobacillus rhamnosus R0011 | 418072660 | ZP_12709930.1 | 289 |
| Lactobacillus ruminis ATCC 25644 | 323340068 | ZP_08080334.1 | 290 |
| Lactobacillus salivarius SMXD51 | 418960525 | ZP_13512412.1 | 291 |
| Lactobacillus sanfranciscensis TMW 1.1304 | 503848134 | WP_014082128.1 | 292 |
| Lactobacillus sp. 66c | 408410332 | ZP_11181557.1 | 293 |
| Lactobacillus versmoldensis KCTC 3814 | 365906066 | ZP_09443825.1 | 294 |
| Legionella pneumophila 130b | 307608922 | CBW98322.1 | 295 |
| Legionella pneumophila str. Paris | 499526152 | WP_011212792.1 | 296 |
| Leuconostoc gelidum KCTC 3527 | 333398273 | ZP_08480086.1 | 297 |
| Listeria innocua ATCC 33091 | 423101383 | ZP_17089087.1 | 298 |
| Listeria innocua Clip11262 | 16801805 | WP_010991369.1 | 299 |
| Listeria monocytogenes 10403S | 386044902 | WP_014601172.1 | 300 |
| Listeria monocytogenes FSL JI-194 | 254825045 | ZP_05230046.1 | 301 |
| Listeria monocytogenes FSL J1-208 | 422810631 | ZP_16859042.1 | 302 |
| Listeria monocytogenes FSL N3-165 | 254829042 | ZP_05233729.1 | 303 |
| Listeria monocytogenes FSL R2-503 | 254854201 | ZP_05243549.1 | 304 |
| Listeria monocytogenes str. 1/2a F6854 | 47097148 | ZP_00234715.1 | 305 |
| Listeriaceae bacterium TTU M1-001 | 381184145 | ZP_09892805.1 | 306 |
| Marinilabilia sp. AK2 | 410030899 | ZP_11280729.1 | 307 |
| Megasphaera sp. UPII 135-E | 342218215 | ZP_08710837.1 | 308 |
| Methylocystis sp. ATCC 49242 | 323139312 | ZP_08074365.1 | 309 |
| Methylosinus trichosporium OB3b | 296446027 | ZP_06887976.1 | 310 |
| Mobiluncus curtisii subsp. holmesii ATCC 35242 | 315656340 | ZP_07909231.1 | 311 |
| Mobiluncus mulieris 28-1 | 269977848 | ZP_06184804.1 | 312 |
| Mobiluncus mulieris FB024-16 | 307700167 | ZP_07637211.1 | 313 |
| Mucilaginibacter paludis DSM 18603 | 373954054 | ZP_09614014.1 | 314 |
| Mycoplasma canis PG 14 | 384393286 | EIE39736.1 | 315 |
| Mycoplasma canis PG 14 | 419703974 | ZP_14231525.1 | 316 |
| Mycoplasma canis UF31 | 384937953 | ZP_10029646.1 | 317 |
| Mycoplasma canis UF33 | 419704625 | ZP_14232170.1 | 318 |
| Mycoplasma canis UFG1 | 419705269 | ZP_14232808.1 | 319 |
| Mycoplasma canis UFG4 | 419705920 | ZP_14233452.1 | 320 |
| Mycoplasma cynos C142 | 505100601 | WP_015287703.1 | 321 |
| Mycoplasma gallisepticum NC95_13295-2-2P | 504699247 | WP_014886349.1 | 322 |
| Mycoplasma gallisepticum NY01_2001.047-5-1P | 504699352 | WP_014886454.1 | 323 |
| Mycoplasma gallisepticum str. F | 504387687 | WP_014574789.1 | 324 |
| Mycoplasma gallisepticum str. F | 284931710 | ADC31648.1 | 325 |
| Mycoplasma gallisepticum str. R(low) | 499426471 | WP_011113935.1 | 326 |
| Mycoplasma mobile 163K | 499583770 | WP_011264553.1 | 327 |
| Mycoplasma ovipneumoniae SC01 | 363542550 | ZP_09312133.1 | 328 |
| Mycoplasma synoviae 53 | 499602984 | WP_011283718.1 | 329 |
| Mycoplasma synoviae 53 | 144575181 | AAZ43989.2 | 330 |
| Myroides injenensis M09-0166 | 399927444 | ZP_10784802.1 | 331 |
| Myroides odoratus DSM 2801 | 374597806 | ZP_09670808.1 | 332 |
| Neisseria bacilliformis ATCC BAA-1200 | 329117879 | ZP_08246593.1 | 333 |
| Neisseria cinerea ATCC 14685 | 261378287 | ZP_05982860.1 | 334 |
| Neisseria flavescens SK114 | 241759613 | ZP_04757714.1 | 335 |
| Neisseria lactamica 020-06 | 503214802 | WP_013449463.1 | 336 |
| Neisseria meningitidis 053442 | 501178069 | WP_012221298.1 | 337 |
| Neisseria meningitidis 2007056 | 433531983 | ZP_20488550.1 | 338 |
| Neisseria meningitidis 63049 | 433514137 | ZP_20470921.1 | 339 |
| Neisseria meningitidis 8013 | 504387108 | WP_014574210.1 | 340 |
| Neisseria meningitidis 92045 | 421559784 | ZP_16005652.1 | 341 |
| Neisseria meningitidis 93003 | 421538794 | ZP_15984966.1 | 342 |
| Neisseria meningitidis 93004 | 421541126 | ZP_15987256.1 | 343 |
| Neisseria meningitidis 96023 | 433518260 | ZP_20475000.1 | 344 |
| Neisseria meningitidis 98008 | 421555531 | ZP_16001461.1 | 345 |
| Neisseria meningitidis alpha14 | 254804356 | WP_015815286.1 | 346 |
| Neisseria meningitidis alpha275 | 254672046 | CBA04630.1 | 347 |
| Neisseria meningitidis ATCC 13091 | 304388355 | ZP_07370468.1 | 348 |
| Neisseria meningitidis N1568 | 416164244 | ZP_11607176.1 | 349 |
| Neisseria meningitidis NM140 | 421545139 | ZP_15991204.1 | 350 |
| Neisseria meningitidis NM220 | 418291220 | ZP_12903258.1 | 351 |
| Neisseria meningitidis NM233 | 418288950 | ZP_12901357.1 | 352 |
| Neisseria meningitidis WUE 2594 | 488175202 | WP_002246410.1 | 353 |
| Neisseria meningitidis Z2491 | 488163954 | WP_002235162.1 | 354 |
| Neisseria sp. oral taxon 14 str. F0314 | 298369677 | ZP_06980994.1 | 355 |
| Neisseria wadsworthii 9715 | 350570326 | ZP_08938692.1 | 356 |
| Niabella soli DSM 19437 | 374372722 | ZP_09630384.1 | 357 |
| Nitratifractor salsuginis DSM 16511 | 503319748 | WP_013554409.1 | 358 |
| Nitrobacter hamburgensis X14 | 499824283 | WP_011505017.1 | 359 |
| Nitrosomonas sp. AL212 | 503414024 | WP_013648685.1 | 360 |
| Odoribacter laneus YIT 12061 | 374384763 | ZP_09642280.1 | 361 |
| Oenococcus kitaharae DSM 17330 | 372325145 | ZP_09519734.1 | 362 |
| Oenococcus kitaharae DSM 17330 | 366983953 | EHN59352.1 | 363 |
| Olsenella uli DSM 7084 | 503017123 | WP_013252099.1 | 364 |
| Ornithobacterium rhinotracheale DSM 15997 | 504604717 | WP_014791819.1 | 365 |
| Parabacteroides johnsonii DSM 18315 | 218258638 | ZP_03474966.1 | 366 |
| Parabacteroides sp. D13 | 256840409 | ZP_05545917.1 | 367 |
| Parasutterella excrementihominis YIT 11859 | 331001027 | ZP_08324662.1 | 368 |
| Parvibaculum lavamentivorans DS-1 | 500777975 | WP_011995013.1 | 369 |
| Pasteurella multocida subsp. gallicida X73 | 425063822 | ZP_18466947.1 | 370 |
| Pasteurella multocida subsp. multocida str. P52VAC | 421263876 | ZP_15714893.1 | 371 |
| Pasteurella multocida subsp. multocida str. Pm70 | 499209493 | WP_010907033.1 | 372 |
| Pediococcus acidilactici DSM 20284 | 304386254 | ZP_07368587.1 | 373 |
| Pediococcus acidilactici MA18/5M | 418068659 | ZP_12705941.1 | 374 |
| Peptoniphilus duerdenii ATCC BAA-1640 | 304438954 | ZP_07398877.1 | 375 |
| Phascolarctobacterium succinatutens YIT 12067 | 323142435 | ZP_08077256.1 | 376 |
| Planococcus antarcticus DSM 14505 | 389815359 | ZP_10206685.1 | 377 |
| Porphyromonas sp. oral taxon 279 str. F0450 | 402847315 | ZP_10895610.1 | 378 |
| Prevotella bivia JCVIHMP010 | 282858617 | ZP_06267779.1 | 379 |
| Prevotella buccae ATCC 33574 | 315607525 | ZP_07882520.1 | 380 |
| Prevotella buccalis ATCC 35310 | 282878504 | ZP_06287286.1 | 381 |
| Prevotella denticola CRIS 18C-A | 325859619 | ZP_08172752.1 | 382 |
| Prevotella histicola F0411 | 357042839 | ZP_09104541.1 | 383 |
| Prevotella intermedia 17 | 504521832 | WP_014708934.1 | 384 |
| Prevotella micans F0438 | 373501184 | ZP_09591549.1 | 385 |
| Prevotella nigrescens ATCC 33563 | 340351024 | ZP_08673992.1 | 386 |
| Prevotella nigrescens F0103 | 445119230 | ZP_21379155.1 | 387 |
| Prevotella oralis ATCC 33269 | 323344874 | ZP_08085098.1 | 388 |
| Prevotella ruminicola 23 | 502828855 | WP_013063831.1 | 389 |
| Prevotella sp. C561 | 345885718 | ZP_08837074.1 | 390 |
| Prevotella sp. MSX73 | 402307189 | ZP_10826216.1 | 391 |
| Prevotella sp. oral taxon 306 str. F0472 | 383811446 | ZP_09966911.1 | 392 |
| Prevotella stercorea DSM 18206 | 359406728 | ZP_09199391.1 | 393 |
| Prevotella tannerae ATCC 51259 | 258648111 | ZP_05735580.1 | 394 |
| Prevotella timonensis CRIS 5C-B1 | 282881485 | ZP_06290156.1 | 395 |
| Prevotella timonensis CRIS 5C-B1 | 282880052 | ZP_06288774.1 | 396 |
| Prevotella veroralis F0319 | 260592128 | ZP_05857586.1 | 397 |
| Ralstonia syzygii R24 | 344171927 | CCA84553.1 | 398 |
| Rhodopseudomonas palustris BisB18 | 499794158 | WP_011474892.1 | 399 |
| Rhodopseudomonas palustris BisB5 | 499820718 | WP_011501452.1 | 400 |
| Rhodospirillum rubrum ATCC 11170 | 83591793 | YP_425545.1 | 401 |
| Rhodovulum sp. PH10 | 402849997 | ZP_10898214.1 | 402 |
| Riemerella anatipestifer RA-CH-1 | 504750935 | WP_014938037.1 | 403 |
| Riemerella anatipestifer RA-GD | 491056565 | WP_004918207.1 | 404 |
| Roseburia intestinalis L1-82 | 257413184 | ZP_04742247.2 | 405 |
| Roseburia intestinalis M50/1 | 291537230 | CBL10342.1 | 406 |
| Roseburia inulinivorans DSM 16841 | 225377804 | ZP_03755025.1 | 407 |
| Ruminococcus albus 8 | 325677756 | ZP_08157403.1 | 408 |
| Ruminococcus lactaris ATCC 29176 | 197301447 | ZP_03166527.1 | 409 |
| Scardovia wiggsiae F0424 | 423349694 | ZP_17327350.1 | 410 |
| Scardovia inopinata F0304 | 294790575 | ZP_06755733.1 | 411 |
| Simonsiella muelleri ATCC 29453 | 404379108 | ZP_10984177.1 | 412 |
| Solobacterium moorei F0204 | 320528778 | ZP_08029929.1 | 413 |
| Sphaerochaeta globus str. Buddy | 503373188 | WP_013607849.1 | 414 |
| Sphingobacterium spiritivorum ATCC 33861 | 300771242 | ZP_07081118.1 | 415 |
| Sphingobium sp. AP49 | 398385143 | ZP_10543169.1 | 416 |
| Sphingomonas sp. S17 | 332188827 | ZP_08390536.1 | 417 |
| Sporolactobacillus vineae DSM 21990 = SL153 | 404330915 | ZP_10971363.1 | 418 |
| Staphylococcus aureus subsp. aureus | 403411236 | CCK74173.1 | 419 |
| Staphylococcus lugdunensis M23590 | 315659848 | ZP_07912707.1 | 420 |
| Staphylococcus pseudintermedius ED99 | 504426157 | WP_014613259.1 | 421 |
| Staphylococcus pseudintermedius ED99 | 323463801 | ADX75954.1 | 422 |
| Staphylococcus simulans ACS-120-V-Sch1 | 414160476 | ZP_11416743.1 | 423 |
| Streptococcus agalactiae 2603V/R | 22537057 | NP_687908.1 | 424 |
| Streptococcus agalactiae 515 | 77413160 | ZP_00789359.1 | 425 |
| Streptococcus agalactiae A909 | 446962831 | WP_001040087.1 | 426 |
| Streptococcus agalactiae ATCC 13813 | 339301617 | ZP_08650712.1 | 427 |
| Streptococcus agalactiae CJB111 | 77411010 | ZP_00787365.1 | 428 |
| Streptococcus agalactiae COH1 | 77407964 | ZP_00784714.1 | 429 |
| Streptococcus agalactiae FSL S3-026 | 417005168 | ZP_11943761.1 | 430 |
| Streptococcus agalactiae GB00112 | 421147428 | ZP_15607118.1 | 431 |
| Streptococcus agalactiae H36B | 77405721 | ZP_00782807.1 | 432 |
| Streptococcus agalactiae NEM316 | 446962847 | WP_001040103.1 | 433 |
| Streptococcus agalactiae SA20-06 | 504871421 | WP_015058523.1 | 434 |
| Streptococcus agalactiae STIR-CD-17 | 421532069 | ZP_15978441.1 | 435 |
| Streptococcus anginosus 1_2_62CV | 319939170 | ZP_08013534.1 | 436 |
| Streptococcus anginosus F0211 | 315223162 | ZP_07865023.1 | 437 |
| Streptococcus anginosus SK1138 | 421490579 | ZP_15937951.1 | 438 |
| Streptococcus anginosus SK52 = DSM 20563 | 335031483 | ZP_08524916.1 | 439 |
| Streptococcus bovis ATCC 700338 | 306833855 | ZP_07466980.1 | 440 |
| Streptococcus canis FSL Z3-227 | 392329410 | ZP_10274026.1 | 441 |
| Streptococcus constellatus subsp. constellatus SK53 | 418965022 | ZP_13516809.1 | 442 |
| Streptococcus dysgalactiae subsp. equisimilis AC- | 410494913 | WP_015057649.1 | 443 |
| 2713 | | | |
| Streptococcus dysgalactiae subsp. equisimilis ATCC | 504425231 | WP_014612333.1 | 444 |
| 12394 | | | |
| Streptococcus dysgalactiae subsp. equisimilis | 502337426 | WP_012767106.1 | 445 |
| GGS_124 | | | |
| Streptococcus dysgalactiae subsp. equisimilis RE378 | 408401787 | WP_015017095.1 | 446 |
| Streptococcus equi subsp. zooepidemicus | 195978435 | WP_012515931.1 | 447 |
| MGCS10565 | | | |
| Streptococcus equinus ATCC 9812 | 320547102 | ZP_08041398.1 | 448 |
| Streptococcus gallolyticus subsp. gallolyticus ATCC | 497540342 | WP_009854540.1 | 449 |
| BAA-2069 | | | |
| Streptococcus gallolyticus subsp. gallolyticus | 306831733 | ZP_07464890.1 | 450 |
| TX20005 | | | |
| Streptococcus gallolyticus UCN34 | 502727190 | WP_012962174.1 | 451 |
| Streptococcus gallolyticus UCN34 | 502727185 | WP_012962169.1 | 452 |
| Streptococcus gordonii str. Challis substr. CH1 | 157150687 | WP_012130469.1 | 453 |
| Streptococcus infantarius ATCC BAA-102 | 171779984 | ZP_02920888.1 | 454 |
| Streptococcus infantarius subsp. infantarius CJ18 | 504100992 | WP_014334983.1 | 455 |
| Streptococcus iniae 9117 | 406658208 | ZP_11066348.1 | 456 |
| Streptococcus macacae NCTC 11558 | 357636406 | ZP_09134281.1 | 457 |
| Streptococcus macedonicus ACA-DC 198 | 504060913 | WP_014294907.1 | 458 |
| Streptococcus mitis ATCC 6249 | 306829274 | ZP_07462464.1 | 459 |
| Streptococcus mitis SK321 | 307710946 | ZP_07647371.1 | 460 |
| Streptococcus mutans 11SSST2 | 449165720 | EMB68700.1 | 461 |
| Streptococcus mutans 11SSST2 | 449951835 | ZP_21808822.1 | 462 |
| Streptococcus mutans 11VS1 | 449976542 | ZP_21816259.1 | 463 |
| Streptococcus mutans 14D | 450149988 | ZP_21876385.1 | 464 |
| Streptococcus mutans 15VF2 | 449170557 | EMB73257.1 | 465 |
| Streptococcus mutans 15VF2 | 449965974 | ZP_21812137.1 | 466 |
| Streptococcus mutans 1SM1 | 449158457 | EMB61872.1 | 467 |
| Streptococcus mutans 1SM1 | 449920643 | ZP_21798589.1 | 468 |
| Streptococcus mutans 24 | 449247589 | EMC45865.1 | 469 |
| Streptococcus mutans 24 | 450180942 | ZP_21887525.1 | 470 |
| Streptococcus mutans 2VS1 | 449174812 | EMB77280.1 | 471 |
| Streptococcus mutans 2VS1 | 449968746 | ZP_21812810.1 | 472 |
| Streptococcus mutans 3SN1 | 449162653 | EMB65780.1 | 473 |
| Streptococcus mutans 3SN1 | 449931425 | ZP_21802366.1 | 474 |
| Streptococcus mutans 4SM1 | 449159838 | EMB63138.1 | 475 |
| Streptococcus mutans 4SM1 | 449927152 | ZP_21801094.1 | 476 |
| Streptococcus mutans 4VF1 | 449167132 | EMB70037.1 | 477 |
| Streptococcus mutans 4VF1 | 449961027 | ZP_21810754.1 | 478 |
| Streptococcus mutans 5SM3 | 449176693 | EMB79025.1 | 479 |
| Streptococcus mutans 5SM3 | 449980571 | ZP_21817280.1 | 480 |
| Streptococcus mutans 66-2A | 449240165 | EMC38854.1 | 481 |
| Streptococcus mutans 66-2A | 450160342 | ZP_21879935.1 | 482 |
| Streptococcus mutans 8ID3 | 449154769 | EMB58325.1 | 483 |
| Streptococcus mutans 8ID3 | 449872064 | ZP_21781351.1 | 484 |
| Streptococcus mutans A19 | 449187668 | EMB89435.1 | 485 |
| Streptococcus mutans A19 | 450013175 | ZP_21829913.1 | 486 |
| Streptococcus mutans B | 450166294 | ZP_21882263.1 | 487 |
| Streptococcus mutans G123 | 450029806 | ZP_21832884.1 | 488 |
| Streptococcus mutans GS-5 | 488208651 | WP_002279859.1 | 489 |
| Streptococcus mutans LJ23 | 504490807 | WP_014677909.1 | 490 |
| Streptococcus mutans M21 | 449194333 | EMB95692.1 | 491 |
| Streptococcus mutans M21 | 450036249 | ZP_21835412.1 | 492 |
| Streptococcus mutans M230 | 449260994 | EMC58483.1 | 493 |
| Streptococcus mutans M230 | 449903532 | ZP_21792176.1 | 494 |
| Streptococcus mutans M2A | 449209586 | EMC10100.1 | 495 |
| Streptococcus mutans M2A | 450074072 | ZP_21849235.1 | 496 |
| Streptococcus mutans N29 | 449182997 | EMB84997.1 | 497 |
| Streptococcus mutans N29 | 450003067 | ZP_21826084.1 | 498 |
| Streptococcus mutans N3209 | 449210660 | EMC11099.1 | 499 |
| Streptococcus mutans N3209 | 450077860 | ZP_21850689.1 | 500 |
| Streptococcus mutans N66 | 449212466 | EMC12833.1 | 501 |
| Streptococcus mutans N66 | 450083993 | ZP_21853156.1 | 502 |
| Streptococcus mutans NFSM1 | 449202104 | EMC03050.1 | 503 |
| Streptococcus mutans NFSM1 | 450051112 | ZP_21840661.1 | 504 |
| Streptococcus mutans NLML1 | 450140393 | ZP_21872901.1 | 505 |
| Streptococcus mutans NLML4 | 449202681 | EMC03581.1 | 506 |
| Streptococcus mutans NLML4 | 450059882 | ZP_21843564.1 | 507 |
| Streptococcus mutans NLML5 | 449203378 | EMC04242.1 | 508 |
| Streptococcus mutans NLML5 | 450064617 | ZP_21845425.1 | 509 |
| Streptococcus mutans NLML8 | 449151037 | EMB54782.1 | 510 |
| Streptococcus mutans NLML8 | 450133520 | ZP_21870663.1 | 511 |
| Streptococcus mutans NLML9 | 449209148 | EMC09685.1 | 512 |
| Streptococcus mutans NLML9 | 450066176 | ZP_21845833.1 | 513 |
| Streptococcus mutans NMT4863 | 449186850 | EMB88660.1 | 514 |
| Streptococcus mutans NMT4863 | 450007078 | ZP_21827582.1 | 515 |
| Streptococcus mutans NN2025 | 502762704 | WP_012997688.1 | 516 |
| Streptococcus mutans NV1996 | 450086338 | ZP_21853591.1 | 517 |
| Streptococcus mutans NVAB | 449181424 | EMB83523.1 | 518 |
| Streptococcus mutans NVAB | 449990810 | ZP_21821726.1 | 519 |
| Streptococcus mutans R221 | 449258042 | EMC55644.1 | 520 |
| Streptococcus mutans R221 | 449899675 | ZP_21791159.1 | 521 |
| Streptococcus mutans S1B | 449251227 | EMC49247.1 | 522 |
| Streptococcus mutans S1B | 449877120 | ZP_21783133.1 | 523 |
| Streptococcus mutans SF1 | 450098705 | ZP_21858128.1 | 524 |
| Streptococcus mutans SF14 | 449221374 | EMC21157.1 | 525 |
| Streptococcus mutans SF14 | 450107816 | ZP_21861188.1 | 526 |
| Streptococcus mutans SM1 | 449245264 | EMC43607.1 | 527 |
| Streptococcus mutans SM1 | 450176410 | ZP_21885778.1 | 528 |
| Streptococcus mutans SM4 | 449246010 | EMC44327.1 | 529 |
| Streptococcus mutans SM4 | 450170248 | ZP_21883419.1 | 530 |
| Streptococcus mutans SM6 | 449223000 | EMC22710.1 | 531 |
| Streptococcus mutans SM6 | 450112022 | ZP_21863007.1 | 532 |
| Streptococcus mutans ST1 | 449228751 | EMC28103.1 | 533 |
| Streptococcus mutans ST1 | 450114718 | ZP_21863466.1 | 534 |
| Streptococcus mutans ST6 | 449227252 | EMC26687.1 | 535 |
| Streptococcus mutans ST6 | 450123011 | ZP_21867014.1 | 536 |
| Streptococcus mutans U2A | 449232458 | EMC31572.1 | 537 |
| Streptococcus mutans U2A | 450125471 | ZP_21867675.1 | 538 |
| Streptococcus mutans UA159 | 24379809 | NP_721764.1 | 539 |
| Streptococcus mutans W6 | 450094364 | ZP_21857006.1 | 540 |
| Streptococcus oralis SK1074 | 418974877 | ZP_13522786.1 | 541 |
| Streptococcus oralis SK304 | 421488030 | ZP_15935426.1 | 542 |
| Streptococcus oralis SK313 | 417940002 | ZP_12583290.1 | 543 |
| Streptococcus oralis SK610 | 419782534 | ZP_14308334.1 | 544 |
| Streptococcus parasanguinis F0449 | 419799964 | ZP_14325278.1 | 545 |
| Streptococcus pasteurianus ATCC 43144 | 503617972 | WP_013852048.1 | 546 |
| Streptococcus pseudoporcinus LQ 940-04 | 416852857 | ZP_11910002.1 | 547 |
| Streptococcus pyogenes MGAS10270 | 94543903 | ABF33951.1 | 548 |
| Streptococcus pyogenes MGAS10750 | 499847849 | WP_011528583.1 | 549 |
| Streptococcus pyogenes MGAS15252 | 504220439 | WP_014407541.1 | 550 |
| Streptococcus pyogenes MGAS2096 | 489080018 | WP_002989955.1 | 551 |
| Streptococcus pyogenes MGAS315 | 499366838 | WP_011054416.1 | 552 |
| Streptococcus pyogenes MGAS5005 | 499604772 | WP_011285506.1 | 553 |
| Streptococcus pyogenes MGAS6180 | 499604011 | WP_011284745.1 | 554 |
| Streptococcus pyogenes MGAS9429 | 499846885 | WP_011527619.1 | 555 |
| Streptococcus pyogenes NZ131 | 501556167 | WP_012560673.1 | 556 |
| Streptococcus pyogenes SF370 (M1 GAS) | 13622193 | AAK33936.1 | 557 |
| Streptococcus pyogenes SSI-1 | 499366838 | WP_011054416.1 | 558 |
| Streptococcus ratti FA-1 = DSM 20564 | 400290495 | ZP_10792522.1 | 559 |
| Streptococcus salivarius JIM8777 | 504447093 | WP_014634195.1 | 560 |
| Streptococcus salivarius K12 | 421452908 | ZP_15902264.1 | 561 |
| Streptococcus salivarius PS4 | 419707401 | ZP_14234885.1 | 562 |
| Streptococcus sanguinis SK115 | 422848603 | ZP_16895279.1 | 563 |
| Streptococcus sanguinis SK330 | 422860049 | ZP_16906693.1 | 564 |
| Streptococcus sanguinis SK353 | 422821159 | ZP_16869352.1 | 565 |
| Streptococcus sanguinis SK49 | 422884106 | ZP_16930555.1 | 566 |
| Streptococcus sp. BS35b | 401684660 | ZP_10816536.1 | 567 |
| Streptococcus sp. C150 | 322372617 | ZP_08047153.1 | 568 |
| Streptococcus sp. C300 | 322375978 | ZP_08050488.1 | 569 |
| Streptococcus sp. F0441 | 414157437 | ZP_11413735.1 | 570 |
| Streptococcus sp. GMD6S | 406576934 | ZP_11052556.1 | 571 |
| Streptococcus sp. M334 | 322378004 | ZP_08052491.1 | 572 |
| Streptococcus sp. oral taxon 56 str. F0418 | 339640839 | ZP_08662283.1 | 573 |
| Streptococcus sp. oral taxon 71 str. 73H25AP | 306826314 | ZP_07459648.1 | 574 |
| Streptococcus suis 89/1591 | 223932525 | ZP_03624526.1 | 575 |
| Streptococcus suis D9 | 504449965 | WP_014637067.1 | 576 |
| Streptococcus suis ST1 | 504548968 | WP_014736070.1 | 577 |
| Streptococcus suis ST3 | 489025190 | WP_002935602.1 | 578 |
| Streptococcus thermophilus | 343794781 | AEM62887.1 | 579 |
| Streptococcus thermophilus CNRZ1066 | 499546245 | WP_011227028.1 | 580 |
| Streptococcus thermophilus JIM 8232 | 504434277 | WP_014621379.1 | 581 |
| Streptococcus thermophilus LMD-9 | 500000752 | WP_011681470.1 | 582 |
| Streptococcus thermophilus LMD-9 | 500000239 | WP_011680957.1 | 583 |
| Streptococcus thermophilus LMG 18311 | 499544942 | WP_011225725.1 | 584 |
| Streptococcus thermophilus MN-ZLW-002 | 504540549 | WP_014727651.1 | 585 |
| Streptococcus thermophilus MN-ZLW-002 | 504540286 | WP_014727388.1 | 586 |
| Streptococcus thermophilus MTCC 5460 | 445374534 | ZP_21426414.1 | 587 |
| Streptococcus thermophilus ND03 | 504421032 | WP_014608134.1 | 588 |
| Streptococcus thermophilus ND03 | 504421493 | WP_014608595.1 | 589 |
| Streptococcus vestibularis ATCC 49124 | 322517104 | ZP_08069989.1 | 590 |
| Subdoligranulum sp. 4_3_54A2FAA | 365132400 | ZP_09342166.1 | 591 |
| Sutterella wadsworthensis 3_1_45B | 319941583 | ZP_08015909.1 | 592 |
| Tistrella mobilis KA081020-065 | 504561015 | WP_014748117.1 | 593 |
| Treponema denticola AL-2 | 449103686 | ZP_21740430.1 | 594 |
| Treponema denticola ASLM | 449106292 | ZP_21742960.1 | 595 |
| Treponema denticola ATCC 35405 | 42525843 | NP_970941.1 | 596 |
| Treponema denticola H1-T | 449118593 | ZP_21754998.1 | 597 |
| Treponema denticola H-22 | 449117322 | ZP_21753764.1 | 598 |
| Treponema denticola OTK | 449125136 | ZP_21761452.1 | 599 |
| Treponema denticola SP37 | 449130155 | ZP_21766379.1 | 600 |
| Treponema sp. JC4 | 384109266 | ZP_10010146.1 | 601 |
| uncultured delta proteobacterium HF0070_07E19 | 297182908 | ADI19058.1 | 602 |
| uncultured Termite group 1 bacterium phylotype Rs- | 189485059 | YP_001956000.1 | 603 |
| D17 | | | |
| Veillonella atypica ACS-134-V-Col7a | 303229466 | ZP_07316256.1 | 604 |
| Veillonella parvula ATCC 17745 | 282849530 | ZP_06258914.1 | 605 |
| Veillonella sp. 6_1_27 | 294792465 | ZP_06757612.1 | 606 |
| Veillonella sp. oral taxon 780 str. F0422 | 342213964 | ZP_08706676.1 | 607 |
| Verminephrobacter eiseniae EF01-2 | 500133006 | WP_011809011.1 | 608 |
| Weeksella virosa DSM 16922 | 503364269 | WP_013598930.1 | 609 |
| Wolinella succinogenes DSM 1740 | 499451967 | WP_011139431.1 | 610 |
| Wolinella succinogenes DSM 1740 | 499451825 | WP_011139289.1 | 611 |
| Zunongwangia profunda SM-A87 | 502838808 | WP_013073784.1 | 612 |

Provided herein are methods for treating a patient with Wiskott-Aldrich Syndrome (WAS). An aspect of such method is an ex vivo cell-based therapy. For example, a patient specific induced pluripotent stem cell (iPSC) can be created. Then, the chromosomal DNA of these iPS cells can be edited using the materials and methods described herein. Next, the genome-edited iPSCs can be differentiated into other cells. Finally, the differentiated cells are implanted into the patient.

Another aspect of such method is an ex vivo cell-based therapy. For example, a biopsy of the patient's liver is performed. Then, a liver specific progenitor cell or primary hepatocyte is isolated from the biopsied material. Next, the chromosomal DNA of these progenitor cells or primary hepatocytes is corrected using the materials and methods described herein. Finally, the progenitor cells or primary hepatocytes are implanted into the patient. Any source or type of cell may be used as the progenitor cell.

Yet another aspect of such method is an ex vivo cell-based therapy. For example, a mesenchymal stem cell can be isolated from the patient, which can be isolated from the patient's bone marrow or peripheral blood. Next, the chromosomal DNA of these mesenchymal stem cells can be edited using the materials and methods described herein. Next, the genome-edited mesenchymal stem cells can be differentiated into any type of cell, e.g., hepatocytes. Finally, the differentiated cells, e.g., hepatocytes are implanted into the patient.

Yet another aspect of such method is an ex vivo cell-based therapy. For example, a biological sample can be obtained from a subject (e.g., patient) having or suspected of having WAS and cells (e.g., CD34⁺ hematopoietic stem cell). Next, the chromosomal DNA of these hematopoietic stem cells can be edited using the materials and methods described herein. Next, the genome-edited hematopoietic stem cells can be differentiated into any type of cell. Finally, the differentiated cells are implanted into the patient. Alternatively, the genome-edited hematopoietic stem cells can be implanted into the patient, without a further differentiation process. In some embodiments, the genome-edited hematopoietic stem cells can be cultured to increase the cell number that is sufficient for the treatment.

One advantage of an ex vivo cell therapy approach is the ability to conduct a comprehensive analysis of the therapeutic prior to administration. Nuclease-based therapeutics can have some level of off-target effects. Performing gene correction ex vivo allows one to characterize the corrected cell population prior to implantation. The present disclosure includes sequencing the entire genome of the corrected cells to ensure that the off-target effects, if any, can be in genomic locations associated with minimal risk to the patient. Furthermore, populations of specific cells, including clonal populations, can be isolated prior to implantation.

Another advantage of ex vivo cell therapy relates to genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability. In contrast, other primary cells, such as hepatocytes, are viable for only a few passages and difficult to clonally expand. Thus, manipulation of iPSCs for the treatment of Wiskott-Aldrich Syndrome (WAS) can be much easier, and can shorten the amount of time needed to make the desired genetic correction.

Methods can also include an in vivo based therapy. Chromosomal DNA of the cells in the patient is edited using the materials and methods described herein.

Although certain cells present an attractive target for ex vivo treatment and therapy, increased efficacy in delivery may permit direct in vivo delivery to such cells. Ideally the targeting and editing would be directed to the relevant cells. Cleavage in other cells can also be prevented by the use of promoters only active in certain cells and or developmental stages. Additional promoters are inducible, and therefore can be temporally controlled if the nuclease is delivered as a plasmid. The amount of time that delivered RNA and protein remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy will have the potential to be used to treat more than one patient, for example a number of patients who share the same or similar genotype or allele. In contrast, ex vivo cell therapy typically requires using a patient's own cells, which are isolated, manipulated and returned to the same patient.

Also provided herein is a cellular method for editing the WAS gene in a cell by genome editing. For example, a cell can be isolated from a patient or animal. Then, the chromosomal DNA of the cell can be edited using the materials and methods described herein.

The methods provided herein, regardless of whether a cellular or ex vivo or in vivo method, can involve one or a combination of the following: 1) correcting, by insertions or deletions that arise due to the imprecise NHEJ pathway, one or more mutations at, within, or near the WAS gene or other DNA sequences that encode regulatory elements of the WAS gene, 2) correcting, by HDR, one or more mutations at, within, or near the WAS gene or other DNA sequences that encode regulatory elements of the WAS gene, or 3) knocking-in WAS gene cDNA or a minigene (which may have one or more exons or introns or natural or synthetic introns) into the gene locus or at a heterologous location in the genome (such as a safe harbor site, such as AAVS1). In some embodiments, use of a safe harbor locus may include targeting an AAVS1 (PPP1R12C), an ALB gene, an Angpt13 gene, an ApoC3 gene, an ASGR2 gene, a CCR5 gene, a FIX (F9) gene, a G6PC gene, a Gys2 gene, an HGD gene, a Lp(a) gene, a Pcsk9 gene, a Serpinal gene, a TF gene, and a TTR gene). Assessment of efficiency of HDR mediated knock-in of cDNA into the first exon can utilize cDNA knock-in into “safe harbor” sites such as: single-stranded or double-stranded DNA having homologous arms to one of the following regions, for example: ApoC3 (chr11:116829908-116833071), Angpt13 (chr1:62,597,487-62,606,305), Serpinal (chr14:94376747-94390692), Lp(a) (chr6:160531483-160664259), Pcsk9 (chr1:55,039,475-55,064,852), FIX (chrX: 139,530,736-139,563,458), ALB (chr4:73,404,254-73,421,411), TTR (chr18:31,591,766-31,599,023), TF (chr3:133,661,997-133,779,005), G6PC (chr17:42,900,796-42,914,432), Gys2 (chr12:21,536,188-21,604,857), AAVS1(PPP1R12C) (chr19:55,090,912-55,117,599), HGD (chr3:120,628,167-120,682,570). CCR5 (chr3:46,370.854-46,376,206), ASGR2 (chr17:7,101,322-7,114,310). Both the HDR and knock-in strategies utilize a donor DNA template in Homology-Directed Repair (HDR). HDR in either strategy may be accomplished by making one or more single-stranded breaks (SSBs) or double-stranded breaks (DSBs) at specific sites in the genome by using one or more endonucleases.

For example, the NHEJ correction strategy can involve restoring the reading frame in the WAS gene by inducing one single stranded break or double stranded break in the gene of interest with one or more CRISPR endonucleases and a gRNA (e.g., crRNA+tracrRNA, or sgRNA), or two or more single stranded breaks or double stranded breaks in the gene of interest with two or more CRISPR endonucleases and two or more sgRNAs. This approach can require development and optimization of sgRNAs for the WAS gene.

For example, the HDR correction strategy can involve restoring the reading frame in the WAS gene by inducing one single stranded break or double stranded break in the gene of interest with one or more CRISPR endonucleases and a gRNA (e.g., crRNA+tracrRNA, or sgRNA), or two or more single stranded breaks or double stranded breaks in the gene of interest with one or more CRISPR endonucleases and two or more gRNAs, in the presence of a donor DNA template introduced exogenously to direct the cellular DSB response to Homology-Directed Repair (the donor DNA template can be a short single stranded oligonucleotide, a short double stranded oligonucleotide, a long single or double stranded DNA molecule). This approach can require development and optimization of gRNAs and donor DNA molecules for the WAS gene.

For example, the knock-in strategy involves knocking-in WAS gene cDNA or a minigene (which may have natural or synthetic enhancer and promoter, one or more exons, and natural or synthetic introns, and natural or synthetic 3′UTR and polyadenylation signal) into the locus of the gene using a gRNA (e.g., crRNA+tracrRNA, or sgRNA) or a pair of gRNAs targeting upstream of or in the first or other exon and/or intron of the WAS gene, or in a safe harbor site (such as AAVS1). The donor DNA can be single or double stranded DNA.

The advantages for the above strategies (correction and knock-in) are similar, including in principle both short and long term beneficial clinical and laboratory effects. The knock-in approach does provide one advantage over the correction approach—the ability to treat all patients versus only a subset of patients.

In addition to the above genome editing strategies, another strategy involves modulating expression, function, or activity of WAS gene by editing in the regulatory sequence.

In addition to the editing options listed above, Cas9 or similar proteins can be used to target effector domains to the same target sites that can be identified for editing, or additional target sites within range of the effector domain. A range of chromatin modifying enzymes, methylases or demethlyases can be used to alter expression of the target gene. One possibility is increasing the expression of the WAS protein if the mutation leads to lower activity. These types of epigenetic regulation have some advantages, particularly as they are limited in possible off-target effects.

A number of types of genomic target sites can be present in addition to mutations in the coding and splicing sequences.

The regulation of transcription and translation implicates a number of different classes of sites that interact with cellular proteins or nucleotides. Often the DNA binding sites of transcription factors or other proteins can be targeted for mutation or deletion to study the role of the site, though they can also be targeted to change gene expression. Sites can be added through non-homologous end joining NHEJ or direct genome editing by homology directed repair (HDR). Increased use of genome sequencing, RNA expression and genome-wide studies of transcription factor binding have increased our ability to identify how the sites lead to developmental or temporal gene regulation. These control systems can be direct or can involve extensive cooperative regulation that can require the integration of activities from multiple enhancers. Transcription factors typically bind 6-12 bp-long degenerate DNA sequences. The low level of specificity provided by individual sites suggests that complex interactions and rules are involved in binding and the functional outcome. Binding sites with less degeneracy can provide simpler means of regulation. Artificial transcription factors can be designed to specify longer sequences that have less similar sequences in the genome and have lower potential for off-target cleavage. Any of these types of binding sites can be mutated, deleted or even created to enable changes in gene regulation or expression (Canver, M. C, et al., Nature (2015)).

Another class of gene regulatory regions having these features is microRNA (miRNA) binding sites. miRNAs are non-coding RNAs that play key roles in post-transcriptional gene regulation. miRNA can regulate the expression of 30% of all mammalian protein-encoding genes. Specific and potent gene silencing by double stranded RNA (RNAi) was discovered, plus additional small noncoding RNA (Canver, M. C, et al., Nature (2015)). The largest class of noncoding RNAs important for gene silencing are miRNAs. In mammals, miRNAs are first transcribed as a long RNA transcript, which can be separate transcriptional units, part of protein introns, or other transcripts. The long transcripts are called primary miRNA (pri-miRNA) that include imperfectly base-paired hairpin structures. These pri-miRNA can be cleaved into one or more shorter precursor miRNAs (pre-miRNAs) by Microprocessor, a protein complex in the nucleus, involving Drosha.

Pre-miRNAs are short stem loops ˜70 nucleotides in length with a 2-nucleotide 3′-overhang that are exported, into the mature 19-25 nucleotide miRNA:miRNA* duplexes. The miRNA strand with lower base pairing stability (the guide strand) can be loaded onto the RNA-induced silencing complex (RISC). The passenger strand (marked with *), can be functional, but is usually degraded. The mature miRNA tethers RISC to partly complementary sequence motifs in target mRNAs predominantly found within the 3′ untranslated regions (UTRs) and induces posttranscriptional gene silencing (Bartel, D. P. Cell 136, 215-233 (2009); Saj, A. & Lai, E. C. Curr Opin Genet Dev 21, 504-510 (2011)).

miRNAs can be important in development, differentiation, cell cycle and growth control, and in virtually all biological pathways in mammals and other multicellular organisms. miRNAs can also be involved in cell cycle control, apoptosis and stem cell differentiation, hematopoiesis, hypoxia, muscle development, neurogenesis, insulin secretion, cholesterol metabolism, aging, viral replication and immune responses.

A single miRNA can target hundreds of different mRNA transcripts, while an individual transcript can be targeted by many different miRNAs. More than 28645 microRNAs have been annotated in the latest release of miRBase (v.21). Some miRNAs can be encoded by multiple loci, some of which can be expressed from tandemly co-transcribed clusters. The features allow for complex regulatory networks with multiple pathways and feedback controls. miRNAs can be integral parts of these feedback and regulatory circuits and can help regulate gene expression by keeping protein production within limits (Herranz, H. & Cohen, S. M. Genes Dev 24, 1339-1344 (2010); Posadas, D. M. & Carthew, R. W. Curr Opin Genet Dev 27, 1-6 (2014)).

miRNA can also be important in a large number of human diseases that are associated with abnormal miRNA expression. This association underscores the importance of the miRNA regulatory pathway. Recent miRNA deletion studies have linked miRNA with regulation of the immune responses (Stem-Ginossar. N, et al., Science 317, 376-381 (2007)).

miRNA also have a strong link to cancer and can play a role in different types of cancer. miRNAs have been found to be downregulated in a number of tumors. miRNA can be important in the regulation of key cancer-related pathways, such as cell cycle control and the DNA damage response, and can therefore be used in diagnosis and can be targeted clinically. MicroRNAs can delicately regulate the balance of angiogenesis, such that experiments depleting all microRNAs suppresses tumor angiogenesis (Chen, S, et al., Genes Dev 28, 1054-1067 (2014)).

As has been shown for protein coding genes, miRNA genes can also be subject to epigenetic changes occurring with cancer. Many miRNA loci can be associated with CpG islands increasing their opportunity for regulation by DNA methylation (Weber, B., Stresemann, C., Brueckner, B. & Lyko, F. Cell Cycle 6, 1001-1005 (2007)). The majority of studies have used treatment with chromatin remodeling drugs to reveal epigenetically silenced miRNAs.

In addition to their role in RNA silencing, miRNA can also activate translation (Posadas, D. M. & Carthew, R. W. Curr Opin Genet Dev 27, 1-6 (2014)). Knocking out these sites may lead to decreased expression of the targeted gene, while introducing these sites may increase expression.

Individual miRNA can be knocked out most effectively by mutating the seed sequence (bases 2-8 of the microRNA), which can be important for binding specificity. Cleavage in this region, followed by mis-repair by NHEJ can effectively abolish miRNA function by blocking binding to target sites. miRNA could also be inhibited by specific targeting of the special loop region adjacent to the palindromic sequence. Catalytically inactive Cas9 can also be used to inhibit shRNA expression (Zhao. Y, et al., Sci Rep 4, 3943 (2014)). In addition to targeting the miRNA, the binding sites can also be targeted and mutated to prevent the silencing by miRNA.

According to the present disclosure, any of the microRNA (miRNA) or their binding sites may be incorporated into the compositions of the disclosure.

The compositions may have a region such as, but not limited to, a region having the sequence of any of the microRNAs listed in Table 3 the reverse complement of the microRNAs listed in Table 3 or the microRNA anti-seed region of any of the microRNAs listed in Table 3.

The compositions of the disclosure may have one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety. As a non-limiting embodiment, known microRNAs, their sequences and their binding site sequences in the human genome are listed below in Table 3.

A microRNA sequence has a “seed” region, i.e., a sequence in the region of positions 2-8 of the mature microRNA, which sequence has perfect Watson-Crick complementarity to the miRNA target sequence. A microRNA seed may have positions 2-8 or 2-7 of the mature microRNA. In some embodiments, a microRNA seed may have 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may have 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K. Johnston W K, Garrett-Engele P. Lim L P, Bartel D P; Mol Cell. 2007 Jul. 6; 27(1):91-105. The bases of the microRNA seed have complete complementarity with the target sequence.

Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al. Cell, 2007 129:1401-1414; Gentner and Naldini. Tissue Antigens. 2012 80:393-403 and all references therein; each of which is herein incorporated by reference in its entirety).

For example, if the composition is not intended to be delivered to the liver but ends up there, then miR-122, a microRNA abundant in liver, can inhibit the expression of the sequence delivered if one or multiple target sites of miR-122 are engineered into the polynucleotide encoding that target sequence. Introduction of one or multiple binding sites for different microRNA can be engineered to further decrease the longevity, stability, and protein translation hence providing an additional layer of tenability.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the compositions of the present disclosure, microRNA binding sites can be engineered out of (i.e. removed from) sequences in which they naturally occur in order to increase protein expression in specific tissues. For example, miR-122 binding sites may be removed to improve protein expression in the liver.

Specifically, microRNAs are known to be differentially expressed in immune cells (also called hematopoietic cells), such as antigen presenting cells (APCs) (e.g. dendritic cells and macrophages), macrophages, monocytes, B lymphocytes, T lymphocytes, granulocytes, natural killer cells, etc. Immune cell specific microRNAs are involved in immunogenicity, autoimmunity, the immune-response to infection, inflammation, as well as unwanted immune response after gene therapy and tissue/organ transplantation. Immune cells specific microRNAs also regulate many aspects of development, proliferation, differentiation and apoptosis of hematopoietic cells (immune cells). For example, miR-142 and miR-146 are exclusively expressed in the immune cells, particularly abundant in myeloid dendritic cells. Introducing the miR-142 binding site into the 3′-UTR of a polypeptide of the present disclosure can selectively suppress the gene expression in the antigen presenting cells through miR-142 mediated mRNA degradation, limiting antigen presentation in professional APCs (e.g. dendritic cells) and thereby preventing antigen-mediated immune response after gene delivery (see, Annoni A et al., blood, 2009, 114, 5152-5161, the content of which is herein incorporated by reference in its entirety.)

In one embodiment, microRNAs binding sites that are known to be expressed in immune cells, in particular, the antigen presenting cells, can be engineered into the polynucleotides to suppress the expression of the polynucleotide in APCs through microRNA mediated RNA degradation, subduing the antigen-mediated immune response, while the expression of the polynucleotide is maintained in non-immune cells where the immune cell specific microRNAs are not expressed.

Many microRNA expression studies have been conducted, and are described in the art, to profile the differential expression of microRNAs in various cancer cells/tissues and other diseases. Some microRNAs are abnormally over-expressed in certain cancer cells and others are under-expressed. For example, microRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563, the content of each of which is incorporated herein by reference in their entirety).

Non-limiting examples of microRNA sequences and the targeted tissues and/or cells are described in Table 3.

TABLE 3
microRNA Sequences

| Types of Tissues and/or Cells | microRNA name | miR SEQ ID | miR BS SEQ ID |
|---|---|---|---|
| Abnormal skin (psoriasis) | hsa-miR-3934-3p | 613 | 614 |
| Abnormal skin (psoriasis) | hsa-miR-3934-5p | 615 | 616 |
| Abnormal skin (psoriasis) | hsa-miR-548ay-3p | 617 | 618 |
| Abnormal skin (psoriasis) | hsa-miR-548ay-5p | 619 | 620 |
| Abnormal skin (psoriasis) | hsa-miR-548az-3p | 621 | 622 |
| Abnormal skin (psoriasis) | hsa-miR-548az-5p | 623 | 624 |
| Abnormal skin (psoriasis) | hsa-miR-6499-3p | 625 | 626 |
| Abnormal skin (psoriasis) | hsa-miR-6499-5p | 627 | 628 |
| Abnormal skin (psoriasis) | hsa-miR-6500-3p | 629 | 630 |
| Abnormal skin (psoriasis) | hsa-miR-6500-5p | 631 | 632 |
| Abnormal skin (psoriasis) | hsa-miR-6501-3p | 633 | 634 |
| Abnormal skin (psoriasis) | hsa-miR-6501-5p | 635 | 636 |
| Abnormal skin (psoriasis) | hsa-miR-6502-3p | 637 | 638 |
| Abnormal skin (psoriasis) | hsa-miR-6502-5p | 639 | 640 |
| Abnormal skin (psoriasis) | hsa-miR-6503-3p | 641 | 642 |
| Abnormal skin (psoriasis) | hsa-miR-6503-5p | 643 | 644 |
| Abnormal skin (psoriasis) | hsa-miR-6504-3p | 645 | 646 |
| Abnormal skin (psoriasis) | hsa-miR-6504-5p | 647 | 648 |
| Abnormal skin (psoriasis) | hsa-miR-6505-3p | 649 | 650 |
| Abnormal skin (psoriasis) | hsa-miR-6505-5p | 651 | 652 |
| Abnormal skin (psoriasis) | hsa-miR-6506-3p | 653 | 654 |
| Abnormal skin (psoriasis) | hsa-miR-6506-5p | 655 | 656 |
| Abnormal skin (psoriasis) | hsa-miR-6507-3p | 657 | 658 |
| Abnormal skin (psoriasis) | hsa-miR-6507-5p | 659 | 660 |
| Abnormal skin (psoriasis) | hsa-miR-6508-3p | 661 | 662 |
| Abnormal skin (psoriasis) | hsa-miR-6508-5p | 663 | 664 |
| Abnormal skin (psoriasis) | hsa-miR-6509-3p | 665 | 666 |
| Abnormal skin (psoriasis) | hsa-miR-6509-5p | 667 | 668 |
| Abnormal skin (psoriasis) | hsa-miR-6510-3p | 669 | 670 |
| Abnormal skin (psoriasis) | hsa-miR-6510-5p | 671 | 672 |
| Abnormal skin (psoriasis) | hsa-miR-6512-3p | 673 | 674 |
| Abnormal skin (psoriasis) | hsa-miR-6512-5p | 675 | 676 |
| Abnormal skin (psoriasis) | hsa-miR-6513-3p | 677 | 678 |
| Abnormal skin (psoriasis) | hsa-miR-6513-5p | 679 | 680 |
| Abnormal skin (psoriasis) | hsa-miR-6514-3p | 681 | 682 |
| Abnormal skin (psoriasis) | hsa-miR-6514-5p | 683 | 684 |
| Abnormal skin (psoriasis) and epididymis | hsa-miR-6511a-3p | 685 | 686 |
| Abnormal skin (psoriasis) and epididymis | hsa-miR-6511a-5p | 687 | 688 |
| Abnormal skin (psoriasis) and epididymis | hsa-miR-6515-3p | 689 | 690 |
| Abnormal skin (psoriasis) and epididymis | hsa-miR-6515-5p | 691 | 692 |
| Acute Myeloid Leukemia | hsa-miR-3972 | 693 | 694 |
| Acute Myeloid Leukemia | hsa-miR-3973 | 695 | 696 |
| Acute Myeloid Leukemia | hsa-miR-3974 | 697 | 698 |
| Acute Myeloid Leukemia | hsa-miR-3975 | 699 | 700 |
| Acute Myeloid Leukemia | hsa-miR-3976 | 701 | 702 |
| Acute Myeloid Leukemia | hsa-miR-3977 | 703 | 704 |
| Acute Myeloid Leukemia | hsa-miR-3978 | 705 | 706 |
| Adipocyte | hsa-miR-642a-3p | 707 | 708 |
| Adipose, other tissues/cells, kidney | hsa-miR-204-5p | 709 | 710 |
| Adipose, other tissues/cells, kidney | hsa-miR-204-3p | 711 | 712 |
| Airway smooth muscle | hsa-miR-140-3p | 713 | 714 |
| B cells | hsa-miR-3135b | 715 | 716 |
| B cells | hsa-miR-3155b | 717 | 718 |
| B cells | hsa-miR-3689c | 719 | 720 |
| B cells | hsa-miR-3689d | 721 | 722 |
| B cells | hsa-miR-3689e | 723 | 724 |
| B cells | hsa-miR-3689f | 725 | 726 |
| B cells | hsa-miR-4417 | 727 | 728 |
| B cells | hsa-miR-4418 | 729 | 730 |
| B cells | hsa-miR-4419a | 731 | 732 |
| B cells | hsa-miR-4419b | 733 | 734 |
| B cells | hsa-miR-4420 | 735 | 736 |
| B cells | hsa-miR-4421 | 737 | 738 |
| B cells | hsa-miR-4424 | 739 | 740 |
| B cells | hsa-miR-4425 | 741 | 742 |
| B cells | hsa-miR-4426 | 743 | 744 |
| B cells | hsa-miR-4427 | 745 | 746 |
| B cells | hsa-miR-4428 | 747 | 748 |
| B cells | hsa-miR-4429 | 749 | 750 |
| B cells | hsa-miR-4430 | 751 | 752 |
| B cells | hsa-miR-4431 | 753 | 754 |
| B cells | hsa-miR-4432 | 755 | 756 |
| B cells | hsa-miR-4433-3p | 757 | 758 |
| B cells | hsa-miR-4433-5p | 759 | 760 |
| B cells | hsa-miR-4434 | 761 | 762 |
| B cells | hsa-miR-4435 | 763 | 764 |
| B cells | hsa-miR-4437 | 765 | 766 |
| B cells | hsa-miR-4438 | 767 | 768 |
| B cells | hsa-miR-4439 | 769 | 770 |
| B cells | hsa-miR-4440 | 771 | 772 |
| B cells | hsa-miR-4441 | 773 | 774 |
| B cells | hsa-miR-4442 | 775 | 776 |
| B cells | hsa-miR-4443 | 777 | 778 |
| B cells | hsa-miR-4444 | 779 | 780 |
| B cells | hsa-miR-4445-3p | 781 | 782 |
| B cells | hsa-miR-4445-5p | 783 | 784 |
| B cells | hsa-miR-4447 | 785 | 786 |
| B cells | hsa-miR-4448 | 787 | 788 |
| B cells | hsa-miR-4449 | 789 | 790 |
| B cells | hsa-miR-4450 | 791 | 792 |
| B cells | hsa-miR-4451 | 793 | 794 |
| B cells | hsa-miR-4452 | 795 | 796 |
| B cells | hsa-miR-4453 | 797 | 798 |
| B cells | hsa-miR-4454 | 799 | 800 |
| B cells | hsa-miR-4455 | 801 | 802 |
| B cells | hsa-miR-4456 | 803 | 804 |
| B cells | hsa-miR-4457 | 805 | 806 |
| B cells | hsa-miR-4458 | 807 | 808 |
| B cells | hsa-miR-4459 | 809 | 810 |
| B cells | hsa-miR-4460 | 811 | 812 |
| B cells | hsa-miR-4461 | 813 | 814 |
| B cells | hsa-miR-4462 | 815 | 816 |
| B cells | hsa-miR-4463 | 817 | 818 |
| B cells | hsa-miR-4464 | 819 | 820 |
| B cells | hsa-miR-4465 | 821 | 822 |
| B cells | hsa-miR-4466 | 823 | 824 |
| B cells | hsa-miR-4468 | 825 | 826 |
| B cells | hsa-miR-4470 | 827 | 828 |
| B cells | hsa-miR-4472 | 829 | 830 |
| B cells | hsa-miR-4473 | 831 | 832 |
| B cells | hsa-miR-4475 | 833 | 834 |
| B cells | hsa-miR-4476 | 835 | 836 |
| B cells | hsa-miR-4477a | 837 | 838 |
| B cells | hsa-miR-4477b | 839 | 840 |
| B cells | hsa-miR-4478 | 841 | 842 |
| B cells | hsa-miR-4479 | 843 | 844 |
| B cells | hsa-miR-4480 | 845 | 846 |
| B cells | hsa-miR-4481 | 847 | 848 |
| B cells | hsa-miR-4482-3p | 849 | 850 |
| B cells | hsa-miR-4482-5p | 851 | 852 |
| B cells | hsa-miR-4483 | 853 | 854 |
| B cells | hsa-miR-4484 | 855 | 856 |
| B cells | hsa-miR-4485 | 857 | 858 |
| B cells | hsa-miR-4486 | 859 | 860 |
| B cells | hsa-miR-4487 | 861 | 862 |
| B cells | hsa-miR-4488 | 863 | 864 |
| B cells | hsa-miR-4490 | 865 | 866 |
| B cells | hsa-miR-4491 | 867 | 868 |
| B cells | hsa-miR-4492 | 869 | 870 |
| B cells | hsa-miR-4493 | 871 | 872 |
| B cells | hsa-miR-4494 | 873 | 874 |
| B cells | hsa-miR-4495 | 875 | 876 |
| B cells | hsa-miR-4496 | 877 | 878 |
| B cells | hsa-miR-4497 | 879 | 880 |
| B cells | hsa-miR-4498 | 881 | 882 |
| B cells | hsa-miR-4499 | 883 | 884 |
| B cells | hsa-miR-4500 | 885 | 886 |
| B cells | hsa-miR-4501 | 887 | 888 |
| B cells | hsa-miR-4502 | 889 | 890 |
| B cells | hsa-miR-4503 | 891 | 892 |
| B cells | hsa-miR-4504 | 893 | 894 |
| B cells | hsa-miR-4505 | 895 | 896 |
| B cells | hsa-miR-4506 | 897 | 898 |
| B cells | hsa-miR-4507 | 899 | 900 |
| B cells | hsa-miR-4508 | 901 | 902 |
| B cells | hsa-miR-4509 | 903 | 904 |
| B cells | hsa-miR-4510 | 905 | 906 |
| B cells | hsa-miR-4511 | 907 | 908 |
| B cells | hsa-miR-4512 | 909 | 910 |
| B cells | hsa-miR-4513 | 911 | 912 |
| B cells | hsa-miR-4514 | 913 | 914 |
| B cells | hsa-miR-4515 | 915 | 916 |
| B cells | hsa-miR-4516 | 917 | 918 |
| B cells | hsa-miR-4517 | 919 | 920 |
| B cells | hsa-miR-4518 | 921 | 922 |
| B cells | hsa-miR-4519 | 923 | 924 |
| B cells | hsa-miR-4521 | 925 | 926 |
| B cells | hsa-miR-4522 | 927 | 928 |
| B cells | hsa-miR-4523 | 929 | 930 |
| B cells | hsa-miR-4525 | 931 | 932 |
| B cells | hsa-miR-4527 | 933 | 934 |
| B cells | hsa-miR-4528 | 935 | 936 |
| B cells | hsa-miR-4530 | 937 | 938 |
| B cells | hsa-miR-4531 | 939 | 940 |
| B cells | hsa-miR-4532 | 941 | 942 |
| B cells | hsa-miR-4533 | 943 | 944 |
| B cells | hsa-miR-4534 | 945 | 946 |
| B cells | hsa-miR-4535 | 947 | 948 |
| B cells | hsa-miR-4536-3p | 949 | 950 |
| B cells | hsa-miR-4536-5p | 951 | 952 |
| B cells | hsa-miR-4537 | 953 | 954 |
| B cells | hsa-miR-4538 | 955 | 956 |
| B cells | hsa-miR-4539 | 957 | 958 |
| B cells | hsa-miR-4540 | 959 | 960 |
| B-cells | hsa-miR-1587 | 961 | 962 |
| B-cells | hsa-miR-2392 | 963 | 964 |
| B-cells | hsa-miR-548ab | 965 | 966 |
| B-cells | hsa-miR-548ac | 967 | 968 |
| B-cells | hsa-miR-548ad | 969 | 970 |
| B-cells | hsa-miR-548ae | 971 | 972 |
| B-cells | hsa-miR-548ag | 973 | 974 |
| B-cells | hsa-miR-548ah-3p | 975 | 976 |
| B-cells | hsa-miR-548ah-5p | 977 | 978 |
| B-cells | hsa-miR-548ai | 979 | 980 |
| B-cells | hsa-miR-548aj-3p | 981 | 982 |
| B-cells | hsa-miR-548aj-5p | 983 | 984 |
| B-cells | hsa-miR-548ak | 985 | 986 |
| B-cells | hsa-miR-548al | 987 | 988 |
| B-cells | hsa-miR-548am-3p | 989 | 990 |
| B-cells | hsa-miR-548am-5p | 991 | 992 |
| B-cells | hsa-miR-548an | 993 | 994 |
| Blood | hsa-miR-1538 | 995 | 996 |
| Blood | hsa-miR-202-3p | 997 | 998 |
| Blood | hsa-miR-202-5p | 999 | 1000 |
| Blood | hsa-miR-376b-3p | 1001 | 1002 |
| Blood | hsa-miR-376b-5p | 1003 | 1004 |
| Blood | hsa-miR-496 | 1005 | 1006 |
| Blood | hsa-miR-718 | 1007 | 1008 |
| Blood (myeloid cells), liver, endothelial | hsa-miR-21-5p | 1009 | 1010 |
| cells | | | |
| Blood (immune cells) | hsa-miR-28-3p | 1011 | 1012 |
| Blood (immune cells) | hsa-miR-28-5p | 1013 | 1014 |
| Blood (lymphocytes) | hsa-miR-598 | 1015 | 1016 |
| Blood (myeloid cells) | hsa-miR-574-3p | 1017 | 1018 |
| Blood (myeloid cells), glioblast, liver, | hsa-miR-21-3p | 1019 | 1020 |
| vascular endothelial cells | | | |
| Blood (myeloid cells), other tissues/cells | hsa-miR-197-3p | 1021 | 1022 |
| Blood (myeloid cells), other tissues/cells | hsa-miR-197-5p | 1023 | 1024 |
| Blood (plasma) | hsa-miR-500b | 1025 | 1026 |
| Blood and glia | hsa-miR-32-3p | 1027 | 1028 |
| Blood and glia | hsa-miR-32-5p | 1029 | 1030 |
| Blood and other tissues | hsa-miR-26a-2-3p | 1031 | 1032 |
| Blood and other tissues | hsa-miR-26a-5p | 1033 | 1034 |
| Blood mononuclear cells | hsa-miR-935 | 1035 | 1036 |
| Blood (plasma) | hsa-miR-205-3p | 1037 | 1038 |
| Blood (plasma) | hsa-miR-205-5p | 1039 | 1040 |
| Blood (plasma), ovary | hsa-miR-224-3p | 1041 | 1042 |
| Blood (plasma), ovary | hsa-miR-224-5p | 1043 | 1044 |
| Blood, embryonic stem cells, | hsa-miR-16-1-3p | 1045 | 1046 |
| hematopoietic tissues (spleen) | | | |
| Blood, endothelial cells | hsa-miR-361-3p | 1047 | 1048 |
| Blood, heart (myocardial) | hsa-miR-320a | 1049 | 1050 |
| Blood, tongue, pancreas (islet) | hsa-miR-184 | 1051 | 1052 |
| Bone marrow | hsa-miR-654-5p | 1053 | 1054 |
| Brain | hsa-miR-1271-3p | 1055 | 1056 |
| Brain | hsa-miR-1271-5p | 1057 | 1058 |
| Brain | hsa-miR-137 | 1059 | 1060 |
| Brain | hsa-miR-153 | 1061 | 1062 |
| Brain | hsa-miR-183-3p | 1063 | 1064 |
| Brain | hsa-miR-183-5p | 1065 | 1066 |
| Brain | hsa-miR-190a | 1067 | 1068 |
| Brain | hsa-miR-190b | 1069 | 1070 |
| Brain | hsa-miR-3665 | 1071 | 1072 |
| Brain | hsa-miR-3666 | 1073 | 1074 |
| Brain | hsa-miR-380-3p | 1075 | 1076 |
| Brain | hsa-miR-410 | 1077 | 1078 |
| Brain | hsa-miR-425-3p | 1079 | 1080 |
| Brain | hsa-miR-425-5p | 1081 | 1082 |
| Brain | hsa-miR-510 | 1083 | 1084 |
| Brain | hsa-miR-7-5p | 1085 | 1086 |
| Brain | hsa-miR-9-3p | 1087 | 1088 |
| Brain | hsa-miR-9-5p | 1089 | 1090 |
| Brain and hematopoietic cells | hsa-miR-125a-3p | 1091 | 1092 |
| Brain and hematopoietic cells | hsa-miR-125a-5p | 1093 | 1094 |
| Brain and pancreas | hsa-miR-7-2-3p | 1095 | 1096 |
| Brain and plasma (circulating) | hsa-miR-124-5p | 1097 | 1098 |
| Brain, and plasma (exosomal) | hsa-miR-124-3p | 1099 | 1100 |
| Brain and platelet | hsa-miR-329 | 1101 | 1102 |
| Brain (neuron), immune cells | hsa-miR-132-3p | 1103 | 1104 |
| Brain (neuron), immune cells | hsa-miR-132-5p | 1105 | 1106 |
| Brain (neuron), spleen | hsa-miR-212-3p | 1107 | 1108 |
| Brain (neuron), spleen | hsa-miR-212-5p | 1109 | 1110 |
| Brain, circulating plasma | hsa-miR-342-3p | 1111 | 1112 |
| Brain, embryonic stem cells | hsa-miR-380-5p | 1113 | 1114 |
| Brain, epithelial cells, hepatocytes | hsa-miR-802 | 1115 | 1116 |
| Brain, oligodendrocytes | hsa-miR-219-1-3p | 1117 | 1118 |
| Brain, oligodendrocytes | hsa-miR-219-2-3p | 1119 | 1120 |
| Brain, oligodendrocytes | hsa-miR-219-5p | 1121 | 1122 |
| Brain, other tissues | hsa-miR-135a-3p | 1123 | 1124 |
| Brain, other tissues | hsa-miR-135a-5p | 1125 | 1126 |
| Brain, placenta, other tissues | hsa-miR-135b-3p | 1127 | 1128 |
| Brain, placenta, other tissues | hsa-miR-135b-5p | 1129 | 1130 |
| Brain, stem cells/progenitor | hsa-miR-181c-3p | 1131 | 1132 |
| Brain, stem cells/progenitor | hsa-miR-181c-5p | 1133 | 1134 |
| Breast tumor | hsa-miR-3180-5p | 1135 | 1136 |
| Breast tumor | hsa-miR-3529-3p | 1137 | 1138 |
| Breast tumor | hsa-miR-3529-5p | 1139 | 1140 |
| Breast tumor | hsa-miR-3591-3p | 1141 | 1142 |
| Breast tumor | hsa-miR-3591-5p | 1143 | 1144 |
| Breast tumor | hsa-miR-3688-3p | 1145 | 1146 |
| Breast tumor | hsa-miR-3688-5p | 1147 | 1148 |
| Breast tumor | hsa-miR-3940-3p | 1149 | 1150 |
| Breast tumor | hsa-miR-3940-5p | 1151 | 1152 |
| Breast tumor | hsa-miR-3944-3p | 1153 | 1154 |
| Breast tumor | hsa-miR-3944-5p | 1155 | 1156 |
| Breast tumor | hsa-miR-4436b-3p | 1157 | 1158 |
| Breast tumor | hsa-miR-4436b-5p | 1159 | 1160 |
| Breast tumor | hsa-miR-4520b-3p | 1161 | 1162 |
| Breast tumor | hsa-miR-4520b-5p | 1163 | 1164 |
| Breast tumor | hsa-miR-4632-3p | 1165 | 1166 |
| Breast tumor | hsa-miR-4632-5p | 1167 | 1168 |
| Breast tumor | hsa-miR-4633-3p | 1169 | 1170 |
| Breast tumor | hsa-miR-4633-5p | 1171 | 1172 |
| Breast tumor | hsa-miR-4634 | 1173 | 1174 |
| Breast tumor | hsa-miR-4635 | 1175 | 1176 |
| Breast tumor | hsa-miR-4636 | 1177 | 1178 |
| Breast tumor | hsa-miR-4638-3p | 1179 | 1180 |
| Breast tumor | hsa-miR-4638-5p | 1181 | 1182 |
| Breast tumor | hsa-miR-4639-3p | 1183 | 1184 |
| Breast tumor | hsa-miR-4639-5p | 1185 | 1186 |
| Breast tumor | hsa-miR-4640-3p | 1187 | 1188 |
| Breast tumor | hsa-miR-4640-5p | 1189 | 1190 |
| Breast tumor | hsa-miR-4641 | 1191 | 1192 |
| Breast tumor | hsa-miR-4642 | 1193 | 1194 |
| Breast tumor | hsa-miR-4643 | 1195 | 1196 |
| Breast tumor | hsa-miR-4644 | 1197 | 1198 |
| Breast tumor | hsa-miR-4645-3p | 1199 | 1200 |
| Breast tumor | hsa-miR-4645-5p | 1201 | 1202 |
| Breast tumor | hsa-miR-4646-3p | 1203 | 1204 |
| Breast tumor | hsa-miR-4646-5p | 1205 | 1206 |
| Breast tumor | hsa-miR-4647 | 1207 | 1208 |
| Breast tumor | hsa-miR-4648 | 1209 | 1210 |
| Breast tumor | hsa-miR-4649-3p | 1211 | 1212 |
| Breast tumor | hsa-miR-4649-5p | 1213 | 1214 |
| Breast tumor | hsa-miR-4650-3p | 1215 | 1216 |
| Breast tumor | hsa-miR-4650-5p | 1217 | 1218 |
| Breast tumor | hsa-miR-4651 | 1219 | 1220 |
| Breast tumor | hsa-miR-4652-3p | 1221 | 1222 |
| Breast tumor | hsa-miR-4652-5p | 1223 | 1224 |
| Breast tumor | hsa-miR-4653-3p | 1225 | 1226 |
| Breast tumor | hsa-miR-4653-5p | 1227 | 1228 |
| Breast tumor | hsa-miR-4654 | 1229 | 1230 |
| Breast tumor | hsa-miR-4655-3p | 1231 | 1232 |
| Breast tumor | hsa-miR-4655-5p | 1233 | 1234 |
| Breast tumor | hsa-miR-4656 | 1235 | 1236 |
| Breast tumor | hsa-miR-4657 | 1237 | 1238 |
| Breast tumor | hsa-miR-4658 | 1239 | 1240 |
| Breast tumor | hsa-miR-4659a-3p | 1241 | 1242 |
| Breast tumor | hsa-miR-4659a-5p | 1243 | 1244 |
| Breast tumor | hsa-miR-4659b-3p | 1245 | 1246 |
| Breast tumor | hsa-miR-4659b-5p | 1247 | 1248 |
| Breast tumor | hsa-miR-4660 | 1249 | 1250 |
| Breast tumor | hsa-miR-4661-3p | 1251 | 1252 |
| Breast tumor | hsa-miR-4661-5p | 1253 | 1254 |
| Breast tumor | hsa-miR-4662b | 1255 | 1256 |
| Breast tumor | hsa-miR-4663 | 1257 | 1258 |
| Breast tumor | hsa-miR-4664-3p | 1259 | 1260 |
| Breast tumor | hsa-miR-4664-5p | 1261 | 1262 |
| Breast tumor | hsa-miR-4665-3p | 1263 | 1264 |
| Breast tumor | hsa-miR-4665-5p | 1265 | 1266 |
| Breast tumor | hsa-miR-4666a-3p | 1267 | 1268 |
| Breast tumor | hsa-miR-4666a-5p | 1269 | 1270 |
| Breast tumor | hsa-miR-4667-3p | 1271 | 1272 |
| Breast tumor | hsa-miR-4667-5p | 1273 | 1274 |
| Breast tumor | hsa-miR-4668-3p | 1275 | 1276 |
| Breast tumor | hsa-miR-4668-5p | 1277 | 1278 |
| Breast tumor | hsa-miR-4669 | 1279 | 1280 |
| Breast tumor | hsa-miR-4670-3p | 1281 | 1282 |
| Breast tumor | hsa-miR-4670-5p | 1283 | 1284 |
| Breast tumor | hsa-miR-4671-3p | 1285 | 1286 |
| Breast tumor | hsa-miR-4671-5p | 1287 | 1288 |
| Breast tumor | hsa-miR-4672 | 1289 | 1290 |
| Breast tumor | hsa-miR-4673 | 1291 | 1292 |
| Breast tumor | hsa-miR-4674 | 1293 | 1294 |
| Breast tumor | hsa-miR-4675 | 1295 | 1296 |
| Breast tumor | hsa-miR-4676-3p | 1297 | 1298 |
| Breast tumor | hsa-miR-4676-5p | 1299 | 1300 |
| Breast tumor | hsa-miR-4678 | 1301 | 1302 |
| Breast tumor | hsa-miR-4679 | 1303 | 1304 |
| Breast tumor | hsa-miR-4680-3p | 1305 | 1306 |
| Breast tumor | hsa-miR-4680-5p | 1307 | 1308 |
| Breast tumor | hsa-miR-4681 | 1309 | 1310 |
| Breast tumor | hsa-miR-4682 | 1311 | 1312 |
| Breast tumor | hsa-miR-4683 | 1313 | 1314 |
| Breast tumor | hsa-miR-4684-3p | 1315 | 1316 |
| Breast tumor | hsa-miR-4684-5p | 1317 | 1318 |
| Breast tumor | hsa-miR-4685-3p | 1319 | 1320 |
| Breast tumor | hsa-miR-4685-5p | 1321 | 1322 |
| Breast tumor | hsa-miR-4686 | 1323 | 1324 |
| Breast tumor | hsa-miR-4687-3p | 1325 | 1326 |
| Breast tumor | hsa-miR-4687-5p | 1327 | 1328 |
| Breast tumor | hsa-miR-4688 | 1329 | 1330 |
| Breast tumor | hsa-miR-4689 | 1331 | 1332 |
| Breast tumor | hsa-miR-4690-3p | 1333 | 1334 |
| Breast tumor | hsa-miR-4690-5p | 1335 | 1336 |
| Breast tumor | hsa-miR-4691-3p | 1337 | 1338 |
| Breast tumor | hsa-miR-4691-5p | 1339 | 1340 |
| Breast tumor | hsa-miR-4692 | 1341 | 1342 |
| Breast tumor | hsa-miR-4693-3p | 1343 | 1344 |
| Breast tumor | hsa-miR-4693-5p | 1345 | 1346 |
| Breast tumor | hsa-miR-4694-3p | 1347 | 1348 |
| Breast tumor | hsa-miR-4694-5p | 1349 | 1350 |
| Breast tumor | hsa-miR-4695-3p | 1351 | 1352 |
| Breast tumor | hsa-miR-4695-5p | 1353 | 1354 |
| Breast tumor | hsa-miR-4696 | 1355 | 1356 |
| Breast tumor | hsa-miR-4697-3p | 1357 | 1358 |
| Breast tumor | hsa-miR-4697-5p | 1359 | 1360 |
| Breast tumor | hsa-miR-4698 | 1361 | 1362 |
| Breast tumor | hsa-miR-4699-3p | 1363 | 1364 |
| Breast tumor | hsa-miR-4699-5p | 1365 | 1366 |
| Breast tumor | hsa-miR-4700-3p | 1367 | 1368 |
| Breast tumor | hsa-miR-4700-5p | 1369 | 1370 |
| Breast tumor | hsa-miR-4701-3p | 1371 | 1372 |
| Breast tumor | hsa-miR-4701-5p | 1373 | 1374 |
| Breast tumor | hsa-miR-4703-3p | 1375 | 1376 |
| Breast tumor | hsa-miR-4703-5p | 1377 | 1378 |
| Breast tumor | hsa-miR-4704-3p | 1379 | 1380 |
| Breast tumor | hsa-miR-4704-5p | 1381 | 1382 |
| Breast tumor | hsa-miR-4705 | 1383 | 1384 |
| Breast tumor | hsa-miR-4706 | 1385 | 1386 |
| Breast tumor | hsa-miR-4707-3p | 1387 | 1388 |
| Breast tumor | hsa-miR-4707-5p | 1389 | 1390 |
| Breast tumor | hsa-miR-4708-3p | 1391 | 1392 |
| Breast tumor | hsa-miR-4708-5p | 1393 | 1394 |
| Breast tumor | hsa-miR-4709-3p | 1395 | 1396 |
| Breast tumor | hsa-miR-4709-5p | 1397 | 1398 |
| Breast tumor | hsa-miR-4710 | 1399 | 1400 |
| Breast tumor | hsa-miR-4711-3p | 1401 | 1402 |
| Breast tumor | hsa-miR-4711-5p | 1403 | 1404 |
| Breast tumor | hsa-miR-4712-3p | 1405 | 1406 |
| Breast tumor | hsa-miR-4712-5p | 1407 | 1408 |
| Breast tumor | hsa-miR-4713-3p | 1409 | 1410 |
| Breast tumor | hsa-miR-4713-5p | 1411 | 1412 |
| Breast tumor | hsa-miR-4714-3p | 1413 | 1414 |
| Breast tumor | hsa-miR-4714-5p | 1415 | 1416 |
| Breast tumor | hsa-miR-4715-3p | 1417 | 1418 |
| Breast tumor | hsa-miR-4715-5p | 1419 | 1420 |
| Breast tumor | hsa-miR-4716-3p | 1421 | 1422 |
| Breast tumor | hsa-miR-4716-5p | 1423 | 1424 |
| Breast tumor | hsa-miR-4717-3p | 1425 | 1426 |
| Breast tumor | hsa-miR-4717-5p | 1427 | 1428 |
| Breast tumor | hsa-miR-4718 | 1429 | 1430 |
| Breast tumor | hsa-miR-4719 | 1431 | 1432 |
| Breast tumor | hsa-miR-4720-3p | 1433 | 1434 |
| Breast tumor | hsa-miR-4720-5p | 1435 | 1436 |
| Breast tumor | hsa-miR-4721 | 1437 | 1438 |
| Breast tumor | hsa-miR-4722-3p | 1439 | 1440 |
| Breast tumor | hsa-miR-4722-5p | 1441 | 1442 |
| Breast tumor | hsa-miR-4723-3p | 1443 | 1444 |
| Breast tumor | hsa-miR-4723-5p | 1445 | 1446 |
| Breast tumor | hsa-miR-4724-3p | 1447 | 1448 |
| Breast tumor | hsa-miR-4724-5p | 1449 | 1450 |
| Breast tumor | hsa-miR-4725-3p | 1451 | 1452 |
| Breast tumor | hsa-miR-4725-5p | 1453 | 1454 |
| Breast tumor | hsa-miR-4726-3p | 1455 | 1456 |
| Breast tumor | hsa-miR-4726-5p | 1457 | 1458 |
| Breast tumor | hsa-miR-4727-3p | 1459 | 1460 |
| Breast tumor | hsa-miR-4727-5p | 1461 | 1462 |
| Breast tumor | hsa-miR-4728-3p | 1463 | 1464 |
| Breast tumor | hsa-miR-4728-5p | 1465 | 1466 |
| Breast tumor | hsa-miR-4729 | 1467 | 1468 |
| Breast tumor | hsa-miR-4730 | 1469 | 1470 |
| Breast tumor | hsa-miR-4731-3p | 1471 | 1472 |
| Breast tumor | hsa-miR-4731-5p | 1473 | 1474 |
| Breast tumor | hsa-miR-4732-3p | 1475 | 1476 |
| Breast tumor | hsa-miR-4732-5p | 1477 | 1478 |
| Breast tumor | hsa-miR-4733-3p | 1479 | 1480 |
| Breast tumor | hsa-miR-4733-5p | 1481 | 1482 |
| Breast tumor | hsa-miR-4734 | 1483 | 1484 |
| Breast tumor | hsa-miR-4735-3p | 1485 | 1486 |
| Breast tumor | hsa-miR-4735-5p | 1487 | 1488 |
| Breast tumor | hsa-miR-4736 | 1489 | 1490 |
| Breast tumor | hsa-miR-4737 | 1491 | 1492 |
| Breast tumor | hsa-miR-4738-3p | 1493 | 1494 |
| Breast tumor | hsa-miR-4738-5p | 1495 | 1496 |
| Breast tumor | hsa-miR-4739 | 1497 | 1498 |
| Breast tumor | hsa-miR-4740-3p | 1499 | 1500 |
| Breast tumor | hsa-miR-4740-5p | 1501 | 1502 |
| Breast tumor | hsa-miR-4742-5p | 1503 | 1504 |
| Breast tumor | hsa-miR-4743-3p | 1505 | 1506 |
| Breast tumor | hsa-miR-4743-5p | 1507 | 1508 |
| Breast tumor | hsa-miR-4744 | 1509 | 1510 |
| Breast tumor | hsa-miR-4745-3p | 1511 | 1512 |
| Breast tumor | hsa-miR-4745-5p | 1513 | 1514 |
| Breast tumor | hsa-miR-4746-3p | 1515 | 1516 |
| Breast tumor | hsa-miR-4746-5p | 1517 | 1518 |
| Breast tumor | hsa-miR-4747-3p | 1519 | 1520 |
| Breast tumor | hsa-miR-4747-5p | 1521 | 1522 |
| Breast tumor | hsa-miR-4748 | 1523 | 1524 |
| Breast tumor | hsa-miR-4749-3p | 1525 | 1526 |
| Breast tumor | hsa-miR-4749-5p | 1527 | 1528 |
| Breast tumor | hsa-miR-4750-3p | 1529 | 1530 |
| Breast tumor | hsa-miR-4750-5p | 1531 | 1532 |
| Breast tumor | hsa-miR-4751 | 1533 | 1534 |
| Breast tumor | hsa-miR-4752 | 1535 | 1536 |
| Breast tumor | hsa-miR-4753-3p | 1537 | 1538 |
| Breast tumor | hsa-miR-4753-5p | 1539 | 1540 |
| Breast tumor | hsa-miR-4754 | 1541 | 1542 |
| Breast tumor | hsa-miR-4755-3p | 1543 | 1544 |
| Breast tumor | hsa-miR-4755-5p | 1545 | 1546 |
| Breast tumor | hsa-miR-4756-3p | 1547 | 1548 |
| Breast tumor | hsa-miR-4756-5p | 1549 | 1550 |
| Breast tumor | hsa-miR-4757-3p | 1551 | 1552 |
| Breast tumor | hsa-miR-4757-5p | 1553 | 1554 |
| Breast tumor | hsa-miR-4758-3p | 1555 | 1556 |
| Breast tumor | hsa-miR-4758-5p | 1557 | 1558 |
| Breast tumor | hsa-miR-4759 | 1559 | 1560 |
| Breast tumor | hsa-miR-4760-3p | 1561 | 1562 |
| Breast tumor | hsa-miR-4760-5p | 1563 | 1564 |
| Breast tumor | hsa-miR-4761-3p | 1565 | 1566 |
| Breast tumor | hsa-miR-4761-5p | 1567 | 1568 |
| Breast tumor | hsa-miR-4762-3p | 1569 | 1570 |
| Breast tumor | hsa-miR-4762-5p | 1571 | 1572 |
| Breast tumor | hsa-miR-4763-3p | 1573 | 1574 |
| Breast tumor | hsa-miR-4763-5p | 1575 | 1576 |
| Breast tumor | hsa-miR-4764-3p | 1577 | 1578 |
| Breast tumor | hsa-miR-4764-5p | 1579 | 1580 |
| Breast tumor | hsa-miR-4765 | 1581 | 1582 |
| Breast tumor | hsa-miR-4766-3p | 1583 | 1584 |
| Breast tumor | hsa-miR-4766-5p | 1585 | 1586 |
| Breast tumor | hsa-miR-4767 | 1587 | 1588 |
| Breast tumor | hsa-miR-4768-3p | 1589 | 1590 |
| Breast tumor | hsa-miR-4768-5p | 1591 | 1592 |
| Breast tumor | hsa-miR-4769-3p | 1593 | 1594 |
| Breast tumor | hsa-miR-4769-5p | 1595 | 1596 |
| Breast tumor | hsa-miR-4770 | 1597 | 1598 |
| Breast tumor | hsa-miR-4771 | 1599 | 1600 |
| Breast tumor | hsa-miR-4773 | 1601 | 1602 |
| Breast tumor | hsa-miR-4775 | 1603 | 1604 |
| Breast tumor | hsa-miR-4776-3p | 1605 | 1606 |
| Breast tumor | hsa-miR-4776-5p | 1607 | 1608 |
| Breast tumor | hsa-miR-4777-3p | 1609 | 1610 |
| Breast tumor | hsa-miR-4777-5p | 1611 | 1612 |
| Breast tumor | hsa-miR-4778-3p | 1613 | 1614 |
| Breast tumor | hsa-miR-4778-5p | 1615 | 1616 |
| Breast tumor | hsa-miR-4779 | 1617 | 1618 |
| Breast tumor | hsa-miR-4780 | 1619 | 1620 |
| Breast tumor | hsa-miR-4781-3p | 1621 | 1622 |
| Breast tumor | hsa-miR-4781-5p | 1623 | 1624 |
| Breast tumor | hsa-miR-4782-3p | 1625 | 1626 |
| Breast tumor | hsa-miR-4782-5p | 1627 | 1628 |
| Breast tumor | hsa-miR-4783-3p | 1629 | 1630 |
| Breast tumor | hsa-miR-4783-5p | 1631 | 1632 |
| Breast tumor | hsa-miR-4784 | 1633 | 1634 |
| Breast tumor | hsa-miR-4785 | 1635 | 1636 |
| Breast tumor | hsa-miR-4786-3p | 1637 | 1638 |
| Breast tumor | hsa-miR-4786-5p | 1639 | 1640 |
| Breast tumor | hsa-miR-4787-3p | 1641 | 1642 |
| Breast tumor | hsa-miR-4787-5p | 1643 | 1644 |
| Breast tumor | hsa-miR-4788 | 1645 | 1646 |
| Breast tumor | hsa-miR-4789-3p | 1647 | 1648 |
| Breast tumor | hsa-miR-4789-5p | 1649 | 1650 |
| Breast tumor | hsa-miR-4790-3p | 1651 | 1652 |
| Breast tumor | hsa-miR-4790-5p | 1653 | 1654 |
| Breast tumor | hsa-miR-4791 | 1655 | 1656 |
| Breast tumor | hsa-miR-4792 | 1657 | 1658 |
| Breast tumor | hsa-miR-4793-3p | 1659 | 1660 |
| Breast tumor | hsa-miR-4793-5p | 1661 | 1662 |
| Breast tumor | hsa-miR-4794 | 1663 | 1664 |
| Breast tumor | hsa-miR-4795-3p | 1665 | 1666 |
| Breast tumor | hsa-miR-4795-5p | 1667 | 1668 |
| Breast tumor | hsa-miR-4796-3p | 1669 | 1670 |
| Breast tumor | hsa-miR-4796-5p | 1671 | 1672 |
| Breast tumor | hsa-miR-4797-3p | 1673 | 1674 |
| Breast tumor | hsa-miR-4797-5p | 1675 | 1676 |
| Breast tumor | hsa-miR-4798-3p | 1677 | 1678 |
| Breast tumor | hsa-miR-4798-5p | 1679 | 1680 |
| Breast tumor | hsa-miR-4799-3p | 1681 | 1682 |
| Breast tumor | hsa-miR-4799-5p | 1683 | 1684 |
| Breast tumor | hsa-miR-4800-3p | 1685 | 1686 |
| Breast tumor | hsa-miR-4800-5p | 1687 | 1688 |
| Breast tumor | hsa-miR-4801 | 1689 | 1690 |
| Breast tumor | hsa-miR-4803 | 1691 | 1692 |
| Breast tumor | hsa-miR-4804-3p | 1693 | 1694 |
| Breast tumor | hsa-miR-4804-5p | 1695 | 1696 |
| Breast tumor and B cells | hsa-miR-4422 | 1697 | 1698 |
| Breast tumor and B cells | hsa-miR-4436a | 1699 | 1700 |
| Breast tumor and B cells | hsa-miR-4446-3p | 1701 | 1702 |
| Breast tumor and B cells | hsa-miR-4446-5p | 1703 | 1704 |
| Breast tumor and B cells | hsa-miR-4467 | 1705 | 1706 |
| Breast tumor and B cells | hsa-miR-4469 | 1707 | 1708 |
| Breast tumor and B cells | hsa-miR-4471 | 1709 | 1710 |
| Breast tumor and B cells | hsa-miR-4489 | 1711 | 1712 |
| Breast tumor and B cells | hsa-miR-4526 | 1713 | 1714 |
| Breast tumor and B cells | hsa-miR-4529-3p | 1715 | 1716 |
| Breast tumor and B cells | hsa-miR-4529-5p | 1717 | 1718 |
| Breast tumor and B cells, skin (psoriasis) | hsa-miR-4520a-3p | 1719 | 1720 |
| Breast tumor and B cells, skin (psoriasis) | hsa-miR-4520a-5p | 1721 | 1722 |
| Breast tumor and B cells, skin (psoriasis) | hsa-miR-4524a-3p | 1723 | 1724 |
| Breast tumor and B cells, skin (psoriasis) | hsa-miR-4524a-5p | 1725 | 1726 |
| Breast tumor and B cells, skin (psoriasis) | hsa-miR-4524b-3p | 1727 | 1728 |
| Breast tumor and B cells, skin (psoriasis) | hsa-miR-4524b-5p | 1729 | 1730 |
| Breast tumor and female reproductive | hsa-miR-3911 | 1731 | 1732 |
| tract | | | |
| Breast tumor and female reproductive | hsa-miR-3913-3p | 1733 | 1734 |
| tract | | | |
| Breast tumor and female reproductive | hsa-miR-3913-5p | 1735 | 1736 |
| tract | | | |
| Breast tumor and female reproductive | hsa-miR-3914 | 1737 | 1738 |
| tract | | | |
| Breast tumor and female reproductive | hsa-miR-3922-3p | 1739 | 1740 |
| tract | | | |
| Breast tumor and female reproductive | hsa-miR-3922-5p | 1741 | 1742 |
| tract | | | |
| Breast tumor and female reproductive | hsa-miR-3925-3p | 1743 | 1744 |
| tract | | | |
| Breast tumor and female reproductive | hsa-miR-3925-5p | 1745 | 1746 |
| tract | | | |
| Breast tumor and lymphoblastic leukemia | hsa-miR-3936 | 1747 | 1748 |
| Breast tumor and lymphoblastic leukemia | hsa-miR-3942-3p | 1749 | 1750 |
| Breast tumor and lymphoblastic leukemia | hsa-miR-3942-5p | 1751 | 1752 |
| Breast tumor and lymphoblastic leukemia | hsa-miR-4637 | 1753 | 1754 |
| Breast tumor and lymphoblastic leukemia | hsa-miR-4774-3p | 1755 | 1756 |
| Breast tumor and lymphoblastic leukemia | hsa-miR-4774-5p | 1757 | 1758 |
| Breast tumor, B cells and skin (psoriasis) | hsa-miR-4423-5p | 1759 | 1760 |
| Breast tumor, B cells and skin (psoriasis) | hsa-miR-4423-3p | 1761 | 1762 |
| Breast tumor, blood mononuclear cells | hsa-miR-4772-3p | 1763 | 1764 |
| Breast tumor, blood mononuclear cells | hsa-miR-4772-5p | 1765 | 1766 |
| Breast tumor, lymphoblastic leukemia and | hsa-miR-4474-3p | 1767 | 1768 |
| B cells | | | |
| Breast tumor, lymphoblastic leukemia and | hsa-miR-4474-5p | 1769 | 1770 |
| B cells | | | |
| Breast tumor, psoriasis | hsa-miR-4662a-3p | 1771 | 1772 |
| Breast tumor, psoriasis | hsa-miR-4662a-5p | 1773 | 1774 |
| Breast tumor, psoriasis | hsa-miR-4677-3p | 1775 | 1776 |
| Breast tumor, psoriasis | hsa-miR-4677-5p | 1777 | 1778 |
| Breast tumor, psoriasis | hsa-miR-4741 | 1779 | 1780 |
| Breast tumor, psoriasis | hsa-miR-4742-3p | 1781 | 1782 |
| Breast tumor, psoriasis | hsa-miR-4802-3p | 1783 | 1784 |
| Breast tumor, psoriasis | hsa-miR-4802-5p | 1785 | 1786 |
| Breast tumor | hsa-miR-3619-3p | 1787 | 1788 |
| Breast tumor | hsa-miR-3619-5p | 1789 | 1790 |
| Breast tumor | hsa-miR-3622a-3p | 1791 | 1792 |
| Breast tumor | hsa-miR-3622a-5p | 1793 | 1794 |
| Breast tumor | hsa-miR-3659 | 1795 | 1796 |
| Breast tumor | hsa-miR-3660 | 1797 | 1798 |
| Breast tumor | hsa-miR-3661 | 1799 | 1800 |
| Breast tumor | hsa-miR-3664-3p | 1801 | 1802 |
| Breast tumor | hsa-miR-3664-5p | 1803 | 1804 |
| Breast tumor | hsa-miR-3180-3p | 1805 | 1806 |
| Breast, myeloid cells, ciliated epithelial | hsa-miR-34a-3p | 1807 | 1808 |
| cells | | | |
| Breast, myeloid cells, ciliated epithelial | hsa-miR-34a-5p | 1809 | 1810 |
| cells | | | |
| Breast, pancreas (islet) | hsa-miR-195-3p | 1811 | 1812 |
| Breast, pancreas (islet) | hsa-miR-195-5p | 1813 | 1814 |
| Cardiomyocytes | hsa-miR-590-3p | 1815 | 1816 |
| Cardiomyocytes | hsa-miR-590-5p | 1817 | 1818 |
| Cartilage (chondrocyte), fetal brain | hsa-miR-483-5p | 1819 | 1820 |
| Cartilage (chondrocytes) | hsa-miR-140-5p | 1821 | 1822 |
| Cartilage (chondrocytes) | hsa-miR-576-5p | 1823 | 1824 |
| Cartilage (chondrocytes) | hsa-miR-634 | 1825 | 1826 |
| Cartilage (chondrocytes) | hsa-miR-641 | 1827 | 1828 |
| Cartilage (chondrocytes) | hsa-miR-582-3p | 1829 | 1830 |
| Cartilage (chondrocytes) | hsa-miR-1227-3p | 1831 | 1832 |
| Cartilage (chondrocytes) | hsa-miR-1227-5p | 1833 | 1834 |
| Central nervous system (CNS) | hsa-miR-320b | 1835 | 1836 |
| Central nervous system (CNS) | hsa-miR-198 | 1837 | 1838 |
| Cervical and breast tumors | hsa-miR-3614-3p | 1839 | 1840 |
| Cervical and breast tumors | hsa-miR-3614-5p | 1841 | 1842 |
| Cervical cancer | hsa-miR-933 | 1843 | 1844 |
| Cervical cancer | hsa-miR-934 | 1845 | 1846 |
| Cervical cancer | hsa-miR-940 | 1847 | 1848 |
| Cervical cancer | hsa-miR-943 | 1849 | 1850 |
| Cervical tumor | hsa-miR-548aa | 1851 | 1852 |
| Cervical tumor | hsa-miR-548z | 1853 | 1854 |
| Cervical tumor | hsa-miR-550b-2-5p | 1855 | 1856 |
| Cervical tumor | hsa-miR-550b-3p | 1857 | 1858 |
| Cervical tumor | hsa-miR-642b-3p | 1859 | 1860 |
| Cervical tumor | hsa-miR-642b-5p | 1861 | 1862 |
| Cervical tumor | hsa-miR-3606-3p | 1863 | 1864 |
| Cervical tumor | hsa-miR-3606-5p | 1865 | 1866 |
| Cervical tumor | hsa-miR-3607-3p | 1867 | 1868 |
| Cervical tumor | hsa-miR-3607-5p | 1869 | 1870 |
| Cervical tumor | hsa-miR-3609 | 1871 | 1872 |
| Cervical tumor | hsa-miR-3610 | 1873 | 1874 |
| Cervical tumor | hsa-miR-3611 | 1875 | 1876 |
| Cervical tumor | hsa-miR-3612 | 1877 | 1878 |
| Cervical tumor | hsa-miR-3613-3p | 1879 | 1880 |
| Cervical tumor | hsa-miR-3613-5p | 1881 | 1882 |
| Cervical tumor | hsa-miR-3615 | 1883 | 1884 |
| Cervical tumor | hsa-miR-3616-3p | 1885 | 1886 |
| Cervical tumor | hsa-miR-3616-5p | 1887 | 1888 |
| Cervical tumor | hsa-miR-3618 | 1889 | 1890 |
| Cervical tumor | hsa-miR-3620-3p | 1891 | 1892 |
| Cervical tumor | hsa-miR-3620-5p | 1893 | 1894 |
| Cervical tumor | hsa-miR-3621 | 1895 | 1896 |
| Cervical tumor | hsa-miR-3622b-3p | 1897 | 1898 |
| Cervical tumor | hsa-miR-3622b-5p | 1899 | 1900 |
| Cervical tumor and psoriasis | hsa-miR-3617-3p | 1901 | 1902 |
| Cervical tumor and psoriasis | hsa-miR-3617-5p | 1903 | 1904 |
| Cholesterol regulation and brain | hsa-miR-758-3p | 1905 | 1906 |
| Cholesterol regulation and brain | hsa-miR-758-5p | 1907 | 1908 |
| Chondrocyte | hsa-miR-320c | 1909 | 1910 |
| Chondrocyte | hsa-miR-624-3p | 1911 | 1912 |
| Chondrocyte | hsa-miR-624-5p | 1913 | 1914 |
| Chondrocyte | hsa-miR-630 | 1915 | 1916 |
| Chondrocyte, ciliated epithelial cells | hsa-miR-449a | 1917 | 1918 |
| Chondrogenesis, lung, brain | hsa-miR-381-3p | 1919 | 1920 |
| Chondrogenesis, lung, brain | hsa-miR-381-5p | 1921 | 1922 |
| Ciliated epithelial cells | hsa-miR-34b-3p | 1923 | 1924 |
| Ciliated epithelial cells | hsa-miR-34b-5p | 1925 | 1926 |
| Ciliated epithelial cells, other tissues | hsa-miR-449b-3p | 1927 | 1928 |
| Ciliated epithelial cells, other tissues | hsa-miR-449b-5p | 1929 | 1930 |
| Ciliated epithelial cells, placenta | hsa-miR-34c-3p | 1931 | 1932 |
| Ciliated epithelial cells, placenta | hsa-miR-34c-5p | 1933 | 1934 |
| Circulating micrornas (in Plasma) | hsa-miR-572 | 1935 | 1936 |
| Circulating micrornas (in Plasma) | hsa-miR-614 | 1937 | 1938 |
| Circulating micrornas (in Plasma) | hsa-miR-648 | 1939 | 1940 |
| Circulating micrornas (in Plasma) | hsa-miR-342-5p | 1941 | 1942 |
| CNS (prefrontal cortex) | hsa-miR-30d-3p | 1943 | 1944 |
| CNS (prefrontal cortex), embryoid body | hsa-miR-30d-5p | 1945 | 1946 |
| cells | | | |
| CNS (prefrontal cortex), other tissues | hsa-miR-30a-5p | 1947 | 1948 |
| Colorectal micrornaome | hsa-miR-548a | 1949 | 1950 |
| Colorectal micrornaome | hsa-miR-548a-3p | 1951 | 1952 |
| Colorectal micrornaome | hsa-miR-548a-5p | 1953 | 1954 |
| Colorectal micrornaome | hsa-miR-548b-3p | 1955 | 1956 |
| Colorectal micrornaome | hsa-miR-548c-3p | 1957 | 1958 |
| Colorectal micrornaome | hsa-miR-548d-3p | 1959 | 1960 |
| Colorectal micrornaome | hsa-miR-548d-5p | 1961 | 1962 |
| Colorectal micrornaome | hsa-miR-549a | 1963 | 1964 |
| Colorectal micrornaome | hsa-miR-552 | 1965 | 1966 |
| Colorectal micrornaome | hsa-miR-553 | 1967 | 1968 |
| Colorectal micrornaome | hsa-miR-554 | 1969 | 1970 |
| Colorectal micrornaome | hsa-miR-555 | 1971 | 1972 |
| Colorectal micrornaome | hsa-miR-556-3p | 1973 | 1974 |
| Colorectal micrornaome | hsa-miR-556-5p | 1975 | 1976 |
| Colorectal micrornaome | hsa-miR-563 | 1977 | 1978 |
| Colorectal micrornaome | hsa-miR-568 | 1979 | 1980 |
| Colorectal micrornaome | hsa-miR-573 | 1981 | 1982 |
| Colorectal micrornaome | hsa-miR-576-3p | 1983 | 1984 |
| Colorectal micrornaome | hsa-miR-577 | 1985 | 1986 |
| Colorectal micrornaome | hsa-miR-578 | 1987 | 1988 |
| Colorectal micrornaome | hsa-miR-586 | 1989 | 1990 |
| Colorectal micrornaome | hsa-miR-587 | 1991 | 1992 |
| Colorectal micrornaome | hsa-miR-588 | 1993 | 1994 |
| Colorectal micrornaome | hsa-miR-597 | 1995 | 1996 |
| Colorectal micrornaome | hsa-miR-600 | 1997 | 1998 |
| Colorectal micrornaome | hsa-miR-604 | 1999 | 2000 |
| Colorectal micrornaome | hsa-miR-605 | 2001 | 2002 |
| Colorectal micrornaome | hsa-miR-606 | 2003 | 2004 |
| Colorectal micrornaome | hsa-miR-607 | 2005 | 2006 |
| Colorectal micrornaome | hsa-miR-6070 | 2007 | 2008 |
| Colorectal micrornaome | hsa-miR-609 | 2009 | 2010 |
| Colorectal micrornaome | hsa-miR-619 | 2011 | 2012 |
| Colorectal micrornaome | hsa-miR-620 | 2013 | 2014 |
| Colorectal micrornaome | hsa-miR-626 | 2015 | 2016 |
| Colorectal micrornaome | hsa-miR-631 | 2017 | 2018 |
| Colorectal micrornaome | hsa-miR-635 | 2019 | 2020 |
| Colorectal micrornaome | hsa-miR-637 | 2021 | 2022 |
| Colorectal micrornaome | hsa-miR-639 | 2023 | 2024 |
| Colorectal micrornaome | hsa-miR-642a-5p | 2025 | 2026 |
| Colorectal micrornaome | hsa-miR-643 | 2027 | 2028 |
| Colorectal micrornaome | hsa-miR-651 | 2029 | 2030 |
| Colorectal micrornaome | hsa-miR-653 | 2031 | 2032 |
| Colorectal micrornaome | hsa-miR-654-3p | 2033 | 2034 |
| Corneal epithelial cells | hsa-miR-762 | 2035 | 2036 |
| Dendritic cells | hsa-let-7c | 2037 | 2038 |
| Dendritic cells and macrophages | hsa-miR-511 | 2039 | 2040 |
| Embryoid body cells | hsa-miR-1247-3p | 2041 | 2042 |
| Embryoid body cells | hsa-miR-1247-5p | 2043 | 2044 |
| Embryoid body cells | hsa-miR-1262 | 2045 | 2046 |
| Embryoid body cells | hsa-miR-1269a | 2047 | 2048 |
| Embryoid body cells | hsa-miR-1269b | 2049 | 2050 |
| Embryoid body cells | hsa-miR-1277-3p | 2051 | 2052 |
| Embryoid body cells | hsa-miR-1277-5p | 2053 | 2054 |
| Embryoid body cells | hsa-miR-1287 | 2055 | 2056 |
| Embryoid body cells | hsa-miR-1290 | 2057 | 2058 |
| Embryoid body cells | hsa-miR-340-5p | 2059 | 2060 |
| Embryoid body cells, central nervous | hsa-miR-454-3p | 2061 | 2062 |
| system, monocytes | | | |
| Embryoid body cells, central nervous | hsa-miR-454-5p | 2063 | 2064 |
| system, monocytes | | | |
| Embryoid body cells | hsa-miR-302e | 2065 | 2066 |
| Embryonic stem cells | hsa-miR-1234-3p | 2067 | 2068 |
| Embryonic stem cells | hsa-miR-1234-5p | 2069 | 2070 |
| Embryonic stem cells | hsa-let-7d-3p | 2071 | 2072 |
| Embryonic stem cells | hsa-let-7d-5p | 2073 | 2074 |
| Embryonic stem cells | hsa-miR-106b-3p | 2075 | 2076 |
| Embryonic stem cells | hsa-miR-106b-5p | 2077 | 2078 |
| Embryonic stem cells | hsa-miR-1243 | 2079 | 2080 |
| Embryonic stem cells | hsa-miR-1244 | 2081 | 2082 |
| Embryonic stem cells | hsa-miR-1245a | 2083 | 2084 |
| Embryonic stem cells | hsa-miR-1245b-3p | 2085 | 2086 |
| Embryonic stem cells | hsa-miR-1245b-5p | 2087 | 2088 |
| Embryonic stem cells | hsa-miR-1251 | 2089 | 2090 |
| Embryonic stem cells | hsa-miR-1252 | 2091 | 2092 |
| Embryonic stem cells | hsa-miR-1253 | 2093 | 2094 |
| Embryonic stem cells | hsa-miR-1254 | 2095 | 2096 |
| Embryonic stem cells | hsa-miR-1255a | 2097 | 2098 |
| Embryonic stem cells | hsa-miR-1255b-2-3p | 2099 | 2100 |
| Embryonic stem cells | hsa-miR-1255b-5p | 2101 | 2102 |
| Embryonic stem cells | hsa-miR-1256 | 2103 | 2104 |
| Embryonic stem cells | hsa-miR-1257 | 2105 | 2106 |
| Embryonic stem cells | hsa-miR-1258 | 2107 | 2108 |
| Embryonic stem cells | hsa-miR-1261 | 2109 | 2110 |
| Embryonic stem cells | hsa-miR-1263 | 2111 | 2112 |
| Embryonic stem cells | hsa-miR-1264 | 2113 | 2114 |
| Embryonic stem cells | hsa-miR-1265 | 2115 | 2116 |
| Embryonic stem cells | hsa-miR-1266 | 2117 | 2118 |
| Embryonic stem cells | hsa-miR-1267 | 2119 | 2120 |
| Embryonic stem cells | hsa-miR-1268a | 2121 | 2122 |
| Embryonic stem cells | hsa-miR-1268b | 2123 | 2124 |
| Embryonic stem cells | hsa-miR-1270 | 2125 | 2126 |
| Embryonic stem cells | hsa-miR-1272 | 2127 | 2128 |
| Embryonic stem cells | hsa-miR-1273a | 2129 | 2130 |
| Embryonic stem cells | hsa-miR-1273d | 2131 | 2132 |
| Embryonic stem cells | hsa-miR-1275 | 2133 | 2134 |
| Embryonic stem cells | hsa-miR-1276 | 2135 | 2136 |
| Embryonic stem cells | hsa-miR-1278 | 2137 | 2138 |
| Embryonic stem cells | hsa-miR-1282 | 2139 | 2140 |
| Embryonic stem cells | hsa-miR-1288 | 2141 | 2142 |
| Embryonic stem cells | hsa-miR-1293 | 2143 | 2144 |
| Embryonic stem cells | hsa-miR-1294 | 2145 | 2146 |
| Embryonic stem cells | hsa-miR-1297 | 2147 | 2148 |
| Embryonic stem cells | hsa-miR-1299 | 2149 | 2150 |
| Embryonic stem cells | hsa-miR-1305 | 2151 | 2152 |
| Embryonic stem cells | hsa-miR-1306-3p | 2153 | 2154 |
| Embryonic stem cells | hsa-miR-1306-5p | 2155 | 2156 |
| Embryonic stem cells | hsa-miR-1307-3p | 2157 | 2158 |
| Embryonic stem cells | hsa-miR-1307-5p | 2159 | 2160 |
| Embryonic stem cells | hsa-miR-146b-5p | 2161 | 2162 |
| Embryonic stem cells | hsa-miR-154-3p | 2163 | 2164 |
| Embryonic stem cells | hsa-miR-154-5p | 2165 | 2166 |
| Embryonic stem cells | hsa-miR-1910 | 2167 | 2168 |
| Embryonic stem cells | hsa-miR-1913 | 2169 | 2170 |
| Embryonic stem cells | hsa-miR-1914-3p | 2171 | 2172 |
| Embryonic stem cells | hsa-miR-1914-5p | 2173 | 2174 |
| Embryonic stem cells | hsa-miR-1915-3p | 2175 | 2176 |
| Embryonic stem cells | hsa-miR-1915-5p | 2177 | 2178 |
| Embryonic stem cells | hsa-miR-2113 | 2179 | 2180 |
| Embryonic stem cells | hsa-miR-2355-3p | 2181 | 2182 |
| Embryonic stem cells | hsa-miR-2355-5p | 2183 | 2184 |
| Embryonic stem cells | hsa-miR-301a-3p | 2185 | 2186 |
| Embryonic stem cells | hsa-miR-301a-5p | 2187 | 2188 |
| Embryonic stem cells | hsa-miR-302b-3p | 2189 | 2190 |
| Embryonic stem cells | hsa-miR-302b-5p | 2191 | 2192 |
| Embryonic stem cells | hsa-miR-302c-3p | 2193 | 2194 |
| Embryonic stem cells | hsa-miR-302c-5p | 2195 | 2196 |
| Embryonic stem cells | hsa-miR-302d-3p | 2197 | 2198 |
| Embryonic stem cells | hsa-miR-302d-5p | 2199 | 2200 |
| Embryonic stem cells | hsa-miR-367-3p | 2201 | 2202 |
| Embryonic stem cells | hsa-miR-367-5p | 2203 | 2204 |
| Embryonic stem cells | hsa-miR-423-3p | 2205 | 2206 |
| Embryonic stem cells | hsa-miR-548e | 2207 | 2208 |
| Embryonic stem cells | hsa-miR-548f | 2209 | 2210 |
| Embryonic stem cells | hsa-miR-548g-3p | 2211 | 2212 |
| Embryonic stem cells | hsa-miR-548g-5p | 2213 | 2214 |
| Embryonic stem cells | hsa-miR-548h-3p | 2215 | 2216 |
| Embryonic stem cells | hsa-miR-548h-5p | 2217 | 2218 |
| Embryonic stem cells | hsa-miR-548k | 2219 | 2220 |
| Embryonic stem cells | hsa-miR-548l | 2221 | 2222 |
| Embryonic stem cells | hsa-miR-548m | 2223 | 2224 |
| Embryonic stem cells | hsa-miR-548o-3p | 2225 | 2226 |
| Embryonic stem cells | hsa-miR-548o-5p | 2227 | 2228 |
| Embryonic stem cells | hsa-miR-548p | 2229 | 2230 |
| Embryonic stem cells | hsa-miR-6086 | 2231 | 2232 |
| Embryonic stem cells | hsa-miR-6087 | 2233 | 2234 |
| Embryonic stem cells | hsa-miR-6088 | 2235 | 2236 |
| Embryonic stem cells | hsa-miR-6089 | 2237 | 2238 |
| Embryonic stem cells | hsa-miR-6090 | 2239 | 2240 |
| Embryonic stem cells | hsa-miR-664a-3p | 2241 | 2242 |
| Embryonic stem cells | hsa-miR-664a-5p | 2243 | 2244 |
| Embryonic stem cells | hsa-miR-664b-3p | 2245 | 2246 |
| Embryonic stem cells | hsa-miR-664b-5p | 2247 | 2248 |
| Embryonic stem cells | hsa-miR-766-3p | 2249 | 2250 |
| Embryonic stem cells | hsa-miR-766-5p | 2251 | 2252 |
| Embryonic stem cells | hsa-miR-885-3p | 2253 | 2254 |
| Embryonic stem cells | hsa-miR-885-5p | 2255 | 2256 |
| Embryonic stem cells | hsa-miR-93-3p | 2257 | 2258 |
| Embryonic stem cells | hsa-miR-93-5p | 2259 | 2260 |
| Embryonic stem cells | hsa-miR-941 | 2261 | 2262 |
| Embryonic stem cells and a variety of | hsa-miR-103a-2-5p | 2263 | 2264 |
| cells and tissues | | | |
| Embryonic stem cells and a variety of | hsa-miR-103a-3p | 2265 | 2266 |
| cells and tissues | | | |
| Embryonic stem cells and neural | hsa-miR-4251 | 2267 | 2268 |
| precursors | | | |
| Embryonic stem cells and neural | hsa-miR-4252 | 2269 | 2270 |
| precursors | | | |
| Embryonic stem cells and neural | hsa-miR-4253 | 2271 | 2272 |
| precursors | | | |
| Embryonic stem cells and neural | hsa-miR-4254 | 2273 | 2274 |
| precursors | | | |
| Embryonic stem cells and neural | hsa-miR-4255 | 2275 | 2276 |
| precursors | | | |
| Embryonic stem cells and neural | hsa-miR-4256 | 2277 | 2278 |
| precursors | | | |
| Embryonic stem cells and neural | hsa-miR-4257 | 2279 | 2280 |
| precursors | | | |
| Embryonic stem cells and neural | hsa-miR-4258 | 2281 | 2282 |
| precursors | | | |
| Embryonic stem cells and neural | hsa-miR-4259 | 2283 | 2284 |
| precursors | | | |
| Embryonic stem cells and neural | hsa-miR-4260 | 2285 | 2286 |
| precursors | | | |
| Embryonic stem cells and neural | hsa-miR-4261 | 2287 | 2288 |
| precursors | | | |
| Embryonic stem cells and neural | hsa-miR-4262 | 2289 | 2290 |
| precursors | | | |
| Embryonic stem cells and neural | hsa-miR-4263 | 2291 | 2292 |
| precursors | | | |
| Embryonic stem cells and neural | hsa-miR-4264 | 2293 | 2294 |
| precursors | | | |
| Embryonic stem cells and neural | hsa-miR-4265 | 2295 | 2296 |
| precursors | | | |
| Embryonic stem cells and neural | hsa-miR-4266 | 2297 | 2298 |
| precursors | | | |
| Embryonic stem cells and neural | hsa-miR-4267 | 2299 | 2300 |
| precursors | | | |
| Embryonic stem cells and neural | hsa-miR-4268 | 2301 | 2302 |
| precursors | | | |
| Embryonic stem cells and neural | hsa-miR-4269 | 2303 | 2304 |
| precursors | | | |
| Embryonic stem cells and neural | hsa-miR-4270 | 2305 | 2306 |
| precursors | | | |
| Embryonic stem cells and neural | hsa-miR-4271 | 2307 | 2308 |
| precursors | | | |
| Embryonic stem cells and neural | hsa-miR-4272 | 2309 | 2310 |
| precursors | | | |
| Embryonic stem cells and neural | hsa-miR-4274 | 2311 | 2312 |
| precursors | | | |
| Embryonic stem cells and neural | hsa-miR-4275 | 2313 | 2314 |
| precursors | | | |
| Embryonic stem cells and neural | hsa-miR-4276 | 2315 | 2316 |
| precursors | | | |
| Embryonic stem cells and neural | hsa-miR-4277 | 2317 | 2318 |
| precursors | | | |
| Embryonic stem cells and neural | hsa-miR-4278 | 2319 | 2320 |
| precursors | | | |
| Embryonic stem cells and neural | hsa-miR-4279 | 2321 | 2322 |
| precursors | | | |
| Embryonic stem cells and neural | hsa-miR-4280 | 2323 | 2324 |
| precursors | | | |
| Embryonic stem cells and neural | hsa-miR-4281 | 2325 | 2326 |
| precursors | | | |
| Embryonic stem cells and neural | hsa-miR-4282 | 2327 | 2328 |
| precursors | | | |
| Embryonic stem cells and neural | hsa-miR-4283 | 2329 | 2330 |
| precursors | | | |
| Embryonic stem cells and neural | hsa-miR-4284 | 2331 | 2332 |
| precursors | | | |
| Embryonic stem cells and neural | hsa-miR-4285 | 2333 | 2334 |
| precursors | | | |
| Embryonic stem cells and neural | hsa-miR-4286 | 2335 | 2336 |
| precursors | | | |
| Embryonic stem cells and neural | hsa-miR-4287 | 2337 | 2338 |
| precursors | | | |
| Embryonic stem cells and neural | hsa-miR-4288 | 2339 | 2340 |
| precursors | | | |
| Embryonic stem cells and neural | hsa-miR-4289 | 2341 | 2342 |
| precursors | | | |
| Embryonic stem cells and neural | hsa-miR-4290 | 2343 | 2344 |
| precursors | | | |
| Embryonic stem cells and neural | hsa-miR-4291 | 2345 | 2346 |
| precursors | | | |
| Embryonic stem cells and neural | hsa-miR-4292 | 2347 | 2348 |
| precursors | | | |
| Embryonic stem cells and neural | hsa-miR-4293 | 2349 | 2350 |
| precursors | | | |
| Embryonic stem cells and neural | hsa-miR-4294 | 2351 | 2352 |
| precursors | | | |
| Embryonic stem cells and neural | hsa-miR-4295 | 2353 | 2354 |
| precursors | | | |
| Embryonic stem cells and neural | hsa-miR-4296 | 2355 | 2356 |
| precursors | | | |
| Embryonic stem cells and neural | hsa-miR-4297 | 2357 | 2358 |
| precursors | | | |
| Embryonic stem cells and neural | hsa-miR-4298 | 2359 | 2360 |
| precursors | | | |
| Embryonic stem cells and neural | hsa-miR-4299 | 2361 | 2362 |
| precursors | | | |
| Embryonic stem cells and neural | hsa-miR-4300 | 2363 | 2364 |
| precursors | | | |
| Embryonic stem cells and neural | hsa-miR-4301 | 2365 | 2366 |
| precursors | | | |
| Embryonic stem cells and neural | hsa-miR-4302 | 2367 | 2368 |
| precursors | | | |
| Embryonic stem cells and neural | hsa-miR-4303 | 2369 | 2370 |
| precursors | | | |
| Embryonic stem cells and neural | hsa-miR-4304 | 2371 | 2372 |
| precursors | | | |
| Embryonic stem cells and neural | hsa-miR-4305 | 2373 | 2374 |
| precursors | | | |
| Embryonic stem cells and neural | hsa-miR-4306 | 2375 | 2376 |
| precursors | | | |
| Embryonic stem cells and neural | hsa-miR-4307 | 2377 | 2378 |
| precursors | | | |
| Embryonic stem cells and neural | hsa-miR-4308 | 2379 | 2380 |
| precursors | | | |
| Embryonic stem cells and neural | hsa-miR-4309 | 2381 | 2382 |
| precursors | | | |
| Embryonic stem cells and neural | hsa-miR-4310 | 2383 | 2384 |
| precursors | | | |
| Embryonic stem cells and neural | hsa-miR-4311 | 2385 | 2386 |
| precursors | | | |
| Embryonic stem cells and neural | hsa-miR-4312 | 2387 | 2388 |
| precursors | | | |
| Embryonic stem cells and neural | hsa-miR-4313 | 2389 | 2390 |
| precursors | | | |
| Embryonic stem cells and neural | hsa-miR-4314 | 2391 | 2392 |
| precursors | | | |
| Embryonic stem cells and neural | hsa-miR-4315 | 2393 | 2394 |
| precursors | | | |
| Embryonic stem cells and neural | hsa-miR-4316 | 2395 | 2396 |
| precursors | | | |
| Embryonic stem cells and neural | hsa-miR-4317 | 2397 | 2398 |
| precursors | | | |
| Embryonic stem cells and neural | hsa-miR-4318 | 2399 | 2400 |
| precursors | | | |
| Embryonic stem cells and neural | hsa-miR-4319 | 2401 | 2402 |
| precursors | | | |
| Embryonic stem cells and neural | hsa-miR-4320 | 2403 | 2404 |
| precursors | | | |
| Embryonic stem cells and neural | hsa-miR-4321 | 2405 | 2406 |
| precursors | | | |
| Embryonic stem cells and neural | hsa-miR-4322 | 2407 | 2408 |
| precursors | | | |
| Embryonic stem cells and neural | hsa-miR-4323 | 2409 | 2410 |
| precursors | | | |
| Embryonic stem cells and neural | hsa-miR-4324 | 2411 | 2412 |
| precursors | | | |
| Embryonic stem cells and neural | hsa-miR-4325 | 2413 | 2414 |
| precursors | | | |
| Embryonic stem cells and neural | hsa-miR-4326 | 2415 | 2416 |
| precursors | | | |
| Embryonic stem cells and neural | hsa-miR-4327 | 2417 | 2418 |
| precursors | | | |
| Embryonic stem cells and neural | hsa-miR-4328 | 2419 | 2420 |
| precursors | | | |
| Embryonic stem cells and neural | hsa-miR-4329 | 2421 | 2422 |
| precursors | | | |
| Embryonic stem cells and neural | hsa-miR-4330 | 2423 | 2424 |
| precursors | | | |
| Embryonic stem cells, airway smooth | hsa-miR-25-3p | 2425 | 2426 |
| muscle | | | |
| Embryonic stem cells, airway smooth | hsa-miR-25-5p | 2427 | 2428 |
| muscle | | | |
| Embryonic stem cells, Blood and other | hsa-miR-26a-1-3p | 2429 | 2430 |
| tissues | | | |
| Embryonic stem cells, epithelial cells | hsa-miR-1246 | 2431 | 2432 |
| Embryonic stem cells, heart | hsa-miR-744-5p | 2433 | 2434 |
| Embryonic stem cells, immune cells | hsa-miR-548i | 2435 | 2436 |
| Embryonic stem cells, immune cells | hsa-miR-548n | 2437 | 2438 |
| Embryonic stem cells, lipid metabolism | hsa-miR-302a-3p | 2439 | 2440 |
| Embryonic stem cells, lipid metabolism | hsa-miR-302a-5p | 2441 | 2442 |
| Embryonic stem cells, lung | hsa-let-7a-3p | 2443 | 2444 |
| Embryonic stem cells, lung | hsa-let-7a-5p | 2445 | 2446 |
| Embryonic stem cells, lung, myeloid cells | hsa-let-7a-2-3p | 2447 | 2448 |
| Embryonic stem cells, neural precursor | hsa-miR-1911-3p | 2449 | 2450 |
| Embryonic stem cells, neural precursor | hsa-miR-1911-5p | 2451 | 2452 |
| Embryonic stem cells, neural precursor | hsa-miR-1912 | 2453 | 2454 |
| Embryonic stem cells, placenta | hsa-miR-512-3p | 2455 | 2456 |
| Embryonic stem cells, placenta | hsa-miR-512-5p | 2457 | 2458 |
| Endometrial tissues | hsa-miR-196b-3p | 2459 | 2460 |
| Endometrial tissues | hsa-miR-196b-5p | 2461 | 2462 |
| Endothelial cells | hsa-miR-101-3p | 2463 | 2464 |
| Endothelial cells | hsa-miR-101-5p | 2465 | 2466 |
| Endothelial cells | hsa-miR-19a-3p | 2467 | 2468 |
| Endothelial cells | hsa-miR-19a-5p | 2469 | 2470 |
| Endothelial cells | hsa-miR-19b-1-5p | 2471 | 2472 |
| Endothelial cells | hsa-miR-19b-2-5p | 2473 | 2474 |
| Endothelial cells | hsa-miR-19b-3p | 2475 | 2476 |
| Endothelial cells | hsa-miR-217 | 2477 | 2478 |
| Endothelial cells | hsa-miR-218-1-3p | 2479 | 2480 |
| Endothelial cells | hsa-miR-222-3p | 2481 | 2482 |
| Endothelial cells | hsa-miR-222-5p | 2483 | 2484 |
| Endothelial cells | hsa-miR-361-5p | 2485 | 2486 |
| Endothelial cells | hsa-miR-421 | 2487 | 2488 |
| Endothelial cells | hsa-miR-424-3p | 2489 | 2490 |
| Endothelial cells | hsa-miR-424-5p | 2491 | 2492 |
| Endothelial cells | hsa-miR-513a-5p | 2493 | 2494 |
| Endothelial cells | hsa-miR-5739 | 2495 | 2496 |
| Endothelial cells | hsa-miR-6068 | 2497 | 2498 |
| Endothelial cells | hsa-miR-6069 | 2499 | 2500 |
| Endothelial cells | hsa-miR-6071 | 2501 | 2502 |
| Endothelial cells | hsa-miR-6072 | 2503 | 2504 |
| Endothelial cells | hsa-miR-6073 | 2505 | 2506 |
| Endothelial cells | hsa-miR-6074 | 2507 | 2508 |
| Endothelial cells | hsa-miR-6075 | 2509 | 2510 |
| Endothelial cells | hsa-miR-6076 | 2511 | 2512 |
| Endothelial cells | hsa-miR-6077 | 2513 | 2514 |
| Endothelial cells | hsa-miR-6078 | 2515 | 2516 |
| Endothelial cells | hsa-miR-6079 | 2517 | 2518 |
| Endothelial cells | hsa-miR-6080 | 2519 | 2520 |
| Endothelial cells | hsa-miR-6081 | 2521 | 2522 |
| Endothelial cells | hsa-miR-6082 | 2523 | 2524 |
| Endothelial cells | hsa-miR-6083 | 2525 | 2526 |
| Endothelial cells | hsa-miR-6084 | 2527 | 2528 |
| Endothelial cells | hsa-miR-6085 | 2529 | 2530 |
| Endothelial cells | hsa-miR-92a-1-5p | 2531 | 2532 |
| Endothelial cells | hsa-miR-92a-2-5p | 2533 | 2534 |
| Endothelial cells and CNS | hsa-miR-92a-3p | 2535 | 2536 |
| Endothelial cells and heart | hsa-miR-92b-3p | 2537 | 2538 |
| Endothelial cells and heart | hsa-miR-92b-5p | 2539 | 2540 |
| Endothelial cells, brain (astrocyte), blood | hsa-miR-23a-3p | 2541 | 2542 |
| (erythroid) | | | |
| Endothelial cells, brain (astrocyte), blood | hsa-miR-23a-5p | 2543 | 2544 |
| (erythroid) | | | |
| Endothelial cells, embryonic stem cells | hsa-miR-17-3p | 2545 | 2546 |
| Endothelial cells, immune cells | hsa-miR-221-3p | 2547 | 2548 |
| Endothelial cells, immune cells | hsa-miR-221-5p | 2549 | 2550 |
| Endothelial cells, lung | hsa-miR-126-3p | 2551 | 2552 |
| Endothelial cells, lung | hsa-miR-126-5p | 2553 | 2554 |
| Endothelial progenitor cells (epcs) | hsa-miR-508-5p | 2555 | 2556 |
| Endothelial progenitor cells, oocytes | hsa-miR-662 | 2557 | 2558 |
| Epididymis | hsa-miR-6511b-3p | 2559 | 2560 |
| Epididymis | hsa-miR-6511b-5p | 2561 | 2562 |
| Epididymis | hsa-miR-6715a-3p | 2563 | 2564 |
| Epididymis | hsa-miR-6715b-3p | 2565 | 2566 |
| Epididymis | hsa-miR-6715b-5p | 2567 | 2568 |
| Epididymis | hsa-miR-6716-3p | 2569 | 2570 |
| Epididymis | hsa-miR-6716-5p | 2571 | 2572 |
| Epididymis | hsa-miR-6717-5p | 2573 | 2574 |
| Epididymis | hsa-miR-6718-5p | 2575 | 2576 |
| Epididymis | hsa-miR-6719-3p | 2577 | 2578 |
| Epididymis | hsa-miR-6720-3p | 2579 | 2580 |
| Epididymis | hsa-miR-6721-5p | 2581 | 2582 |
| Epididymis | hsa-miR-6722-3p | 2583 | 2584 |
| Epididymis | hsa-miR-6722-5p | 2585 | 2586 |
| Epididymis | hsa-miR-6723-5p | 2587 | 2588 |
| Epididymis | hsa-miR-6724-5p | 2589 | 2590 |
| Epididymis | hsa-miR-890 | 2591 | 2592 |
| Epididymis | hsa-miR-891a | 2593 | 2594 |
| Epididymis | hsa-miR-891b | 2595 | 2596 |
| Epididymis | hsa-miR-892a | 2597 | 2598 |
| Epididymis | hsa-miR-892b | 2599 | 2600 |
| Epididymis | hsa-miR-892c-3p | 2601 | 2602 |
| Epididymis | hsa-miR-892c-5p | 2603 | 2604 |
| Epithelial cells | hsa-miR-384 | 2605 | 2606 |
| Epithelial cells | hsa-miR-429 | 2607 | 2608 |
| Epithelial cells | hsa-miR-494 | 2609 | 2610 |
| Epithelial cells, endothelial cells | hsa-let-7b-3p | 2611 | 2612 |
| (vascular) | | | |
| Epithelial cells, endothelial cells | hsa-let-7b-5p | 2613 | 2614 |
| (vascular) | | | |
| Epithelial cells, many other tissues | hsa-miR-200a-3p | 2615 | 2616 |
| Epithelial cells, many other tissues | hsa-miR-200a-5p | 2617 | 2618 |
| Epithelial cells, many other tissues | hsa-miR-200b-3p | 2619 | 2620 |
| Epithelial cells, many other tissues | hsa-miR-200b-5p | 2621 | 2622 |
| Epithelial cells, many other tissues, | hsa-miR-200c-3p | 2623 | 2624 |
| embryonic stem cells | | | |
| Epithelial cells, many other tissues, | hsa-miR-200c-5p | 2625 | 2626 |
| embryonic stem cells | | | |
| Epithelial cells, oligodendrocytes | hsa-miR-338-3p | 2627 | 2628 |
| Erythroid cells | hsa-miR-144-3p | 2629 | 2630 |
| Erythroid cells | hsa-miR-144-5p | 2631 | 2632 |
| Erythroid cells | hsa-miR-486-3p | 2633 | 2634 |
| Esophageal cell line KYSE-150R | hsa-miR-1237-3p | 2635 | 2636 |
| Esophageal cell line KYSE-150R | hsa-miR-1237-5p | 2637 | 2638 |
| Esophageal cell line KYSE-150R | hsa-miR-1539 | 2639 | 2640 |
| Female reproductive tract | hsa-miR-2115-3p | 2641 | 2642 |
| Female reproductive tract | hsa-miR-2115-5p | 2643 | 2644 |
| Female reproductive tract | hsa-miR-2277-3p | 2645 | 2646 |
| Female reproductive tract | hsa-miR-2277-5p | 2647 | 2648 |
| Female reproductive tract | hsa-miR-3689a-3p | 2649 | 2650 |
| Female reproductive tract | hsa-miR-3689b-5p | 2651 | 2652 |
| Female reproductive tract | hsa-miR-3907 | 2653 | 2654 |
| Female reproductive tract | hsa-miR-3908 | 2655 | 2656 |
| Female reproductive tract | hsa-miR-3909 | 2657 | 2658 |
| Female reproductive tract | hsa-miR-3910 | 2659 | 2660 |
| Female reproductive tract | hsa-miR-3912 | 2661 | 2662 |
| Female reproductive tract | hsa-miR-3915 | 2663 | 2664 |
| Female reproductive tract | hsa-miR-3916 | 2665 | 2666 |
| Female reproductive tract | hsa-miR-3917 | 2667 | 2668 |
| Female reproductive tract | hsa-miR-3918 | 2669 | 2670 |
| Female reproductive tract | hsa-miR-3919 | 2671 | 2672 |
| Female reproductive tract | hsa-miR-3920 | 2673 | 2674 |
| Female reproductive tract | hsa-miR-3921 | 2675 | 2676 |
| Female reproductive tract | hsa-miR-3923 | 2677 | 2678 |
| Female reproductive tract | hsa-miR-3924 | 2679 | 2680 |
| Female reproductive tract | hsa-miR-3926 | 2681 | 2682 |
| Female reproductive tract | hsa-miR-3928 | 2683 | 2684 |
| Female reproductive tract | hsa-miR-3929 | 2685 | 2686 |
| Female reproductive tract | hsa-miR-676-3p | 2687 | 2688 |
| Female reproductive tract | hsa-miR-676-5p | 2689 | 2690 |
| Female reproductive tract and peripheral | hsa-miR-3689a-5p | 2691 | 2692 |
| blood | | | |
| Female reproductive tract and peripheral | hsa-miR-3689b-3p | 2693 | 2694 |
| blood | | | |
| Female reproductive tract and psoriasis | hsa-miR-3927-3p | 2695 | 2696 |
| Female reproductive tract and psoriasis | hsa-miR-3927-5p | 2697 | 2698 |
| Fibroblast | hsa-miR-5787 | 2699 | 2700 |
| Frontal cortex | hsa-miR-516a-3p | 2701 | 2702 |
| Frontal cortex | hsa-miR-571 | 2703 | 2704 |
| Glia cells | hsa-miR-181d | 2705 | 2706 |
| Glioblast, brain | hsa-miR-128 | 2707 | 2708 |
| Glioblast, brain, pancreas | hsa-miR-7-1-3p | 2709 | 2710 |
| Glioblast, Embryonic stem cells, | hsa-miR-181b-3p | 2711 | 2712 |
| epidermal (keratinocytes) | | | |
| Glioblast, Embryonic stem cells, | hsa-miR-181b-5p | 2713 | 2714 |
| epidermal (keratinocytes) | | | |
| Glioblast, myeloid cells, embryonic stem | hsa-miR-181a-3p | 2715 | 2716 |
| cells | | | |
| Glioblast, myeloid cells, embryonic stem | hsa-miR-181a-5p | 2717 | 2718 |
| cells | | | |
| Glioblast, stem cells | hsa-miR-181a-2-3p | 2719 | 2720 |
| Heart (cardiomyocyte) | hsa-miR-744-3p | 2721 | 2722 |
| Heart (cardiomyocyte), muscle | hsa-miR-208a | 2723 | 2724 |
| Heart (cardiomyocyte), muscle | hsa-miR-208b | 2725 | 2726 |
| Heart and brain | hsa-miR-149-3p | 2727 | 2728 |
| Heart and brain | hsa-miR-149-5p | 2729 | 2730 |
| Heart and muscle | hsa-miR-1 | 2731 | 2732 |
| Heart, cardiac stem cells | hsa-miR-499a-3p | 2733 | 2734 |
| Heart, cardiac stem cells | hsa-miR-499a-5p | 2735 | 2736 |
| Heart, cardiac stem cells | hsa-miR-499b-3p | 2737 | 2738 |
| Heart, cardiac stem cells | hsa-miR-499b-5p | 2739 | 2740 |
| Heart, central nervous system, epithelial | hsa-miR-451a | 2741 | 2742 |
| cells | | | |
| Heart, central nervous system, epithelial | hsa-miR-451b | 2743 | 2744 |
| cells | | | |
| Heart, embryonic stem cells | hsa-miR-423-5p | 2745 | 2746 |
| Hemapoietic cells | hsa-miR-99a-3p | 2747 | 2748 |
| Hemapoietic cells | hsa-miR-99a-5p | 2749 | 2750 |
| Hemapoietic cells and embryonic stem | hsa-miR-99b-3p | 2751 | 2752 |
| cells | | | |
| Hemapoietic cells and embryonic stem | hsa-miR-99b-5p | 2753 | 2754 |
| cells | | | |
| Hematocytes, brain | hsa-miR-139-3p | 2755 | 2756 |
| Hematocytes, brain | hsa-miR-139-5p | 2757 | 2758 |
| Hematopoeitic cells | hsa-miR-10a-3p | 2759 | 2760 |
| Hematopoeitic cells | hsa-miR-10a-5p | 2761 | 2762 |
| Hematopoietic cells | hsa-miR-1184 | 2763 | 2764 |
| Hematopoietic cells | hsa-miR-148a-3p | 2765 | 2766 |
| Hematopoietic cells | hsa-miR-148a-5p | 2767 | 2768 |
| Hematopoietic cells | hsa-miR-26b-3p | 2769 | 2770 |
| Hematopoietic cells | hsa-miR-26b-5p | 2771 | 2772 |
| Hematopoietic cells | hsa-miR-345-3p | 2773 | 2774 |
| Hematopoietic cells | hsa-miR-345-5p | 2775 | 2776 |
| Hematopoietic cells | hsa-miR-377-3p | 2777 | 2778 |
| Hematopoietic cells | hsa-miR-377-5p | 2779 | 2780 |
| Hematopoietic cells (erythroid, platelet, | hsa-miR-376a-2-5p | 2781 | 2782 |
| and lymphoma) | | | |
| Hematopoietic cells (erythroid, platelet, | hsa-miR-376a-3p | 2783 | 2784 |
| and lymphoma) | | | |
| Hematopoietic cells (erythroid, platelet, | hsa-miR-376a-5p | 2785 | 2786 |
| and lymphoma) | | | |
| Hematopoietic cells (lymphoid) | hsa-miR-150-3p | 2787 | 2788 |
| Hematopoietic cells (lymphoid) | hsa-miR-150-5p | 2789 | 2790 |
| Hematopoietic cells (monocytes), brain | hsa-miR-125b-1-3p | 2791 | 2792 |
| (neuron) | | | |
| Hematopoietic cells (monocytes), brain | hsa-miR-125b-2-3p | 2793 | 2794 |
| (neuron) | | | |
| Hematopoietic cells and endothelial cells | hsa-miR-100-5p | 2795 | 2796 |
| Hematopoietic cells, adipose, smooth | hsa-let-7g-3p | 2797 | 2798 |
| muscle cells | | | |
| Hematopoietic cells, adipose, smooth | hsa-let-7g-5p | 2799 | 2800 |
| muscle cells | | | |
| Hematopoietic cells, brain (neuron) | hsa-miR-125b-5p | 2801 | 2802 |
| Hematopoietic cells, endothelial cells | hsa-miR-100-3p | 2803 | 2804 |
| Hematopoietic cells, lung, placental | hsa-miR-372 | 2805 | 2806 |
| (blood) | | | |
| Hepatocytes | hsa-miR-1303 | 2807 | 2808 |
| Hepatocytes | hsa-miR-1291 | 2809 | 2810 |
| Hepatocytes | hsa-miR-551b-3p | 2811 | 2812 |
| Hepatocytes | hsa-miR-551b-5p | 2813 | 2814 |
| Hepatocytes | hsa-miR-939-3p | 2815 | 2816 |
| Hepatocytes | hsa-miR-939-5p | 2817 | 2818 |
| Human testis | hsa-miR-920 | 2819 | 2820 |
| Human testis | hsa-miR-921 | 2821 | 2822 |
| Human testis | hsa-miR-924 | 2823 | 2824 |
| Human testis, neuronal tissues | hsa-miR-922 | 2825 | 2826 |
| Immune cells | hsa-miR-339-3p | 2827 | 2828 |
| Immune cells | hsa-miR-339-5p | 2829 | 2830 |
| Immune cells | hsa-let-7e-3p | 2831 | 2832 |
| Immune cells | hsa-let-7e-5p | 2833 | 2834 |
| Immune cells | hsa-let-7i-3p | 2835 | 2836 |
| Immune cells | hsa-let-7i-5p | 2837 | 2838 |
| Immune cells | hsa-miR-146b-3p | 2839 | 2840 |
| Immune cells | hsa-miR-182-3p | 2841 | 2842 |
| Immune cells | hsa-miR-346 | 2843 | 2844 |
| Immune cells | hsa-miR-548j | 2845 | 2846 |
| Immune cells (B-cells) | hsa-miR-151b | 2847 | 2848 |
| Immune cells (T cells) | hsa-let-7f-1-3p | 2849 | 2850 |
| Immune cells (T cells) | hsa-let-7f-2-3p | 2851 | 2852 |
| Immune cells (T cells) | hsa-let-7f-5p | 2853 | 2854 |
| Immune cells, frontal cortex | hsa-miR-548b-5p | 2855 | 2856 |
| Immune cells, frontal cortex | hsa-miR-548c-5p | 2857 | 2858 |
| Immune cells, hematopoiesis | hsa-miR-146a-3p | 2859 | 2860 |
| Immune cells, hematopoiesis | hsa-miR-146a-5p | 2861 | 2862 |
| Immune cells, pancreas | hsa-miR-214-3p | 2863 | 2864 |
| Immune cells, pancreas | hsa-miR-214-5p | 2865 | 2866 |
| Immune system | hsa-miR-29a-3p | 2867 | 2868 |
| Immune system | hsa-miR-29a-5p | 2869 | 2870 |
| Immune system | hsa-miR-29b-1-5p | 2871 | 2872 |
| Immune system | hsa-miR-29b-2-5p | 2873 | 2874 |
| Immune system | hsa-miR-29b-3p | 2875 | 2876 |
| Immune system | hsa-miR-29c-3p | 2877 | 2878 |
| Immune system | hsa-miR-29c-5p | 2879 | 2880 |
| Keratinocytes | hsa-miR-668 | 2881 | 2882 |
| Kidney | hsa-miR-192-3p | 2883 | 2884 |
| Kidney | hsa-miR-192-5p | 2885 | 2886 |
| Kidney | hsa-miR-324-3p | 2887 | 2888 |
| Kidney and liver/hepatocytes | hsa-miR-122-3p | 2889 | 2890 |
| Kidney and pancreatic cells | hsa-miR-30a-3p | 2891 | 2892 |
| Kidney stem cell, blood cells | hsa-miR-363-3p | 2893 | 2894 |
| Kidney stem cell, blood cells | hsa-miR-363-5p | 2895 | 2896 |
| Kidney, adipose, CNS (prefrontal cortex) | hsa-miR-30b-3p | 2897 | 2898 |
| Kidney, adipose, CNS (prefrontal cortex) | hsa-miR-30b-5p | 2899 | 2900 |
| Kidney, adipose, CNS (prefrontal cortex) | hsa-miR-30c-1-3p | 2901 | 2902 |
| Kidney, adipose, CNS (prefrontal cortex) | hsa-miR-30c-2-3p | 2903 | 2904 |
| Kidney, adipose, CNS (prefrontal cortex) | hsa-miR-30c-5p | 2905 | 2906 |
| Kidney, breast | hsa-miR-335-3p | 2907 | 2908 |
| Kidney, breast | hsa-miR-335-5p | 2909 | 2910 |
| Kidney, breast and endothelial cells | hsa-miR-17-5p | 2911 | 2912 |
| Kidney, cartilage, vascular smooth muscle | hsa-miR-145-3p | 2913 | 2914 |
| Kidney, cartilage, vascular smooth muscle | hsa-miR-145-5p | 2915 | 2916 |
| Kidney, endothelial cells, osteogenic cells | hsa-miR-20a-3p | 2917 | 2918 |
| Kidney, endothelial cells, osteogenic cells | hsa-miR-20a-5p | 2919 | 2920 |
| Kidney, heart, lung, endothelial cells | hsa-miR-296-3p | 2921 | 2922 |
| Kidney, heart, vascular endothelial cells | hsa-miR-210 | 2923 | 2924 |
| Kidney, liver | hsa-miR-194-3p | 2925 | 2926 |
| Kidney, liver | hsa-miR-194-5p | 2927 | 2928 |
| Kidney, pancreas | hsa-miR-216a-3p | 2929 | 2930 |
| Kidney, pancreas | hsa-miR-216a-5p | 2931 | 2932 |
| Lipid metabolism | hsa-miR-33b-3p | 2933 | 2934 |
| Lipid metabolism | hsa-miR-33b-5p | 2935 | 2936 |
| Lipid metabolism | hsa-miR-378b | 2937 | 2938 |
| Lipid metabolism | hsa-miR-378c | 2939 | 2940 |
| Lipid metabolism | hsa-miR-378d | 2941 | 2942 |
| Lipid metabolism | hsa-miR-378e | 2943 | 2944 |
| Lipid metabolism | hsa-miR-378f | 2945 | 2946 |
| Lipid metabolism | hsa-miR-378g | 2947 | 2948 |
| Lipid metabolism | hsa-miR-378h | 2949 | 2950 |
| Lipid metabolism | hsa-miR-378i | 2951 | 2952 |
| Lipid metabolism | hsa-miR-378j | 2953 | 2954 |
| Lipid metabolism | hsa-miR-613 | 2955 | 2956 |
| Liver (hepatocytes) | hsa-miR-152 | 2957 | 2958 |
| Liver (hepatocytes) | hsa-miR-1228-3p | 2959 | 2960 |
| Liver (hepatocytes) | hsa-miR-1228-5p | 2961 | 2962 |
| Liver (hepatocytes) | hsa-miR-1249 | 2963 | 2964 |
| Liver (hepatocytes) | hsa-miR-448 | 2965 | 2966 |
| Liver (hepatocytes) | hsa-miR-557 | 2967 | 2968 |
| Liver (hepatocytes), circulating (blood) | hsa-miR-625-3p | 2969 | 2970 |
| Liver (hepatocytes), circulating (blood) | hsa-miR-625-5p | 2971 | 2972 |
| Liver (hepatocytes) | hsa-miR-129-5p | 2973 | 2974 |
| Liver (hepatocytes) | hsa-miR-581 | 2975 | 2976 |
| Liver, cardiomyocytes | hsa-miR-199a-5p | 2977 | 2978 |
| Liver, embryonic body cells, | hsa-miR-199a-3p | 2979 | 2980 |
| cardiomyocytes | | | |
| Liver, osteoblast | hsa-miR-199b-3p | 2981 | 2982 |
| Liver, osteoblast | hsa-miR-199b-5p | 2983 | 2984 |
| Liver (hepatocytes) | hsa-miR-122-5p | 2985 | 2986 |
| Lung (epithelial) | hsa-miR-18b-3p | 2987 | 2988 |
| Lung (epithelial) | hsa-miR-18b-5p | 2989 | 2990 |
| Lung (epithelial) | hsa-miR-337-3p | 2991 | 2992 |
| Lung (epithelial) | hsa-miR-337-5p | 2993 | 2994 |
| Lung (epithelial) | hsa-miR-134 | 2995 | 2996 |
| Lung and endothelial cells | hsa-miR-18a-3p | 2997 | 2998 |
| Lung and endothelial cells | hsa-miR-18a-5p | 2999 | 3000 |
| Lung, epidermal cells (keratinocytes) | hsa-miR-130b-3p | 3001 | 3002 |
| Lung, epidermal cells (keratinocytes) | hsa-miR-130b-5p | 3003 | 3004 |
| Lung, immune cells | hsa-miR-182-5p | 3005 | 3006 |
| Lung, liver, endothelial cells | hsa-miR-296-5p | 3007 | 3008 |
| Lung, monocytes, vascular endothelial | hsa-miR-130a-3p | 3009 | 3010 |
| cells | | | |
| Lung, monocytes, vascular endothelial | hsa-miR-130a-5p | 3011 | 3012 |
| cells | | | |
| Lung, placenta | hsa-miR-127-3p | 3013 | 3014 |
| Lung, placenta (islet) | hsa-miR-127-5p | 3015 | 3016 |
| Lymphatic endothelial cells | hsa-miR-1236-3p | 3017 | 3018 |
| Lymphatic endothelial cells | hsa-miR-1236-5p | 3019 | 3020 |
| Lymphoblastic leukemia | hsa-miR-5000-3p | 3021 | 3022 |
| Lymphoblastic leukemia | hsa-miR-5000-5p | 3023 | 3024 |
| Lymphoblastic leukemia | hsa-miR-5006-3p | 3025 | 3026 |
| Lymphoblastic leukemia | hsa-miR-5006-5p | 3027 | 3028 |
| Lymphoblastic leukemia | hsa-miR-5186 | 3029 | 3030 |
| Lymphoblastic leukemia | hsa-miR-5188 | 3031 | 3032 |
| Lymphoblastic leukemia | hsa-miR-5189 | 3033 | 3034 |
| Lymphoblastic leukemia | hsa-miR-5190 | 3035 | 3036 |
| Lymphoblastic leukemia | hsa-miR-5191 | 3037 | 3038 |
| Lymphoblastic leukemia | hsa-miR-5192 | 3039 | 3040 |
| Lymphoblastic leukemia | hsa-miR-5193 | 3041 | 3042 |
| Lymphoblastic leukemia | hsa-miR-5194 | 3043 | 3044 |
| Lymphoblastic leukemia | hsa-miR-5195-3p | 3045 | 3046 |
| Lymphoblastic leukemia | hsa-miR-5195-5p | 3047 | 3048 |
| Lymphoblastic leukemia | hsa-miR-5196-3p | 3049 | 3050 |
| Lymphoblastic leukemia | hsa-miR-5196-5p | 3051 | 3052 |
| Lymphoblastic leukemia | hsa-miR-5197-3p | 3053 | 3054 |
| Lymphoblastic leukemia | hsa-miR-5197-5p | 3055 | 3056 |
| Lymphoblastic leukemia, skin (psoriasis) | hsa-miR-5187-3p | 3057 | 3058 |
| Lymphoblastic leukemia, skin (psoriasis) | hsa-miR-5187-5p | 3059 | 3060 |
| Lymphocyte, blood, hematopoietic tissues | hsa-miR-15a-3p | 3061 | 3062 |
| (spleen) | | | |
| Lymphocyte, blood, hematopoietic tissues | hsa-miR-15a-5p | 3063 | 3064 |
| (spleen) | | | |
| Lymphocyte, blood, hematopoietic tissues | hsa-miR-15b-3p | 3065 | 3066 |
| (spleen) | | | |
| Lymphocyte, blood, hematopoietic tissues | hsa-miR-15b-5p | 3067 | 3068 |
| (spleen) | | | |
| Lymphocyte, blood, hematopoietic tissues | hsa-miR-16-2-3p | 3069 | 3070 |
| (spleen) | | | |
| Lymphocytes | hsa-miR-331-5p | 3071 | 3072 |
| Macrophage | hsa-miR-147a | 3073 | 3074 |
| Macrophage | hsa-miR-147b | 3075 | 3076 |
| Many tissues and brain hepatocytes/liver | hsa-miR-107 | 3077 | 3078 |
| Many tissues/cells, semen | hsa-miR-193b-3p | 3079 | 3080 |
| Many tissues/cells, semen | hsa-miR-193b-5p | 3081 | 3082 |
| Melanocytes | hsa-miR-211-3p | 3083 | 3084 |
| Melanocytes | hsa-miR-211-5p | 3085 | 3086 |
| Melanoma miRNAome | hsa-miR-548s | 3087 | 3088 |
| Melanoma miRNAome | hsa-miR-548t-3p | 3089 | 3090 |
| Melanoma miRNAome | hsa-miR-548t-5p | 3091 | 3092 |
| Melanoma miRNAome | hsa-miR-548u | 3093 | 3094 |
| Melanoma miRNAome | hsa-miR-548w | 3095 | 3096 |
| Melanoma miRNAome | hsa-miR-3116 | 3097 | 3098 |
| Melanoma miRNAome | hsa-miR-3117-3p | 3099 | 3100 |
| Melanoma miRNAome | hsa-miR-3117-5p | 3101 | 3102 |
| Melanoma miRNAome | hsa-miR-3118 | 3103 | 3104 |
| Melanoma miRNAome | hsa-miR-3119 | 3105 | 3106 |
| Melanoma miRNAome | hsa-miR-3120-3p | 3107 | 3108 |
| Melanoma miRNAome | hsa-miR-3120-5p | 3109 | 3110 |
| Melanoma miRNAome | hsa-miR-3121-3p | 3111 | 3112 |
| Melanoma miRNAome | hsa-miR-3121-5p | 3113 | 3114 |
| Melanoma miRNAome | hsa-miR-3122 | 3115 | 3116 |
| Melanoma miRNAome | hsa-miR-3123 | 3117 | 3118 |
| Melanoma miRNAome | hsa-miR-3125 | 3119 | 3120 |
| Melanoma miRNAome | hsa-miR-3127-3p | 3121 | 3122 |
| Melanoma miRNAome | hsa-miR-3127-5p | 3123 | 3124 |
| Melanoma miRNAome | hsa-miR-3128 | 3125 | 3126 |
| Melanoma miRNAome | hsa-miR-3131 | 3127 | 3128 |
| Melanoma miRNAome | hsa-miR-3132 | 3129 | 3130 |
| Melanoma miRNAome | hsa-miR-3133 | 3131 | 3132 |
| Melanoma miRNAome | hsa-miR-3134 | 3133 | 3134 |
| Melanoma miRNAome | hsa-miR-3135a | 3135 | 3136 |
| Melanoma miRNAome | hsa-miR-3136-3p | 3137 | 3138 |
| Melanoma miRNAome | hsa-miR-3136-5p | 3139 | 3140 |
| Melanoma miRNAome | hsa-miR-3137 | 3141 | 3142 |
| Melanoma miRNAome | hsa-miR-3139 | 3143 | 3144 |
| Melanoma miRNAome | hsa-miR-3141 | 3145 | 3146 |
| Melanoma miRNAome | hsa-miR-3143 | 3147 | 3148 |
| Melanoma miRNAome | hsa-miR-3145-3p | 3149 | 3150 |
| Melanoma miRNAome | hsa-miR-3145-5p | 3151 | 3152 |
| Melanoma miRNAome | hsa-miR-3146 | 3153 | 3154 |
| Melanoma miRNAome | hsa-miR-3147 | 3155 | 3156 |
| Melanoma miRNAome | hsa-miR-3148 | 3157 | 3158 |
| Melanoma miRNAome | hsa-miR-3150a-3p | 3159 | 3160 |
| Melanoma miRNAome | hsa-miR-3150a-5p | 3161 | 3162 |
| Melanoma miRNAome | hsa-miR-3150b-3p | 3163 | 3164 |
| Melanoma miRNAome | hsa-miR-3150b-5p | 3165 | 3166 |
| Melanoma miRNAome | hsa-miR-3151 | 3167 | 3168 |
| Melanoma miRNAome | hsa-miR-3153 | 3169 | 3170 |
| Melanoma miRNAome | hsa-miR-3154 | 3171 | 3172 |
| Melanoma miRNAome | hsa-miR-3155a | 3173 | 3174 |
| Melanoma miRNAome | hsa-miR-3156-3p | 3175 | 3176 |
| Melanoma miRNAome | hsa-miR-3156-5p | 3177 | 3178 |
| Melanoma miRNAome | hsa-miR-3157-3p | 3179 | 3180 |
| Melanoma miRNAome | hsa-miR-3157-5p | 3181 | 3182 |
| Melanoma miRNAome | hsa-miR-3159 | 3183 | 3184 |
| Melanoma miRNAome | hsa-miR-3160-3p | 3185 | 3186 |
| Melanoma miRNAome | hsa-miR-3160-5p | 3187 | 3188 |
| Melanoma miRNAome | hsa-miR-3161 | 3189 | 3190 |
| Melanoma miRNAome | hsa-miR-3162-3p | 3191 | 3192 |
| Melanoma miRNAome | hsa-miR-3162-5p | 3193 | 3194 |
| Melanoma miRNAome | hsa-miR-3163 | 3195 | 3196 |
| Melanoma miRNAome | hsa-miR-3164 | 3197 | 3198 |
| Melanoma miRNAome | hsa-miR-3165 | 3199 | 3200 |
| Melanoma miRNAome | hsa-miR-3166 | 3201 | 3202 |
| Melanoma miRNAome | hsa-miR-3168 | 3203 | 3204 |
| Melanoma miRNAome | hsa-miR-3169 | 3205 | 3206 |
| Melanoma miRNAome | hsa-miR-3170 | 3207 | 3208 |
| Melanoma miRNAome | hsa-miR-3173-3p | 3209 | 3210 |
| Melanoma miRNAome | hsa-miR-3173-5p | 3211 | 3212 |
| Melanoma miRNAome | hsa-miR-3174 | 3213 | 3214 |
| Melanoma miRNAome | hsa-miR-3176 | 3215 | 3216 |
| Melanoma miRNAome | hsa-miR-3177-3p | 3217 | 3218 |
| Melanoma miRNAome | hsa-miR-3177-5p | 3219 | 3220 |
| Melanoma miRNAome | hsa-miR-3178 | 3221 | 3222 |
| Melanoma miRNAome | hsa-miR-3179 | 3223 | 3224 |
| Melanoma miRNAome | hsa-miR-3181 | 3225 | 3226 |
| Melanoma miRNAome | hsa-miR-3182 | 3227 | 3228 |
| Melanoma miRNAome | hsa-miR-3183 | 3229 | 3230 |
| Melanoma miRNAome | hsa-miR-3184-3p | 3231 | 3232 |
| Melanoma miRNAome | hsa-miR-3184-5p | 3233 | 3234 |
| Melanoma miRNAome | hsa-miR-3185 | 3235 | 3236 |
| Melanoma miRNAome | hsa-miR-3187-3p | 3237 | 3238 |
| Melanoma miRNAome | hsa-miR-3187-5p | 3239 | 3240 |
| Melanoma miRNAome | hsa-miR-3188 | 3241 | 3242 |
| Melanoma miRNAome | hsa-miR-3189-3p | 3243 | 3244 |
| Melanoma miRNAome | hsa-miR-3189-5p | 3245 | 3246 |
| Melanoma miRNAome | hsa-miR-3190-3p | 3247 | 3248 |
| Melanoma miRNAome | hsa-miR-3190-5p | 3249 | 3250 |
| Melanoma miRNAome | hsa-miR-3191-3p | 3251 | 3252 |
| Melanoma miRNAome | hsa-miR-3191-5p | 3253 | 3254 |
| Melanoma miRNAome | hsa-miR-3192 | 3255 | 3256 |
| Melanoma miRNAome | hsa-miR-3193 | 3257 | 3258 |
| Melanoma miRNAome | hsa-miR-3194-3p | 3259 | 3260 |
| Melanoma miRNAome | hsa-miR-3194-5p | 3261 | 3262 |
| Melanoma miRNAome | hsa-miR-3195 | 3263 | 3264 |
| Melanoma miRNAome | hsa-miR-3197 | 3265 | 3266 |
| Melanoma miRNAome | hsa-miR-3198 | 3267 | 3268 |
| Melanoma miRNAome | hsa-miR-3199 | 3269 | 3270 |
| Melanoma miRNAome, | hsa-miR-3201 | 3271 | 3272 |
| Melanoma miRNAome, epithelial cell | hsa-miR-3202 | 3273 | 3274 |
| BEAS2B | | | |
| Melanoma miRNAome, ovary | hsa-miR-3124-3p | 3275 | 3276 |
| Melanoma miRNAome, ovary | hsa-miR-3124-5p | 3277 | 3278 |
| Melanoma miRNAome, ovary | hsa-miR-3126-3p | 3279 | 3280 |
| Melanoma miRNAome, ovary | hsa-miR-3126-5p | 3281 | 3282 |
| Melanoma miRNAome, ovary | hsa-miR-3129-3p | 3283 | 3284 |
| Melanoma miRNAome, ovary | hsa-miR-3129-5p | 3285 | 3286 |
| Melanoma miRNAome, ovary | hsa-miR-3130-3p | 3287 | 3288 |
| Melanoma miRNAome, ovary | hsa-miR-3130-5p | 3289 | 3290 |
| Melanoma miRNAome, ovary | hsa-miR-3138 | 3291 | 3292 |
| Melanoma miRNAome, ovary | hsa-miR-3140-3p | 3293 | 3294 |
| Melanoma miRNAome, ovary | hsa-miR-3140-5p | 3295 | 3296 |
| Melanoma miRNAome, ovary | hsa-miR-3144-3p | 3297 | 3298 |
| Melanoma miRNAome, ovary | hsa-miR-3144-5p | 3299 | 3300 |
| Melanoma miRNAome, ovary | hsa-miR-3149 | 3301 | 3302 |
| Melanoma miRNAome, ovary | hsa-miR-3152-3p | 3303 | 3304 |
| Melanoma miRNAome, ovary | hsa-miR-3152-5p | 3305 | 3306 |
| Melanoma miRNAome, ovary | hsa-miR-3158-3p | 3307 | 3308 |
| Melanoma miRNAome, ovary | hsa-miR-3158-5p | 3309 | 3310 |
| Melanoma miRNAome, ovary | hsa-miR-3167 | 3311 | 3312 |
| Melanoma miRNAome, ovary | hsa-miR-3171 | 3313 | 3314 |
| Melanoma miRNAome, ovary | hsa-miR-3175 | 3315 | 3316 |
| Melanoma miRNAome, ovary | hsa-miR-3180 | 3317 | 3318 |
| Melanoma miRNAome, ovary | hsa-miR-3186-3p | 3319 | 3320 |
| Melanoma miRNAome, ovary | hsa-miR-3186-5p | 3321 | 3322 |
| Melanoma miRNAome, ovary | hsa-miR-3200-3p | 3323 | 3324 |
| Melanoma miRNAome, ovary | hsa-miR-3200-5p | 3325 | 3326 |
| Melanoma miRNAome, immune cells | hsa-miR-3142 | 3327 | 3328 |
| Mesenchymal stem cells | hsa-miR-489 | 3329 | 3330 |
| Mesothelial cells | hsa-miR-589-3p | 3331 | 3332 |
| Mesothelial cells | hsa-miR-589-5p | 3333 | 3334 |
| Monocytes | hsa-miR-1279 | 3335 | 3336 |
| Monocytes | hsa-miR-542-3p | 3337 | 3338 |
| Multiple cell types | hsa-miR-1289 | 3339 | 3340 |
| Multiple cell types | hsa-miR-129-1-3p | 3341 | 3342 |
| Multiple cell types | hsa-miR-129-2-3p | 3343 | 3344 |
| Muscle (cardiac and skeletal) | hsa-miR-206 | 3345 | 3346 |
| Muscle (myoblasts) | hsa-miR-374a-3p | 3347 | 3348 |
| Muscle (myoblasts) | hsa-miR-374a-5p | 3349 | 3350 |
| Muscle (myoblasts) | hsa-miR-374b-3p | 3351 | 3352 |
| Muscle (myoblasts) | hsa-miR-374b-5p | 3353 | 3354 |
| Muscle (myoblasts) | hsa-miR-374c-3p | 3355 | 3356 |
| Muscle (myoblasts) | hsa-miR-374c-5p | 3357 | 3358 |
| Muscle, heart, epithelial cells (lung) | hsa-miR-133a | 3359 | 3360 |
| Muscle, heart, epithelial cells (lung) | hsa-miR-133b | 3361 | 3362 |
| Myeloid cell and glia cells | hsa-miR-30e-5p | 3563 | 3364 |
| Myeloid cells | hsa-miR-223-3p | 3365 | 3366 |
| Myeloid cells | hsa-miR-223-5p | 3367 | 3368 |
| Myeloid cells | hsa-miR-27a-3p | 3369 | 3370 |
| Myeloid cells | hsa-miR-27a-5p | 3371 | 3372 |
| Myeloid cells and blood | hsa-miR-23b-3p | 3373 | 3374 |
| Myeloid cells and blood | hsa-miR-23b-5p | 3375 | 3376 |
| Myeloid cells and glia cells | hsa-miR-30e-3p | 3377 | 3378 |
| Myeloid cells and lung | hsa-miR-24-1-5p | 3379 | 3380 |
| Myeloid cells and lung | hsa-miR-24-2-5p | 3381 | 3382 |
| Myeloid cells and lung | hsa-miR-24-3p | 3383 | 3384 |
| Myeloid cells and vascular endothelial | hsa-miR-27b-3p | 3385 | 3386 |
| cells | | | |
| Myeloid cells and vascular endothelial | hsa-miR-27b-5p | 3387 | 3388 |
| cells | | | |
| Myeloid cells, hematopoiesis, ARC cells | hsa-miR-142-3p | 3389 | 3390 |
| Myeloid cells, hematopoiesis, ARC cells | hsa-miR-142-5p | 3391 | 3392 |
| Myeloid cells, pancreas (islet) | hsa-miR-493-3p | 3393 | 3394 |
| Myeloid cells, pancreas (islet) | hsa-miR-493-5p | 3395 | 3396 |
| Myoblast | hsa-miR-432-3p | 3397 | 3398 |
| Myoblast | hsa-miR-432-5p | 3399 | 3400 |
| Myoblast | hsa-miR-452-3p | 3401 | 3402 |
| Myoblast | hsa-miR-452-5p | 3403 | 3404 |
| Myoblast | hsa-miR-659-3p | 3405 | 3406 |
| Myoblast | hsa-miR-659-5p | 3407 | 3408 |
| Myoblast | hsa-miR-660-3p | 3409 | 3410 |
| Myoblast | hsa-miR-660-5p | 3411 | 3412 |
| Neural cells | hsa-miR-320e | 3413 | 3414 |
| Neuroblastoma | hsa-miR-3713 | 3415 | 3416 |
| Neuroblastoma | hsa-miR-3714 | 3417 | 3418 |
| Neurons | hsa-miR-148b-3p | 3419 | 3420 |
| Neurons | hsa-miR-148b-5p | 3421 | 3422 |
| Neuron, blood | hsa-miR-328 | 3423 | 3424 |
| Neuron, fetal liver | hsa-miR-151a-3p | 3425 | 3426 |
| Neuron, fetal liver | hsa-miR-151a-5p | 3427 | 3428 |
| Neurons | hsa-miR-323a-3p | 3429 | 3430 |
| Neurons | hsa-miR-323a-5p | 3431 | 3432 |
| Neurons | hsa-miR-324-5p | 3433 | 3434 |
| Neurons | hsa-miR-326 | 3435 | 3436 |
| Neurons, placenta | hsa-miR-325 | 3437 | 3438 |
| Oligodendrocytes | hsa-miR-1250 | 3439 | 3440 |
| Oligodendrocytes | hsa-miR-3065-3p | 3441 | 3442 |
| Oligodendrocytes | hsa-miR-3065-5p | 3443 | 3444 |
| Oligodendrocytes | hsa-miR-338-5p | 3445 | 3446 |
| Oligodendrocytes | hsa-miR-657 | 3447 | 3448 |
| Oocyte | hsa-miR-602 | 3449 | 3450 |
| Oocyte and prostate | hsa-miR-297 | 3451 | 3452 |
| Osteoblasts | hsa-miR-300 | 3453 | 3454 |
| Osteoblasts | hsa-miR-3960 | 3455 | 3456 |
| Osteoblasts | hsa-miR-764 | 3457 | 3458 |
| Osteoblasts | hsa-miR-2861 | 3459 | 3460 |
| Osteoblasts, heart | hsa-miR-186-3p | 3461 | 3462 |
| Osteoblasts, heart | hsa-miR-186-5p | 3463 | 3464 |
| Osteogenic cells | hsa-miR-106a-3p | 3465 | 3466 |
| Osteogenic cells | hsa-miR-106a-5p | 3467 | 3468 |
| Osteogenic cells | hsa-miR-20b-3p | 3469 | 3470 |
| Osteogenic cells | hsa-miR-20b-5p | 3471 | 3472 |
| Ovary | hsa-miR-503-3p | 3473 | 3474 |
| Ovary | hsa-miR-503-5p | 3475 | 3476 |
| Ovary, female reproductive tract | hsa-miR-2114-3p | 3477 | 3478 |
| Ovary, female reproductive tract | hsa-miR-2114-5p | 3479 | 3480 |
| Ovary, lipid metabolism | hsa-miR-378a-3p | 3481 | 3482 |
| Ovary, placenta/trophoblast, lipid | hsa-miR-378a-5p | 3483 | 3484 |
| metabolism | | | |
| Pancreas (islet) | hsa-miR-375 | 3485 | 3486 |
| Pancreatic cells | hsa-miR-105-3p | 3487 | 3488 |
| Pancreatic cells | hsa-miR-105-5p | 3489 | 3490 |
| Pancreatic cells, endometrial tissues, | hsa-miR-196a-3p | 3491 | 3492 |
| mesenchymal stem cells | | | |
| Pancreatic cells, endometrial tissues, | hsa-miR-196a-5p | 3493 | 3494 |
| mesenchymal stem cells | | | |
| Pancreatic islet, lipid metabolism | hsa-miR-33a-3p | 3495 | 3496 |
| Pancreatic islet, lipid metabolism | hsa-miR-33a-5p | 3497 | 3498 |
| Periodontal tissue | hsa-miR-1260a | 3499 | 3500 |
| Periodontal tissue | hsa-miR-1260b | 3501 | 3502 |
| Peripheral blood | hsa-miR-3667-3p | 3503 | 3504 |
| Peripheral blood | hsa-miR-3667-5p | 3505 | 3506 |
| Peripheral blood | hsa-miR-3668 | 3507 | 3508 |
| Peripheral blood | hsa-miR-3669 | 3509 | 3510 |
| Peripheral blood | hsa-miR-3670 | 3511 | 3512 |
| Peripheral blood | hsa-miR-3671 | 3513 | 3514 |
| Peripheral blood | hsa-miR-3672 | 3515 | 3516 |
| Peripheral blood | hsa-miR-3673 | 3517 | 3518 |
| Peripheral blood | hsa-miR-3674 | 3519 | 3520 |
| Peripheral blood | hsa-miR-3675-3p | 3521 | 3522 |
| Peripheral blood | hsa-miR-3675-5p | 3523 | 3524 |
| Peripheral blood | hsa-miR-3676-3p | 3525 | 3526 |
| Peripheral blood | hsa-miR-3676-5p | 3527 | 3528 |
| Peripheral blood | hsa-miR-3677-3p | 3529 | 3530 |
| Peripheral blood | hsa-miR-3677-5p | 3531 | 3532 |
| Peripheral blood | hsa-miR-3678-3p | 3533 | 3534 |
| Peripheral blood | hsa-miR-3678-5p | 3535 | 3536 |
| Peripheral blood | hsa-miR-3679-3p | 3537 | 3538 |
| Peripheral blood | hsa-miR-3679-5p | 3539 | 3540 |
| Peripheral blood | hsa-miR-3680-3p | 3541 | 3542 |
| Peripheral blood | hsa-miR-3680-5p | 3543 | 3544 |
| Peripheral blood | hsa-miR-3681-3p | 3545 | 3546 |
| Peripheral blood | hsa-miR-3681-5p | 3547 | 3548 |
| Peripheral blood | hsa-miR-3682-3p | 3549 | 3550 |
| Peripheral blood | hsa-miR-3682-5p | 3551 | 3552 |
| Peripheral blood | hsa-miR-3683 | 3553 | 3554 |
| Peripheral blood | hsa-miR-3684 | 3555 | 3556 |
| Peripheral blood | hsa-miR-3685 | 3557 | 3558 |
| Peripheral blood | hsa-miR-3686 | 3559 | 3560 |
| Peripheral blood | hsa-miR-3687 | 3561 | 3562 |
| Peripheral blood | hsa-miR-3690 | 3563 | 3564 |
| Peripheral blood | hsa-miR-3691-3p | 3565 | 3566 |
| Peripheral blood | hsa-miR-3691-5p | 3567 | 3568 |
| Peripheral blood | hsa-miR-3692-3p | 3569 | 3570 |
| Peripheral blood | hsa-miR-3692-5p | 3571 | 3572 |
| Placenta | hsa-miR-1182 | 3573 | 3574 |
| Placenta | hsa-miR-1185-1-3p | 3575 | 3576 |
| Placenta | hsa-miR-1185-2-3p | 3577 | 3578 |
| Placenta | hsa-miR-1185-5p | 3579 | 3580 |
| Placenta | hsa-miR-1283 | 3581 | 3582 |
| Placenta | hsa-miR-1323 | 3583 | 3584 |
| Placenta | hsa-miR-515-5p | 3585 | 3586 |
| Placenta | hsa-miR-516a-5p | 3587 | 3588 |
| Placenta | hsa-miR-517-5p | 3589 | 3590 |
| Placenta | hsa-miR-517a-3p | 3591 | 3592 |
| Placenta | hsa-miR-517b-3p | 3593 | 3594 |
| Placenta | hsa-miR-517c-3p | 3595 | 3596 |
| Placenta | hsa-miR-518b | 3597 | 3598 |
| Placenta | hsa-miR-518c-3p | 3599 | 3600 |
| Placenta | hsa-miR-518c-5p | 3601 | 3602 |
| Placenta | hsa-miR-518f-3p | 3603 | 3604 |
| Placenta | hsa-miR-518f-5p | 3605 | 3606 |
| Placenta | hsa-miR-519a-3p | 3607 | 3608 |
| Placenta | hsa-miR-519a-5p | 3609 | 3610 |
| Placenta | hsa-miR-519d | 3611 | 3612 |
| Placenta | hsa-miR-519e-3p | 3613 | 3614 |
| Placenta | hsa-miR-519e-5p | 3615 | 3616 |
| Placenta | hsa-miR-520a-3p | 3617 | 3618 |
| Placenta | hsa-miR-520a-5p | 3619 | 3620 |
| Placental specific | hsa-miR-520h | 3621 | 3622 |
| Placental specific | hsa-miR-524-5p | 3623 | 3624 |
| Placental specific | hsa-miR-525-3p | 3625 | 3626 |
| Placental specific | hsa-miR-525-5p | 3627 | 3628 |
| Placental specific | hsa-miR-526a | 3629 | 3630 |
| Placental specific | hsa-miR-526b-3p | 3631 | 3632 |
| Placental specific | hsa-miR-526b-5p | 3633 | 3634 |
| Plasma | hsa-miR-422a | 3635 | 3636 |
| Platelet | hsa-miR-495-3p | 3637 | 3638 |
| Platelet | hsa-miR-495-5p | 3639 | 3640 |
| Renal epithelial cells | hsa-miR-382-3p | 3641 | 3642 |
| Renal epithelial cells | hsa-miR-382-5p | 3643 | 3644 |
| Reproductive tracts | hsa-miR-3605-3p | 3645 | 3646 |
| Reproductive tracts | hsa-miR-3605-5p | 3647 | 3648 |
| Salivary gland | hsa-miR-5100 | 3649 | 3650 |
| Salivary gland | hsa-miR-5571-3p | 3651 | 3652 |
| Salivary gland | hsa-miR-5571-5p | 3653 | 3654 |
| Salivary gland | hsa-miR-5572 | 3655 | 3656 |
| Sarcoma | hsa-miR-1180 | 3657 | 3658 |
| Semen | hsa-miR-574-5p | 3659 | 3660 |
| Serum | hsa-miR-1233-1-5p | 3661 | 3662 |
| Serum | hsa-miR-1233-3p | 3663 | 3664 |
| Serum | hsa-miR-371a-3p | 3665 | 3666 |
| Serum | hsa-miR-371a-5p | 3667 | 3668 |
| Serum | hsa-miR-371b-3p | 3669 | 3670 |
| Serum | hsa-miR-371b-5p | 3671 | 3672 |
| Serum | hsa-miR-649 | 3673 | 3674 |
| Skin (epithelium) | hsa-miR-936 | 3675 | 3676 |
| Skin (epithelium) | hsa-miR-203a | 3677 | 3678 |
| Skin (epithelium) | hsa-miR-203b-3p | 3679 | 3680 |
| Skin (epithelium) | hsa-miR-203b-5p | 3681 | 3682 |
| Smooth muscle | hsa-miR-1286 | 3683 | 3684 |
| Smooth muscle, central nervous system | hsa-miR-188-3p | 3685 | 3686 |
| Smooth muscle, central nervous system | hsa-miR-188-5p | 3687 | 3688 |
| Solid tumor | hsa-miR-3646 | 3689 | 3690 |
| Solid tumor | hsa-miR-3648 | 3691 | 3692 |
| Solid tumor | hsa-miR-3649 | 3693 | 3694 |
| Solid tumor | hsa-miR-3650 | 3695 | 3696 |
| Solid tumor | hsa-miR-3651 | 3697 | 3698 |
| Solid tumor | hsa-miR-3652 | 3699 | 3700 |
| Solid tumor | hsa-miR-3653 | 3701 | 3702 |
| Solid tumor | hsa-miR-3654 | 3703 | 3704 |
| Solid tumor | hsa-miR-3655 | 3705 | 3706 |
| Solid tumor | hsa-miR-3656 | 3707 | 3708 |
| Solid tumor | hsa-miR-3657 | 3709 | 3710 |
| Solid tumor | hsa-miR-3658 | 3711 | 3712 |
| Stem cells (adipose) | hsa-miR-138-2-3p | 3713 | 3714 |
| Stem cells (adipose) | hsa-miR-138-5p | 3715 | 3716 |
| Stem cells (adipose) | hsa-miR-369-3p | 3717 | 3718 |
| Stem cells (adipose) | hsa-miR-369-5p | 3719 | 3720 |
| Stem cells (adipose) | hsa-miR-96-3p | 3721 | 3722 |
| Stem cells (adipose) | hsa-miR-96-5p | 3723 | 3724 |
| Stem cells (adipose) | hsa-miR-486-5p | 3725 | 3726 |
| Stem cells, epidermal cells (keratinocytes) | hsa-miR-138-1-3p | 3727 | 3728 |
| Stem cells, placenta | hsa-miR-136-3p | 3729 | 3730 |
| Stem cells, placenta | hsa-miR-136-5p | 3731 | 3732 |
| T/B cells, monocytes, breast | hsa-miR-155-3p | 3733 | 3734 |
| T/B cells, monocytes, breast | hsa-miR-155-5p | 3735 | 3736 |
| Testes, brain (medulla) | hsa-miR-383 | 3737 | 3738 |
| Testis | hsa-miR-509-3-5p | 3739 | 3740 |
| T-Lymphocytes | hsa-miR-2909 | 3741 | 3742 |
| Trophoblast | hsa-miR-376c-3p | 3743 | 3744 |
| Trophoblast | hsa-miR-376c-5p | 3745 | 3746 |
| Variety of cells and tissues | hsa-miR-103b | 3747 | 3748 |
| Variety of cells and tissues | hsa-miR-141-3p | 3749 | 3750 |
| Variety of cells and tissues | hsa-miR-141-5p | 3751 | 3752 |
| Variety of cells and tissues | hsa-miR-193a-3p | 3753 | 3754 |
| Variety of cells and tissues | hsa-miR-193a-5p | 3755 | 3756 |
| Variety of cells and tissues | hsa-miR-215 | 3757 | 3758 |
| Variety of cells and tissues | hsa-miR-22-3p | 3759 | 3760 |
| Variety of cells and tissues | hsa-miR-22-5p | 3761 | 3762 |
| Variety of tissues and cells | hsa-miR-10b-3p | 3763 | 3764 |
| Variety of tissues and cells | hsa-miR-10b-5p | 3765 | 3766 |
| Variety of tissues, blood | hsa-miR-16-5p | 3767 | 3768 |
| Vascular smooth muscle | hsa-miR-143-3p | 3769 | 3770 |
| Vascular smooth muscle, T-cells | hsa-miR-143-5p | 3771 | 3772 |
| — | hsa-miR-1178-3p | 3773 | 3774 |
| — | hsa-miR-1178-5p | 3775 | 3776 |
| — | hsa-miR-1179 | 3777 | 3778 |
| — | hsa-miR-1181 | 3779 | 3780 |
| — | hsa-miR-1183 | 3781 | 3782 |
| — | hsa-miR-1193 | 3783 | 3784 |
| — | hsa-miR-1197 | 3785 | 3786 |
| — | hsa-miR-1200 | 3787 | 3788 |
| — | hsa-miR-1202 | 3789 | 3790 |
| — | hsa-miR-1203 | 3791 | 3792 |
| — | hsa-miR-1204 | 3793 | 3794 |
| — | hsa-miR-1205 | 3795 | 3796 |
| — | hsa-miR-1206 | 3797 | 3798 |
| — | hsa-miR-1207-3p | 3799 | 3800 |
| — | hsa-miR-1207-5p | 3801 | 3802 |
| — | hsa-miR-1208 | 3803 | 3804 |
| — | hsa-miR-1224-3p | 3805 | 3806 |
| — | hsa-miR-1224-5p | 3807 | 3808 |
| — | hsa-miR-1225-3p | 3809 | 3810 |
| — | hsa-miR-1225-5p | 3811 | 3812 |
| — | hsa-miR-1226-3p | 3813 | 3814 |
| — | hsa-miR-1226-5p | 3815 | 3816 |
| — | hsa-miR-1229-3p | 3817 | 3818 |
| — | hsa-miR-1229-5p | 3819 | 3820 |
| — | hsa-miR-1231 | 3821 | 3822 |
| — | hsa-miR-1238-3p | 3823 | 3824 |
| — | hsa-miR-1238-5p | 3825 | 3826 |
| — | hsa-miR-1248 | 3827 | 3828 |
| — | hsa-miR-1273c | 3829 | 3830 |
| — | hsa-miR-1273e | 3831 | 3832 |
| — | hsa-miR-1273f | 3833 | 3834 |
| — | hsa-miR-1273g-3p | 3835 | 3836 |
| — | hsa-miR-1273g-5p | 3837 | 3838 |
| — | hsa-miR-1281 | 3839 | 3840 |
| — | hsa-miR-1284 | 3841 | 3842 |
| — | hsa-miR-1285-3p | 3843 | 3844 |
| — | hsa-miR-1285-5p | 3845 | 3846 |
| — | hsa-miR-1292-3p | 3847 | 3848 |
| — | hsa-miR-1292-5p | 3849 | 3850 |
| — | hsa-miR-1295a | 3851 | 3852 |
| — | hsa-miR-1295b-3p | 3853 | 3854 |
| — | hsa-miR-1295b-5p | 3855 | 3856 |
| — | hsa-miR-1296 | 3857 | 3858 |
| — | hsa-miR-1298 | 3859 | 3860 |
| — | hsa-miR-1301 | 3861 | 3862 |
| — | hsa-miR-1302 | 3863 | 3864 |
| — | hsa-miR-1304-3p | 3865 | 3866 |
| — | hsa-miR-1304-5p | 3867 | 3868 |
| — | hsa-miR-1321 | 3869 | 3870 |
| — | hsa-miR-1322 | 3871 | 3872 |
| — | hsa-miR-1324 | 3873 | 3874 |
| — | hsa-miR-1343 | 3875 | 3876 |
| — | hsa-miR-1468 | 3877 | 3878 |
| — | hsa-miR-1469 | 3879 | 3880 |
| — | hsa-miR-1470 | 3881 | 3882 |
| — | hsa-miR-1471 | 3883 | 3884 |
| — | hsa-miR-1537 | 3885 | 3886 |
| — | hsa-miR-1825 | 3887 | 3888 |
| — | hsa-miR-1827 | 3889 | 3890 |
| — | hsa-miR-185-3p | 3891 | 3892 |
| — | hsa-miR-185-5p | 3893 | 3894 |
| — | hsa-miR-187-3p | 3895 | 3896 |
| — | hsa-miR-187-5p | 3897 | 3898 |
| — | hsa-miR-1908 | 3899 | 3900 |
| — | hsa-miR-1909-3p | 3901 | 3902 |
| — | hsa-miR-1909-5p | 3903 | 3904 |
| — | hsa-miR-191-3p | 3905 | 3906 |
| — | hsa-miR-191-5p | 3907 | 3908 |
| — | hsa-miR-1972 | 3909 | 3910 |
| — | hsa-miR-1973 | 3911 | 3912 |
| — | hsa-miR-1976 | 3913 | 3914 |
| — | hsa-miR-2052 | 3915 | 3916 |
| — | hsa-miR-2053 | 3917 | 3918 |
| — | hsa-miR-2054 | 3919 | 3920 |
| — | hsa-miR-2110 | 3921 | 3922 |
| — | hsa-miR-2116-3p | 3923 | 3924 |
| — | hsa-miR-2116-5p | 3925 | 3926 |
| — | hsa-miR-2117 | 3927 | 3928 |
| — | hsa-miR-216b | 3929 | 3930 |
| — | hsa-miR-218-2-3p | 3931 | 3932 |
| — | hsa-miR-218-5p | 3933 | 3934 |
| — | hsa-miR-2276 | 3935 | 3936 |
| — | hsa-miR-2278 | 3937 | 3938 |
| — | hsa-miR-23c | 3939 | 3940 |
| — | hsa-miR-2467-3p | 3941 | 3942 |
| — | hsa-miR-2467-5p | 3943 | 3944 |
| — | hsa-miR-2681-3p | 3945 | 3946 |
| — | hsa-miR-2681-5p | 3947 | 3948 |
| — | hsa-miR-2682-3p | 3949 | 3950 |
| — | hsa-miR-2682-5p | 3951 | 3952 |
| — | hsa-miR-2964a-3p | 3953 | 3954 |
| — | hsa-miR-2964a-5p | 3955 | 3956 |
| — | hsa-miR-298 | 3957 | 3958 |
| — | hsa-miR-299-3p | 3959 | 3960 |
| — | hsa-miR-299-5p | 3961 | 3962 |
| — | hsa-miR-301b | 3963 | 3964 |
| — | hsa-miR-302f | 3965 | 3966 |
| — | hsa-miR-3064-3p | 3967 | 3968 |
| — | hsa-miR-3064-5p | 3969 | 3970 |
| — | hsa-miR-3074-3p | 3971 | 3972 |
| — | hsa-miR-3074-5p | 3973 | 3974 |
| — | hsa-miR-3115 | 3975 | 3976 |
| — | hsa-miR-31-3p | 3977 | 3978 |
| — | hsa-miR-31-5p | 3979 | 3980 |
| — | hsa-miR-3196 | 3981 | 3982 |
| — | hsa-miR-320d | 3983 | 3984 |
| — | hsa-miR-323b-3p | 3985 | 3986 |
| — | hsa-miR-323b-5p | 3987 | 3988 |
| — | hsa-miR-330-3p | 3989 | 3990 |
| — | hsa-miR-330-5p | 3991 | 3992 |
| — | hsa-miR-331-3p | 3993 | 3994 |
| — | hsa-miR-340-3p | 3995 | 3996 |
| — | hsa-miR-362-3p | 3997 | 3998 |
| — | hsa-miR-362-5p | 3999 | 4000 |
| — | hsa-miR-365a-3p | 4001 | 4002 |
| — | hsa-miR-365a-5p | 4003 | 4004 |
| — | hsa-miR-365b-3p | 4005 | 4006 |
| — | hsa-miR-365b-5p | 4007 | 4008 |
| — | hsa-miR-3662 | 4009 | 4010 |
| — | hsa-miR-3663-3p | 4011 | 4012 |
| — | hsa-miR-3663-5p | 4013 | 4014 |
| — | hsa-miR-370 | 4015 | 4016 |
| — | hsa-miR-373-3p | 4017 | 4018 |
| — | hsa-miR-373-5p | 4019 | 4020 |
| — | hsa-miR-379-3p | 4021 | 4022 |
| — | hsa-miR-379-5p | 4023 | 4024 |
| — | hsa-miR-3935 | 4025 | 4026 |
| — | hsa-miR-3937 | 4027 | 4028 |
| — | hsa-miR-3938 | 4029 | 4030 |
| — | hsa-miR-3939 | 4031 | 4032 |
| — | hsa-miR-3941 | 4033 | 4034 |
| — | hsa-miR-3943 | 4035 | 4036 |
| — | hsa-miR-3945 | 4037 | 4038 |
| — | hsa-miR-409-3p | 4039 | 4040 |
| — | hsa-miR-409-5p | 4041 | 4042 |
| — | hsa-miR-411-3p | 4043 | 4044 |
| — | hsa-miR-411-5p | 4045 | 4046 |
| — | hsa-miR-412 | 4047 | 4048 |
| — | hsa-miR-4273 | 4049 | 4050 |
| — | hsa-miR-431-3p | 4051 | 4052 |
| — | hsa-miR-431-5p | 4053 | 4054 |
| — | hsa-miR-433 | 4055 | 4056 |
| — | hsa-miR-449c-3p | 4057 | 4058 |
| — | hsa-miR-449c-5p | 4059 | 4060 |
| — | hsa-miR-450a-3p | 4061 | 4062 |
| — | hsa-miR-450a-5p | 4063 | 4064 |
| — | hsa-miR-450b-3p | 4065 | 4066 |
| — | hsa-miR-450b-5p | 4067 | 4068 |
| — | hsa-miR-455-3p | 4069 | 4070 |
| — | hsa-miR-455-5p | 4071 | 4072 |
| — | hsa-miR-466 | 4073 | 4074 |
| — | hsa-miR-4666b | 4075 | 4076 |
| — | hsa-miR-483-3p | 4077 | 4078 |
| — | hsa-miR-484 | 4079 | 4080 |
| — | hsa-miR-485-3p | 4081 | 4082 |
| — | hsa-miR-485-5p | 4083 | 4084 |
| — | hsa-miR-487a | 4085 | 4086 |
| — | hsa-miR-487b | 4087 | 4088 |
| — | hsa-miR-488-3p | 4089 | 4090 |
| — | hsa-miR-488-5p | 4091 | 4092 |
| — | hsa-miR-490-3p | 4093 | 4094 |
| — | hsa-miR-490-5p | 4095 | 4096 |
| — | hsa-miR-491-3p | 4097 | 4098 |
| — | hsa-miR-491-5p | 4099 | 4100 |
| — | hsa-miR-492 | 4101 | 4102 |
| — | hsa-miR-497-3p | 4103 | 4104 |
| — | hsa-miR-497-5p | 4105 | 4106 |
| — | hsa-miR-498 | 4107 | 4108 |
| — | hsa-miR-4999-3p | 4109 | 4110 |
| — | hsa-miR-4999-5p | 4111 | 4112 |
| — | hsa-miR-5001-3p | 4113 | 4114 |
| — | hsa-miR-5001-5p | 4115 | 4116 |
| — | hsa-miR-5002-3p | 4117 | 4118 |
| — | hsa-miR-5002-5p | 4119 | 4120 |
| — | hsa-miR-5003-3p | 4121 | 4122 |
| — | hsa-miR-5003-5p | 4123 | 4124 |
| — | hsa-miR-5004-3p | 4125 | 4126 |
| — | hsa-miR-5004-5p | 4127 | 4128 |
| — | hsa-miR-5007-3p | 4129 | 4130 |
| — | hsa-miR-5007-5p | 4131 | 4132 |
| — | hsa-miR-5008-3p | 4133 | 4134 |
| — | hsa-miR-5008-5p | 4135 | 4136 |
| — | hsa-miR-5009-3p | 4137 | 4138 |
| — | hsa-miR-5009-5p | 4139 | 4140 |
| — | hsa-miR-500a-3p | 4141 | 4142 |
| — | hsa-miR-500a-5p | 4143 | 4144 |
| — | hsa-miR-5010-3p | 4145 | 4146 |
| — | hsa-miR-5010-5p | 4147 | 4148 |
| — | hsa-miR-5011-3p | 4149 | 4150 |
| — | hsa-miR-5011-5p | 4151 | 4152 |
| — | hsa-miR-501-3p | 4153 | 4154 |
| — | hsa-miR-501-5p | 4155 | 4156 |
| — | hsa-miR-502-3p | 4157 | 4158 |
| — | hsa-miR-502-5p | 4159 | 4160 |
| — | hsa-miR-504 | 4161 | 4162 |
| — | hsa-miR-5047 | 4163 | 4164 |
| — | hsa-miR-505-3p | 4165 | 4166 |
| — | hsa-miR-505-5p | 4167 | 4168 |
| — | hsa-miR-506-3p | 4169 | 4170 |
| — | hsa-miR-506-5p | 4171 | 4172 |
| — | hsa-miR-507 | 4173 | 4174 |
| — | hsa-miR-508-3p | 4175 | 4176 |
| — | hsa-miR-5087 | 4177 | 4178 |
| — | hsa-miR-5088 | 4179 | 4180 |
| — | hsa-miR-5089-3p | 4181 | 4182 |
| — | hsa-miR-5089-5p | 4183 | 4184 |
| — | hsa-miR-5090 | 4185 | 4186 |
| — | hsa-miR-5091 | 4187 | 4188 |
| — | hsa-miR-5092 | 4189 | 4190 |
| — | hsa-miR-5093 | 4191 | 4192 |
| — | hsa-miR-509-3p | 4193 | 4194 |
| — | hsa-miR-5094 | 4195 | 4196 |
| — | hsa-miR-5095 | 4197 | 4198 |
| — | hsa-miR-509-5p | 4199 | 4200 |
| — | hsa-miR-5096 | 4201 | 4202 |
| — | hsa-miR-513a-3p | 4203 | 4204 |
| — | hsa-miR-513b | 4205 | 4206 |
| — | hsa-miR-513c-3p | 4207 | 4208 |
| — | hsa-miR-513c-5p | 4209 | 4210 |
| — | hsa-miR-514a-3p | 4211 | 4212 |
| — | hsa-miR-514a-5p | 4213 | 4214 |
| — | hsa-miR-514b-3p | 4215 | 4216 |
| — | hsa-miR-514b-5p | 4217 | 4218 |
| — | hsa-miR-515-3p | 4219 | 4220 |
| — | hsa-miR-516b-3p | 4221 | 4222 |
| — | hsa-miR-516b-5p | 4223 | 4224 |
| — | hsa-miR-518a-3p | 4225 | 4226 |
| — | hsa-miR-518a-5p | 4227 | 4228 |
| — | hsa-miR-518d-3p | 4229 | 4230 |
| — | hsa-miR-518d-5p | 4231 | 4232 |
| — | hsa-miR-518e-3p | 4233 | 4234 |
| — | hsa-miR-518e-5p | 4235 | 4236 |
| — | hsa-miR-519b-3p | 4237 | 4238 |
| — | hsa-miR-519b-5p | 4239 | 4240 |
| — | hsa-miR-519c-3p | 4241 | 4242 |
| — | hsa-miR-519c-5p | 4243 | 4244 |
| — | hsa-miR-520b | 4245 | 4246 |
| — | hsa-miR-520c-3p | 4247 | 4248 |
| — | hsa-miR-520c-5p | 4249 | 4250 |
| — | hsa-miR-520d-3p | 4251 | 4252 |
| — | hsa-miR-520d-5p | 4253 | 4254 |
| — | hsa-miR-520e | 4255 | 4256 |
| — | hsa-miR-520f | 4257 | 4258 |
| — | hsa-miR-520g | 4259 | 4260 |
| — | hsa-miR-521 | 4261 | 4262 |
| — | hsa-miR-522-3p | 4263 | 4264 |
| — | hsa-miR-522-5p | 4265 | 4266 |
| — | hsa-miR-523-3p | 4267 | 4268 |
| — | hsa-miR-523-5p | 4269 | 4270 |
| — | hsa-miR-524-3p | 4271 | 4272 |
| — | hsa-miR-527 | 4273 | 4274 |
| — | hsa-miR-532-3p | 4275 | 4276 |
| — | hsa-miR-532-5p | 4277 | 4278 |
| — | hsa-miR-539-3p | 4279 | 4280 |
| — | hsa-miR-539-5p | 4281 | 4282 |
| — | hsa-miR-541-3p | 4283 | 4284 |
| — | hsa-miR-541-5p | 4285 | 4286 |
| — | hsa-miR-542-5p | 4287 | 4288 |
| — | hsa-miR-543 | 4289 | 4290 |
| — | hsa-miR-544a | 4291 | 4292 |
| — | hsa-miR-544b | 4293 | 4294 |
| — | hsa-miR-545-3p | 4295 | 4296 |
| — | hsa-miR-545-5p | 4297 | 4298 |
| — | hsa-miR-548 | 4299 | 4300 |
| — | hsa-miR-548-3p | 4301 | 4302 |
| — | hsa-miR-548-5p | 4303 | 4304 |
| — | hsa-miR-548ao-3p | 4305 | 4306 |
| — | hsa-miR-548ao-5p | 4307 | 4308 |
| — | hsa-miR-548ap-3p | 4309 | 4310 |
| — | hsa-miR-548ap-5p | 4311 | 4312 |
| — | hsa-miR-548aq-3p | 4313 | 4314 |
| — | hsa-miR-548aq-5p | 4315 | 4316 |
| — | hsa-miR-548ar-3p | 4317 | 4318 |
| — | hsa-miR-548ar-5p | 4319 | 4320 |
| — | hsa-miR-548as-3p | 4321 | 4322 |
| — | hsa-miR-548as-5p | 4323 | 4324 |
| — | hsa-miR-548at-3p | 4325 | 4326 |
| — | hsa-miR-548at-5p | 4327 | 4328 |
| — | hsa-miR-548au-3p | 4329 | 4330 |
| — | hsa-miR-548au-5p | 4331 | 4332 |
| — | hsa-miR-548av-3p | 4333 | 4334 |
| — | hsa-miR-548av-5p | 4335 | 4336 |
| — | hsa-miR-548aw | 4337 | 4338 |
| — | hsa-miR-548q | 4339 | 4340 |
| — | hsa-miR-548y | 4341 | 4342 |
| — | hsa-miR-550a-3-5p | 4343 | 4344 |
| — | hsa-miR-550a-3p | 4345 | 4346 |
| — | hsa-miR-550a-5p | 4347 | 4348 |
| — | hsa-miR-551a | 4349 | 4350 |
| — | hsa-miR-5579-3p | 4351 | 4352 |
| — | hsa-miR-5579-5p | 4353 | 4354 |
| — | hsa-miR-558 | 4355 | 4356 |
| — | hsa-miR-5580-3p | 4357 | 4358 |
| — | hsa-miR-5580-5p | 4359 | 4360 |
| — | hsa-miR-5581-3p | 4361 | 4362 |
| — | hsa-miR-5581-5p | 4363 | 4364 |
| — | hsa-miR-5582-3p | 4365 | 4366 |
| — | hsa-miR-5582-5p | 4367 | 4368 |
| — | hsa-miR-5583-3p | 4369 | 4370 |
| — | hsa-miR-5583-5p | 4371 | 4372 |
| — | hsa-miR-5584-3p | 4373 | 4374 |
| — | hsa-miR-5584-5p | 4375 | 4376 |
| — | hsa-miR-5585-3p | 4377 | 4378 |
| — | hsa-miR-5585-5p | 4379 | 4380 |
| — | hsa-miR-5586-3p | 4381 | 4382 |
| — | hsa-miR-5586-5p | 4383 | 4384 |
| — | hsa-miR-5587-3p | 4385 | 4386 |
| — | hsa-miR-5587-5p | 4387 | 4388 |
| — | hsa-miR-5588-3p | 4389 | 4390 |
| — | hsa-miR-5588-5p | 4391 | 4392 |
| — | hsa-miR-5589-3p | 4393 | 4394 |
| — | hsa-miR-5589-5p | 4395 | 4396 |
| — | hsa-miR-559 | 4397 | 4398 |
| — | hsa-miR-5590-3p | 4399 | 4400 |
| — | hsa-miR-5590-5p | 4401 | 4402 |
| — | hsa-miR-5591-3p | 4403 | 4404 |
| — | hsa-miR-5591-5p | 4405 | 4406 |
| — | hsa-miR-561-3p | 4407 | 4408 |
| — | hsa-miR-561-5p | 4409 | 4410 |
| — | hsa-miR-562 | 4411 | 4412 |
| — | hsa-miR-564 | 4413 | 4414 |
| — | hsa-miR-566 | 4415 | 4416 |
| — | hsa-miR-567 | 4417 | 4418 |
| — | hsa-miR-5680 | 4419 | 4420 |
| — | hsa-miR-5681a | 4421 | 4422 |
| — | hsa-miR-5681b | 4423 | 4424 |
| — | hsa-miR-5682 | 4425 | 4426 |
| — | hsa-miR-5683 | 4427 | 4428 |
| — | hsa-miR-5684 | 4429 | 4430 |
| — | hsa-miR-5685 | 4431 | 4432 |
| — | hsa-miR-5686 | 4433 | 4434 |
| — | hsa-miR-5687 | 4435 | 4436 |
| — | hsa-miR-5688 | 4437 | 4438 |
| — | hsa-miR-5689 | 4439 | 4440 |
| — | hsa-miR-569 | 4441 | 4442 |
| — | hsa-miR-5690 | 4443 | 4444 |
| — | hsa-miR-5691 | 4445 | 4446 |
| — | hsa-miR-5692a | 4447 | 4448 |
| — | hsa-miR-5692b | 4449 | 4450 |
| — | hsa-miR-5692c | 4451 | 4452 |
| — | hsa-miR-5693 | 4453 | 4454 |
| — | hsa-miR-5694 | 4455 | 4456 |
| — | hsa-miR-5695 | 4457 | 4458 |
| — | hsa-miR-5696 | 4459 | 4460 |
| — | hsa-miR-5697 | 4461 | 4462 |
| — | hsa-miR-5698 | 4463 | 4464 |
| — | hsa-miR-5699 | 4465 | 4466 |
| — | hsa-miR-5700 | 4467 | 4468 |
| — | hsa-miR-5701 | 4469 | 4470 |
| — | hsa-miR-5702 | 4471 | 4472 |
| — | hsa-miR-5703 | 4473 | 4474 |
| — | hsa-miR-570-3p | 4475 | 4476 |
| — | hsa-miR-5704 | 4477 | 4478 |
| — | hsa-miR-5705 | 4479 | 4480 |
| — | hsa-miR-570-5p | 4481 | 4482 |
| — | hsa-miR-5706 | 4483 | 4484 |
| — | hsa-miR-5707 | 4485 | 4486 |
| — | hsa-miR-5708 | 4487 | 4488 |
| — | hsa-miR-575 | 4489 | 4490 |
| — | hsa-miR-579 | 4491 | 4492 |
| — | hsa-miR-580 | 4493 | 4494 |
| — | hsa-miR-582-5p | 4495 | 4496 |
| — | hsa-miR-583 | 4497 | 4498 |
| — | hsa-miR-584-3p | 4499 | 4500 |
| — | hsa-miR-584-5p | 4501 | 4502 |
| — | hsa-miR-585 | 4503 | 4504 |
| — | hsa-miR-591 | 4505 | 4506 |
| — | hsa-miR-592 | 4507 | 4508 |
| — | hsa-miR-593-3p | 4509 | 4510 |
| — | hsa-miR-593-5p | 4511 | 4512 |
| — | hsa-miR-595 | 4513 | 4514 |
| — | hsa-miR-596 | 4515 | 4516 |
| — | hsa-miR-599 | 4517 | 4518 |
| — | hsa-miR-601 | 4519 | 4520 |
| — | hsa-miR-603 | 4521 | 4522 |
| — | hsa-miR-608 | 4523 | 4524 |
| — | hsa-miR-610 | 4525 | 4526 |
| — | hsa-miR-611 | 4527 | 4528 |
| — | hsa-miR-612 | 4529 | 4530 |
| — | hsa-miR-6124 | 4531 | 4532 |
| — | hsa-miR-6125 | 4533 | 4534 |
| — | hsa-miR-6126 | 4535 | 4536 |
| — | hsa-miR-6127 | 4537 | 4538 |
| — | hsa-miR-6128 | 4539 | 4540 |
| — | hsa-miR-6129 | 4541 | 4542 |
| — | hsa-miR-6130 | 4543 | 4544 |
| — | hsa-miR-6131 | 4545 | 4546 |
| — | hsa-miR-6132 | 4547 | 4548 |
| — | hsa-miR-6133 | 4549 | 4550 |
| — | hsa-miR-6134 | 4551 | 4552 |
| — | hsa-miR-615-3p | 4553 | 4554 |
| — | hsa-miR-615-5p | 4555 | 4556 |
| — | hsa-miR-616-3p | 4557 | 4558 |
| — | hsa-miR-6165 | 4559 | 4560 |
| — | hsa-miR-616-5p | 4561 | 4562 |
| — | hsa-miR-617 | 4563 | 4564 |
| — | hsa-miR-618 | 4565 | 4566 |
| — | hsa-miR-621 | 4567 | 4568 |
| — | hsa-miR-622 | 4569 | 4570 |
| — | hsa-miR-623 | 4571 | 4572 |
| — | hsa-miR-627 | 4573 | 4574 |
| — | hsa-miR-628-3p | 4575 | 4576 |
| — | hsa-miR-628-5p | 4577 | 4578 |
| — | hsa-miR-629-3p | 4579 | 4580 |
| — | hsa-miR-629-5p | 4581 | 4582 |
| — | hsa-miR-632 | 4583 | 4584 |
| — | hsa-miR-633 | 4585 | 4586 |
| — | hsa-miR-636 | 4587 | 4588 |
| — | hsa-miR-638 | 4589 | 4590 |
| — | hsa-miR-640 | 4591 | 4592 |
| — | hsa-miR-644a | 4593 | 4594 |
| — | hsa-miR-645 | 4595 | 4596 |
| — | hsa-miR-646 | 4597 | 4598 |
| — | hsa-miR-647 | 4599 | 4600 |
| — | hsa-miR-650 | 4601 | 4602 |
| — | hsa-miR-652-3p | 4603 | 4604 |
| — | hsa-miR-652-5p | 4605 | 4606 |
| — | hsa-miR-655 | 4607 | 4608 |
| — | hsa-miR-656 | 4609 | 4610 |
| — | hsa-miR-658 | 4611 | 4612 |
| — | hsa-miR-661 | 4613 | 4614 |
| — | hsa-miR-663a | 4615 | 4616 |
| — | hsa-miR-663b | 4617 | 4618 |
| — | hsa-miR-665 | 4619 | 4620 |
| — | hsa-miR-670 | 4621 | 4622 |
| — | hsa-miR-671-3p | 4623 | 4624 |
| — | hsa-miR-671-5p | 4625 | 4626 |
| — | hsa-miR-675-3p | 4627 | 4628 |
| — | hsa-miR-675-5p | 4629 | 4630 |
| — | hsa-miR-708-3p | 4631 | 4632 |
| — | hsa-miR-708-5p | 4633 | 4634 |
| — | hsa-miR-711 | 4635 | 4636 |
| — | hsa-miR-759 | 4637 | 4638 |
| — | hsa-miR-760 | 4639 | 4640 |
| — | hsa-miR-761 | 4641 | 4642 |
| — | hsa-miR-765 | 4643 | 4644 |
| — | hsa-miR-767-3p | 4645 | 4646 |
| — | hsa-miR-767-5p | 4647 | 4648 |
| — | hsa-miR-769-3p | 4649 | 4650 |
| — | hsa-miR-769-5p | 4651 | 4652 |
| — | hsa-miR-770-5p | 4653 | 4654 |
| — | hsa-miR-873-3p | 4655 | 4656 |
| — | hsa-miR-873-5p | 4657 | 4658 |
| — | hsa-miR-874 | 4659 | 4660 |
| — | hsa-miR-875-3p | 4661 | 4662 |
| — | hsa-miR-875-5p | 4663 | 4664 |
| — | hsa-miR-876-3p | 4665 | 4666 |
| — | hsa-miR-876-5p | 4667 | 4668 |
| — | hsa-miR-877-3p | 4669 | 4670 |
| — | hsa-miR-877-5p | 4671 | 4672 |
| — | hsa-miR-887 | 4673 | 4674 |
| — | hsa-miR-888-3p | 4675 | 4676 |
| — | hsa-miR-888-5p | 4677 | 4678 |
| — | hsa-miR-889 | 4679 | 4680 |
| — | hsa-miR-937-3p | 4681 | 4682 |
| — | hsa-miR-937-5p | 4683 | 4684 |
| — | hsa-miR-938 | 4685 | 4686 |
| — | hsa-miR-942 | 4687 | 4688 |
| — | hsa-miR-944 | 4689 | 4690 |
| — | hsa-miR-95 | 4691 | 4692 |
| — | hsa-miR-98-3p | 4693 | 4694 |
| — | hsa-miR-98-5p | 4695 | 4696 |

### Human Cells

For ameliorating Wiskott-Aldrich Syndrome (WAS) or any disorder associated with WAS gene, as described and illustrated herein, the principal targets for gene editing are human cells. For example, in the ex vivo methods, the human cells can be somatic cells, which after being modified using the techniques as described, can give rise to differentiated cells, e.g., hepatocytes or progenitor cells. For example, in the in vivo methods, the human cells may be hepatocytes, renal cells or cells from other affected organs. In some aspects, the human cells that are edited are autologous. In other aspects, the human cells that are edited are non-autologous (e.g., allogeneic).

By performing gene editing in autologous cells that are derived from and therefore already completely matched with the patient in need, it is possible to generate cells that can be safely re-introduced into the patient, and effectively give rise to a population of cells that will be effective in ameliorating one or more clinical conditions associated with the patient's disease.

Progenitor cells (also referred to as stem cells herein) are capable of both proliferation and giving rise to more progenitor cells, these in turn having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell that itself is derived from a multipotent cell, and so on. While each of these multipotent cells may be considered stem cells, the range of cell types that each can give rise to may vary considerably. Some differentiated cells also have the capacity to give rise to cells of greater developmental potential. Such capacity may be natural or may be induced artificially upon treatment with various factors. In many biological instances, stem cells can also be “multipotent” because they can produce progeny of more than one distinct cell type, but this is not required for “stem-ness.”

Self-renewal can be another important aspect of the stem cell. In theory, self-renewal can occur by either of two major mechanisms. Stem cells can divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated,” or “differentiating” is a relative term. A “differentiated cell” is a cell that has progressed further down the developmental pathway than the cell to which it is being compared. Thus, stem cells can differentiate into lineage-restricted precursor cells (such as a myocyte progenitor cell), which in turn can differentiate into other types of precursor cells further down the pathway (such as a myocyte precursor), and then to an end-stage differentiated cell, such as a myocyte, which plays a characteristic role in a certain tissue type, and may or may not retain the capacity to proliferate further.

The term “hematopoietic progenitor cell” refers to cells of a stem cell lineage that give rise to all the blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells).

In some embodiments, the hematopoietic progenitor cell expresses at least one of the following cell surface markers characteristic of hematopoietic progenitor cells: CD34+, CD59+, Thy1/CD90+, CD381o/−, and C-kit/CD1 17+. In some embodiments, the hematopoietic progenitors are CD34+.

In some embodiments, the hematopoietic progenitor cell is a peripheral blood stem cell obtained from the patient after the patient has been treated with one or more factors such as granulocyte colony stimulating factor (optionally in combination with Plerixaflor). In illustrative embodiments, CD34+ cells are enriched using CliniMACS® Cell Selection System (Miltenyi Biotec). In some embodiments, CD34+ cells are stimulated in serum-free medium (e.g., CellGrow SCGM media, CellGenix) with cytokines (e.g., SCF, rhTPO, rhFLT3) before genome editing. In some embodiments, addition of SRI and dmPGE2 and/or other factors is contemplated to improve long-term engraftment.

Hematopoietic stem cells (HSCs) are an important target for gene therapy as they provide a prolonged source of the corrected cells. HSCs give rise to both the myeloid and lymphoid lineages of blood cells. Mature blood cells have a finite life-span and must be continuously replaced throughout life. Blood cells are continually produced by the proliferation and differentiation of a population of pluripotent HSCs that can replenished by self-renewal. Bone marrow (BM) is the major site of hematopoiesis in humans and a good source for hematopoietic stem and progenitor cells (HSPCs). HSPCs can be found in small numbers in the peripheral blood (PB). In some indications or treatments their numbers increase. The progeny of HSCs mature through stages, generating multi-potential and lineage-committed progenitor cells including the lymphoid progenitor cells giving rise to the cells expressing WAS. Certain progenitor cells, e.g. B and T cells, could be edited at the stages prior to re-arrangement, though correcting progenitors has the advantage of continuing to be a source of corrected cells. Treated cells, such as CD34+ cells would be returned to the patient. The level of engraftment is important, as is the ability of the cells' multilineage engraftment of gene-edited cells following CD34+ infusion in vivo. In some aspects, the HSCs that can be used are autologous. In other aspects, the HSCs that can be used are allogeneic or non-autologous.

### Induced Pluripotent Stem Cells

The genetically engineered human cells described herein can be induced pluripotent stem cells (iPSCs). An advantage of using iPSCs is that the cells can be derived from the same subject to which the progenitor cells are to be administered. That is, a somatic cell can be obtained from a subject, reprogrammed to an induced pluripotent stem cell, and then re-differentiated into a progenitor cell to be administered to the subject (e.g., autologous cells). Because the progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Although differentiation is generally irreversible under physiological contexts, several methods have been recently developed to reprogram somatic cells to iPSCs. Exemplary methods are known to those of skill in the art and are described briefly herein below.

The term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Reprogramming can encompass complete reversion of the differentiation state of a differentiated cell (e.g., a somatic cell) to a pluripotent state or a multipotent state. Reprogramming can encompass complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain examples described herein, reprogramming of a differentiated cell (e.g., a somatic cell) can cause the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a myogenic stem cell). Reprogramming is also distinct from promoting the self-renewal or proliferation of cells that are already pluripotent or multipotent, although the compositions and methods described herein can also be of use for such purposes, in some examples.

Many methods are known in the art that can be used to generate pluripotent stem cells from somatic cells. Any such method that reprograms a somatic cell to the pluripotent phenotype would be appropriate for use in the methods described herein.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, Cell 126(4): 663-76 (2006). iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission [see, e.g., Maherali and Hochedlinger. Cell Stem Cell. 3(6):595-605 (2008)], and tetraploid complementation.

Human iPSCs can be obtained using similar transduction methods, and the transcription factor trio, OCT4, SOX2, and NANOG, has been established as the core set of transcription factors that govern pluripotency; see, e.g., Budniatzky and Gepstein, Stem Cells Transl Med. 3(4):448-57 (2014); Barrett et al., Stem Cells Trans Med 3:1-6 sctm.2014-0121 (2014); Focosi et al., Blood Cancer Journal 4; e211 (2014); and references cited therein. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 7(5):618-30 (2010). Reprogramming can be achieved by introducing a combination of nucleic acids encoding stem cell-associated genes, including, for example, Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. Reprogramming using the methods and compositions described herein can further have introducing one or more of Oct-3/4, a member of the Sox family, a member of the Klf family, and a member of the Myc family to a somatic cell. The methods and compositions described herein can further have introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not effected by a method that alters the genome. Thus, in such examples, reprogramming can be achieved, e.g., without the use of viral or plasmid vectors.

The efficiency of reprogramming (i.e., the number of reprogrammed cells) derived from a population of starting cells can be enhanced by the addition of various agents, e.g., small molecules, as shown by Shi et al., Cell-Stem Cell 2:525-528 (2008); Huangfu et al., Nature Biotechnology 26(7):795-797 (2008) and Marson et al., Cell-Stem Cell 3: 132-135 (2008). Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetlase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide). Phenylbutyrate (e.g., sodium phenylbutyrate) and Valproic Acid ((VP A) and other short chain fatty acids). Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9, 10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences. Novartis, Gloucester Pharmaceuticals, Titan Pharmaceuticals, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utfl, and Natl. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but can also include detection of protein markers. Intracellular markers may be best identified via RT-PCR, or protein detection methods such as immunocytochemistry, while cell surface markers are readily identified, e.g., by immunocytochemistry.

The pluripotent stem cell character of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor having cells from all three germ layers, for example, further indicates that the cells are pluripotent stem cells.

### Hepatocytes

In some embodiments, the genetically engineered human cells described herein are hepatocytes. A hepatocyte is a cell of the main parenchymal tissue of the liver. Hepatocytes make up 70-85% of the liver's mass. These cells are involved in: protein synthesis; protein storage; transformation of carbohydrates; synthesis of cholesterol, bile salts and phospholipids; detoxification, modification, and excretion of exogenous and endogenous substances; and initiation of formation and secretion of bile.

Creating Patient Specific iPSCs

One step of the ex vivo methods of the present disclosure can involve creating a patient specific iPS cell, patient specific iPS cells, or a patient specific iPS cell line. There are many established methods in the art for creating patient specific iPS cells, as described in Takahashi and Yamanaka 2006; Takahashi, Tanabe et al. 2007. For example, the creating step can have: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

### Performing a Biopsy or Aspirate of the Patient's Liver or Bone Marrow

A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate may be performed according to any of the known methods in the art. For example, in a biopsy, a needle is injected into the liver through the skin of the belly, capturing the liver tissue. For example, in a bone marrow aspirate, a large needle is used to enter the pelvis bone to collect bone marrow.

### Isolating a Liver Specific Progenitor Cell or Primary Hepatocyte

Liver specific progenitor cells and primary hepatocytes may be isolated according to any method known in the art. For example, human hepatocytes are isolated from fresh surgical specimens. Healthy liver tissue is used to isolate hepatocytes by collagenase digestion. The obtained cell suspension is filtered through a 100-mm nylon mesh and sedimented by centrifugation at 50 g for 5 minutes, resuspended, and washed two to three times in cold wash medium. Human liver stem cells are obtained by culturing under stringent conditions of hepatocytes obtained from fresh liver preparations. Hepatocytes seeded on collagen-coated plates are cultured for 2 weeks. After 2 weeks, surviving cells are removed, and characterized for expression of stem cells markers (Herrera et al., STEM CELLS 2006; 24: 2840-2850).

### Isolating a White Blood Cell

White blood cells may be isolated according to any method known in the art. For example, white blood cells can be isolated from a liquid sample by centrifugation and cell culturing. In some cases, white blood cells can be isolated from a whole blood sample by centrifugation through Ficoll.

### Isolating a Mesenchymal Stem Cell

Mesenchymal stem cells can be isolated according to any method known in the art, such as from a patient's bone marrow or peripheral blood. For example, marrow aspirate can be collected into a syringe with heparin. Cells can be washed and centrifuged on a Percoll. The cells can be cultured in Dulbecco's modified Eagle's medium (DMEM) (low glucose) containing 10% fetal bovine serum (FBS) (Pittinger M F, Mackay A M, Beck S C et al., Science 1999; 284:143-147).

Isolating a Hematopoietic Progenitor Cell from a Patient

A hematopoietic progenitor cell may be isolated from a patient by any method known in the art. CD34+ cells are enriched using CliniMACS® Cell Selection System (Miltenyi Biotec). In some embodiments, CD34+ cells are stimulated in serum-free medium (e.g., CellGrow SCGM media, CellGenix) with cytokines (e.g., SCF, rhTPO, rhFLT3) before genome editing.

### Site-Directed Polypeptides (Endonucleases, Enzymes)

A site-directed polypeptide is a nuclease used in genome editing to cleave DNA. The site-directed nuclease can be administered to a cell or a patient as either: one or more polypeptides, or one or more mRNAs encoding the polypeptide. Any of the enzymes or orthologs listed in Tables 1 or 2 or disclosed herein may be utilized in the methods herein.

In the context of a CRISPR/Cas or CRISPR/Cpf1 system, the site-directed polypeptide can bind to a guide RNA that, in turn, specifies the site in the target DNA to which the polypeptide is directed. In the CRISPR/Cas or CRISPR/Cpf1 systems disclosed herein, the site-directed polypeptide can be an endonuclease, such as a DNA endonuclease.

A site-directed polypeptide can have a plurality of nucleic acid-cleaving (i.e., nuclease) domains. Two or more nucleic acid-cleaving domains can be linked together via a linker. For example, the linker can have a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

Naturally-occurring wild-type Cas9 enzymes have two nuclease domains, a HNH nuclease domain and a RuvC domain. Herein, the “Cas9” refers to both naturally-occurring and recombinant Cas9s. Cas9 enzymes contemplated herein can have a HNH or HNH-like nuclease domain, and/or a RuvC or RuvC-like nuclease domain.

HNH or HNH-like domains have a McrA-like fold. HNH or HNH-like domains has two antiparallel β-strands and an α-helix. HNH or HNH-like domains has a metal binding site (e.g, a divalent cation binding site). HNH or HNH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 β-strands surrounded by a plurality of α-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

Site-directed polypeptides can introduce double-strand breaks or single-strand breaks in nucleic acids, e.g., genomic DNA. The double-strand break can stimulate a cell's endogenous DNA-repair pathways (e.g., homology-dependent repair (HDR) or NHEJ or alternative non-homologous end joining (A-NHEJ) or microhomology-mediated end joining (MMEJ)). NHEJ can repair cleaved target nucleic acid without the need for a homologous template. This can sometimes result in small deletions or insertions (InDels) in the target nucleic acid at the site of cleavage, and can lead to disruption or alteration of gene expression. HDR can occur when a homologous repair template, or donor, is available. The homologous donor template can have sequences that are homologous to sequences flanking the target nucleic acid cleavage site. The sister chromatid can be used by the cell as the repair template. However, for the purposes of genome editing, the repair template can be supplied as an exogenous nucleic acid, such as a plasmid, duplex oligonucleotide, single-strand oligonucleotide or viral nucleic acid. With exogenous donor templates, an additional nucleic acid sequence (such as a transgene) or modification (such as a single or multiple base change or a deletion) can be introduced between the flanking regions of homology so that the additional or altered nucleic acid sequence also becomes incorporated into the target locus. MMEJ can result in a genetic outcome that is similar to NHEJ in that small deletions and insertions can occur at the cleavage site. MMEJ can make use of homologous sequences of a few base pairs flanking the cleavage site to drive a favored end-joining DNA repair outcome. In some instances, it may be possible to predict likely repair outcomes based on analysis of potential microhomologies in the nuclease target regions.

Thus, in some cases, homologous recombination can be used to insert an exogenous polynucleotide sequence into the target nucleic acid cleavage site. An exogenous polynucleotide sequence is termed a donor polynucleotide (or donor or donor sequence) herein. The donor polynucleotide, a portion of the donor polynucleotide, a copy of the donor polynucleotide, or a portion of a copy of the donor polynucleotide can be inserted into the target nucleic acid cleavage site. The donor polynucleotide can be an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

The site-directed polypeptide can have an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acid Res, 39(21): 9275-9282 (2011)], and various other site-directed polypeptides. The site-directed polypeptide can have at least 70, 75, 80, 85, 90, 95, 97, 99, or 100% identity to a wild-type site-directed polypeptide (e.g., Cas9 from S. pyogenes, supra) over 10 contiguous amino acids. The site-directed polypeptide can have at most: 70, 75, 80, 85, 90, 95, 97, 99, or 100% identity to a wild-type site-directed polypeptide (e.g., Cas9 from S. pyogenes, supra) over 10 contiguous amino acids. The site-directed polypeptide can have at least: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. The site-directed polypeptide can have at most: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. The site-directed polypeptide can have at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. The site-directed polypeptide can have at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

The site-directed polypeptide can have a modified form of a wild-type exemplary site-directed polypeptide. The modified form of the wild-type exemplary site-directed polypeptide can have a mutation that reduces the nucleic acid-cleaving activity of the site-directed polypeptide. The modified form of the wild-type exemplary site-directed polypeptide can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

The modified form of the site-directed polypeptide can have a mutation such that it can induce a single-strand break (SSB) on a target nucleic acid (e.g., by cutting only one of the sugar-phosphate backbones of a double-strand target nucleic acid). The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). The mutation can result in one or more of the plurality of nucleic acid-cleaving domains retaining the ability to cleave the complementary strand of the target nucleic acid, but reducing its ability to cleave the non-complementary strand of the target nucleic acid. The mutation can result in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp 10, His840, Asn854 and Asn856, are mutated to inactivate one or more of the plurality of nucleic acid-cleaving domains (e.g., nuclease domains). The residues to be mutated can correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. One skilled in the art will recognize that mutations other than alanine substitutions can be suitable.

A D10A mutation can be combined with one or more of H840A, N854A, or N856A mutations to produce a site-directed polypeptide substantially lacking DNA cleavage activity. A H840A mutation can be combined with one or more of D10A, N854A, or N856A mutations to produce a site-directed polypeptide substantially lacking DNA cleavage activity. A N854A mutation can be combined with one or more of H840A, D10A, or N856A mutations to produce a site-directed polypeptide substantially lacking DNA cleavage activity. A N856A mutation can be combined with one or more of H840A. N854A, or D 10A mutations to produce a site-directed polypeptide substantially lacking DNA cleavage activity. Site-directed polypeptides that have one substantially inactive nuclease domain are referred to as “nickases”.

Nickase variants of RNA-guided endonucleases, for example Cas9, can be used to increase the specificity of CRISPR-mediated genome editing. Wild type Cas9 is typically guided by a single guide RNA designed to hybridize with a specified ˜20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes.

Mutations contemplated can include substitutions, additions, and deletions, or any combination thereof. The mutation converts the mutated amino acid to alanine. The mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation converts the mutated amino acid to a non-natural amino acid (e.g., selenomethionine). The mutation converts the mutated amino acid to amino acid mimics (e.g., phosphomimics). The mutation can be a conservative mutation. For example, the mutation converts the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

The site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target DNA. The site-directed polypeptide (e.g., variant, mutated, enzymatically inactive and/or conditionally enzymatically inactive endoribonuclease) can target RNA.

The site-directed polypeptide can have one or more non-native sequences (e.g., the site-directed polypeptide is a fusion protein).

The site-directed polypeptide can have an amino acid sequence having at least 15% amino acid identity to a Cas9 from a bacterium (e.g., S. pyogenes), a nucleic acid binding domain, and two nucleic acid cleaving domains (i.e., a HNH domain and a RuvC domain).

The site-directed polypeptide can have an amino acid sequence having at least 15% amino acid identity to a Cas9 from a bacterium (e.g., S. pyogenes), and two nucleic acid cleaving domains (i.e., a HNH domain and a RuvC domain).

The site-directed polypeptide can have an amino acid sequence having at least 15% amino acid identity to a Cas9 from a bacterium (e.g., S. pyogenes), and two nucleic acid cleaving domains, wherein one or both of the nucleic acid cleaving domains have at least 50% amino acid identity to a nuclease domain from Cas9 from a bacterium (e.g., S. pyogenes).

The site-directed polypeptide can have an amino acid sequence having at least 15% amino acid identity to a Cas9 from a bacterium (e.g., S. pyogenes), two nucleic acid cleaving domains (i.e., a HNH domain and a RuvC domain), and non-native sequence (for example, a nuclear localization signal) or a linker linking the site-directed polypeptide to a non-native sequence.

The site-directed polypeptide can have an amino acid sequence having at least 15% amino acid identity to a Cas9 from a bacterium (e.g., S. pyogenes), two nucleic acid cleaving domains (i.e., a HNH domain and a RuvC domain), wherein the site-directed polypeptide has a mutation in one or both of the nucleic acid cleaving domains that reduces the cleaving activity of the nuclease domains by at least 50%.

The site-directed polypeptide can have an amino acid sequence having at least 15% amino acid identity to a Cas9 from a bacterium (e.g., S. pyogenes), and two nucleic acid cleaving domains (i.e., a HNH domain and a RuvC domain), wherein one of the nuclease domains has mutation of aspartic acid 10, and/or wherein one of the nuclease domains can have a mutation of histidine 840, and wherein the mutation reduces the cleaving activity of the nuclease domain(s) by at least 50%.

The one or more site-directed polypeptides, e.g. DNA endonucleases, can have two nickases that together effect one double-strand break at a specific locus in the genome, or four nickases that together effect or cause two double-strand breaks at specific loci in the genome. Alternatively, one site-directed polypeptide, e.g. DNA endonuclease, can effect or cause one double-strand break at a specific locus in the genome.

### Genome-Targeting Nucleic Acid

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. A genome-targeting RNA is referred to as a “guide RNA” or “gRNA” herein. A guide RNA can have at least a spacer sequence that hybridizes to a target nucleic acid sequence of interest, and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex can bind a site-directed polypeptide, such that the guide RNA and site-direct polypeptide form a complex. The genome-targeting nucleic acid can provide target specificity to the complex by virtue of its association with the site-directed polypeptide. The genome-targeting nucleic acid thus can direct the activity of the site-directed polypeptide.

Exemplary guide RNAs include the spacer sequences 15-200 bases wherein the genome location is based on the GRCh38 human genome assembly. As is understood by the person of ordinary skill in the art, each guide RNA can be designed to include a spacer sequence complementary to its genomic target sequence. For example, each of the spacer sequences can be put into a single RNA chimera or a crRNA (along with a corresponding tracrRNA). See Jinek et al., Science, 337, 816-821 (2012) and Deltcheva et al., Nature, 471, 602-607 (2011).

The genome-targeting nucleic acid can be a double-molecule guide RNA. The genome-targeting nucleic acid can be a single-molecule guide RNA.

A double-molecule guide RNA can have two strands of RNA. The first strand has in the 5′ to 3′ direction, an optional spacer extension sequence, a spacer sequence and a minimum CRISPR repeat sequence. The second strand can have a minimum tracrRNA sequence (complementary to the minimum CRISPR repeat sequence), a 3′ tracrRNA sequence and an optional tracrRNA extension sequence.

A single-molecule guide RNA (sgRNA) in a Type II system can have, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can have elements that contribute additional functionality (e.g., stability) to the guide RNA. The single-molecule guide linker can link the minimum CRISPR repeat and the minimum tracrRNA sequence to form a hairpin structure. The optional tracrRNA extension can have one or more hairpins.

A single-molecule guide RNA (sgRNA) in a Type V system can have, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Extension Sequence

In some examples of genome-targeting nucleic acids, a spacer extension sequence can modify activity, provide stability and/or provide a location for modifications of a genome-targeting nucleic acid. A spacer extension sequence can modify on- or off-target activity or specificity. In some examples, a spacer extension sequence can be provided. The spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. The spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. The spacer extension sequence can be less than 10 nucleotides in length. The spacer extension sequence can be between 10-30 nucleotides in length. The spacer extension sequence can be between 30-70 nucleotides in length.

The spacer extension sequence can have another moiety (e.g., a stability control sequence, an endoribonuclease binding sequence, a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

### Spacer Sequence

The spacer sequence hybridizes to a sequence in a target nucleic acid of interest. The spacer of a genome-targeting nucleic acid can interact with a target nucleic acid in a sequence-specific manner via hybridization (i.e., base pairing). The nucleotide sequence of the spacer can vary depending on the sequence of the target nucleic acid of interest.

In a CRISPR/Cas system herein, the spacer sequence can be designed to hybridize to a target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

The target nucleic acid sequence can have 20 nucleotides. The target nucleic acid can have less than 20 nucleotides. The target nucleic acid can have more than 20 nucleotides. The target nucleic acid can have at least: 5, 10, 15, 16, 17, 18, 19, 20, 21, 22, 23, 24, 25, 30 or more nucleotides. The target nucleic acid can have at most: 5, 10, 15, 16, 17, 18, 19, 20, 21, 22, 23, 24, 25, 30 or more nucleotides. The target nucleic acid sequence can have 20 bases immediately 5′ of the first nucleotide of the PAM.

The spacer sequence that hybridizes to the target nucleic acid can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can have 20 nucleotides. In some examples, the spacer can have 19 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

The spacer sequence can be designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

Table 4 provides some of non-limiting and illustrative examples of spacer sequences that can be used in certain embodiments of the disclosure. The efficiency indicated in Table 4 represents a frequency of cut at the target site in the genome when each gRNA is introduced with DNA endonuclease. For example, if one gRNA is introduced to a cell with DNA endonuclease and it cuts the genome at the intended target site every time of delivery, the efficiency will be scored as 100%. Therefore, the higher the efficiency value is the more efficient and accurate the cutting by the gRNA is at the target site. The sequences from Table 4 are designed to target sites at, at, within, or near the WAS gene locus. In particular, the sequences targeted by the gRNAs from Table 4 are in the intergenic sequence that is upstream of the promoter of the endogenous WAS promoter. In some embodiments, the intergenic sequence may be at least 500 bp or about 500 bp upstream of the first exon of the endogenous WAS gene. In some embodiments, the intergenic sequence may be at least 500 bp or about 500 bp to about 2000 bp upstream of the first exon of the endogenous WAS gene. In some embodiments, the spacer sequence can be any sequences from Table 4 or any variants thereof having at least 30%, at least 40%, at least 50%, at least 60%, at least 70%, at least 80%, at least 85%, at least 90%, at least 95%, at least 96%, at least 97%, at least 98%, at least 99% or about 100% identity to the sequences from Table 4. In some embodiments, the spacer sequence can be 15 bases to 20 bases in length.

TABLE 4
Examplary gRNA Sequences

| | | | Efficiency | |
|---|---|---|---|---|
| Name | Guide (or spacer) | PAM | (%) | SEQ ID NO. |
| WASp_T91 | GCCAAGAGTGAAGGCGTGGA | GGG | 91.8 | 5267 |
| WASp_T140 | AAAGTAATTTGGGAGCTGCG | GGG | 87.3 | 5268 |
| WASp_T245 | GGGGTGTGACTGACATTCCC | AGG | 87.2 | 5269 |
| WASp_T18 | GGCCAGTAGTGCTTACTTTG | TGG | 87 | 5270 |
| WASp_T26 | AACACGTGCAATGGCCTTGA | AGG | 85.8 | 5271 |
| WASp_T250 | GGAACGGCACATCTCCAGTT | GGG | 85.7 | 5272 |
| WASp_T103 | TAAAACTAGGATGTCCAGTG | GGG | 82.7 | 5273 |
| WASp_T263 | CTGAGCAGTCAGTGTGTGCT | AGG | 81.5 | 5274 |
| WASp_T20 | CCAGTAGTGCTTACTTTGTG | GGG | 81.1 | 5275 |
| WASp_T267 | GGTATTGAAGCATTGAATGA | AGG | 78.3 | 5276 |
| WASp_T110 | CTTTAAAAAAGGGATGGGCT | GGG | 78.3 | 5277 |
| WASp_T246 | TGACATTCCCAGGTACCTGA | AGG | 78.2 | 5278 |
| WASp_T224 | ACACATCTCCTGGTCCACAT | AGG | 77.9 | 5279 |
| WASp_T230 | ACGGGAATCTGAGGGCTGTA | GGG | 77.3 | 5280 |
| WASp_T94 | GGCCTGCCTGGCTCGCACTC | CGG | 76.5 | 5281 |
| WASp_T44 | TTGGAACTGCCACTGCAGAA | GGG | 76.1 | 5282 |
| WASp_T227 | GACTCTCAGACGGGAATCTG | AGG | 76.1 | 5283 |
| WASp_T74 | TGAGAGTCTGCATGCCTATG | TGG | 74.2 | 5284 |
| WASp_T145 | CTGCGGGGAGGCAAGGGTAA | GGG | 72.6 | 5285 |
| WASp_T252 | GTCCTTCCTGGAAACTTCCA | TGG | 72.2 | 5286 |
| WASp_T32 | TGCCCTGATCAGCAGGTTGG | AGG | 71.8 | 5287 |
| WASp_T237 | CAGATACAGGAACAGCAGTT | TGG | 71.7 | 5288 |
| WASp_T35 | CCCGAAGCTTAGCTGTGAGT | GGG | 70.8 | 5289 |
| WASp_T19 | GCCAGTAGTGCTTACTTTGT | GGG | 70.3 | 5290 |
| WASp_T266 | TTGGACAGATGACACTAAAT | GGG | 70.3 | 5291 |
| WASp_T156 | ACGATAGGCGTGGATCACAG | GGG | 69.7 | 5292 |
| WASp_T88 | ACAGCAACAGCCAAGAGTGA | AGG | 68.5 | 5293 |
| WASp_T99 | AAAGGGGTCAGAACAGTGAC | TGG | 68.3 | 5294 |
| WASp_T239 | GATACAGGAACAGCAGTTTG | GGG | 68.2 | 5295 |
| WASp_T34 | GCCCGAAGCTTAGCTGTGAG | TGG | 66.9 | 5296 |
| WASp_T244 | GGGGGAGCTGAGCAGGTCTG | GGG | 66.7 | 5297 |
| WASp_T55 | GAAGGACTGCAGGTCCCAAC | TGG | 66.6 | 5298 |
| WASp_T226 | AGGCATGCAGACTCTCAGAC | GGG | 64.8 | 5299 |
| WASp_T77 | GTGGACCAGGAGATGTGTGC | GGG | 64.8 | 5300 |
| WASp_T79 | AGAACCAGAAGCAGCCCAAA | GGG | 63.6 | 5301 |
| WASp_T233 | CCTCAGTTTCCCCATGTATG | GGG | 63.4 | 5302 |
| WASp_T236 | GACTTGAAGCTCTCAGATAC | AGG | 63.2 | 5303 |
| WASp_T212 | GCAGGCCAGGTGTGAGTGCA | TGG | 61.8 | 5304 |
| WASp_T147 | GGGAGGCAAGGGTAAGGGAT | GGG | 61.8 | 5305 |
| WASp_T231 | GTGAATGCTAGCTCAGTCCC | CGG | 61.4 | 5306 |
| WASp_T97 | TGGCTCGCACTCCGGGCAAA | GGG | 61.2 | 5307 |
| WASp_T100 | GTGAGTGTTAAGATAAAACT | AGG | 60.8 | 5308 |
| WASp_T269 | ATTTGATTCAGTGGTTCCAG | AGG | 59.8 | 5309 |
| WASp_T260 | CACCTCCAACCTGCTGATCA | GGG | 59.7 | 5310 |
| WASp_T222 | TGCTTCTGGTTCTTGCTGTT | GGG | 59.4 | 5311 |
| WASP_T89 | AACAGCCAAGAGTGAAGGCG | TGG | 59 | 5312 |
| WASp_T144 | GCTGCGGGGAGGCAAGGGTA | AGG | 58.9 | 5313 |
| WASp_T73 | GGAAATAGTCTGGCATCATC | TGG | 57.8 | 5314 |
| WASp_T45 | TGGAACTGCCACTGCAGAAG | GGG | 5315 | 5315 |
| WASp_T98 | GGCTCGCACTCCGGGCAAAG | GGG | 57.4 | 5316 |
| WASp_T102 | ATAAAACTAGGATGTCCAGT | GGG | 57.2 | 5317 |
| WASp_T59 | GAGAGCTTCAAGTCTCCAAA | TGG | 56.9 | 5318 |
| WASp_T33 | CAGCAGGTTGGAGGTGCTCC | AGG | 56.6 | 5319 |
| WASp_T52 | GGGAAGCCATGGAAGTTTCC | AGG | 56.6 | 5320 |
| WASp_T210 | ATACAGGAACAGCAGTTTGG | GGG | 56.4 | 5321 |
| WASp_T53 | AGCCATGGAAGTTTCCAGGA | AGG | 56 | 5322 |
| WASp_T251 | TTGGGACCTGCAGTCCTTCC | TGG | 55.4 | 5323 |
| WASp_T162 | GTCTTGCTCTGTCATCATCC | AGG | 55.2 | 5324 |
| WASp_T101 | GATAAAACTAGGATGTCCAG | TGG | 54.6 | 5325 |
| WASp_T23 | CTGGAACCACTGAATCAAAT | TGG | 54.5 | 5326 |
| WASp_T51 | CCTGCACGTGTGGGAAGCCA | TGG | 54.4 | 5327 |
| WASp_T21 | ATGTGAAATGAGTTAATAAA | TGG | 54.3 | 5328 |
| WASp_T39 | GCTGTGAGTGGGGACACGGG | AGG | 54.2 | 5329 |
| WASp_T90 | AGCCAAGAGTGAAGGCGTGG | AGG | 53.4 | 5330 |
| WASp_T108 | CCTTTCTTTAAAAAAGGGAT | GGG | 53 | 5331 |
| WASp_T138 | AGAAAGTAATTTGGGAGCTG | CGG | 52.7 | 5332 |
| WASp_T92 | CGAGACCATGCACTCACACC | TGG | 51.6 | 5333 |
| WASp_T206 | TAAAAGCACATAATCTTCAA | AGG | 51.4 | 5334 |
| WASp_T265 | TTTGGACAGATGACACTAAA | TGG | 51.1 | 5335 |
| WASp_T60 | CAAATGGCCTACCTCATACA | TGG | 50 | 5336 |
| WASP_T96 | CTGGCTCGCACTCCGGGCAA | AGG | 49.7 | 5337 |
| WASp_T249 | AGGAACGGCACATCTCCAGT | TGG | 48.4 | 5338 |
| WASp_T50 | GGCTTTGAACCTGCACGTGT | GGG | 48.1 | 5339 |
| WASp_T214 | TGATTCCTGTTTGCCTATAG | TGG | 47.6 | 5340 |
| WASp_T137 | TGACAAGCAGAAAGTAATTT | GGG | 47.6 | 5341 |
| WASp_T24 | AGTGTCATCTGTCCAAATGC | TGG | 47.2 | 5342 |
| WASp_T78 | AAGAACCAGAAGCAGCCCAA | AGG | 47.2 | 5343 |
| WASp_T157 | GGGCTCGCTCTGTAATTAAA | AGG | 47.2 | 5344 |
| WASp_T46 | GCAGAAGGGGTTCTGAACCT | AGG | 46.9 | 5345 |
| WASp_T255 | GTTCAGAACCCCTTCTGCAG | AGG | 46.6 | 5346 |
| WASp_T72 | ATTCACTTGTGGAAATAGTC | TGG | 46.2 | 5347 |
| WASp_T254 | AAAGCCTCTCTCCTGAACCT | AGG | 46.1 | 5348 |
| WASp_T213 | TCCCTCCACGCCTTCACTCT | TGG | 45.9 | 5349 |
| WASp_T104 | AACTAAACAAGATGTTGTTC | AGG | 45.8 | 5350 |
| WASp_T218 | ATTCTCCTGTAACAGCCCTT | TGG | 45.4 | 5351 |
| WASp_T248 | CCAGGTACCTGAAGGAGGAA | CGG | 44.8 | 5352 |
| WASp_T22 | AAAGCACTTAGAAAAGCCTC | TGG | 13.3 | 5353 |
| WASp_T256 | CCCCACTCACAGCTAAGCTT | CGG | 42.9 | 5354 |
| WASp_T238 | AGATACAGGAACAGCAGTTT | GGG | 42.6 | 5355 |
| WASp_T257 | CCCACTCACAGCTAAGCTTC | GGG | 42 | 5356 |
| WASp_T25 | GTCACAATGAACACGTGCAA | TGG | 40.8 | 5357 |
| WASp_T153 | CTAAGCACTCACGATAGGCG | TGG | 39.5 | 5358 |
| WASp_T155 | CACGATAGGCGTGGATCACA | GGG | 39.2 | 5359 |
| WASp_T38 | TTAGCTGTGAGTGGGGACAC | GGG | 38.7 | 5360 |
| WASp_T207 | AAGGCTTGCTTTCTCCCCAC | TGG | 38.7 | 5361 |
| WASp_T247 | CATTCCCAGGTACCTGAAGG | AGG | 38.3 | 5362 |
| WASp_T63 | CCTCATACATGGGGAAACTG | AGG | 38.3 | 5363 |
| WASp_T186 | GCCAATGACGCATATGCCTC | TGG | 38.2 | 5364 |
| WASp_T270 | CCCCACAAAGTAAGCACTAC | TGG | 37.9 | 5365 |
| WASp_T54 | AAGTTTCCAGGAAGGACTGC | AGG | 37.5 | 5366 |
| WASp_T163 | TGCTCTGTCATCATCCAGGC | TGG | 37.1 | 5367 |
| WASp_T264 | CTAGGCGTATCTCCAGCATT | TGG | 36.8 | 5368 |
| WASp_T31 | GCTTGCCCTGATCAGCAGGT | TGG | 36.5 | 5369 |
| WASp_T43 | GTTGGAACTGCCACTGCAGA | TGG | 36.5 | 5370 |
| WASp_T232 | GCTCAGTCCCCGGCCTCCCC | AGG | 36.2 | 5371 |
| WASp_T71 | ACTGAGCTAGCATTCACTTG | TGG | 35.3 | 5372 |
| WASp_T36 | CCGAAGCTTAGCTGTGAGTG | GGG | 35 | 5373 |
| WASp_T95 | GCCTGCCTGGCTCGCACTCC | GGG | 34 | 5374 |
| WASp_T47 | GGGGTTCTGAACCTAGGTTC | AGG | 33.7 | 5375 |
| WASp_T259 | GCACCTCCAACCTGCTGATC | AGG | 33.6 | 5376 |
| WASp_T142 | TTGGGAGCTGCGGGGAGGCA | AGG | 33.1 | 5377 |
| WASp_T208 | ACTGTTCTGACCCCTTTGCC | CGG | 32.6 | 5378 |
| WASp_T210 | GCCCGGAGTGCGAGCCAGGC | AGG | 32.1 | 5379 |
| WASp_T234 | AGTTTCCCCATGTATGAGGT | AGG | 31.4 | 5380 |
| WASp_T211 | GAGTGCGAGCCAGGCAGGCC | AGG | 31.2 | 5381 |
| WASp_T58 | CGTTCCTCCTTCAGGTACCT | GGG | 31.1 | 5382 |
| WASp_T61 | AAATGGCCTACCTCATACAT | AGG | 31.1 | 5383 |
| WASp_T151 | ACCAGAGGCATATGCGTCAT | TGG | 31.1 | 5384 |
| WASp_T75 | TCTGCATGCCTATGTGGACC | AGG | 30.8 | 5385 |
| WASp_T235 | CATGTATGAGGTAGGCCATT | TGG | 30.6 | 5386 |
| WASp_T37 | CTTAGCTGTGAGTGGGGACA | CGG | 29.9 | 5387 |
| WASp_T229 | GACGGGAATCTGAGGGCTGT | AGG | 29.9 | 5388 |
| WASp _T243 | TGGGGGAGCTGAGCAGGTCT | GGG | 29.8 | 5389 |
| WASp_T253 | CCATGGCTTCCCACACGTGC | TGG | 29.7 | 5390 |
| WASp_T48 | TGAACCTAGGTTCAGGAGAG | AGG | 29.5 | 5391 |
| WASp_T216 | CATGGTGTGCATGTGCAGCC | TGG | 29.4 | 5392 |
| WASp_T148 | GGAGGCAAGGGTAAGGGATG | GGG | 29.3 | 5393 |
| WASp_T225 | TAGGCATGCAGACTCTCAGA | CGG | 28.9 | 5394 |
| WASp_T261 | GATCAGGGCAAGCCCCGATG | TGG | 28 | 5395 |
| WASp_T271 | ACAAAGTAAGCACTACTGGC | CGG | 27.5 | 5396 |
| WASp_T57 | CCGTTCCTCCTTCAGGTACC | TGG | 27 | 5397 |
| WASp_T40 | CTGTGAGTGGGGACACGGGA | GGG | 26.7 | 5398 |
| WASp_T158 | GCTCTGTAATTAAAAGGAAA | AGG | 26.7 | 5399 |
| WASp_T268 | TGATATCCAATTTGATTCAG | TGG | 25.8 | 5400 |
| WASp_T223 | TCACTCCCGCACACATCTCC | TGG | 25.6 | 5401 |
| WASp_T241 | GCAGTTTGGGGGAGCTGAGC | AGG | 25.1 | 5402 |
| WASp_T150 | GGGATGGGGAAGTGGACCAG | AGG | 25.1 | 5403 |
| WASp_T258 | TCACAGCTAAGCTTCGGGCC | TGG | 24.3 | 5404 |
| WASp_T62 | AATGGCCTACCTCATACATG | GGG | 24 | 5405 |
| WASp_T152 | AGTGTCTAAGCACTCACGAT | AGG | 22.4 | 5406 |
| WASp_T242 | TTGGGGGAGCTGAGCAGGTC | TGG | 21.3 | 5407 |
| WASp_T221 | CTGCTTCTGGTTCTTGCTGT | TGG | 20.4 | 5408 |
| WASp_T154 | TCACGATAGGCGTGGATCAC | AGG | 20.3 | 5409 |
| WASp_T56 | AGATGTGCCGTTCCTCCTTC | AGG | 20.1 | 5410 |
| WASp_T12 | GAACTCCTGACCTCGTGATC | TGG | 19.2 | 5411 |
| WASp_T93 | GCACTCACACCTGGCCTGCC | TGG | 18 | 5412 |
| WASp_T149 | AAGGGTAAGGGATGGGGAAG | TGG | 18 | 5413 |
| WASp_T159 | CTCTGTAATTAAAAGGAAAA | GGG | 17.6 | 5414 |
| WASp_T41 | TGAGTGGGGACACGGGAGGG | AGG | 17.5 | 5415 |
| WASp_T27 | AATGGCCTTGAAGGCCACAT | CGG | 15.9 | 5416 |
| WASp_T205 | CCCATCCCTTTTTTAAAGAA | AGG | 15.8 | 5417 |
| WASp_T49 | AGGCTTTGAACCTGCACGTG | AGG | 14.4 | 5418 |
| WASp_T262 | AAGCCCCGATGTGGCCTTCA | AGG | 12 | 5419 |
| WASp_T5 | AGGTGTGCACCACCACACCA | GGG | 11.3 | 5420 |
| WASp_T80 | GCAGCCCAAAGGGCTGTTAC | AGG | 11 | 5421 |
| WASp_T272 | CAAAGTAAGCACTACTGGCC | GGG | 10.8 | 5422 |
| WASp_T209 | CTTTGCCCGGAGTGCGAGCC | AGG | 9.9 | 5423 |
| WASp_T109 | TCTTTAAAAAAGGGATGGGC | TGG | 9.7 | 5424 |
| WASp T29 | TGGCCTTGAAGGCCACATCG | GGG | 9.5 | 5425 |
| WASp_T67 | GGGGAAACTGAGGCCTGGGG | AGG | 9.2 | 5426 |
| WASp_T30 | CGGGGCTTGCCCTGATCAGC | AGG | 9 | 5427 |
| WASp_T70 | ACTGAGGCCTGGGGAGGCCG | GGG | 8.7 | 5428 |
| WASp_T273 | TAAGCACTACTGGCCGGGTG | CGG | 8.1 | 5429 |
| WASp_T76 | TGTGGACCAGGAGATGTGTG | CGG | 7.7 | 5430 |
| WASp_T42 | TGGGGACACGGGAGGGAGGT | TGG | 7.5 | 5431 |
| WASp_T69 | AACTGAGGCCTGGGGAGGCC | GGG | 7.5 | 5432 |
| WASp_T28 | ATGGCCTTGAAGGCCACATC | GGG | 7 | 5433 |
| WASp_T106 | AAGTTCCTTTCTTTAAAAAA | GGG | 6.9 | 5434 |
| WASp_T141 | GTAATTTGGGAGCTGCGGGG | AGG | 6.9 | 5435 |
| WASp_T161 | TGTTGCTGTTTTTGAGACAA | GGG | 6.7 | 5436 |
| WASp_T217 | ATGGTGTGCATGTGCAGCCT | GGG | 5 | 5437 |
| WASp_T107 | TCCTTTCTTTAAAAAAGGGA | TGG | 4.3 | 5438 |
| WASp_T139 | GAAAGTAATTTGGGAGCTGC | GGG | 3.9 | 5439 |
| WASp_T105 | AAAGTTCCTTTCTTTAAAAA | AGG | 3.6 | 5440 |
| WASp_T136 | ATGACAAGCAGAAAGTAATT | TGG | 3.5 | 5441 |
| WASp_T68 | AAACTGAGGCCTGGGGAGGC | CGG | 2.5 | 5442 |
| WASp_T220 | ACAGCCCTTTGGGCTGCTTC | TGG | 2.2 | 5443 |
| WASp_T219 | TTCTCCTGTAACAGCCCTTT | GGG | 1.9 | 5444 |
| WASp_T228 | ACTCTCAGACGGGAATCTGA | GGG | TBD | 5445 |
| WASp_T81 | AGGGCTGTTACAGGAGAATA | TGG | TBD | 5446 |
| WASp_T82 | ACAGGAGAATATGGACACCC | AGG | TBD | 5447 |
| WASp T83 | CAGGCTGCACATGCACACCA | TGG | TBD | 5448 |
| WASp_T215 | CACTGCCATACAGCATTCCA | TGG | TBD | 5449 |
| WASp_T84 | GCACACCATGGAATGCTGTA | TGG | TBD | 5450 |
| WASp_T85 | CATGGAATGCTGTATGGCAG | TGG | TBD | 5451 |
| WASp_T86 | AAATGAACAGCTACCACTAT | AGG | TBD | 5452 |
| WASp_T87 | AGCTACCACTATAGGCAAAC | AGG | TBD | 5453 |
| WASp_T143 | TGGGAGCTGCGGGGAGGCAA | GGG | TBD | 5454 |

In some embodiments, gRNAs that can be used for genome-edition in accordance with the present disclosures can be a nucleic acid sequence that can target sites at, within, or near the WAS gene. The gRNA can target any sites, e.g, from any introns, any exons, any sequence junctioning an exon and intron (or vice versa) and any regulatory sequence such as upstream and downstream sequences of the WAS gene locus.

In some embodiments, gRNAs that can be used for genome-edition in accordance with the present disclosures can be a nucleic acid sequence that can target sites at, within, or near a safe harbor locus or a safe harbor site. In some embodiments, a safe-harbor locus is selected from the group consisting of albumin gene, an AAVS1 gene, an HRPT gene, a CCR5 gene, a globin gene, TTR gene, TF gene, F9 gene, Alb gene, Gys2 gene and PCSK9 gene. In some embodiments, a safe harbor site is selected from the group consisting of the following regions: AAVS1 19q13.4-qter, HRPT 1q31.2, CCR5 3p21.31. Globin 11p15.4, TTR 18q12.1, TF 3q22.1, F9 Xq27.1, Alb 4q13.3, Gys2 12p12.1 and PCSK9 1p32.3.

In some embodiments, gRNAs that can be used for genome-edition in accordance with the present disclosures can be a nucleic acid sequence that can target sites at, within, or near the AAVS1 gene. The gRNA can target any sites, e.g, from any introns, any exons, any sequence junctioning an exon and intron (or vice versa) and any regulatory sequence such as upstream and downstream sequences of the AAVS1 gene locus.

### Minimum CRISPR Repeat Sequence

A minimum CRISPR repeat sequence can be a sequence with at least about 300%, about 40%, about 50%, about 60%, about 65%, about 70%, about 75%, about 800%, about 85%, about 90%, about 95%, or 100% sequence identity to a reference CRISPR repeat sequence (e.g., crRNA from S. pyogenes).

A minimum CRISPR repeat sequence can have nucleotides that can hybridize to a minimum tracrRNA sequence in a cell. The minimum CRISPR repeat sequence and a minimum tracrRNA sequence can form a duplex, i.e, a base-paired double-stranded structure. Together, the minimum CRISPR repeat sequence and the minimum tracrRNA sequence can bind to the site-directed polypeptide. At least a part of the minimum CRISPR repeat sequence can hybridize to the minimum tracrRNA sequence. At least a part of the minimum CRISPR repeat sequence can have at least about 30%, about 40%, about 50%, about 60%, about 65%, about 70%, about 75%, about 80%, about 85%, about 90.6, about 95%, or 100% complementary to the minimum tracrRNA sequence. At least a part of the minimum CRISPR repeat sequence can have at most about 30%, about 40%, about 50%, about 60%, about 65%, about 70%, about 75%, about 80%, about 85%, about 90%, about 95%, or 100% complementary to the minimum tracrRNA sequence.

The minimum CRISPR repeat sequence can have a length from about 7 nucleotides to about 100 nucleotides. For example, the length of the minimum CRISPR repeat sequence is from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. In some examples, the minimum CRISPR repeat sequence can be approximately 9 nucleotides in length. The minimum CRISPR repeat sequence can be approximately 12 nucleotides in length.

The minimum CRISPR repeat sequence can be at least about 60% identical to a reference minimum CRISPR repeat sequence (e.g., wild-type crRNA from S. pyogenes) over a stretch of at least 6, 7, or 8 contiguous nucleotides. For example, the minimum CRISPR repeat sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence can be a sequence with at least about 30%, about 40%, about 50%, about 60%, about 65%, about 70%, about 75%, about 80%, about 85%, about 90%, about 95%, or 100% sequence identity to a reference tracrRNA sequence (e.g., wild type tracrRNA from S. pyogenes).

A minimum tracrRNA sequence can have nucleotides that hybridize to a minimum CRISPR repeat sequence in a cell. A minimum tracrRNA sequence and a minimum CRISPR repeat sequence form a duplex, i.e, a base-paired double-stranded structure. Together, the minimum tracrRNA sequence and the minimum CRISPR repeat can bind to a site-directed polypeptide. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides.

The duplex between the minimum CRISPR RNA and the minimum tracrRNA can have a double helix. The duplex between the minimum CRISPR RNA and the minimum tracrRNA can have at least about 1, 2, 3, 4, 5, 6, 7, 8, 9, or 10 or more nucleotides. The duplex between the minimum CRISPR RNA and the minimum tracrRNA can have at most about 1, 2, 3, 4, 5, 6, 7, 8, 9, or 10 or more nucleotides.

The duplex can have a mismatch (i.e., the two strands of the duplex are not 100% complementary). The duplex can have at least about 1, 2, 3, 4, or 5 or mismatches. The duplex can have at most about 1, 2, 3, 4, or 5 or mismatches. The duplex can have no more than 2 mismatches.

### Bulges

In some cases, there can be a “bulge” in the duplex between the minimum CRISPR RNA and the minimum tracrRNA. A bulge is an unpaired region of nucleotides within the duplex. A bulge can contribute to the binding of the duplex to the site-directed polypeptide. The bulge can have, on one side of the duplex, an unpaired 5′-XXXY-3′ where X is any purine and Y has a nucleotide that can form a wobble pair with a nucleotide on the opposite strand, and an unpaired nucleotide region on the other side of the duplex. The number of unpaired nucleotides on the two sides of the duplex can be different.

In one example, the bulge can have an unpaired purine (e.g., adenine) on the minimum CRISPR repeat strand of the bulge. In some examples, the bulge can have an unpaired 5′-AAGY-3′ of the minimum tracrRNA sequence strand of the bulge, where Y has a nucleotide that can form a wobble pairing with a nucleotide on the minimum CRISPR repeat strand.

bulge on the minimum CRISPR repeat side of the duplex can have at least 1, 2, 3, 4, or 5 or more unpaired nucleotides. A bulge on the minimum CRISPR repeat side of the duplex can have at most 1, 2, 3, 4, or 5 or more unpaired nucleotides. A bulge on the minimum CRISPR repeat side of the duplex can have 1 unpaired nucleotide.

A bulge on the minimum tracrRNA sequence side of the duplex can have at least 1, 2, 3, 4, 5, 6, 7, 8, 9, or 10 or more unpaired nucleotides. A bulge on the minimum tracrRNA sequence side of the duplex can have at most 1, 2, 3, 4, 5, 6, 7, 8, 9, or 10 or more unpaired nucleotides. A bulge on a second side of the duplex (e.g., the minimum tracrRNA sequence side of the duplex) can have 4 unpaired nucleotides.

A bulge can have at least one wobble pairing. In some examples, a bulge can have at most one wobble pairing. A bulge can have at least one purine nucleotide. A bulge can have at least 3 purine nucleotides. A bulge sequence can have at least 5 purine nucleotides. A bulge sequence can have at least one guanine nucleotide. In some examples, a bulge sequence can have at least one adenine nucleotide.

### Hairpins

In various examples, one or more hairpins can be located 3′ to the minimum tracrRNA in the 3′ tracrRNA sequence.

The hairpin can start at least about 1, 2, 3, 4, 5, 6, 7, 8, 9, 10, 15, or 20 or more nucleotides 3′ from the last paired nucleotide in the minimum CRISPR repeat and minimum tracrRNA sequence duplex. The hairpin can start at most about 1, 2, 3, 4, 5, 6, 7, 8, 9 or 10 or more nucleotides 3′ of the last paired nucleotide in the minimum CRISPR repeat and minimum tracrRNA sequence duplex.

The hairpin can have at least about 1, 2, 3, 4, 5, 6, 7, 8, 9, 10, 15, or 20 or more consecutive nucleotides. The hairpin can have at most about 1, 2, 3, 4, 5, 6, 7, 8, 9, 10, 15, or more consecutive nucleotides.

The hairpin can have a CC dinucleotide (i.e., two consecutive cytosine nucleotides).

The hairpin can have duplexed nucleotides (e.g., nucleotides in a hairpin, hybridized together). For example, a hairpin can have a CC dinucleotide that is hybridized to a GG dinucleotide in a hairpin duplex of the 3′ tracrRNA sequence.

One or more of the hairpins can interact with guide RNA-interacting regions of a site-directed polypeptide.

In some examples, there are two or more hairpins, and in other examples there are three or more hairpins.

3′ tracrRNA Sequence

A 3′ tracrRNA sequence can have a sequence with at least about 30%, about 40%, about 50%, about 60%6, about 65%, about 70%, about 75%, about 80%, about 85%, about 90%, about 95%, or 100% sequence identity to a reference tracrRNA sequence (e.g., a tracrRNA from S. pyogenes).

The 3′ tracrRNA sequence can have a length from about 6 nucleotides to about 100 nucleotides. For example, the 3′ tracrRNA sequence can have a length from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt, or from about 15 nt to about 25 nt. The 3′ tracrRNA sequence can have a length of approximately 14 nucleotides.

The 3′ tracrRNA sequence can be at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence can be at least about 60% identical, about 65% identical, about 70% identical, about 75% identical, about 80% identical, about 85% identical, about 90% identical, about 95% identical, about 98% identical, about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

The 3′ tracrRNA sequence can have more than one duplexed region (e.g., hairpin, hybridized region). The 3′ tracrRNA sequence can have two duplexed regions.

The 3′ tracrRNA sequence can have a stem loop structure. The stem loop structure in the 3′ tracrRNA can have at least 1, 2, 3, 4, 5, 6, 7, 8, 9, 10, 15 or 20 or more nucleotides. The stem loop structure in the 3′ tracrRNA can have at most 1, 2, 3, 4, 5, 6, 7, 8, 9 or 10 or more nucleotides. The stem loop structure can have a functional moiety. For example, the stem loop structure can have an aptamer, a ribozyme, a protein-interacting hairpin, a CRISPR array, an intron, or an exon. The stem loop structure can have at least about 1, 2, 3, 4, or 5 or more functional moieties. The stem loop structure can have at most about 1, 2, 3, 4, or 5 or more functional moieties.

The hairpin in the 3′ tracrRNA sequence can have a P-domain. In some examples, the P-domain can have a double-stranded region in the hairpin.

tracrRNA Extension Sequence

A tracrRNA extension sequence may be provided whether the tracrRNA is in the context of single-molecule guides or double-molecule guides. The tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, or 400 nucleotides. The tracrRNA extension sequence can have a length from about 20 to about 5000 or more nucleotides. The tracrRNA extension sequence can have a length of more than 1000 nucleotides. The tracrRNA extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence can have a length of less than 1000 nucleotides. The tracrRNA extension sequence can have less than 10 nucleotides in length. The tracrRNA extension sequence can be 10-30 nucleotides in length. The tracrRNA extension sequence can be 30-70 nucleotides in length.

The tracrRNA extension sequence can have a functional moiety (e.g., a stability control sequence, ribozyme, endoribonuclease binding sequence). The functional moiety can have a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt. The functional moiety can function in a eukaryotic cell. The functional moiety can function in a prokaryotic cell. The functional moiety can function in both eukaryotic and prokaryotic cells.

Non-limiting examples of suitable tracrRNA extension functional moieties include a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors. DNA methyltransferases, DNA demethylases, histone acetyltransferases, histone deacetylases, and the like). The tracrRNA extension sequence can have a primer binding site or a molecular index (e.g., barcode sequence). The tracrRNA extension sequence can have one or more affinity tags.

### Signal-Molecule Guide Linker Sequence

The linker sequence of a single-molecule guide nucleic acid can have a length from about 3 nucleotides to about 100 nucleotides. In Jinek et al., supra, for example, a simple 4 nucleotide “tetraloop” (-GAAA-) was used, Science, 337(6096):816-821 (2012). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can have any of a variety of sequences, although in some examples the linker will not have sequences that have extensive regions of homology with other portions of the guide RNA, which might cause intramolecular binding that could interfere with other functional regions of the guide. In Jinek et al., supra, a simple 4 nucleotide sequence -GAAA- was used, Science, 337(6096):816-821 (2012), but numerous other sequences, including longer sequences can likewise be used.

The linker sequence can have a functional moiety. For example, the linker sequence can have one or more features, including an aptamer, a ribozyme, a protein-interacting hairpin, a protein binding site, a CRISPR array, an intron, or an exon. The linker sequence can have at least about 1, 2, 3, 4, or 5 or more functional moieties. In some examples, the linker sequence can have at most about 1, 2, 3, 4, or 5 or more functional moieties.

Genome engineering strategies include correcting cells by insertion or correction of one or more mutations at, within, or near the WAS gene, or by knocking-in WAS gene cDNA into the locus of the corresponding WAS gene or safe harbor site.

The methods of the present disclosure can involve correction of one or both of the mutant alleles. Gene editing to correct the mutation has the advantage of restoration of correct expression levels and temporal control. Sequencing the patient's WAS gene alleles allows for design of the gene editing strategy to best correct the identified mutation(s).

A step of the ex vivo methods of the present disclosure can have editing/correcting the patient specific iPSC cells using genome engineering. Alternatively, a step of the ex vivo methods of the present disclosure can have editing/correcting the progenitor cell, primary hepatocyte, or mesenchymal stem cell. Likewise, a step of the in vivo methods of the disclosure involves editing/correcting the cells in Wiskott-Aldrich Syndrome (WAS) patient using genome engineering. Similarly, a step in the cellular methods of the present disclosure can have editing/correcting the WAS gene in a human cell by genome engineering.

Wiskott-Aldrich Syndrome (WAS) patients exhibit a wide range of mutations in the WAS gene. Therefore, different patients will generally require different correction strategies. Any CRISPR endonuclease may be used in the methods of the present disclosure, each CRISPR endonuclease having its own associated PAM, which may or may not be disease specific.

For example, the mutation can be corrected by the insertions or deletions that arise due to the imprecise NHEJ repair pathway. If the patient's WAS gene has an inserted or deleted base, a targeted cleavage can result in a NHEJ-mediated insertion or deletion that restores the frame. Missense mutations can also be corrected through NHEJ-mediated correction using one or more guide RNA. The ability or likelihood of the cut(s) to correct the mutation can be designed or evaluated based on the local sequence and micro-homologies. NHEJ can also be used to delete segments of the gene, either directly or by altering splice donor or acceptor sites through cleavage by one gRNA targeting several locations, or several gRNAs. This may be useful if an amino acid, domain or exon contains the mutations and can be removed or inverted, or if the deletion otherwise restored function to the protein. Pairs of guide strands have been used for deletions and corrections of inversions.

Alternatively, the donor for correction by HDR contains the corrected sequence with small or large flanking homology arms to allow for annealing. HDR is essentially an error-free mechanism that uses a supplied homologous DNA sequence as a template during DSB repair. The rate of homology directed repair (HDR) is a function of the distance between the mutation and the cut site so choosing overlapping or nearest target sites is important. Templates can include extra sequences flanked by the homologous regions or can contain a sequence that differs from the genomic sequence, thus allowing sequence editing.

In addition to correcting mutations by NHEJ or HDR, a range of other options are possible. If there are small or large deletions or multiple mutations, a cDNA can be knocked in that contains the exons affected. A full length cDNA can be knocked into any “safe harbor”, but must use a supplied or other promoter. If this construct is knocked into the correct location, it will have physiological control, similar to the normal gene. Pairs of nucleases can be used to delete mutated gene regions, though a donor would usually have to be provided to restore function. In this case two gRNA would be supplied and one donor sequence.

Some genome engineering strategies involve correction of one or more mutations in or near the WAS gene, or knocking-in WAS gene cDNA into the locus of the corresponding gene or a safe harbor locus by homology directed repair (HDR), which is also known as homologous recombination (HR). Homology directed repair can be one strategy for treating patients that have one or more mutations in or near the WAS gene. These strategies can restore the WAS gene and reverse, treat, and/or mitigate the diseased state. These strategies can require a more custom approach based on the location of the patient's mutation(s). Donor nucleotides for correcting mutations often are small (<300 bp). This is advantageous, as HDR efficiencies may be inversely related to the size of the donor molecule. Also, it is expected that the donor templates can fit into size constrained adeno-associated virus (AAV) molecules, which have been shown to be an effective means of donor template delivery.

Homology direct repair is a cellular mechanism for repairing double-stranded breaks (DSBs). The most common form is homologous recombination. There are additional pathways for HDR, including single-strand annealing and alternative-HDR. Genome engineering tools allow researchers to manipulate the cellular homologous recombination pathways to create site-specific modifications to the genome. It has been found that cells can repair a double-stranded break using a synthetic donor molecule provided in trans. Therefore, by introducing a double-stranded break near a specific mutation and providing a suitable donor, targeted changes can be made in the genome. Specific cleavage increases the rate of HDR more than 1,000 fold above the rate of 1 in 10⁶ cells receiving a homologous donor alone. The rate of homology directed repair (HDR) at a particular nucleotide is a function of the distance to the cut site, so choosing overlapping or nearest target sites is important. Gene editing offers the advantage over gene addition, as correcting in situ leaves the rest of the genome unperturbed.

Supplied donors for editing by HDR vary markedly but can contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors have been used. These oligonucleotides range in size from less than 100 nt to over many kb, though longer ssDNA can also be generated and used. Double-stranded donors can be used, including PCR amplicons, plasmids, and mini-circles. In general, it has been found that an AAV vector can be a very effective means of delivery of a donor template, though the packaging limits for individual donors is <5 kb. Active transcription of the donor increased HDR three-fold, indicating the inclusion of promoter may increase conversion. Conversely, CpG methylation of the donor decreased gene expression and HDR

In addition to wildtype endonucleases, such as Cas9, nickase variants exist that have one or the other nuclease domain inactivated resulting in cutting of only one DNA strand. HDR can be directed from individual Cas nickases or using pairs of nickases that flank the target area. Donors can be single-stranded, nicked, or dsDNA.

The donor DNA can be supplied with the nuclease or independently by a variety of different methods, for example by transfection, nano-particle, micro-injection, or viral transduction. The method of transfection may include reagent or chemical transfection which include lipid based or salt/chemical based transfection. A range of tethering options have been proposed to increase the availability of the donors for HDR. Examples include attaching the donor to the nuclease, attaching to DNA binding proteins that bind nearby, or attaching to proteins that are involved in DNA end binding or repair.

The repair pathway choice can be guided by a number of culture conditions, such as those that influence cell cycling, or by targeting of DNA repair and associated proteins. For example, to increase HDR key NHEJ molecules can be suppressed, such as KU70, KU80 or DNA ligase IV.

Without a donor present, the ends from a DNA break or ends from different breaks can be joined using the several nonhomologous repair pathways in which the DNA ends are joined with little or no base-pairing at the junction. In addition to canonical NHEJ, there are similar repair mechanisms, such as alt-NHEJ. If there are two breaks, the intervening segment can be deleted or inverted. NHEJ repair pathways can lead to insertions, deletions or mutations at the joints.

NHEJ was used to insert a 15-kb inducible gene expression cassette into a defined locus in human cell lines after nuclease cleavage. Maresca, M., Lin, V. G., Guo, N. & Yang, Y., Genome Res 23, 539-546 (2013).

In addition to genome editing by NHEJ or HDR, site-specific gene insertions have been conducted that use both the NHEJ pathway and HR. A combination approach may be applicable in certain settings, possibly including intron/exon borders. NHEJ may prove effective for ligation in the intron, while the error-free HDR may be better suited in the coding region.

The WAS gene contains a number of exons. Any one or more of these exons or nearby introns can be repaired in order to correct a mutation and restore WAS protein activity. Alternatively, there are various mutations associated with Wiskott-Aldrich Syndrome (WAS), which are a combination of insertions, deletions, missense, nonsense, frameshift and other mutations, with the common effect of inactivating WAS gene. Any one or more of the mutations can be repaired in order to restore the inactive WAS gene. As a further alternative, WAS gene cDNA or minigene (which may have natural or synthetic enhancer and promoter, one or more exons, and natural or synthetic introns, and natural or synthetic 3′UTR and polyadenylation signal) can be knocked-in to the locus of the corresponding gene or knocked-in to a safe harbor site, such as AAVS1(PPP1R12C), ALB, Angpt13, ApoC3, ASGR2, CCR5, FIX (F9), G6PC, Gys2, HGD, Lp(a), Pcsk9, Serpinal, TF, and/or TTR. The safe harbor locus can be selected from the group consisting of: AAVS1 (PPP1R12C), ALB, Angpt13, ApoC3, ASGR2, CCR5, FIX (F9), G6PC, Gys2, HGD, Lp(a), Pcsk9, Serpinal, TF, and TTR. In some examples, the methods can provide one gRNA or a pair of gRNAs that can be used to facilitate incorporation of a new sequence from a polynucleotide donor template to correct one or more mutations or to knock-in a part of or the entire WAS gene or cDNA.

The methods can provide gRNA pairs that make a deletion by cutting the gene twice, one gRNA cutting at the 5′ end of one or more mutations and the other gRNA cutting at the 3′ end of one or more mutations that facilitates insertion of a new sequence from a polynucleotide donor template to replace the one or more mutations. The cutting can be accomplished by a pair of DNA endonucleases that each makes a DSB in the genome, or by multiple nickases that together make a DSB in the genome.

Alternatively, the methods can provide one gRNA to make one double-strand cut around one or more mutations that facilitates insertion of a new sequence from a polynucleotide donor template to replace the one or more mutations. The double-strand cut can be made by a single DNA endonuclease or multiple nickases that together make a DSB in the genome.

Illustrative modifications within the WAS gene include replacements at, within, or near (proximal) to the mutations referred to above, such as within the region of less than 3 kb, less than 2 kb, less than 1 kb, less than 0.5 kb upstream or downstream of the specific mutation. Given the relatively wide variations of mutations in the WAS gene, it will be appreciated that numerous variations of the replacements referenced above (including without limitation larger as well as smaller deletions), would be expected to result in restoration of the WAS gene.

Such variants can include replacements that are larger in the 5′ and/or 3′ direction than the specific mutation in question, or smaller in either direction. Accordingly, by “near” or “proximal” with respect to specific replacements, it is intended that the SSB or DSB locus associated with a desired replacement boundary (also referred to herein as an endpoint) can be within a region that is less than about 3 kb from the reference locus noted. The SSB or DSB locus can be more proximal and within 2 kb, within 1 kb, within 0.5 kb, or within 0.1 kb. In the case of small replacement, the desired endpoint can be at or “adjacent to” the reference locus, by which it is intended that the endpoint can be within 100 bp, within 50 bp, within 25 bp, or less than about 10 bp to 5 bp from the reference locus.

Examples having larger or smaller replacements can be expected to provide the same benefit, as long as the WAS protein activity is restored. It is thus expected that many variations of the replacements described and illustrated herein can be effective for ameliorating Wiskott-Aldrich Syndrome (WAS).

In order to ensure that the pre-mRNA is properly processed following deletion, the surrounding splicing signals can be deleted. Splicing donor and acceptors are generally within 100 base pairs of the neighboring intron. Therefore, in some examples, methods can provide all gRNAs that cut approximately +/−100-3100 bp with respect to each exon/intron junction of interest.

For any of the genome editing strategies, gene editing can be confirmed by sequencing or PCR analysis.

### Target Sequence Selection

Shifts in the location of the 5′ boundary and/or the 3′ boundary relative to particular reference loci can be used to facilitate or enhance particular applications of gene editing, which depend in part on the endonuclease system selected for the editing, as further described and illustrated herein.

In a first non-limiting example of such target sequence selection, many endonuclease systems have rules or criteria that can guide the initial selection of potential target sites for cleavage, such as the requirement of a PAM sequence motif in a particular position adjacent to the DNA cleavage sites in the case of CRISPR Type II or Type V endonucleases.

In another non-limiting example of target sequence selection or optimization, the frequency of off-target activity for a particular combination of target sequence and gene editing endonuclease (i.e. the frequency of DSBs occurring at sites other than the selected target sequence) can be assessed relative to the frequency of on-target activity. In some cases, cells that have been correctly edited at the desired locus can have a selective advantage relative to other cells. Illustrative, but nonlimiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some cases, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

Whether any selective advantage is applicable or any directed selection is to be applied in a particular case, target sequence selection can also be guided by consideration of off-target frequencies in order to enhance the effectiveness of the application and/or reduce the potential for undesired alterations at sites other than the desired target. As described further and illustrated herein and in the art, the occurrence of off-target activity can be influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs can be regularly induced and repaired in normal cells. During repair, the original sequence can be reconstructed with complete fidelity, however, in some cases, small insertions or deletions (referred to as “InDels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB can be introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which may or may not be desired given the particular circumstances.

The examples provided herein further illustrate the selection of various target regions for the creation of DSBs designed to induce replacements that result in restoration of WAS protein activity, as well as the selection of specific target sequences within such regions that are designed to minimize off-target events relative to on-target events.

### Nucleic Acid Modifications (Chemical and Structural Modifications)

In some cases, polynucleotides introduced into cells can have one or more modifications that can be used individually or in combination, for example, to enhance activity, stability or specificity, alter delivery, reduce innate immune responses in host cells, or for other enhancements, as further described herein and known in the art.

In certain examples, modified polynucleotides can be used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified, as described and illustrated below. Such modified polynucleotides can be used in the CRISPR/Cas9/Cpf1 system to edit any one or more genomic loci.

Using the CRISPR/Cas9/Cpf1 system for purposes of nonlimiting illustrations of such uses, modifications of guide RNAs can be used to enhance the formation or stability of the CRISPR/Cas9/Cpf1 genome editing complex having guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in aspects in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNases present in the cell), modifications that enhance translation of the resulting product (i.e. the endonuclease), and/or modifications that decrease the likelihood or degree to which the RNAs introduced into cells elicit innate immune responses.

Combinations of modifications, such as the foregoing and others, can likewise be used. In the case of CRISPR/Cas9/Cpf1, for example, one or more types of modifications can be made to guide RNAs (including those exemplified above), and/or one or more types of modifications can be made to RNAs encoding Cas endonuclease (including those exemplified above).

By way of illustration, guide RNAs used in the CRISPR/Cas9/Cpf1 system, or other smaller RNAs can be readily synthesized by chemical means, enabling a number of modifications to be readily incorporated, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach that can be used for generating chemically-modified RNAs of greater length is to produce two or more molecules that are ligated together. Much longer RNAs, such as those encoding a Cas9 endonuclease, are more readily generated enzymatically. While fewer types of modifications are available for use in enzymatically produced RNAs, there are still modifications that can be used to, e.g., enhance stability, reduce the likelihood or degree of innate immune response, and/or enhance other attributes, as described further below and in the art; and new types of modifications are regularly being developed.

By way of illustration of various types of modifications, especially those used frequently with smaller chemically synthesized RNAs, modifications can have one or more nucleotides modified at the 2′ position of the sugar, in some aspects a 2′-O-alkyl, 2′-O-alkyl-O-alkyl, or 2′-fluoro-modified nucleotide. In some examples, RNA modifications can have 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those having modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones [see De Mesmaeker et al., Ace. Chem. Res., 28:366-374 (1995)]; morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates having 3′alkylene phosphonates and chiral phosphonates, phosphinates, phosphoramidates having 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, Biochemistry, 41(14): 4503-4510 (2002); Genesis, Volume 30, Issue 3, (2001); Heasman, Dev. Biol., 243: 209-214 (2002); Nasevicius et al., Nat. Genet., 26:216-220 (2000); Lacerra et al., Proc. Natl. Acad. Sci., 97: 9591-9596 (2000); and U.S. Pat. No. 5,034,506, issued Jul. 23, 1991.

Cyclohexenyl nucleic acid oligonucleotide mimetics are described in Wang et al., J. Am. Chem. Soc., 122: 8595-8602 (2000).

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These have those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂, or O(CH₂)n CH₃, where n is from 1 to about 10; C1 to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al, Helv. Chim. Acta, 1995, 78, 486). Other modifications include 2′-methoxy (2′-O—CH₃), 2′-propoxy (2′-OCH₂CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage, i.e., the backbone, of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative United States patents that teach the preparation of PNA compounds have, but are not limited to, U.S. Pat. Nos. 5,539,082; 5,714,331; and 5,719,262. Further teaching of PNA compounds can be found in Nielsen et al, Science, 254: 1497-1500 (1991).

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine, and 2,6-diaminopurine. Komberg, A., DNA Replication, W. H. Freeman & Co., San Francisco, pp 75-77 (1980); Gebeyehu et al., Nucl. Acids Res. 15:4513 (1997). A “universal” base known in the art, e.g., inosine, can also be included, 5-Me-C substitutions have been shown to increase nucleic acid duplex stability by 0.6-1.2° C. (Sanghvi, Y. S., in Crooke, S. T, and Lebleu. B., eds., Antisense Research and Applications. CRC Press, Boca Raton, 1993, pp. 276-278) and are aspects of base substitutions.

Modified nucleobases can have other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can have those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications’, pages 289-302, Crooke, S. T, and Lebleu. B, ea., CRC Press, 1993. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, having 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T, and Lebleu, B., eds, ‘Antisense Research and Applications’, CRC Press, Boca Raton, 1993, pp. 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and US Patent Application Publication 2003/0158403.

Thus, the term “modified” refers to a non-natural sugar, phosphate, or base that is incorporated into a guide RNA, an endonuclease, or both a guide RNA and an endonuclease. It is not necessary for all positions in a given oligonucleotide to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties have, but are not limited to, lipid moieties such as a cholesterol moiety [Letsinger et al., Proc. Natl. Acad. Sci. USA, 86: 6553-6556 (1989)]; cholic acid [Manoharan et al., Bioorg. Med. Chem. Let., 4: 1053-1060 (1994)]; a thioether, e.g., hexyl-S-tritylthiol [Manoharan et al, Ann. N. Y. Acad. Sci., 660: 306-309 (1992) and Manoharan et al., Bioorg. Med. Chem. Let., 3: 2765-2770 (1993)]; a thiocholesterol [Oberhauser et al., Nucl. Acids Res., 20: 533-538 (1992)]; an aliphatic chain, e.g., dodecandiol or undecyl residues [Kabanov et al., FEBS Lett., 259: 327-330 (1990) and Svinarchuk et al., Biochimie, 75: 49-54 (1993)]; a phospholipid, e.g., di-hexadecyl-rac-glycerol or triethylammonium 1,2-di-O-hexadecyl-rac-glycero-3-H-phosphonate [Manoharan et al., Tetrahedron Lett., 36: 3651-3654 (1995) and Shea et al., Nucl. Acids Res., 18: 3777-3783 (1990)]; a polyamine or a polyethylene glycol chain [Mancharan et al., Nucleosides & Nucleotides, 14: 969-973 (1995)]; adamantane acetic acid [Manoharan et al., Tetrahedron Lett., 36: 3651-3654 (1995)]; a palmityl moiety [(Mishra et al., Biochim. Biophys. Acta, 1264: 229-237 (1995)]; or an octadecylamine or hexylamino-carbonyl-t oxycholesterol moiety [Crooke et al., J. Pharmacol. Exp. Ther., 277: 923-937 (1996)]. See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; the contents of each of which are herein incorporated by reference in their entirety.

Sugars and other moieties can be used to target proteins and complexes having nucleotides, such as cationic polysomes and liposomes, to particular sites. For example, hepatic cell directed transfer can be mediated via asialoglycoprotein receptors (ASGPRs); see, e.g., Hu, et al., Protein Pept Lett. 21(10); 1025-30 (2014). Other systems known in the art and regularly developed can be used to target biomolecules of use in the present case and/or complexes thereof to particular target cells of interest.

These targeting moieties or conjugates can include conjugate groups covalently bound to functional groups, such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of certain embodiments of the present disclosure, include groups that improve uptake, distribution, metabolism or excretion of the compounds of the present disclosure. Representative conjugate groups are disclosed in International Patent Application No. PCT/US92/09196, filed Oct. 23, 1992 (published as WO1993007883), and U.S. Pat. No. 6,287,860, the contents of each of which are herein incorporated by reference in their entirety. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the contents of each of which are herein incorporated by reference in their entirety.

Longer polynucleotides that are less amenable to chemical synthesis and are typically produced by enzymatic synthesis can also be modified by various means. Such modifications can include, for example, the introduction of certain nucleotide analogs, the incorporation of particular sequences or other moieties at the 5′ or 3′ ends of molecules, and other modifications. By way of illustration, the mRNA encoding Cas9 is approximately 4 kb in length and can be synthesized by in vitro transcription. Modifications to the mRNA can be applied to, e.g., increase its translation or stability (such as by increasing its resistance to degradation with a cell), or to reduce the tendency of the RNA to elicit an innate immune response that is often observed in cells following introduction of exogenous RNAs, particularly longer RNAs such as that encoding Cas9.

Numerous such modifications have been described in the art, such as polyA tails, 5′ cap analogs (e.g., Anti Reverse Cap Analog (ARCA) or m7G(5′)ppp(5′)G (mCAP)), modified 5′ or 3′ untranslated regions (UTRs), use of modified bases (such as Pseudo-UTP, 2-Thio-UTP, 5-Methylcytidine-5′-Triphosphate (5-Methyl-CTP) or N6-Methyl-ATP), or treatment with phosphatase to remove 5′ terminal phosphates. These and other modifications are known in the art, and new modifications of RNAs are regularly being developed.

There are numerous commercial suppliers of modified RNAs, including for example, TriLink Biotech. AxoLabs, Bio-Synthesis Inc., Dharmacon and many others. As described by TriLink, for example, 5-Methyl-CTP can be used to impart desirable characteristics, such as increased nuclease stability, increased translation or reduced interaction of innate immune receptors with in vitro transcribed RNA. 5-Methylcytidine-5′-Triphosphate (5-Methyl-CTP), N6-Methyl-ATP, as well as Pseudo-UTP and 2-Thio-UTP, have also been shown to reduce innate immune stimulation in culture and in vivo while enhancing translation, as illustrated in publications by Kormann et al, and Warren et al. referred to below.

It has been shown that chemically modified mRNA delivered in vivo can be used to achieve improved therapeutic effects; see, e.g., Kormann et al., Nature Biotechnology 29, 154-157 (2011). Such modifications can be used, for example, to increase the stability of the RNA molecule and/or reduce its immunogenicity. Using chemical modifications such as Pseudo-U, N6-Methyl-A, 2-Thio-U and 5-Methyl-C, it was found that substituting just one quarter of the uridine and cytidine residues with 2-Thio-U and 5-Methyl-C respectively resulted in a significant decrease in toll-like receptor (TLR) mediated recognition of the mRNA in mice. By reducing the activation of the innate immune system, these modifications can be used to effectively increase the stability and longevity of the mRNA in vivo; see, e.g., Kormann et al., supra.

It has also been shown that repeated administration of synthetic messenger RNAs incorporating modifications designed to bypass innate anti-viral responses can reprogram differentiated human cells to pluripotency. See, e.g., Warren, et al., Cell Stem Cell, 7(5):618-30 (2010). Such modified mRNAs that act as primary reprogramming proteins can be an efficient means of reprogramming multiple human cell types. Such cells are referred to as induced pluripotency stem cells (iPSCs), and it was found that enzymatically synthesized RNA incorporating 5-Methyl-CTP, Pseudo-UTP and an Anti Reverse Cap Analog (ARCA) could be used to effectively evade the cell's antiviral response; see, e.g., Warren et al., supra.

Other modifications of polynucleotides described in the art include, for example, the use of polyA tails, the addition of 5′ cap analogs (such as m7G(5′)ppp(5′)G (mCAP)), modifications of 5′ or 3′ untranslated regions (UTRs), or treatment with phosphatase to remove 5′ terminal phosphates—and new approaches are regularly being developed.

A number of compositions and techniques applicable to the generation of modified RNAs for use herein have been developed in connection with the modification of RNA interference (RNAi), including small-interfering RNAs (siRNAs). siRNAs present particular challenges in vivo because their effects on gene silencing via mRNA interference are generally transient, which can require repeat administration. In addition, siRNAs are double-stranded RNAs (dsRNA) and mammalian cells have immune responses that have evolved to detect and neutralize dsRNA, which is often a by-product of viral infection. Thus, there are mammalian enzymes such as PKR (dsRNA-responsive kinase), and potentially retinoic acid-inducible gene I (RIG-I), that can mediate cellular responses to dsRNA, as well as Toll-like receptors (such as TLR3, TLR7 and TLR8) that can trigger the induction of cytokines in response to such molecules; see, e.g., the reviews by Angart et al., Pharmaceuticals (Basel) 6(4): 440-468 (2013): Kanasty et al., Molecular Therapy 20(3): 513-524 (2012); Burnett et al., Biotechnol J. 6(9): 1130-46 (2011); Judge and MacLachlan, Hum Gene Ther 19(2): 111-24 (2008); and references cited therein.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., Annual Review of Chemical and Biomolecular Engineering, 2: 77-96 (2011); Gaglione and Messere, Mini Rev Med Chem, 10(7):578-95 (2010); Chemolovskaya et al, Curr Opin Mol Ther., 12(2): 158-67 (2010); Deleavey et al., Curr Protoc Nucleic Acid Chem Chapter 16: Unit 16.3 (2009); Behlke, Oligonucleotides 18(4):305-19 (2008); Fucini et al., Nucleic Acid Ther 22(3): 205-210 (2012); Bremsen et al., Front Genet 3:154 (2012).

As noted above, there are a number of commercial suppliers of modified RNAs, many of which have specialized in modifications designed to improve the effectiveness of siRNAs. A variety of approaches are offered based on various findings reported in the literature. For example, Dharmacon notes that replacement of a non-bridging oxygen with sulfur (phosphorothioate, PS) has been extensively used to improve nuclease resistance of siRNAs, as reported by Kole, Nature Reviews Drug Discovery 11:125-140 (2012). Modifications of the 2′-position of the ribose have been reported to improve nuclease resistance of the internucleotide phosphate bond while increasing duplex stability (Tm), which has also been shown to provide protection from immune activation. A combination of moderate PS backbone modifications with small, well-tolerated 2′-substitutions (2′-O-Methyl, 2′-Fluoro, 2′-Hydro) have been associated with highly stable siRNAs for applications in vivo, as reported by Soutschek et al. Nature 432:173-178 (2004); and 2′-O-Methyl modifications have been reported to be effective in improving stability as reported by Volkov, Oligonucleotides 19:191-202 (2009). With respect to decreasing the induction of innate immune responses, modifying specific sequences with 2′-O-Methyl, 2′-Fluoro, 2′-Hydro have been reported to reduce TLR7/TLR8 interaction while generally preserving silencing activity; see, e.g., Judge et al., Mol. Ther. 13:494-505 (2006); and Cekaite et al., J. Mol. Biol. 365:90-108 (2007). Additional modifications, such as 2-thiouracil, pseudouracil, 5-methylcytosine, 5-methyluracil, and N6-methyladenosine have also been shown to minimize the immune effects mediated by TLR3, TLR7, and TLR8; see, e.g., Kariko, K et al., Immunity 23:165-175 (2005).

As is also known in the art, and commercially available, a number of conjugates can be applied to polynucleotides, such as RNAs, for use herein that can enhance their delivery and/or uptake by cells, including for example, cholesterol, tocopherol and folic acid, lipids, peptides, polymers, linkers and aptamers; see, e.g., the review by Winkler, Ther. Deliv. 4:791-809 (2013), and references cited therein.

### Codon-Optimization

A polynucleotide encoding a site-directed polypeptide can be codon-optimized according to methods standard in the art for expression in the cell containing the target DNA of interest. For example, if the intended target nucleic acid is in a human cell, a human codon-optimized polynucleotide encoding Cas9 is contemplated for use for producing the Cas9 polypeptide.

### Complexes of a Genome-Targeting Nucleic Acid and a Site-Directed Polypeptide

A genome-targeting nucleic acid interacts with a site-directed polypeptide (e.g., a nucleic acid-guided nuclease such as Cas9), thereby forming a complex. The genome-targeting nucleic acid guides the site-directed polypeptide to a target nucleic acid.

### Ribonucleoprotein Complexes (RNPs)

The site-directed polypeptide and genome-targeting nucleic acid can each be administered separately to a cell or a patient. On the other hand, the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

### Nucleic Acids Encoding System Components

The present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the aspects of the methods of the disclosure.

### Specific Methods and Compositions

Accordingly, the present disclosure relates in particular to the following non-limiting methods according to the disclosure: In a first method, Method 1, the present disclosure provides a method for editing a Wiskott-Aldrich syndrome gene (WAS gene) in a human cell by genome editing, the method having the step of: introducing into the human cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and results in restoration of WAS protein activity.

In another method, Method 2, the present disclosure provides an ex vivo method for treating a patient with Wiskott-Aldrich Syndrome (WAS), the method having the steps of: creating a patient specific induced pluripotent stem cell (iPSC); editing at, within, or near a Wiskott-Aldrich syndrome gene (WAS gene) or other DNA sequences that encode regulatory elements of the WAS gene of the iPSC; differentiating the genome-edited iPSC into a hepatocyte; and implanting the hepatocyte into the patient.

In another method, Method 3, the present disclosure provides an ex vivo method for treating a patient with Wiskott-Aldrich Syndrome (WAS) as provided in Method 2 wherein the creating step has: a) isolating a somatic cell from the patient; and b) introducing a set of pluripotency-associated genes into the somatic cell to induce the somatic cell to become a pluripotent stem cell.

In another method, Method 4, the present disclosure provides an ex vivo method for treating a patient with Wiskott-Aldrich Syndrome (WAS) as provided in Method 3, wherein the somatic cell is a fibroblast.

In another method, Method 5, the present disclosure provides an ex vivo method for treating a patient with Wiskott-Aldrich Syndrome (WAS) as provided in Method 3, wherein the set of pluripotency-associated genes is one or more of the genes selected from the group consisting of OCT4, SOX2, KLF4, Lin28, NANOG and cMYC.

In another method, Method 6, the present disclosure provides an ex vivo method for treating a patient with Wiskott-Aldrich Syndrome (WAS) as provided in any one of Methods 2-5, wherein the editing step has introducing into the iPSC one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and results in restoration of WAS protein activity.

In another method, Method 7, the present disclosure provides an ex vivo method for treating a patient with Wiskott-Aldrich Syndrome (WAS) as provided in any one of Methods 2-6, wherein the differentiating step has one or more of the following to differentiate the genome-edited iPSC into a hepatocyte, contacting the genome-edited iPSC with one or more of activin, B27 supplement, FGF4, HGF, BMP2, BMP4, Oncostatin M, Dexametason.

In another method, Method 8, the present disclosure provides an ex vivo method for treating a patient with Wiskott-Aldrich Syndrome (WAS) as provided in any one of Methods 2-7, wherein the implanting step has implanting the hepatocyte into the patient by local injection, systemic infusion, or combinations thereof.

In another method, Method 9, the present disclosure provides an ex vivo method for treating a patient with Wiskott-Aldrich Syndrome (WAS), the method having the steps of: performing a biopsy of the patient's liver; isolating a liver specific progenitor cell or primary hepatocyte; editing at, within, or near a Wiskott-Aldrich syndrome gene (WAS gene) or other DNA sequences that encode regulatory elements of the WAS gene of the progenitor cell or primary hepatocyte; and implanting the progenitor cell or primary hepatocyte into the patient.

In another method, Method 10, the present disclosure provides an ex vivo method for treating a patient with Wiskott-Aldrich Syndrome (WAS) as provided in Method 9, wherein the isolating step has: perfusion of fresh liver tissues with digestion enzymes, cell differential centrifugation, cell culturing, or combinations thereof.

In another method, Method 11, the present disclosure provides an ex vivo method for treating a patient with Wiskott-Aldrich Syndrome (WAS) as provided in Methods 9 or 10, wherein the editing step has introducing into the progenitor cell or primary hepatocyte one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity.

In another method, Method 12, the present disclosure provides an ex vivo method for treating a patient with Wiskott-Aldrich Syndrome (WAS) as provided in any one of Methods 9-11, wherein the implanting the progenitor cell or primary hepatocyte into the patient by local injection, systemic infusion, or combinations thereof.

In another method, Method 13, the present disclosure provides an ex vivo method for treating a patient with Wiskott-Aldrich Syndrome (WAS), the method having the steps of: isolating a mesenchymal stem cell from the patient; editing at, within, or near a Wiskott-Aldrich syndrome gene (WAS gene) or other DNA sequences that encode regulatory elements of the WAS gene of the mesenchymal stem cell from the patient; differentiating the genome-edited mesenchymal stem cell into a heptocyte; and implanting the hepatocyte into the patient.

In another method, Method 14, the present disclosure provides an ex vivo method for treating a patient with Wiskott-Aldrich Syndrome (WAS) as provided in Method 13, wherein the mesenchymal stem cell is isolated from the patient's bone marrow by performing a biopsy of the patient's bone marrow or the mesenchymal stem cell is isolated from peripheral blood.

In another method, Method 15, the present disclosure provides an ex vivo method for treating a patient with Wiskott-Aldrich Syndrome (WAS) as provided in Method 13, wherein the isolating step has: aspiration of bone marrow and isolation of mesenchymal cells by density centrifugation using Percoll™.

In another method, Method 16, the present disclosure provides an ex vivo method for treating a patient with Wiskott-Aldrich Syndrome (WAS) as provided in any one of Methods 13-15, wherein the editing step has introducing into the mesenchymal stem cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity.

In another method, Method 17, the present disclosure provides an ex vivo method for treating a patient with Wiskott-Aldrich Syndrome (WAS) as provided in any one of Methods 13-16, wherein the differentiating step has one or more of the following to differentiate the genome-edited stem cell into a hepatocyte: contacting the genome-edited stem cell with one or more of insulin, transferrin, FGF4, HGF, or bile acids.

In another method, Method 18, the present disclosure provides an ex vivo method for treating a patient with Wiskott-Aldrich Syndrome (WAS) as provided in any one of Methods 13-17, wherein the implanting step has implanting the hepatocyte into the patient by local injection, systemic infusion, or combinations thereof.

In another method, Method 19, the present disclosure provides an in vivo method for treating a patient with Wiskott-Aldrich Syndrome (WAS), the method having the step of editing a Wiskott-Aldrich syndrome gene (WAS gene) in a cell of the patient.

In another method, Method 20, the present disclosure provides an in vivo method for treating a patient with Wiskott-Aldrich Syndrome (WAS) as provided in Method 19, wherein the editing step has introducing into the cell one or more deoxyribonucleic acid (DNA) endonucleases to effect one or more single-strand breaks (SSBs) or double-strand breaks (DSBs) at, within, or near the WAS gene or other DNA sequences that encode regulatory elements of the WAS gene that results in a permanent insertion, correction, or modulation of expression or function of one or more mutations at, within, or near or affecting the expression or function of the WAS gene and restoration of WAS protein activity.

In another method, Method 21, the present disclosure provides a method according to any one of Methods 1, 6, 11, 16, or 20, wherein the one or more DNA endonucleases is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease; or a homolog, recombination of the naturally occurring molecule, codon-optimized, or modified version thereof, and combinations thereof.

In another method, Method 22, the present disclosure provides a method as provided in Method 21, wherein the method has introducing into the cell one or more polynucleotides encoding the one or more DNA endonucleases.

In another method, Method 23, the present disclosure provides a method as provided in Method 21, wherein the method has introducing into the cell one or more ribonucleic acids (RNAs) encoding the one or more DNA endonucleases.

In another method, Method 24, the present disclosure provides a method as provided in Methods 22 or 23, wherein the one or more polynucleotides or one or more RNAs is one or more modified polynucleotides or one or more modified RNAs.

In another method, Method 25, the present disclosure provides a method as provided in Method 21, wherein the DNA endonuclease is a protein or polypeptide.

In another method, Method 26, the present disclosure provides a method as provided in any one of Methods 1-25, wherein the method further has introducing into the cell one or more guide ribonucleic acids (gRNAs).

In another method, Method 27, the present disclosure provides a method as provided in Method 26, wherein the one or more gRNAs are single-molecule guide RNA (sgRNAs).

In another method, Method 28, the present disclosure provides a method as provided in Methods 26 or 27, wherein the one or more gRNAs or one or more sgRNAs is one or more modified gRNAs or one or more modified sgRNAs.

In another method, Method 29, the present disclosure provides a method as provided in any one of Methods 26-28, wherein the one or more DNA endonucleases is pre-complexed with one or more gRNAs or one or more sgRNAs.

In another method, Method 30, the present disclosure provides a method as provided in any one of Methods 1-29, wherein the method further has introducing into the cell a polynucleotide donor template having at least a portion of the wild-type WAS gene or cDNA.

In another method, Method 31, the present disclosure provides a method as provided in Method 30, wherein the at least a portion of the wild-type WAS gene or cDNA can be any of the exons or introns as defined herein. Such portions may include more than one intron or exon as well as sequence regions bridging exons and introns, e.g., intron:exon junctions, intronic regions, fragments or combinations thereof, or the entire WAS gene or cDNA.

In another method, Method 32, the present disclosure provides a method as provided in any one of Methods 30 or 31, wherein the donor template is either a single or double stranded polynucleotide.

In another method, Method 34, the present disclosure provides a method as provided in any one of Methods 1, 6, 11, 16, or 20, wherein the method further has introducing into the cell one guide ribonucleic acid (gRNA) and a polynucleotide donor template having at least a portion of the wild-type WAS gene, and wherein the one or more DNA endonucleases is one or more Cas9 or Cpf1 endonucleases that effect one single-strand break (SSB) or double-strand break (DSB) at a locus at, within, or near the WAS gene or other DNA sequences that encode regulatory elements of the WAS gene that facilitates insertion of a new sequence from the polynucleotide donor template into the chromosomal DNA at the locus that results in a permanent insertion or correction of a part of the chromosomal DNA of the WAS gene or other DNA sequences that encode regulatory elements of the WAS gene proximal to the locus, and wherein the gRNA has a spacer sequence that is complementary to a segment of the locus.

In another method, Method 35, the present disclosure provides a method as provided in Method 34, wherein proximal means nucleotides both upstream and downstream of the locus.

In another method, Method 36, the present disclosure provides a method as provided in any one of Methods 1, 6, 11, 16 or 20, wherein the method further has introducing into the cell two guide ribonucleic acid (gRNAs) and a polynucleotide donor template having at least a portion of the wild-type WAS gene, and wherein the one or more DNA endonucleases is one or more Cas9 or Cpf1 endonucleases that effect a pair of single-strand breaks (SSBs) or double-strand breaks (DSBs), the first at a 5′ locus and the second at a 3′ locus, at, within, or near the WAS gene or other DNA sequences that encode regulatory elements of the WAS gene that facilitates insertion of a new sequence from the polynucleotide donor template into the chromosomal DNA between the 5′ locus and the 3′ locus that results in a permanent insertion or correction of the chromosomal DNA between the 5′ locus and the 3′ locus at, within, or near the WAS gene or other DNA sequences that encode regulatory elements of the WAS gene, and wherein the first guide RNA has a spacer sequence that is complementary to a segment of the 5′ locus and the second guide RNA has a spacer sequence that is complementary to a segment of the 3′ locus.

In another method, Method 37, the present disclosure provides a method as provided in any one of Methods 34-36, wherein the one or two gRNAs are one or two single-molecule guide RNA (sgRNAs).

In another method, Method 38, the present disclosure provides a method as provided in any one of Methods 34-37, wherein the one or two gRNAs or one or two sgRNAs is one or two modified gRNAs or one or two modified sgRNAs.

In another method, Method 39, the present disclosure provides a method as provided in any one of Methods 34-38, wherein the one or more DNA endonucleases is pre-complexed with one or two gRNAs or one or two sgRNAs.

In another method, Method 40, the present disclosure provides a method as provided in any one of Methods 33-38, wherein the at least a portion of the wild-type WAS gene or cDNA can be any of the exons or introns as defined herein. Such portions may include more than one intron or exon as well as sequence regions bridging exons and introns, e.g., intron:exon junctions, intronic regions, fragments or combinations thereof, or the entire WAS gene or cDNA.

In another method, Method 41, the present disclosure provides a method as provided in any one of Methods 34-40, wherein the donor template is either a single or double stranded polynucleotide.

In another method, Method 43, the present disclosure provides a method as provided in any one of Methods 34-42, wherein the SSB, DSB, or 5′ DSB and 3′ DSB are in any intron, exon or junction thereof of the WAS gene.

In another method, Method 44, the present disclosure provide a method as provided in any one of Methods 1, 6, 11, 16, 20, 26-29, or 37-39, wherein the gRNA or sgRNA is directed to one or more SNPs.

In another method, Method 45, the present disclosure provides a method as provided in any one of Methods 1, 6, 11, 16, or 20-44, wherein the insertion or correction is by homology directed repair (HDR).

In another method, Method 46, the present disclosure provides a method as provided in any one of Methods 1, 6, 11, 16, or 20-45, wherein the Cas9 or Cpf1 mRNA, gRNA, and donor template are either each formulated into separate lipid nanoparticles or all co-formulated into a lipid nanoparticle.

In another method, Method 47, the present disclosure provides a method as provided in any one of Methods 1, 6, 11, 16, or 20-46, wherein the Cas9 or Cpf1 mRNA is formulated into a lipid nanoparticle, and both the gRNA and donor template are delivered to the cell by an adeno-associated virus (AAV) vector.

In another method, Method 48, the present disclosure provides a method as provided in any one of Methods 1, 6, 11, 16, or 20-47, wherein the Cas9 or Cpf1 mRNA is formulated into a lipid nanoparticle, and the gRNA is delivered to the cell by electroporation and donor template is delivered to the cell by an adeno-associated virus (AAV) vector.

In another method, Method 50, the present disclosure provides a method as provided in any one of Methods 1, 6, 11, 16, or 20, wherein the restoration of WAS protein activity is compared to wild-type or normal WAS protein activity.

In a first composition, Composition 1, the present disclosure provides one or more guide ribonucleic acids (gRNAs) for editing a WAS gene in a cell from a patient with Wiskott-Aldrich Syndrome (WAS), the one or more gRNAs having a spacer sequence.

In another composition, Composition 2, the present disclosure provides the one or more gRNAs of Composition 1, wherein the one or more gRNAs are one or more single-molecule guide RNAs (sgRNAs).

In another composition, Composition 3, the present disclosure provides the one or more gRNAs or sgRNAs of Compositions 1 or 2, wherein the one or more gRNAs or one or more sgRNAs is one or more modified gRNAs or one or more modified sgRNAs.

The nucleic acid encoding a genome-targeting nucleic acid of the disclosure, a site-directed polypeptide of the disclosure, and/or any nucleic acid or proteinaceous molecule necessary to carry out the aspects of the methods of the disclosure can have a vector (e.g., a recombinant expression vector).

The term “vector” refers to a nucleic acid molecule capable of transporting another nucleic acid to which it has been linked. One type of vector is a “plasmid”, which refers to a circular double-stranded DNA loop into which additional nucleic acid segments can be ligated.

Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operatively linked. Such vectors are referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3, which are described in FIGS. 1A to 1C. Other vectors can be used so long as they are compatible with the host cell.

In some examples, a vector can have one or more transcription and/or translation control elements. Depending on the host/vector system utilized, any of a number of suitable transcription and translation control elements, including constitutive and inducible promoters, transcription enhancer elements, transcription terminators, etc, can be used in the expression vector. The vector can be a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase 111 promoters, including for example U6 and H1, can be advantageous. Descriptions of and parameters for enhancing the use of such promoters are known in art, and additional information and approaches are regularly being described; see, e.g., Ma, H, et, al., Molecular Therapy—Nucleic Acids 3, e161 (2014) doi:10.1038/mtna.2014.12.

The expression vector can also contain a ribosome binding site for translation initiation and a transcription terminator. The expression vector can also have appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter, etc.).

The nucleic acid encoding a genome-targeting nucleic acid of the disclosure and/or a site-directed polypeptide can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### III. Formulations and Delivery

### Pharmaceutically Acceptable Carriers

The ex vivo methods of administering progenitor cells to a subject contemplated herein involve the use of therapeutic compositions having progenitor cells.

Therapeutic compositions can contain a physiologically tolerable carrier together with the cell composition, and optionally at least one additional bioactive agent as described herein, dissolved or dispersed therein as an active ingredient. In some cases, the therapeutic composition is not substantially immunogenic when administered to a mammal or human patient for therapeutic purposes, unless so desired.

In general, the progenitor cells described herein can be administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation having cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the progenitor cells, as described herein, using routine experimentation.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

### Guide RNA Formulation

Guide RNAs of the present disclosure can be formulated with pharmaceutically acceptable excipients such as carriers, solvents, stabilizers, adjuvants, diluents, etc., depending upon the particular mode of administration and dosage form. Guide RNA compositions can be formulated to achieve a physiologically compatible pH, and range from a pH of about 3 to a pH of about 11, about pH 3 to about pH 7, depending on the formulation and route of administration. In some cases, the pH can be adjusted to a range from about pH 5.0 to about pH 8. In some cases, the compositions can have a therapeutically effective amount of at least one compound as described herein, together with one or more pharmaceutically acceptable excipients. Optionally, the compositions can have a combination of the compounds described herein, or can include a second active ingredient useful in the treatment or prevention of bacterial growth (for example and without limitation, anti-bacterial or anti-microbial agents), or can include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

### Delivery

Guide RNA polynucleotides (RNA or DNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 18: 1127-1133 (2011) (which focuses on non-viral delivery vehicles for siRNA that are also useful for delivery of other polynucleotides).

For polynucleotides of the disclosure, the formulation may be selected from any of those taught, for example, in International Application PCT/US2012069610, the contents of which are incorporated herein by reference in its entirety

Polynucleotides, such as guide RNA, sgRNA, and mRNA encoding an endonuclease, can be delivered to a cell or a patient by a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses.

LNPs can also have hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MDI, and 7C1. Examples of neutral lipids are: DPSC, DPPC, POPC, DOPE, and SM. Examples of PEG-modified lipids are: PEG-DMG, PEG-CerC14, and PEG-CerC20.

The lipids can be combined in any number of molar ratios to produce a LNP. In addition, the polynucleotide(s) can be combined with lipid(s) in a wide range of molar ratios to produce a LNP.

As stated previously, the site-directed polypeptide and genome-targeting nucleic acid can each be administered separately to a cell or a patient. On the other hand, the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

RNA is capable of forming specific interactions with RNA or DNA. While this property is exploited in many biological processes, it also comes with the risk of promiscuous interactions in a nucleic acid-rich cellular environment. One solution to this problem is the formation of ribonucleoprotein particles (RNPs), in which the RNA is pre-complexed with an endonuclease. Another benefit of the RNP is protection of the RNA from degradation.

The endonuclease in the RNP can be modified or unmodified. Likewise, the gRNA, crRNA, tracrRNA, or sgRNA can be modified or unmodified. Numerous modifications are known in the art and can be used.

The endonuclease and sgRNA can be generally combined in a 1:1 molar ratio. Alternatively, the endonuclease, crRNA and tracrRNA can be generally combined in a 1:1:1 molar ratio. However, a wide range of molar ratios can be used to produce a RNP.

### AAV (Adeno Associated Virus)

A recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived, and may be from a different AAV serotype than the rAAV genome ITRs, including, but not limited to, AAV serotypes described herein. Production of pseudotyped rAAV is disclosed in, for example, international patent application publication number WO 01/83692.

### AAV Serotypes

AAV particles packaging polynucleotides encoding compositions of the disclosure, e.g., endonucleases, donor sequences, or RNA guide molecules, of the present disclosure may have or be derived from any natural or recombinant AAV serotype. According to the present disclosure, the AAV particles may utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3. AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08. AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC 12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV10, true type AAV (ttAAV), UPENN AAV10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In some embodiments, the AAV serotype may be, or have, a mutation in the AAV9 sequence as described by N Pulicherla et al. (Molecular Therapy 19(6): 1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the AAV serotype may be, or have, a sequence as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may have two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may have two mutations: (1) R587Q where arginine (R; Arg) at amino acid 587 is changed to glutamine (Q; Gin) and (2) R590T where arginine (R; Arg) at amino acid 590 is changed to threonine (T; Thr). As another non-limiting example, may have three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In some embodiments, the AAV serotype may be, or have, a sequence as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

According to the present disclosure, AAV capsid serotype selection or use may be from a variety of species. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In one embodiment, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In one embodiment, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In one embodiment, the AAV may be AAV2G9 which has sequences from AAV2 and AAV9. The AAV2G9 AAV serotype may be, or have, a sequence as described in United States Patent Publication No. US20160017005, the contents of which are herein incorporated by reference in its entirety.

In one embodiment, the AAV may be a serotype generated by the AAV9 capsid library with mutations in amino acids 390-627 (VP1 numbering) as described by Pulicherla et al. (Molecular Therapy 19(6): 1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C. T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C. T1549G; G481R, W509R. L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A. T1609A; L511I, L537M), AAV9.55 (T1605A; F535L), AAV9.58 (C1475T, C1579A; T4921, H527N), AAV.59 (T1336C; Y446H), AAV9.61 (A1493T; N4981), AAV9.64 (C1531A. A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R. K5281), AAV9.93 (A1273G, A1421G, A1638C, C1712T, G1732A, A1744T, A1832T; S425G, Q474R, Q546H, P571L, G578R, T582S, D611V), AAV9.94 (A1675T M559L) and AAV9.95 (T1605A; F535L).

In one embodiment, the AAV may be a serotype having at least one AAV capsid CD8+ T-cell epitope. As a non-limiting example, the serotype may be AAV1, AAV2 or AAV8.

In one embodiment, the AAV may be a serotype selected from any of those found in Table 5 and 6.

In one embodiment, the AAV may be encoded by a sequence, fragment or variant as described in Table 5 or 6.

TABLE 5
AAV Serotypes

| Serotype | SEQ ID NO | Reference information for Serotype Sequence |
|---|---|---|
| AAV1.3 | 4697 | US20030138772 SEQ ID NO: 14 |
| AAV16.3 | 4698 | US20030138772 SEQ ID NO: 105 |
| AAV223.10 | 4699 | US20030138772 SEQ ID NO: 75 |
| AAV223.2 | 4700 | US20030138772 SEQ ID NO: 49 |
| AAV223.2 | 4701 | US20030138772 SEQ ID NO: 76 |
| AAV223.4 | 4702 | US20030138772 SEQ ID NO: 50 |
| AAV223.4 | 4703 | US20030138772 SEQ ID NO: 73 |
| AAV223.5 | 4704 | US20030138772 SEQ ID NO: 51 |
| AAV223.5 | 4705 | US20030138772 SEQ ID NO: 74 |
| AAV223.6 | 4706 | US20030138772 SEQ ID NO: 52 |
| AAV223.6 | 4707 | US20030138772 SEQ ID NO: 78 |
| AAV223.7 | 4708 | US20030138772 SEQ ID NO: 53 |
| AAV223.7 | 4709 | US20030138772 SEQ ID NO: 77 |
| AAV24.1 | 4710 | US20030138772 SEQ ID NO: 101 |
| AAV27.3 | 4711 | US20030138772 SEQ ID NO: 104 |
| AAV29.3 | 4712 | US20030138772 SEQ ID NO: 82 |
| AAV29.3 | 4713 | US20030138772 SEQ ID NO: 11 |
| (AAVbb.1) | | |
| AAV29.4 | 4714 | US20030138772 SEQ ID NO: 12 |
| AAV29.5 | 4715 | US20030138772 SEQ ID NO: 83 |
| AAV29.5 | 4716 | US20030138772 SEQ ID NO: 13 |
| (AAVbb.2) | | |
| AAV3 | 4717 | US20150159173 SEQ ID NO: 12 |
| AAV3.3b | 4718 | US20030138772 SEQ ID NO: 72 |
| AAV3-3 | 4719 | US20150315612 SEQ ID NO: 200 |
| AAV3-3 | 4720 | US20150315612 SEQ ID NO: 217 |
| AAV42.10 | 4721 | US20030138772 SEQ ID NO: 106 |
| AAV42.11 | 4722 | US20030138772 SEQ ID NO: 108 |
| AAV42.12 | 4723 | US20030138772 SEQ ID NO: 113 |
| AAV42.13 | 4724 | US20030138772 SEQ ID NO: 86 |
| AAV42.15 | 4725 | US20030138772 SEQ ID NO: 84 |
| AAV42.1B | 4726 | US20030138772 SEQ ID NO: 90 |
| AAV42.2 | 4727 | US20030138772 SEQ ID NO: 9 |
| AAV42.2 | 4728 | US20030138772 SEQ ID NO: 102 |
| AAV42.3A | 4729 | US20030138772 SEQ ID NO: 87 |
| AAV42.3b | 4730 | US20030138772 SEQ ID NO: 36 |
| AAV42.3B | 4731 | US20030138772 SEQ ID NO: 107 |
| AAV42.4 | 4732 | US20030138772 SEQ ID NO: 33 |
| AAV42.4 | 4733 | US20030138772 SEQ ID NO: 88 |
| AAV42.5A | 4734 | US20030138772 SEQ ID NO: 89 |
| AAV42.5B | 4735 | US20030138772 SEQ ID NO: 91 |
| AAV42.6B | 4736 | US20030138772 SEQ ID NO: 112 |
| AAV42.8 | 4737 | US20030138772 SEQ ID NO: 27 |
| AAV42.8 | 4738 | US20030138772 SEQ ID NO: 85 |
| AAV43.1 | 4739 | US20030138772 SEQ ID NO: 39 |
| AAV43.1 | 4740 | US20030138772 SEQ ID NO: 92 |
| AAV43.12 | 4741 | US20030138772 SEQ ID NO: 41 |
| AAV43.12 | 4742 | US20030138772 SEQ ID NO: 93 |
| AAV43.20 | 4743 | US20030138772 SEQ ID NO: 42 |
| AAV43.20 | 4744 | US20030138772 SEQ ID NO: 99 |
| AAV43.21 | 4745 | US20030138772 SEQ ID NO: 43 |
| AAV43.21 | 4746 | US20030138772 SEQ ID NO: 96 |
| AAV43.23 | 4747 | US20030138772 SEQ ID NO: 44 |
| AAV43.23 | 4748 | US20030138772 SEQ ID NO: 98 |
| AAV43.25 | 4749 | US20030138772 SEQ ID NO: 45 |
| AAV43.25 | 4750 | US20030138772 SEQ ID NO: 97 |
| AAV43.5 | 4751 | US20030138772 SEQ ID NO: 40 |
| AAV43.5 | 4752 | US20030138772 SEQ ID NO: 94 |
| AAV4-4 | 4753 | US20150315612 SEQ ID NO: 201 |
| AAV4-4 | 4754 | US20150315612 SEQ ID NO: 218 |
| AAV44.1 | 4755 | US20030138772 SEQ ID NO: 46 |
| AAV44.1 | 4756 | US20030138772 SEQ ID NO: 79 |
| AAV44.2 | 4757 | US20030138772 SEQ ID NO: 59 |
| AAV44.5 | 4758 | US20030138772 SEQ ID NO: 47 |
| AAV44.5 | 4759 | US20030138772 SEQ ID NO: 80 |
| AAV4407 | 4760 | US20150315612 SEQ ID NO: 90 |
| AAV5 | 4761 | US20030138772 SEQ ID NO: 114 |
| AAV6 | 4762 | US20150315612 SEQ ID NO: 220 |
| AAV6.1 | 4763 | US20150159173 |
| AAV6.12 | 4764 | US20150159173 |
| AAV6.2 | 4765 | US20150159173 |
| AAV7.2 | 4766 | US20030138772 SEQ ID NO: 103 |
| AAV9 | 4767 | US20150315612 SEQ ID NO: 3 |
| (AAVhu.14) | | |
| AAV9 | 4768 | US20150315612 SEQ ID NO: 123 |
| (AAVhu.14) | | |
| AAVA3.1 | 4769 | US20030138772 SEQ ID NO: 120 |
| AAVA3.3 | 4770 | US20030138772 SEQ ID NO: 57 |
| AAVA3.3 | 4771 | US20030138772 SEQ ID NO: 66 |
| AAVA3.4 | 4772 | US20030138772 SEQ ID NO: 54 |
| AAVA3.4 | 4773 | US20030138772 SEQ ID NO: 68 |
| AAVA3.5 | 4774 | US20030138772 SEQ ID NO: 55 |
| AAVA3.5 | 4775 | US20030138772 SEQ ID NO: 69 |
| AAVA3.7 | 4776 | US20030138772 SEQ ID NO: 56 |
| AAVA3.7 | 4777 | US20030138772 SEQ ID NO: 67 |
| AAVC1 | 4778 | US20030138772 SEQ ID NO: 60 |
| AAVC2 | 4779 | US20030138772 SEQ ID NO: 61 |
| AAVC5 | 4780 | US20030138772 SEQ ID NO: 62 |
| AAVCh.5 | 4781 | US20150159173 SEQ ID NO: 46, US20150315612 SEQ ID NO: |
| | | 234 |
| AAVcy.2 | 4782 | US20030138772 SEQ ID NO: 15 |
| (AAV13.3) | | |
| AAVcy.3 | 4783 | US20030138772 SEQ ID NO: 16 |
| (AAV24.1) | | |
| AAVcy.4 | 4784 | US20030138772 SEQ ID NO: 17 |
| (AAV27.3) | | |
| AAVcy.5 | 4785 | US20150315612 SEQ ID NO: 227 |
| AAVcy.5 | 4786 | US20150159173 SEQ ID NO: 8 |
| AAVcy.5 | 4787 | US20150159173 SEQ ID NO: 24 |
| AAVcy.5 | 4788 | US20030138772 SEQ ID NO: 18 |
| (AAV7.2) | | |
| AAVCy.5R1 | 4789 | US20150159173 |
| AAVCy.5R2 | 4790 | US20150159173 |
| AAVCy.5R3 | 4791 | US20150159173 |
| AAVCy.5R4 | 4792 | US20150159173 |
| AAVcy.6 | 4793 | US20030138772 SEQ ID NO: 10 |
| (AAV16.3) | | |
| AAVF1 | 4794 | US20030138772 SEQ ID NO: 109 |
| AAVF3 | 4795 | US20030138772 SEQ ID NO: 111 |
| AAVF5 | 4796 | US20030138772 SEQ ID NO: 110 |
| AAVH2 | 4797 | US20030138772 SEQ ID NO: 26 |
| AAVH6 | 4798 | US20030138772 SEQ ID NO: 25 |
| AAVhu.1 | 4799 | US20150315612 SEQ ID NO: 46 |
| AAVhu.1 | 4800 | US20150315612 SEQ ID NO: 144 |
| AAVhu.10 | 4801 | US20150315612 SEQ ID NO: 56 |
| (AAV16.8) | | |
| AAVhu.10 | 4802 | US20150315612 SEQ ID NO: 156 |
| (AAV16.8) | | |
| AAVhu.11 | 4803 | US20150315612 SEQ ID NO: 57 |
| (AAV16.12) | | |
| AAVhu.11 | 4804 | US20150315612 SEQ ID NO: 153 |
| (AAV16.12) | | |
| AAVhu.12 | 4805 | US20150315612 SEQ ID NO: 59 |
| AAVhu.12 | 4806 | US20150315612 SEQ ID NO: 154 |
| AAVhu.13 | 4807 | US20150159173 SEQ ID NO: 16, US20150315612 SEQ ID NO: 71 |
| AAVhu.13 | 4808 | US20150159173 SEQ ID NO: 32, US20150315612 SEQ ID NO: |
| | | 129 |
| AAVhu.136.1 | 4809 | US20150315612 SEQ ID NO: 165 |
| AAVhu.140.1 | 4810 | US20150315612 SEQ ID NO: 166 |
| AAVhu.140.2 | 4811 | US20150315612 SEQ ID NO: 167 |
| AAVhu.145.6 | 4812 | US20150315612 SEQ ID No: 178 |
| AAVhu.15 | 4813 | US20150315612 SEQ ID NO: 147 |
| AAVhu.15 | 4814 | US20150315612 SEQ ID NO: 50 |
| (AAV33.4) | | |
| AAVhu.156.1 | 4815 | US20150315612 SEQ ID No: 179 |
| AAVhu.16 | 4816 | US20150315612 SEQ ID NO: 148 |
| AAVhu.16 | 4817 | US20150315612 SEQ ID NO: 51 |
| (AAV33.8) | | |
| AAVhu.17 | 4818 | US20150315612 SEQ ID NO: 83 |
| AAVhu.17 | 4819 | US20150315612 SEQ ID NO: 4 |
| (AAV33.12) | | |
| AAVhu.172.1 | 4820 | US20150315612 SEQ ID NO: 171 |
| AAVhu.172.2 | 4821 | US20150315612 SEQ ID NO: 172 |
| AAVhu.173.4 | 4822 | US20150315612 SEQ ID NO: 173 |
| AAVhu.173.8 | 4823 | US20150315612 SEQ ID NO: 175 |
| AAVhu.18 | 4824 | US20150315612 SEQ ID NO: 52 |
| AAVhu.18 | 4825 | US20150315612 SEQ ID NO: 149 |
| AAVhu.19 | 4826 | US20150315612 SEQ ID NO: 62 |
| AAVhu.19 | 4827 | US20150315612 SEQ ID NO: 133 |
| AAVhu.2 | 4828 | US20150315612 SEQ ID NO: 48 |
| AAVhu.2 | 4829 | US20150315612 SEQ ID NO: 143 |
| AAVhu.20 | 4830 | US20150315612 SEQ ID NO: 63 |
| AAVhu.20 | 4831 | US20150315612 SEQ ID NO: 134 |
| AAVhu.21 | 4832 | US20150315612 SEQ ID NO: 65 |
| AAVhu.21 | 4833 | US20150315612 SEQ ID NO: 135 |
| AAVhu.22 | 4834 | US20150315612 SEQ ID NO: 67 |
| AAVhu.22 | 4835 | US20150315612 SEQ ID NO: 138 |
| AAVhu.23 | 4836 | US20150315612 SEQ ID NO: 60 |
| AAVhu.23.2 | 4837 | US20150315612 SEQ ID NO: 137 |
| AAVhu.24 | 4838 | US20150315612 SEQ ID NO: 66 |
| AAVhu.24 | 4839 | US20150315612 SEQ ID NO: 136 |
| AAVhu.25 | 4840 | US20150315612 SEQ ID NO: 49 |
| AAVhu.25 | 4841 | US20150315612 SEQ ID NO: 146 |
| AAVhu.26 | 4842 | US20150159173 SEQ ID NO: 17, US20150315612 SEQ ID NO: 61 |
| AAVhu.26 | 4843 | US20150159173 SEQ ID NO: 33, US20150315612 SEQ ID NO: |
| | | 139 |
| AAVhu.27 | 4844 | US20150315612 SEQ ID NO: 64 |
| AAVhu.27 | 4845 | US20150315612 SEQ ID NO: 140 |
| AAVhu.28 | 4846 | US20150315612 SEQ ID NO: 68 |
| AAVhu.28 | 4847 | US20150315612 SEQ ID NO: 130 |
| AAVhu.29 | 4848 | US20150315612 SEQ ID NO: 69 |
| AAVhu.29 | 4849 | US20150159173 SEQ ID NO: 42, US20150315612 SEQ ID NO: |
| | | 132 |
| AAVhu.29 | 4850 | US20150315612 SEQ ID NO: 225 |
| AAVhu.29R | 4851 | US20150159173 |
| AAVhu.3 | 4852 | US20150315612 SEQ ID NO: 44 |
| AAVhu.3 | 4853 | US20150315612 SEQ ID NO: 145 |
| AAVhu.30 | 4854 | US20150315612 SEQ ID NO: 70 |
| AAVhu.30 | 4855 | US20150315612 SEQ ID NO: 131 |
| AAVhu.31 | 4856 | US20150315612 SEQ ID NO: 1 |
| AAVhu.31 | 4857 | US20150315612 SEQ ID NO: 121 |
| AAVhu.32 | 4858 | US20150315612 SEQ ID NO: 2 |
| AAVhu.32 | 4859 | US20150315612 SEQ ID NO: 122 |
| AAVhu.33 | 4860 | US20150315612 SEQ ID NO: 75 |
| AAVhu.33 | 4861 | US20150315612 SEQ ID NO: 124 |
| AAVhu.34 | 4862 | US20150315612 SEQ ID NO: 72 |
| AAVhu.34 | 4863 | US20150315612 SEQ ID NO: 125 |
| AAVhu.35 | 4864 | US20150315612 SEQ ID NO: 73 |
| AAVhu.35 | 4865 | US20150315612 SEQ ID NO: 164 |
| AAVhu.36 | 4866 | US20150315612 SEQ ID NO: 74 |
| AAVhu.36 | 4867 | US20150315612 SEQ ID NO: 126 |
| AAVhu.37 | 4868 | US20150159173 SEQ ID NO: 34, US20150315612 SEQ ID NO: 88 |
| AAVhu.37 | 4869 | US20150315612 SEQ ID NO: 10, US20150159173 SEQ ID NO: 18 |
| (AAV106.1) | | |
| AAVhu.38 | 4870 | US20150315612 SEQ ID NO: 161 |
| AAVhu.39 | 4871 | US20150315612 SEQ ID NO: 102 |
| AAVhu.39 | 4872 | US20150315612 SEQ ID NO: 24 |
| (AAVLG-9) | | |
| AAVhu.4 | 4873 | US20150315612 SEQ ID NO: 47 |
| AAVhu.4 | 4874 | US20150315612 SEQ ID NO: 141 |
| AAVhu.40 | 4875 | US20150315612 SEQ ID NO: 87 |
| AAVhu.40 | 4876 | US20150315612 SEQ ID No: 11 |
| (AAV114.3) | | |
| AAVhu.41 | 4877 | US20150315612 SEQ ID NO: 91 |
| AAVhu.41 | 4878 | US20150315612 SEQ ID NO: 6 |
| (AAV127.2) | | |
| AAVhu.42 | 4879 | US20150315612 SEQ ID NO: 85 |
| AAVhu.42 | 4880 | US20150315612 SEQ ID NO: 8 |
| (AAV127.5) | | |
| AAVhu.43 | 4881 | US20150315612 SEQ ID NO: 160 |
| AAVhu.43 | 4882 | US20150315612 SEQ ID NO: 236 |
| AAVhu.43 | 4883 | US20150315612 SEQ ID NO: 80 |
| (AAV128.1) | | |
| AAVhu.44 | 4884 | US20150159173 SEQ ID NO: 45, US20150315612 SEQ ID NO: |
| | | 158 |
| AAVhu.44 | 4885 | US20150315612 SEQ ID NO: 81 |
| (AAV128.3) | | |
| AAVhu.44R1 | 4886 | US20150159173 |
| AAVhu.44R2 | 4887 | US20150159173 |
| AAVhu.44R3 | 4888 | US20150159173 |
| AAVhu.45 | 4889 | US20150315612 SEQ ID NO: 76 |
| AAVhu.45 | 4890 | US20150315612 SEQ ID NO: 127 |
| AAVhu.46 | 4891 | US20150315612 SEQ ID NO: 82 |
| AAVhu.46 | 4892 | US20150315612 SEQ ID NO: 159 |
| AAVhu.46 | 4893 | US20150315612 SEQ ID NO: 224 |
| AAVhu.47 | 4894 | US20150315612 SEQ ID NO: 77 |
| AAVhu.47 | 4895 | US20150315612 SEQ ID NO: 128 |
| AAVhu.48 | 4896 | US20150159173 SEQ ID NO: 38 |
| AAVhu.48 | 4897 | US20150315612 SEQ ID NO: 157 |
| AAVhu.48 | 4898 | US20150315612 SEQ ID NO: 78 |
| (AAV130.4) | | |
| AAVhu.48R1 | 4899 | US20150159173 |
| AAVhu.48R2 | 4900 | US20150159173 |
| AAVhu.48R3 | 4901 | US20150159173 |
| AAVhu.49 | 4902 | US20150315612 SEQ ID NO: 209 |
| AAVhu.49 | 4903 | US20150315612 SEQ ID NO: 189 |
| AAVhu.5 | 4904 | US20150315612 SEQ ID NO: 45 |
| AAVhu.5 | 4905 | US20150315612 SEQ ID NO: 142 |
| AAVhu.51 | 4906 | US20150315612 SEQ ID NO: 208 |
| AAVhu.51 | 4907 | US20150315612 SEQ ID NO: 190 |
| AAVhu.52 | 4908 | US20150315612 SEQ ID NO: 210 |
| AAVhu.52 | 4909 | US20150315612 SEQ ID NO: 191 |
| AAVhu.53 | 4910 | US20150159173 SEQ ID NO: 19 |
| AAVhu.53 | 4911 | US20150159173 SEQ ID NO: 35 |
| AAVhu.53 | 4912 | US20150315612 SEQ ID NO: 176 |
| (AAV145.1) | | |
| AAVhu.54 | 4913 | US20150315612 SEQ ID NO: 188 |
| AAVhu.54 | 4914 | US20150315612 SEQ ID No: 177 |
| (AAV145.5) | | |
| AAVhu.55 | 4915 | US20150315612 SEQ ID NO: 187 |
| AAVhu.56 | 4916 | US20150315612 SEQ ID NO: 205 |
| AAVhu.56 | 4917 | US20150315612 SEQ ID NO: 168 |
| (AAV145.6) | | |
| AAVhu.56 | 4918 | US20150315612 SEQ ID NO: 192 |
| (AAV145.6) | | |
| AAVhu.57 | 4919 | US20150315612 SEQ ID NO: 206 |
| AAVhu.57 | 4920 | US20150315612 SEQ ID NO: 169 |
| AAVhu.57 | 4921 | US20150315612 SEQ ID NO: 193 |
| AAVhu.58 | 4922 | US20150315612 SEQ ID NO: 207 |
| AAVhu.58 | 4923 | US20150315612 SEQ ID NO: 194 |
| AAVhu.6 | 4924 | US20150315612 SEQ ID NO: 5 |
| (AAV3.1) | | |
| AAVhu.6 | 4925 | US20150315612 SEQ ID NO: 84 |
| (AAV3.1) | | |
| AAVhu.60 | 4926 | US20150315612 SEQ ID NO: 184 |
| AAVhu.60 | 4927 | US20150315612 SEQ ID NO: 170 |
| (AAV161.10) | | |
| AAVhu.61 | 4928 | US20150315612 SEQ ID NO: 185 |
| AAVhu.61 | 4929 | US20150315612 SEQ ID NO: 174 |
| (AAV161.6) | | |
| AAVhu.63 | 4930 | US20150315612 SEQ ID NO: 204 |
| AAVhu.63 | 4931 | US20150315612 SEQ ID NO: 195 |
| AAVhu.64 | 4932 | US20150315612 SEQ ID NO: 212 |
| AAVhu.64 | 4933 | US20150315612 SEQ ID NO: 196 |
| AAVhu.66 | 4934 | US20150315612 SEQ ID NO: 197 |
| AAVhu.67 | 4935 | US20150315612 SEQ ID NO: 215 |
| AAVhu.67 | 4936 | US20150315612 SEQ ID NO: 198 |
| AAVhu.7 | 4937 | US20150315612 SEQ ID NO: 226 |
| AAVhu.7 | 4938 | US20150315612 SEQ ID NO: 150 |
| AAVhu.7 | 4939 | US20150315612 SEQ ID NO: 55 |
| (AAV7.3) | | |
| AAVhu.71 | 4940 | US20150315612 SEQ ID NO: 79 |
| AAVhu.8 | 4941 | US20150315612 SEQ ID NO: 53 |
| AAVhu.8 | 4942 | US20150315612 SEQ ID NO: 12 |
| AAVhu.8 | 4943 | US20150315612 SEQ ID NO: 151 |
| AAVhu.9 | 4944 | US20150315612 SEQ ID NO: 58 |
| (AAV3.1) | | |
| AAVhu.9 | 4945 | US20150315612 SEQ ID NO: 155 |
| (AAV3.1) | | |
| AAVpi.1 | 4946 | US20150315612 SEQ ID NO: 28 |
| AAVpi.1 | 4947 | US20150315612 SEQ ID NO: 93 |
| AAVpi.2 | 4948 | US20150315612 SEQ ID NO: 30 |
| AAVpi.2 | 4949 | US20150315612 SEQ ID NO: 95 |
| AAVpi.3 | 4950 | US20150315612 SEQ ID NO: 29 |
| AAVpi.3 | 4951 | US20150315612 SEQ ID NO: 94 |
| AAVrh.10 | 4952 | US20150159173 SEQ ID NO: 9 |
| AAVrh.10 | 4953 | US20150159173 SEQ ID NO: 25 |
| AAVrh.10 | 4954 | US20030138772 SEQ ID NO: 81 |
| (AAV44.2) | | |
| AAVrh.12 | 4955 | US20030138772 SEQ ID NO: 30 |
| (AAV42.1b) | | |
| AAVrh.13 | 4956 | US20150159173 SEQ ID NO: 10 |
| AAVrh.13 | 4957 | US20150159173 SEQ ID NO: 26 |
| AAVrh.13 | 4958 | US20150315612 SEQ ID NO: 228 |
| AAVrh.13R | 4959 | US20150159173 |
| AAVrh.14 | 4960 | US20030138772 SEQ ID NO: 32 |
| (AAV42.3a) | | |
| AAVrh.17 | 4961 | US20030138772 SEQ ID NO: 34 |
| (AAV42.5a) | | |
| AAVrh.18 | 4962 | US20030138772 SEQ ID NO: 29 |
| (AAV42.5b) | | |
| AAVrh.19 | 4963 | US20030138772 SEQ ID NO: 38 |
| (AAV42.6b) | | |
| AAVrh.2 | 4964 | US20150159173 SEQ ID NO: 39 |
| AAVrh.2 | 4965 | US20150315612 SEQ ID NO: 231 |
| AAVrh.20 | 4966 | US20150159173 SEQ ID NO: 1 |
| AAVrh.21 | 4967 | US20030138772 SEQ ID NO: 35 |
| (AAV42.10) | | |
| AAVrh.22 | 4968 | US20030138772 SEQ ID NO: 37 |
| (AAV42.11) | | |
| AAVrh.23 | 4969 | US20030138772 SEQ ID NO: 58 |
| (AAV42.12) | | |
| AAVrh.24 | 4970 | US20030138772 SEQ ID NO: 31 |
| (AAV42.13) | | |
| AAVrh.25 | 4971 | US20030138772 SEQ ID NO: 28 |
| (AAV42.15) | | |
| AAVrh.2R | 4972 | US20150159173 |
| AAVrh.31 | 4973 | US20030138772 SEQ ID NO: 48 |
| (AAV223.1) | | |
| AAVrh.32 | 4974 | US20030138772 SEQ ID NO: 19 |
| (AAVC1) | | |
| AAVrh.32/33 | 4975 | US20150159173 SEQ ID NO: 2 |
| AAVrh.33 | 4976 | US20030138772 SEQ ID NO: 20 |
| (AAVC3) | | |
| AAVrh.34 | 4977 | US20030138772 SEQ ID NO: 21 |
| (AAVC5) | | |
| AAVrh.35 | 4978 | US20030138772 SEQ ID NO: 22 |
| (AAVF1) | | |
| AAVrh.36 | 4979 | US20030138772 SEQ ID NO: 23 |
| (AAVF3) | | |
| AAVrh.37 | 4980 | US20030138772 SEQ ID NO: 24 |
| AAVrh.37 | 4981 | US20150159173 SEQ ID NO: 40 |
| AAVrh.37 | 4982 | US20150315612 SEQ ID NO: 229 |
| AAVrh.37R2 | 4983 | US20150159173 |
| AAVrh.38 | 4984 | US20150315612 SEQ ID NO: 7 |
| (AAVLG-4) | | |
| AAVrh.38 | 4985 | US20150315612 SEQ ID NO: 86 |
| (AAVLG-4) | | |
| AAVrh.39 | 4986 | US20150159173 SEQ ID NO: 20, US20150315612 SEQ ID NO: 13 |
| AAVrh.39 | 4987 | US20150159173 SEQ ID NO: 3, US20150159173 SEQ ID NO: 36, |
| | | US20150315612 SEQ ID NO: 89 |
| AAVrh.40 | 4988 | US20150315612 SEQ ID NO: 92 |
| AAVrh.40 | 4989 | US20150315612 SEQ ID No: 14 |
| (AAVLG-10) | | |
| AAVrh.43 | 4990 | US20150315612 SEQ ID NO: 43, US20150159173 SEQ ID NO: 21 |
| (AAVN721-8) | | |
| AAVrh.43 | 4991 | US20150315612 SEQ ID NO: 163, US20150159173 SEQ ID NO: |
| (AAVN721-8) | | 37 |
| AAVrh.44 | 4992 | US20150315612 SEQ ID NO: 34 |
| AAVrh.44 | 4993 | US20150315612 SEQ ID NO: 111 |
| AAVrh.45 | 4994 | US20150315612 SEQ ID NO: 41 |
| AAVrh.45 | 4995 | US20150315612 SEQ ID NO: 109 |
| AAVrh.46 | 4996 | US20150159173 SEQ ID NO: 22, US20150315612 SEQ ID NO: 19 |
| AAVrh.46 | 4997 | US20150159173 SEQ ID NO: 4, US20150315612 SEQ ID NO: 101 |
| AAVrh.47 | 4998 | US20150315612 SEQ ID NO: 38 |
| AAVrh.47 | 4999 | US20150315612 SEQ ID NO: 118 |
| AAVrh.48 | 5000 | US20150159173 SEQ ID NO: 44, US20150315612 SEQ ID NO: |
| | | 115 |
| AAVrh.48 (AAV1- | 5001 | US20150315612 SEQ ID NO: 32 |
| 7) | | |
| AAVrh.48.1 | 5002 | US20150159173 |
| AAVrh.48.1.2 | 5003 | US20150159173 |
| AAVrh.48.2 | 5004 | US20150159173 |
| AAVrh.49 (AAV1- | 5005 | US20150315612 SEQ ID NO: 25 |
| 8) | | |
| AAVrh.49 (AAV1- | 5006 | US20150315612 SEQ ID NO: 103 |
| 8) | | |
| AAVrh.50 (AAV2- | 5007 | US20150315612 SEQ ID NO: 23 |
| 4) | | |
| AAVrh.50 (AAV2- | 5008 | US20150315612 SEQ ID NO: 108 |
| 4) | | |
| AAVrh.51 (AAV2- | 5009 | US20150315612 SEQ ID No: 22 |
| 5) | | |
| AAVrh.51 (AAV2- | 5010 | US20150315612 SEQ ID NO: 104 |
| 5) | | |
| AAVrh.52 (AAV3- | 5011 | US20150315612 SEQ ID NO: 18 |
| 9) | | |
| AAVrh.52 (AAV3- | 5012 | US20150315612 SEQ ID NO: 96 |
| 9) | | |
| AAVrh.53 | 5013 | US20150315612 SEQ ID NO: 97 |
| AAVrh.53 (AAV3- | 5014 | US20150315612 SEQ ID NO: 17 |
| 11) | | |
| AAVrh.53 (AAV3- | 5015 | US20150315612 SEQ ID NO: 186 |
| 11) | | |
| AAVrh.54 | 5016 | US20150315612 SEQ ID NO: 40 |
| AAVrh.54 | 5017 | US20150159173 SEQ ID NO: 49, US20150315612 SEQ ID NO: |
| | | 116 |
| AAVrh.55 | 5018 | US20150315612 SEQ ID NO: 37 |
| AAVrh.55 (AAV4- | 5019 | US20150315612 SEQ ID NO: 117 |
| 19) | | |
| AAVrh.56 | 5020 | US20150315612 SEQ ID NO: 54 |
| AAVrh.56 | 5021 | US20150315612 SEQ ID NO: 152 |
| AAVrh.57 | 5022 | US20150315612 SEQ ID NO: 26 |
| AAVrh.57 | 5023 | US20150315612 SEQ ID NO: 105 |
| AAVrh.58 | 5024 | US20150315612 SEQ ID NO: 27 |
| AAVrh.58 | 5025 | US20150159173 SEQ ID NO: 48, US20150315612 SEQ ID NO: |
| | | 106 |
| AAVrh.58 | 5026 | US20150315612 SEQ ID NO: 232 |
| AAVrh.59 | 5027 | US20150315612 SEQ ID NO: 42 |
| AAVrh.59 | 5028 | US20150315612 SEQ ID NO: 110 |
| AAVrh.60 | 5029 | US20150315612 SEQ ID NO: 31 |
| AAVrh.60 | 5030 | US20150315612 SEQ ID NO: 120 |
| AAVrh.61 | 5031 | US20150315612 SEQ ID NO: 107 |
| AAVrh.61 (AAV2- | 5032 | US20150315612 SEQ ID NO: 21 |
| 3) | | |
| AAVrh.62 (AAV2- | 5033 | US20150315612 SEQ ID No: 33 |
| 15) | | |
| AAVrh.62 (AAV2- | 5034 | US20150315612 SEQ ID NO: 114 |
| 15) | | |
| AAVrh.64 | 5035 | US20150315612 SEQ ID No: 15 |
| AAVrh.64 | 5036 | US20150159173 SEQ ID NO: 43, US20150315612 SEQ ID NO: 99 |
| AAVrh.64 | 5037 | US20150315612 SEQ ID NO: 233 |
| AAVRh.64R1 | 5038 | US20150159173 |
| AAVRh.64R2 | 5039 | US20150159173 |
| AAVrh.65 | 5040 | US20150315612 SEQ ID NO: 35 |
| AAVrh.65 | 5041 | US20150315612 SEQ ID NO: 112 |
| AAVrh.67 | 5042 | US20150315612 SEQ ID NO: 36 |
| AAVrh.67 | 5043 | US20150315612 SEQ ID NO: 230 |
| AAVrh.67 | 5044 | US20150159173 SEQ ID NO: 47, US20150315612 SEQ ID NO: |
| | | 113 |
| AAVrh.68 | 5045 | US20150315612 SEQ ID NO: 16 |
| AAVrh.68 | 5046 | US20150315612 SEQ ID NO: 100 |
| AAVrh.69 | 5047 | US20150315612 SEQ ID NO: 39 |
| AAVrh.69 | 5048 | US20150315612 SEQ ID NO: 119 |
| AAVrh.70 | 5049 | US20150315612 SEQ ID NO: 20 |
| AAVrh.70 | 5050 | US20150315612 SEQ ID NO: 98 |
| AAVrh.71 | 5051 | US20150315612 SEQ ID NO: 162 |
| AAVrh.72 | 5052 | US20150315612 SEQ ID NO: 9 |
| AAVrh.73 | 5053 | US20150159173 SEQ ID NO: 5 |
| AAVrh.74 | 5054 | US20150159173 SEQ ID NO: 6 |
| AAVrh.8 | 5055 | US20150159173 SEQ ID NO: 41 |
| AAVrh.8 | 5056 | US20150315612 SEQ ID NO: 235 |
| AAV1 | 5057 | US20150159173 SEQ ID NO: 11, US20150315612 SEQ ID NO: |
| | | 202 |
| AAV1 | 5058 | US20160017295 SEQ ID NO: 1 US20030138772 SEQ ID NO: 64, |
| | | US20150159173 SEQ ID NO: 27, US20150315612 SEQ ID NO: |
| | | 219, U.S. Pat. No. 7,198,951 SEQ ID NO: 5 |
| AAV1 | 5059 | US20030138772 SEQ ID NO: 6 |
| AAV10 | 5060 | US20030138772 SEQ ID NO: 117 |
| AAV10 | 5061 | WO2015121501 SEQ ID NO: 9 |
| AAV10 | 5062 | WO2015121501 SEQ ID NO: 8 |
| AAV11 | 5063 | US20030138772 SEQ ID NO: 118 |
| AAV12 | 5064 | US20030138772 SEQ ID NO: 119 |
| AAV2 | 5065 | US20150159173 SEQ ID NO: 7, US20150315612 SEQ ID NO: 211 |
| AAV2 | 5066 | US20030138772 SEQ ID NO: 70, US20150159173 SEQ ID NO: |
| | | 23, US20150315612 SEQ ID NO: 221, US20160017295 SEQ ID |
| | | NO: 2, U.S. Pat. No. 6,156,303 SEQ ID NO: 4, U.S. Pat. No. 7,198,951 SEQ ID NO: 4, |
| | | WO2015121501 SEQ ID NO: 1 |
| AAV2 | 5067 | U.S. Pat. No. 6,156,303 SEQ ID NO: 8 |
| AAV2 | 5068 | US20030138772 SEQ ID NO: 7 |
| AAV2 | 5069 | U.S. Pat. No. 6,156,303 SEQ ID NO: 3 |
| AAVhE1.1 | 5070 | U.S. Pat. No. 9,233,131 SEQ ID NO: 44 |
| AAVhEr1.14 | 5071 | U.S. Pat. No. 9,233,131 SEQ ID NO: 46 |
| AAVhEr1.16 | 5072 | U.S. Pat. No. 9,233,131 SEQ ID NO: 48 |
| AAVhEr1.18 | 5073 | U.S. Pat. No. 9,233,131 SEQ ID NO: 49 |
| AAVhEr1.23 | 5074 | U.S. Pat. No. 9,233,131 SEQ ID NO: 53 |
| (AAVhEr2.29) | | |
| AAVhEr1.35 | 5075 | U.S. Pat. No. 9,233,131 SEQ ID NO: 50 |
| AAVhEr1.36 | 5076 | U.S. Pat. No. 9,233,131 SEQ ID NO: 52 |
| AAVhEr1.5 | 5077 | U.S. Pat. No. 9,233,131 SEQ ID NO: 45 |
| AAVhEr1.7 | 5078 | U.S. Pat. No. 9,233,131 SEQ ID NO: 51 |
| AAVhEr1.8 | 5079 | U.S. Pat. No. 9,233,131 SEQ ID NO: 47 |
| AAVhEr2.16 | 5080 | U.S. Pat. No. 9,233,131 SEQ ID NO: 55 |
| AAVhEr2.30 | 5081 | U.S. Pat. No. 9,23,3131 SEQ ID NO: 56 |
| AAVhEr2.31 | 5082 | U.S. Pat. No. 9,233,131 SEQ ID NO: 58 |
| AAVhEr2.36 | 5083 | U.S. Pat. No. 9,233,131 SEQ ID NO: 57 |
| AAVhEr2.4 | 5084 | U.S. Pat. No. 9,233,131 SEQ ID NO: 54 |
| AAVhEr3.1 | 5085 | U.S. Pat. No. 9,233,131 SEQ ID NO: 59 |
| AAV3 | 5086 | US20030138772 SEQ ID NO: 71, US20150159173 SEQ ID NO: |
| | | 28, US20160017295 SEQ ID NO: 3, U.S. Pat. No. 7,198,951 SEQ ID NO: 6 |
| AAV3 | 5087 | US20030138772 SEQ ID NO: 8 |
| AAV3a | 5088 | U.S. Pat. No. 6,156,303 SEQ ID NO: 5 |
| AAV3a | 5089 | U.S. Pat. No. 6,156,303 SEQ ID NO: 9 |
| AAV3b | 5090 | U.S. Pat. No. 6,156,303 SEQ ID NO: 6 |
| AAV3b | 5091 | U.S. Pat. No. 6,156,303 SEQ ID NO: 10 |
| AAV3b | 5092 | U.S. Pat. No. 6,156,303 SEQ ID NO: 1 |
| AAV4 | 5093 | US20140348794 SEQ ID NO: 17 |
| AAV4 | 5094 | US20140348794 SEQ ID NO: 5 |
| AAV4 | 5095 | US20140348794 SEQ ID NO: 3 |
| AAV4 | 5096 | US20140348794 SEQ ID NO: 14 |
| AAV4 | 5097 | US20140348794 SEQ ID NO: 15 |
| AAV4 | 5098 | US20140348794 SEQ ID NO: 19 |
| AAV4 | 5099 | US20140348794 SEQ ID NO: 12 |
| AAV4 | 5100 | US20140348794 SEQ ID NO: 13 |
| AAV4 | 5101 | US20140348794 SEQ ID NO: 7 |
| AAV4 | 5102 | US20140348794 SEQ ID NO: 8 |
| AAV4 | 5103 | US20140348794 SEQ ID NO: 9 |
| AAV4 | 5104 | US20140348794 SEQ ID NO: 2 |
| AAV4 | 5105 | US20140348794 SEQ ID NO: 10 |
| AAV4 | 5106 | US20140348794 SEQ ID NO: 11 |
| AAV4 | 5107 | US20140348794 SEQ ID NO: 18 |
| AAV4 | 5108 | US20030138772 SEQ ID NO: 63, US20160017295 SEQ ID NO: 4, |
| | | US20140348794 SEQ ID NO: 4 |
| AAV4 | 5109 | US20140348794 SEQ ID NO: 16 |
| AAV4 | 5110 | US20140348794 SEQ ID NO: 20 |
| AAV4 | 5111 | US20140348794 SEQ ID NO: 6 |
| AAV4 | 5112 | US20140348794 SEQ ID NO: 1 |
| AAV2.5T | 5113 | U.S. Pat. No. 9,233,131 SEQ ID NO: 42 |
| AAV5 | 5114 | US20160017295 SEQ ID NO: 5, U.S. Pat. No. 7,427,396 SEQ ID NO: 2, |
| | | US20150315612 SEQ ID NO: 216 |
| AAV5 | 5115 | US20150315612 SEQ ID NO: 199 |
| AAV6 | 5116 | US20150159173 SEQ ID NO: 13 |
| AAV6 | 5117 | US20030138772 SEQ ID NO: 65, US20150159173 SEQ ID NO: |
| | | 29, US20160017295 SEQ ID NO: 6, U.S. Pat. No. 6,156,303 SEQ ID NO: 7 |
| AAV6 | 5118 | U.S. Pat. No. 6,156,303 SEQ ID NO: 11 |
| AAV6 | 5119 | U.S. Pat. No. 6,156,303 SEQ ID NO: 2 |
| AAV6 | 5120 | US20150315612 SEQ ID NO: 203 |
| AAV7 | 5121 | US20150159173 SEQ ID NO: 14 |
| AAV7 | 5122 | US20150315612 SEQ ID NO: 183 |
| AAV7 | 5123 | US20030138772 SEQ ID NO: 2, US20150159173 SEQ ID NO: 30, |
| | | US20150315612 SEQ ID NO: 181, US20160017295 SEQ ID NO: 7 |
| AAV7 | 5124 | US20030138772 SEQ ID NO: 3 |
| AAV7 | 5125 | US20030138772 SEQ ID NO: 1, US20150315612 SEQ ID NO: 180 |
| AAV7 | 5126 | US20150315612 SEQ ID NO: 213 |
| AAV7 | 5127 | US20150315612 SEQ ID NO: 222 |
| AAV8 | 5128 | US20150159173 SEQ ID NO: 15 |
| AAV8 | 5129 | US20150376240 SEQ ID NO: 7 |
| AAV8 | 5130 | US20030138772 SEQ ID NO: 4, US20150315612 SEQ ID NO: 182 |
| AAV8 | 5131 | US20030138772 SEQ ID NO: 95, US20140359799 SEQ ID NO: 1, |
| | | US20150159173 SEQ ID NO: 31, US20160017295 SEQ ID NO: 8 |
| | | U.S. Pat. No. 7,198,951 SEQ ID NO: 7, US20150315612 SEQ ID NO: 223 |
| AAV8 | 5132 | US20150376240 SEQ ID NO: 8 |
| AAV8 | 5133 | US20150315612 SEQ ID NO: 214 |
| AAV9 | 5134 | US20030138772 SEQ ID NO: 5 |
| AAV9 | 5135 | U.S. Pat. No. 7,198,951 SEQ ID NO: 1 |
| AAV9 | 5136 | US20160017295 SEQ ID NO: 9 |
| AAV9 | 5137 | US20030138772 SEQ ID NO: 100, U.S. Pat. No. 7,198,951 SEQ ID NO: 2 |
| AAV9 | 5138 | U.S. Pat. No. 7,198,951 SEQ ID NO: 3 |
| AAAV (Avian | 5139 | U.S. Pat. No. 9,238,800 SEQ ID NO: 12 |
| AAV) | | |
| AAAV (Avian | 5140 | U.S. Pat. No. 9,238,800 SEQ ID NO: 2 |
| AAV) | | |
| AAAV (Avian | 5141 | U.S. Pat. No. 9,238,800 SEQ ID NO: 6 |
| AAV) | | |
| AAAV (Avian | 5142 | U.S. Pat. No. 9,238,800 SEQ ID NO: 4 |
| AAV) | | |
| AAAV (Avian | 5143 | U.S. Pat. No. 9,238,800 SEQ ID NO: 8 |
| AAV) | | |
| AAAV (Avian | 5144 | U.S. Pat. No. 9,238,800 SEQ ID NO: 14 |
| AAV) | | |
| AAAV (Avian | 5145 | U.S. Pat. No. 9,238,800 SEQ ID NO: 10 |
| AAV) | | |
| AAAV (Avian | 5146 | U.S. Pat. No. 9,238,800 SEQ ID NO: 15 |
| AAV) | | |
| AAAV (Avian | 5147 | U.S. Pat. No. 9,238,800 SEQ ID NO: 5 |
| AAV) | | |
| AAAV (Avian | 5148 | U.S. Pat. No. 9,238,800 SEQ ID NO: 9 |
| AAV) | | |
| AAAV (Avian | 5149 | U.S. Pat. No. 9,238,800 SEQ ID NO: 3 |
| AAV) | | |
| AAAV (Avian | 5150 | U.S. Pat. No. 9,238,800 SEQ ID NO: 7 |
| AAV) | | |
| AAAV (Avian | 5151 | U.S. Pat. No. 9,238,800 SEQ ID NO: 11 |
| AAV) | | |
| AAAV (Avian | 5152 | U.S. Pat. No. 9,238,800 SEQ ID NO: 13 |
| AAV) | | |
| AAAV (Avian | 5153 | U.S. Pat. No. 9,238,800 SEQ ID NO: 1 |
| AAV) | | |
| AAV Shuffle 100-1 | 5154 | US20160017295 SEQ ID NO: 23 |
| AAV Shuffle 100-1 | 5155 | US20160017295 SEQ ID NO: 11 |
| AAV Shuffle 100-2 | 5156 | US20160017295 SEQ ID NO: 37 |
| AAV Shuffle 100-2 | 5157 | US20160017295 SEQ ID NO: 29 |
| AAV Shuffle 100-3 | 5158 | US20160017295 SEQ ID NO: 24 |
| AAV Shuffle 100-3 | 5159 | US20160017295 SEQ ID NO: 12 |
| AAV Shuffle 100-7 | 5160 | US20160017295 SEQ ID NO: 25 |
| AAV Shuffle 100-7 | 5161 | US20160017295 SEQ ID NO: 13 |
| AAV Shuffle 10-2 | 5162 | US20160017295 SEQ ID NO: 34 |
| AAV Shuffle 10-2 | 5163 | US20160017295 SEQ ID NO: 26 |
| AAV Shuffle 10-6 | 5164 | US20160017295 SEQ ID NO: 35 |
| AAV Shuffle 10-6 | 5165 | US20160017295 SEQ ID NO: 27 |
| AAV Shuffle 10-8 | 5166 | US20160017295 SEQ ID NO: 36 |
| AAV Shuffle 10-8 | 5167 | US20160017295 SEQ ID NO: 28 |
| AAV SM 100-10 | 5168 | US20160017295 SEQ ID NO: 41 |
| AAV SM 100-10 | 5169 | US20160017295 SEQ ID NO: 33 |
| AAV SM 100-3 | 5170 | US20160017295 SEQ ID NO: 40 |
| AAV SM 100-3 | 5171 | US20160017295 SEQ ID NO: 32 |
| AAV SM 10-1 | 5172 | US20160017295 SEQ ID NO: 38 |
| AAV SM 10-1 | 5173 | US20160017295 SEQ ID NO: 30 |
| AAV SM 10-2 | 5174 | US20160017295 SEQ ID NO: 10 |
| AAV SM 10-2 | 5175 | US20160017295 SEQ ID NO: 22 |
| AAV SM 10-8 | 5176 | US20160017295 SEQ ID NO: 39 |
| AAV SM 10-8 | 5177 | US20160017295 SEQ ID NO: 31 |
| AAV5 | 5178 | U.S. Pat. No. 7,427,396 SEQ ID NO: 1 |
| AAV-8b | 5179 | US20150376240 SEQ ID NO: 5 |
| AAV-8b | 5180 | US20150376240 SEQ ID NO: 3 |
| AAV-8h | 5181 | US20150376240 SEQ ID NO: 6 |
| AAV-8h | 5182 | US20150376240 SEQ ID NO: 4 |
| AAVDJ | 5183 | US20140359799 SEQ ID NO: 3, U.S. Pat. No. 7,588,772 SEQ ID NO: 2 |
| AAVDJ | 5184 | US20140359799 SEQ ID NO: 2, U.S. Pat. No. 7,588,772 SEQ ID NO: 1 |
| AAVDJ-8 | 5185 | U.S. Pat. No. 7,588,772; Grimm et al 2008 |
| AAVDJ-8 | 5186 | U.S. Pat. No. 7,588,772; Grimm et al 2008 |
| AAV-LK01 | 5187 | US20150376607 SEQ ID NO: 2 |
| AAV-LK01 | 5188 | US20150376607 SEQ ID NO: 29 |
| AAV-LK02 | 5189 | US20150376607 SEQ ID NO: 3 |
| AAV-LK02 | 5190 | US20150376607 SEQ ID NO: 30 |
| AAV-LK03 | 5191 | US20150376607 SEQ ID NO: 4 |
| AAV-LK03 | 5192 | WO2015121501 SEQ ID NO: 12, US20150376607 SEQ ID NO: 31 |
| AAV-LK04 | 5193 | US20150376607 SEQ ID NO: 5 |
| AAV-LK04 | 5194 | US20150376607 SEQ ID NO: 32 |
| AAV-LK05 | 5195 | US20150376607 SEQ ID NO: 6 |
| AAV-LK05 | 5196 | US20150376607 SEQ ID NO: 33 |
| AAV-LK06 | 5197 | US20150376607 SEQ ID NO: 7 |
| AAV-LK06 | 5198 | US20150376607 SEQ ID NO: 34 |
| AAV-LK07 | 5199 | US20150376607 SEQ ID NO: 8 |
| AAV-LK07 | 5200 | US20150376607 SEQ ID NO: 35 |
| AAV-LK08 | 5201 | US20150376607 SEQ ID NO: 9 |
| AAV-LK08 | 5202 | US20150376607 SEQ ID NO: 36 |
| AAV-LK09 | 5203 | US20150376607 SEQ ID NO: 10 |
| AAV-LK09 | 5204 | US20150376607 SEQ ID NO: 37 |
| AAV-LK10 | 5205 | US20150376607 SEQ ID NO: 11 |
| AAV-LK10 | 5206 | US20150376607 SEQ ID NO: 38 |
| AAV-LK11 | 5207 | US20150376607 SEQ ID NO: 12 |
| AAV-LK11 | 5208 | US20150376607 SEQ ID NO: 39 |
| AAV-LK12 | 5209 | US20150376607 SEQ ID NO: 13 |
| AAV-LK12 | 5210 | US20150376607 SEQ ID NO: 40 |
| AAV-LK13 | 5211 | US20150376607 SEQ ID NO: 14 |
| AAV-LK13 | 5212 | US20150376607 SEQ ID NO: 41 |
| AAV-LK14 | 5213 | US20150376607 SEQ ID NO: 15 |
| AAV-LK14 | 5214 | US20150376607 SEQ ID NO: 42 |
| AAV-LK15 | 5215 | US20150376607 SEQ ID NO: 16 |
| AAV-LK15 | 5216 | US20150376607 SEQ ID NO: 43 |
| AAV-LK16 | 5217 | US20150376607 SEQ ID NO: 17 |
| AAV-LK16 | 5218 | US20150376607 SEQ ID NO: 44 |
| AAV-LK17 | 5219 | US20150376607 SEQ ID NO: 18 |
| AAV-LK17 | 5220 | US20150376607 SEQ ID NO: 45 |
| AAV-LK18 | 5221 | US20150376607 SEQ ID NO: 19 |
| AAV-LK18 | 5222 | US20150376607 SEQ ID NO: 46 |
| AAV-LK19 | 5223 | US20150376607 SEQ ID NO: 20 |
| AAV-LK19 | 5224 | US20150376607 SEQ ID NO: 47 |
| AAV-PAEC | 5225 | US20150376607 SEQ ID NO: 1 |
| AAV-PAEC | 5226 | US20150376607 SEQ ID NO: 48 |
| AAV-PAEC11 | 5227 | US20150376607 SEQ ID NO: 26 |
| AAV-PAEC11 | 5228 | US20150376607 SEQ ID NO: 54 |
| AAV-PAEC12 | 5229 | US20150376607 SEQ ID NO: 27 |
| AAV-PAEC12 | 5230 | US20150376607 SEQ ID NO: 51 |
| AAV-PAEC13 | 5231 | US20150376607 SEQ ID NO: 28 |
| AAV-PAEC13 | 5232 | US20150376607 SEQ ID NO: 49 |
| AAV-PAEC2 | 5233 | US20150376607 SEQ ID NO: 21 |
| AAV-PAEC2 | 5234 | US20150376607 SEQ ID NO: 56 |
| AAV-PAEC4 | 5235 | US20150376607 SEQ ID NO: 22 |
| AAV-PAEC4 | 5236 | US20150376607 SEQ ID NO: 55 |
| AAV-PAEC6 | 5237 | US20150376607 SEQ ID NO: 23 |
| AAV-PAEC6 | 5238 | US20150376607 SEQ ID NO: 52 |
| AAV-PAEC7 | 5239 | US20150376607 SEQ ID NO: 24 |
| AAV-PAEC7 | 5240 | US20150376607 SEQ ID NO: 53 |
| AAV-PAEC8 | 5241 | US20150376607 SEQ ID NO: 25 |
| AAV-PAEC8 | 5242 | US20150376607 SEQ ID NO: 50 |
| AAVrh.8R | 5243 | US20150159173, WO2015168666 SEQ ID NO: 9 |
| AAVrh.8R A586R | 5244 | WO2015168666 SEQ ID NO: 10 |
| mutant | | |
| AAVrh.8R R533A | 5245 | WO2015168666 SEQ ID NO: 11 |
| mutant | | |
| BAAV (bovine | 5246 | U.S. Pat. No. 9,193,769 SEQ ID NO: 11 |
| AAV) | | |
| BAAV (bovine | 5247 | U.S. Pat. No. 7,427,396 SEQ ID NO: 5 |
| AAV) | | |
| BAAV (bovine | 5248 | U.S. Pat. No. 7,427,396 SEQ ID NO: 6 |
| AAV) | | |
| BNP61 AAV | 5249 | US20150238550 SEQ ID NO: 1 |
| BNP61 AAV | 5250 | US20150238550 SEQ ID NO: 2 |
| BNP62 AAV | 5251 | US20150238550 SEQ ID NO: 3 |
| BNP63 AAV | 5252 | US20150238550 SEQ ID NO: 4 |
| caprine AAV | 5253 | U.S. Pat. No. 7,427,396 SEQ ID NO: 3 |
| caprine AAV | 5254 | U.S. Pat. No. 7,427,396 SEQ ID NO: 4 |
| true type AAV | 5255 | WO2015121501 SEQ ID NO: 2 |
| (ttAAV) | | |
| BAAV (bovine | 5256 | U.S. Pat. No. 9,193,769 SEQ ID NO: 8 |
| AAV) | | |
| BAAV (bovine | 5257 | U.S. Pat. No. 9,193,769 SEQ ID NO: 10 |
| AAV) | | |
| BAAV (bovine | 5258 | U.S. Pat. No. 9,193,769 SEQ ID NO: 4 |
| AAV) | | |
| BAAV (bovine | 5259 | U.S. Pat. No. 9,193,769 SEQ ID NO: 2 |
| AAV) | | |
| BAAV (bovine | 5260 | U.S. Pat. No. 9,193,769 SEQ ID NO: 6 |
| AAV) | | |
| BAAV (bovine | 5261 | U.S. Pat. No. 9,193,769 SEQ ID NO: 1 |
| AAV) | | |
| BAAV (bovine | 5262 | U.S. Pat. No. 9,193,769 SEQ ID NO: 5 |
| AAV) | | |
| BAAV (bovine | 5263 | U.S. Pat. No. 9,193,769 SEQ ID NO: 3 |
| AAV) | | |
| BAAV (bovine | 5264 | U.S. Pat. No. 9,193,769 SEQ ID NO: 7 |
| AAV) | | |
| BAAV (bovine | 5265 | U.S. Pat. No. 9,193,769 SEQ ID NO: 9 |
| AAV) | | |

Each of the patents, applications and/or publications listed in Table 5 are hereby incorporated by reference in their entirety.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. No. 5,786,211; U.S. Pat. No. 5,871,982, and U.S. Pat. No. 6,258,595, the contents of each of which are herein incorporated by reference in their entireties.

AAV vector serotypes can be matched to target cell types. For example, the following exemplary cell types can be transduced by the indicated AAV serotypes among others.

TABLE 6
Tissue/Cell Types and Serotypes

| Tissue/Cell Type | Serotype |
|---|---|
| Liver | AAV3, AAV8, AAV9 |
| Skeletal muscle | AAV1, AAV7, AAV6, AAV8, AAV9 |
| Central nervous system | AAV5, AAV1, AAV4 |
| RPE | AAV5, AAV4 |
| Photoreceptor cells | AAV5 |
| Lung | AAV9 |
| Heart | AAV8 |
| Pancreas | AAV8 |
| Kidney | AAV2 |

In addition to adeno-associated viral vectors, other viral vectors can be used. Such viral vectors include, but are not limited to, lentivirus, alphavirus, enterovirus, pestivirus, baculovirus, herpesvirus, Epstein Barr virus, papovavirusr, poxvirus, vaccinia virus, and herpes simplex virus.

In some cases, Cas9 mRNA, sgRNA targeting one or two loci in WAS gene, and donor DNA can each be separately formulated into lipid nanoparticles, or are all co-formulated into one lipid nanoparticle.

In some cases, Cas9 mRNA can be formulated in a lipid nanoparticle, while sgRNA and donor DNA can be delivered in an AAV vector.

Options are available to deliver the Cas9 nuclease as a DNA plasmid, as mRNA or as a protein. The guide RNA can be expressed from the same DNA, or can also be delivered as an RNA. The RNA can be chemically modified to alter or improve its half-life, or decrease the likelihood or degree of immune response. The endonuclease protein can be complexed with the gRNA prior to delivery. Viral vectors allow efficient delivery; split versions of Cas9 and smaller orthologs of Cas9 can be packaged in AAV, as can donors for HDR. A range of non-viral delivery methods also exist that can deliver each of these components, or non-viral and viral methods can be employed in tandem. For example, nano-particles can be used to deliver the protein and guide RNA, while AAV can be used to deliver a donor DNA.

### Genetically Modified Cells

The term “genetically modified cell” refers to a cell that has at least one genetic modification introduced by genome editing (e.g., using the CRISPR/Cas9/Cpf1 system). In some ex vivo examples herein, the genetically modified cell can be genetically modified progenitor cell. In some in vivo examples herein, the genetically modified cell can be a genetically modified liver cell. A genetically modified cell having an exogenous genome-targeting nucleic acid and/or an exogenous nucleic acid encoding a genome-targeting nucleic acid is contemplated herein.

The term “control treated population” describes a population of cells that has been treated with identical media, viral induction, nucleic acid sequences, temperature, confluency, flask size, pH, etc., with the exception of the addition of the genome editing components. Any method known in the art can be used to measure restoration of WAS gene or protein expression or activity, for example Western Blot analysis of the WAS protein or quantifying WAS gene mRNA.

The term “isolated cell” refers to a cell that has been removed from an organism in which it was originally found, or a descendant of such a cell. Optionally, the cell can be cultured in vitro, e.g., under defined conditions or in the presence of other cells. Optionally, the cell can be later introduced into a second organism or re-introduced into the organism from which it (or the cell from which it is descended) was isolated.

The term “isolated population” with respect to an isolated population of cells refers to a population of cells that has been removed and separated from a mixed or heterogeneous population of cells. In some cases, the isolated population can be a substantially pure population of cells, as compared to the heterogeneous population from which the cells were isolated or enriched. In some cases, the isolated population can be an isolated population of human progenitor cells, e.g., a substantially pure population of human progenitor cells, as compared to a heterogeneous population of cells having human progenitor cells and cells from which the human progenitor cells were derived.

The term “substantially enhanced,” with respect to a particular cell population, refers to a population of cells in which the occurrence of a particular type of cell is increased relative to pre-existing or reference levels, by at least 2-fold, at least 3-, at least 4-, at least 5-, at least 6-, at least 7-, at least 8-, at least 9, at least 10-, at least 20-, at least 50-, at least 100-, at least 400-, at least 1000-, at least 5000-, at least 20000-, at least 100000- or more fold depending, e.g., on the desired levels of such cells for ameliorating Wiskott-Aldrich Syndrome (WAS).

The term “substantially enriched” with respect to a particular cell population, refers to a population of cells that is at least about 10%, about 20%, about 30%, about 40%, about 50%, about 60%, about 70% or more with respect to the cells making up a total cell population.

The terms “substantially enriched” or “substantially pure” with respect to a particular cell population, refers to a population of cells that is at least about 75%, at least about 85%, at least about 90%, or at least about 95% pure, with respect to the cells making up a total cell population. That is, the terms “substantially pure” or “essentially purified.” with regard to a population of progenitor cells, refers to a population of cells that contain fewer than about 20%, about 15%, about 10%, about 9%, about 8%, about 7%, about 6%, about 5%, about 4%, about 3%, about 2%, about 1%, or less than 1%, of cells that are not progenitor cells as defined by the terms herein.

Differentiation of Genome-Edited iPSCs into Other Cell Types

Another step of the ex vivo methods of the present disclosure can have differentiating the genome-edited iPSCs into hepatocytes. The differentiating step can be performed according to any method known in the art. For example, hiPSC are differentiated into definitive endoderm using various treatments, including activin and B27 supplement (Life Technology). The definitive endoderm is further differentiated into hepatocyte, the treatment includes: FGF4, HGF, BMP2, BMP4, Oncostatin M, Dexametason, etc (Duan et al, STEM CELLS; 2010:28:674-686. Ma et al, STEM CELLS TRANSLATIONAL MEDICINE 2013; 2:409-419).

Differentiation of Genome-Edited Mesenchymal Stem Cells into Hepatocytes

Another step of the ex vivo methods of the present disclosure can have differentiating the genome-edited mesenchymal stem cells into hepatocytes. The differentiating step can be performed according to any method known in the art. For example, hMSC are treated with various factors and hormones, including insulin, transferrin. FGF4, HGF, bile acids (Sawitza I et al, Sci Rep. 2015; 5: 13320).

Implanting Cells into Patients

Another step of the ex vivo methods of the present disclosure can have implanting the hepatocytes into patients. This implanting step can be accomplished using any method of implantation known in the art. For example, the genetically modified cells can be injected directly in the patient's blood or otherwise administered to the patient.

Another step of the ex vivo methods of the disclosure involves implanting the progenitor cells or primary hepatocytes into patients. This implanting step may be accomplished using any method of implantation known in the art. For example, the genetically modified cells may be injected directly in the patient's liver or otherwise administered to the patient.

### IV. Dosing and Administration

The terms “administering,” “introducing” and “transplanting” are used interchangeably in the context of the placement of cells, e.g., progenitor cells, into a subject, by a method or route that results in at least partial localization of the introduced cells at a desired site, such as a site of injury or repair, such that a desired effect(s) is produced. The cells e.g., progenitor cells, or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment. For example, in some aspects described herein, an effective amount of myogenic progenitor cells is administered via a systemic route of administration, such as an intraperitoneal or intravenous route.

The terms “individual,” “subject” and “host” are used interchangeably herein and refer to any subject for whom diagnosis, treatment or therapy is desired. In some aspects, the subject is a mammal. In some aspects, the subject is a human being. In some aspects, the subject is a human patient. In some aspects, the subject can have or is suspected of having WAS and/or has one or more symptoms of WAS. In some aspects, the subject being a human who is diagnosed with a risk of WAS at the time of diagnosis or later. In some cases, the diagnosis with a risk of WAS may be determined based on the presence of one or more mutations in the endogenous WAS gene or genomic sequence near the WAS gene in the genome.

When provided prophylactically, progenitor cells described herein can be administered to a subject in advance of any symptom of Wiskott-Aldrich Syndrome (WAS). Accordingly, the prophylactic administration of a progenitor cell population serves to prevent Wiskott-Aldrich Syndrome (WAS).

In some embodiments described herein, the progenitor cell population being administered according to the methods described herein can have allogeneic progenitor cells obtained from one or more donors. In some embodiments, the allogenic progenitor cells are hematopoietic progenitor cells. Such progenitors may be of any cellular or tissue origin, e.g., liver, muscle, cardiac, etc. “Allogeneic” refers to a progenitor cell or biological samples having progenitor cells obtained from one or more different donors of the same species, where the genes at one or more loci are not identical. For example, a liver progenitor cell population being administered to a subject can be derived from one more unrelated donor subjects, or from one or more non-identical siblings. In some cases, syngeneic progenitor cell populations can be used, such as those obtained from genetically identical animals, or from identical twins. The progenitor cells can be autologous cells; that is, the progenitor cells are obtained or isolated from a subject and administered to the same subject, i.e., the donor and recipient are the same.

The term “effective amount” refers to the amount of a population of progenitor cells or their progeny needed to prevent or alleviate at least one or more signs or symptoms of Wiskott-Aldrich Syndrome (WAS), and relates to a sufficient amount of a composition to provide the desired effect, e.g., to treat a subject having Wiskott-Aldrich Syndrome (WAS). The term “therapeutically effective amount” therefore refers to an amount of progenitor cells or a composition having progenitor cells that is sufficient to promote a particular effect when administered to a typical subject, such as one who has or is at risk for Wiskott-Aldrich Syndrome (WAS). An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

For use in the various aspects described herein, an effective amount of progenitor cells has at least 10² progenitor cells, at least 5×10² progenitor cells, at least 10³ progenitor cells, at least 5×10³ progenitor cells, at least 10⁴ progenitor cells, at least 5×10⁴ progenitor cells, at least 10⁵ progenitor cells, at least 2×10⁵ progenitor cells, at least 3×10⁵ progenitor cells, at least 4×10⁵ progenitor cells, at least 5×10⁵ progenitor cells, at least 6×10⁵ progenitor cells, at least 7×10⁵ progenitor cells, at least 8×10⁵ progenitor cells, at least 9×10⁵ progenitor cells, at least 1×10⁶ progenitor cells, at least 2×10⁶ progenitor cells, at least 3×10⁶ progenitor cells, at least 4×10⁶ progenitor cells, at least 5×10⁶ progenitor cells, at least 6×10⁶ progenitor cells, at least 7×10⁶ progenitor cells, at least 8×10⁶ progenitor cells, at least 9×10⁶ progenitor cells, or multiples thereof. The progenitor cells can be derived from one or more donors, or can be obtained from an autologous source. In some examples described herein, the progenitor cells can be expanded in culture prior to administration to a subject in need thereof.

Modest and incremental increases in the levels of functional WAS protein expressed in cells of patients having Wiskott-Aldrich Syndrome (WAS) can be beneficial for ameliorating one or more symptoms of the disease, for increasing long-term survival, and/or for reducing side effects associated with other treatments. Upon administration of such cells to human patients, the presence of progenitors that are producing increased levels of functional WAS protein is beneficial. In some cases, effective treatment of a subject gives rise to at least about 3%, 5% or 7% functional WAS protein relative to total WAS protein in the treated subject. In some examples, functional WAS protein will be at least about 10% of total WAS gene. In some examples, functional WAS protein will be at least about 20% to 30% of total WAS protein. Similarly, the introduction of even relatively limited subpopulations of cells having significantly elevated levels of functional WAS protein can be beneficial in various patients because in some situations normalized cells will have a selective advantage relative to diseased cells. However, even modest levels of progenitors with elevated levels of functional WAS protein can be beneficial for ameliorating one or more aspects of Wiskott-Aldrich Syndrome (WAS) in patients. In some examples, about 10%, about 20%, about 30%, about 40%, about 50%, about 60%, about 70%, about 80%, about 90% or more of the liver progenitors in patients to whom such cells are administered are producing increased levels of functional WAS protein.

“Administered” refers to the delivery of a progenitor cell composition into a subject by a method or route that results in at least partial localization of the cell composition at a desired site. A cell composition can be administered by any appropriate route that results in effective treatment in the subject, i.e, administration results in delivery to a desired location in the subject where at least a portion of the composition delivered, i.e, at least 1×10⁴ cells are delivered to the desired site for a period of time.

In one aspect of the method, the pharmaceutical composition may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some examples, the route is intravenous. For the delivery of cells, administration by injection or infusion can be made.

The cells can be administered systemically. The phrases “systemic administration,” “administered systemically”, “peripheral administration” and “administered peripherally” refer to the administration of a population of progenitor cells other than directly into a target site, tissue, or organ, such that it enters, instead, the subject's circulatory system and, thus, is subject to metabolism and other like processes.

The efficacy of a treatment having a composition for the treatment of Wiskott-Aldrich Syndrome (WAS) can be determined by the skilled clinician. However, a treatment is considered “effective treatment,” if any one or all of the signs or symptoms of, as but one example, levels of functional WAS protein are altered in a beneficial manner (e.g., increased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

The treatment according to the present disclosure can ameliorate one or more symptoms associated with Wiskott-Aldrich Syndrome (WAS) by increasing, decreasing or altering the amount of functional WAS protein in the individual.

### V. Features and Properties of the Wiskott-Aldrich Syndrome Gene (was Gene)

In one embodiment, the gene may be the Wiskott-Aldrich syndrome gene (WAS gene) which may also be referred to as SCNX, THC1, THC, IMD2, Thrombocytopenia 1 (X-Linked), and Eczema-Thrombocytopenia. The WAS gene has a cytogenetic location of Xp11.23 and the genomic coordinate as seen on Ensemble are on Chromosome X on the forward strand at position 48,676,596-48,691,427. The nucleotide sequence of WAS gene is show n as SEQ ID NO: 5266. VN1R110P is the gene upstream of WAS gene on the forward strand and SUV39H1 is the gene downstream of WAS gene on the forward strand. The WAS gene has a NCBI gene ID of 7454, Uniprot ID of P42768 and Ensembl Gene ID of ENSG00000015285. The WAS gene has 559 SNPs, 29 intron sequences and 33 exon sequences. The exon ID from Ensembl and the start/stop sites of the introns and exons are shown in Table 7.

TABLE 7
Introns and Exons for WAS

| Exon No. | Exon ID | Start/Stop | Intron No. | Intron based on Exon ID | Start/Stop |
|---|---|---|---|---|---|
| Exon 1 | ENSE00001863155 | 48,689,020-48,689,066 | Intron 1 | Intron ENSE00001863155-ENSE00001862355 | 48,689,067-48,689,319 |
| Exon 2 | ENSE00001862355 | 48,689,320-48,689,594 | Intron 2 | Intron ENSE00001621913-ENSE00001608034 | 48,676,749-48,683,267 |
| Exon 3 | ENSE00001621913 | 48,676,596-48,676,748 | Intron 3 | Intron ENSE00001608034-ENSE00001700602 | 48,683,364-48,683,819 |
| Exon 4 | ENSE00001608034 | 48,683,268-48,683,363 | Intron 4 | Intron ENSE00001700602-ENSE00003539440 | 48,683,986-48,684,282 |
| Exon 5 | ENSE00001700602 | 48,683,820-48,683,985 | Intron 5 | Intron ENSE00003539440-ENSE00003612781 | 48,684,424-48,685,546 |
| Exon 6 | ENSE00003539440 | 48,684,283-48,684,423 | Intron 6 | Intron ENSE00003612781-ENSE00003547357 | 48,685,634-48,685,733 |
| Exon 7 | ENSE00003612781 | 48,685,547-48,685,633 | Intron 7 | Intron ENSE00003547357-ENSE00003544043 | 48,685,837-48,685,945 |
| Exon 8 | ENSE00003547357 | 48,685,734-48,685,836 | Intron 8 | Intron ENSE00003544043-ENSE00003543007 | 48,685,988-48,686,080 |
| Exon 9 | ENSE00003544043 | 48,685,946-48,685,987 | Intron 9 | Intron ENSE00003543007-ENSE00001703305 | 48,686,135-48,686,780 |
| Exon 10 | ENSE00003543007 | 48,686,081-48,686,134 | Intron 10 | Intron ENSE00001816247-ENSE00003606084 | 48,683,986-48,684,282 |
| Exon 11 | ENSE00001703305 | 48,686,781-48,686,871 | Intron 11 | Intron ENSE00003606084-ENSE00003460648 | 48,684,424-48,685,546 |
| Exon 12 | ENSE00001816247 | 48,683,828-48,683,985 | Intron 12 | Intron ENSE00003460648-ENSE00003502047 | 48,685,634-48,685,733 |
| Exon 13 | ENSE00003606084 | 48,684,283-48,684,423 | Intron 13 | Intron ENSE00003502047-ENSE00003601472 | 48,685,837-48,685,945 |
| Exon 14 | ENSE00003460648 | 48,685,547-48,685,633 | Intron 14 | Intron ENSE00003601472-ENSE00003584565 | 48,685,988-48,686,080 |
| Exon 15 | ENSE00003502047 | 48,685,734-48,685,836 | Intron 15 | Intron ENSE00003584565-ENSE00003535915 | 48,686,135-48,686,780 |
| Exon 16 | ENSE00003601472 | 48,685,946-48,685,987 | Intron 16 | Intron ENSE00003535915-ENSE00001953989 | 48,686,956-48,688,053 |
| Exon 17 | ENSE00003584565 | 48,686,081-48,686,134 | Intron 17 | Intron ENSE00001919437-ENSE00003601472 | 48,685,837-48,685,945 |
| Exon 18 | ENSE00003535915 | 48,686,781-48,686,955 | Intron 18 | Intron ENSE00003584565-ENSE00001863442 | 48,686,135-48,688,053 |
| Exon 19 | ENSE00001953989 | 48,688,054-48,688,198 | Intron 19 | Intron ENSE00001872072-ENSE00001936429 | 48,688,454-48,688,659 |
| Exon 20 | ENSE00001919437 | 48,685,779-48,685,836 | Intron 20 | Intron ENSE00001840524-ENSE00003606084 | 48,683,986-48,684,282 |
| Exon 21 | ENSE00001863442 | 48,688,054-48,688,146 | Intron 21 | Intron ENSE00003502047-ENSE00001861993 | 48,685,837-48,686,819 |
| Exon 22 | ENSE00001872072 | 48,688,279-48,688,453 | Intron 22 | Intron ENSE00001861993-ENSE00001869250 | 48,686,956-48,688,053 |
| Exon 23 | ENSE00001936429 | 48,688,660-48,688,999 | Intron 23 | Intron ENSE00001707442-ENSE00003539440 | 48,683,986-48,684,282 |
| Exon 24 | ENSE00001840524 | 48,683,819-48,683,985 | Intron 24 | Intron ENSE00003543007-ENSE00003599478 | 48,686,135-48,686,780 |
| Exon 25 | ENSE00001861993 | 48,686,820-48,686,955 | Intron 25 | Intron ENSE00003599478-ENSE00000332771 | 48,686,956-48,688,053 |
| Exon 26 | ENSE00001869250 | 48,688,054-48,688,137 | Intron 26 | Intron ENSE00000332771-ENSE00000669950 | 48,688,097-48,688,299 |
| Exon 27 | ENSE00001707442 | 48,683,779-48,683,985 | Intron 27 | Intron ENSE00000669950-ENSE00001255082 | 48,688,454-48,688,659 |
| Exon 28 | ENSE00003599478 | 48,686,781-48,686,955 | Intron 28 | Intron ENSE00001255082-ENSE00000867103 | 48,689,067-48,689,319 |
| Exon 29 | ENSE00000332771 | 48,688,054-48,688,096 | Intron 29 | Intron ENSE00000867103-ENSE00000867104 | 48,689,435-48,691,106 |
| Exon 30 | ENSE00000669950 | 48,688,300-48,688,453 | | | |
| Exon 31 | ENSE00001255082 | 48,688,660-48,689,066 | | | |
| Exon 32 | ENSE00000867103 | 48,689,320-48,689,434 | | | |
| Exon 33 | ENSE00000867104 | 48,691,107-48,691,427 | | | |

WAS gene has 559 SNPs and the NCBI rs number and/or UniProt VAR number for this WAS gene are VAR_005823, VAR_005825, VAR_005826, VAR_005827, VAR_005828, VAR_005829, VAR_005830, VAR_005831, VAR_005832, VAR_005833, rs146220228, VAR_005835, VAR_005836, VAR_005837, VAR_005838, VAR_005839, VAR_008105, VAR_008106, VAR_008106, VAR_008107, VAR_008108, VAR_008109, VAR_008110, VAR_012710, VAR_012711, VAR_022806, VAR_022807, VAR_033255, VAR_033256, VAR_033257, VAR_074020, rs192092, rs235422, rs235423, rs2735860, rs2737796, rs2737797, rs2737798, rs2737799, rs2737800, rs3215413, rs11545907, rs35351086, rs35359501, rs34164243, rs28379745, rs41298466, rs58371799, rs78375578, rs132630272, rs132630273, rs132630276, rs139857045, rs143825543, rs145072740, rs145299197, rs141722718, rs140238646, rs139265251, rs149941344, rs142231772, rs181677888, rs182012094, rs184380373, rs182671742, rs189073442, rs187071749, rs187207301, rs187614405, rs184217101, rs182475232, rs188698425, rs185865050, rs189579998, rs181475502, rs186835743, rs184064819, rs188356809, rs191 140821, rs180938515, rs201300703, rs368379103, rs150554260, rs150520117, rs149422306, rs181260434, rs182270501, rs368635575, rs150252581, rs371262569, rs375987005, rs149123892, rs373438606, rs201657175, rs144372473, rs149932808, rs369059472, rs148800063, rs141629445, rs371506803, rs141605347, rs191967655, rs143885622, rs138904063, rs191999320, rs376545922, rs376560886, rs143299151, rs145040665, rs200543049, rs376723243, rs140851093, rs369642443, rs369654974, rs139659302, rs374283590, rs372182723, rs193179881, rs139577358, rs112789098, rs132630275, rs377126493, rs132630274, rs132630271, rs132630270, rs132630269, rs132630268, rs138249592, rs370010448, rs370053372, rs377295134, rs372649110, rs57489208, rs377415721, rs374811059, rs59154508, rs377442612, rs55789731, rs370235898, rs370248054, rs187588147, rs201085962, rs185798380, rs186722885, rs189608406, rs375356111, rs387906716, rs387906717, rs190136544, rs188887707, rs587776742, rs368523950, rs587776744, rs587776745, rs201454882, rs781960144, rs781960751, rs371005311, rs781967249, rs371038390, rs371056209, rs190513631, rs376093906, rs373491815, rs373524969, rs781997651, rs200261212, rs782001600, rs782003647, rs376202818, rs369127837, rs371738193, rs371765089, rs782024402, rs374035804, rs192079438, rs781792192, rs200530781, rs200571645, rs374270583, rs781799471, rs369850591, rs374458439, rs781801746, rs781804991, rs782037639, rs267606468, rs374574436, rs782041200, rs782041815, rs781813385, rs193922412, rs782046601, rs782048178, rs193922413, rs193922414, rs781825719, rs193922415, rs782053989, rs193922416, rs781831613, rs781832180, rs199602285, rs370188924, rs372779500, rs781839366, rs781839950, rs367960760, rs375094923, rs782061167, rs368143764, rs781847394, rs368151220, rs375397970, rs375433190, rs782070061, rs587776743, rs781844097, rs782071252, rs781858831, rs781858910, rs375722538, rs781%65107, rs781969149, rs782080179, rs781866378, rs781970390, rs781971050, rs782085606, rs781980332, rs781985914, rs782088555, rs781988138, rs781879472, rs781880558, rs782001133, rs782095743, rs781884443, rs782004305, rs782006738, rs781887004, rs782010302, rs781790001, rs781790075, rs781893580, rs782105907, rs782106008, rs782024423, rs781897383, rs782107409, rs781898144, rs782109432, rs782028654, rs782029725, rs781903491, rs782114028, rs781904212, rs782115211, rs782117696, rs781798149, rs782198852, rs782422248, rs782199885, rs782423704, rs781799705, rs782430218, rs782204870, rs781918613, rs782699945, rs781920984, rs782032826, rs781808308, rs782706620, rs781809829, rs782436940, rs782136935, rs782137826, rs782046227, rs781927248, rs781821301, rs782711732, rs782214680, rs782712522, rs781930194, rs781930795, rs781823025, rs782053821, rs781931808, rs782715112, rs782218044, rs781932560, rs781829884, rs781932766, rs782056506, rs782148048, rs782148427, rs782057219, rs782060209, rs782451336, rs782149918, rs781840404, rs781840555, rs782073716, rs781848217, rs78206889, rs781854985, rs781939354, rs782226009, rs781855402, rs782227473, rs781856200, rs781860824, rs782462041, rs782728401, rs782076656, rs782082850, rs781868934, rs782730052, rs781945347, rs782087200, rs782165032, rs782087798, rs782730988, rs782466836, rs781877730, rs782468076, rs782235068, rs781948181, rs782169070, rs782093868, rs782237502, rs782736731, rs781952808, rs782174975, rs781885441, rs781886957, rs781954340, rs782099535, rs781889846, rs782102387, rs782484186, rs782106604, rs782486251, rs782244081, rs781901235, rs782454496, rs782224455, rs782156141, rs782185805, rs782745511, rs782158640, rs781942437, rs782727292, rs781943866, rs782163145, rs782752881, rs782730039, rs782195195, rs782753744, rs782198309, rs782164078, rs782233413, rs782166068, rs782257002, rs782502982, rs782236380, rs782175829, rs782760255, rs782739686, rs782741936, rs782761426, rs782742185, rs782508491, rs782764521, rs782264561, rs782181574, rs782768055, rs782267862, rs782515058, rs782270626, rs782271252, rs782517333, rs782484571, rs782274825, rs782775268, rs782525805, rs782280376, rs782526978, rs782527416, rs781902189, rs782285538, rs782286374, rs782784813, rs782533836, rs782290152, rs782290433, rs782789778, rs782792123, rs781910412, rs782298971, rs782299198, rs782793722, rs781917584, rs782133298, rs782706383, rs782707287, rs782439867, rs782301435, rs782301829, rs781928233, rs782303075, rs782303232, rs782303344, rs782445816, rs782798030, rs782798054, rs782714823, rs782546323, rs782305649, rs782307200, rs782716215, rs782448330, rs782549811, rs782550850, rs782550903, rs782149318, rs782809428, rs782809471, rs782314065, rs782810555, rs782555164, rs782316674, rs782816042, rs782817462, rs781934834, rs782559216, rs782320595, rs782451927, rs782563873, rs782328659, rs782566749, rs782452466, rs782152560, rs782335953, rs782336329, rs782337048, rs782572275, rs782575181, rs782575503, rs782244198, rs782487210, rs782578703, rs782185441, rs782347339, rs782488331, rs782582468, rs782584950, rs782355555, rs782587262, rs782489472, rs782489564, rs782246817, rs782247964, rs782363149, rs782595017, rs782350319, rs782596670, rs782587623, rs782602857, rs782593697, rs782370903, rs782594040, rs782594221, rs782605668, rs782607150, rs782363926, rs782611357, rs782615103, rs782249573, rs782616668, rs782498750, rs782381708, rs782256212, rs782256229, rs782628139, rs782501696, rs782387981, rs782503796, rs782259006, rs782504692, rs782636781, rs782761074, rs782639067, rs782392284, rs782507290, rs782641390, rs782767521, rs782517747, rs782281905, rs782655607, rs782656368, rs782401032, rs782659259, rs782666797, rs782666810, rs782405509, rs782406499, rs782578064, rs782347054, rs782672601, rs782677027, rs782793103, rs782681903, rs782682607, rs782683720, rs782414304, rs782542818, rs782415242, rs782686805, rs782794812, rs782420124, rs782543411, rs782543817, rs782543830, rs782544992, rs782303992, rs782798363, rs782802310, rs782803240, rs782553062, rs782818249, rs782559657, rs782567663, rs782334266, rs782343875, rs782366146, rs782370797, rs782605030, rs782605575, rs782375661, rs782615912, rs782380914, rs782383513, rs782384890, rs782386990, rs782388030, rs782631956, rs782634241, rs782638115, rs782640255, rs782643256, rs782645822, rs782397366, rs782670327, rs782672389, rs782681671, rs782415042, rs782693720, and rs782694766.

Wiskott-Aldrich Syndrome (WAS) is an X-linked primary immunodeficiency caused by mutations in the WAS gene. This disease is characterized by the classic triad of microthrombocytopenia, eczema, and recurrent infections. Besides the basic features, individuals with WAS are at high risk of developing autoimmune disorders and cancers (Massaad et al., 2013, Ann N Y Acad Sci; the contents of which are herein incorporated by reference in their entireties). WAS affects approximately 1-10 out of a million male births worldwide, with about 36 new cases every year in the United States and Europe. Without treatment, the average life expectancy is 15-20 years (Shin et al., 2012, Bone Marrow Transplant; the contents of which are herein incorporated by reference in their entireties).

X-Linked Thrombocytopenia (XLT) and X-Linked Neutropenia (XLN) are two milder forms of this disease. The defective nature of the WAS mutations correlates with the severity of the disease. While WAS is caused by mutations in the WAS gene that lead to absence of WAS protein (WASp), XLT is caused by mutations resulting in residual function of WASp and XLN is caused by gain-of-function mutations in the WAS gene. Patients with XLT present impaired platelet function and increased risk of severe bleedings, whereas patients with XLN present severe chronic neutropenia with varying levels of neutrophils.

The WAS gene contains 12 exons, spanning 14.8 kb of genomic DNA. The cDNA for WASp is 1.5 kb long. Mutations in WAS gene that lead to WAS are largely patient specific and spread across the whole gene (Massaad et al., 2013, Ann N Y Acad Sci; the contents of which are herein incorporated by reference in their entireties).

WASp is a 502 amino acid protein expressed exclusively in cytoplasm of hematopoietic cells. WASp alone exists in an auto-inhibited conformation and its activation is triggered upon binding of the small GTPase Cell Division Cycle 42 (CDC42) and Phosphatidylinositol-4,5-bisphosphate (PIP2). The activated WASp binds to and activates the Actin-Related Proteins 2/3 (Arp2/3) complex which stimulates actin polymerization by providing a nucleation core. The role of Arp2/3 complex in the regulation of actin cytoskeleton contributes to a variety of essential cellular functions including cell adhesion, shaping, and motility.

Absence of WASp expression leads to dysregulated functions in hematopoietic cells. In white blood cells, the lack of WASp signaling impairs cell movement and ability to form immune synapses at cellular interface with foreign invaders during an infection. This causes WAS patients to be highly susceptible to many bacterial, viral and fungal infections. Platelets that lack WASp have impaired development and early cell death, resulting in reduced platelet size and numbers. The platelet abnormality underlines the severe bleeding problems in WAS. Further, T cell functions are also defective due to the impaired ability to form stable immune synapses in T cell receptor dependent activation. This is a major cause of the immunodeficiency associated with WAS. In addition, absence of WASp in Natural Killer (NK) cells leads to reduced efficiency in phagocytosis and cell lysis, at normal or increased number of NK-cells in the body.

The only curative treatment for WAS is bone marrow transplantation (BMT). When a human leukocyte antigen (HLA)-matched donor is available, BMT can significantly improve survival up to 87% (Filipovich et al., 2001, Blood; the contents of which are herein incorporated by reference in their entireties). However, there is a shortage of HLA-matched donors and BMT often gives rise to acute or chronic graft-versus-host disease that can be life-threatening. Alternative therapies include splenectomy to increase and normalize the platelet counts, and prophylactic use of antibiotics and intravenous immunoglobulin to reduce the risk of infections. These treatments are only supportive and have limited long term effect.

Gene therapy is a promising alternative to BMT. The transplantation of autologous gene corrected CD34⁺ hematopoietic stem and progenitor cells (HSPCs) can lead to normal levels of functional white blood cells and platelets without the risk of graft-vs-host-disease and is independent from the availability of a matched donor. A number of clinical trials have been carried out for WAS in the past decade, and progress had been made (see reviews in Buchbinder et al., 2014, Appl Clin Genet; Martin et al., 2016, Expert Opin Orphan Drugs; Massaad et al., 2013, Ann N Y Acad Sci; the contents of each of which are herein incorporated by reference in their entireties). However, early studies relied on the use of retroviral or lentiviral vectors which are known to integrate randomly into the genome and cause undesirable mutations. For example, in the first clinical trial conducted with gammaretroviral vectors, 7 out of 10 patients developed leukemia due to retroviral integration near oncogenes (Braun et al., 2014, Sci Transl Med; the contents of which are herein incorporated by reference in their entireties).

Genome engineering is an emerging field that develops strategies and technologies for the precise manipulation of genes and genomes. Instead of relying on random insertions via integrative viral vectors, the use of recent genome engineering strategies, such as ZFNs, TALENs, HEs and MegaTALs, enables modifications only at desired locations, greatly improving efficiency and precision. Furthermore, random integration technologies offer little reproducibility, as there is no guarantee that the sequence would be inserted at the same place in two different cells. These newer platforms offer a much larger degree of reproducibility, but still have limitations.

HSPCs can be great target cells for gene therapy of WAS, as the introduction of a few gene-corrected HSPCs can restore all the hematopoietic lineages of the patients. Gene correction using the specific nucleases relies on the cellular repair mechanism of homologous directed repair (HDR). Although HSPCs are known to have low HDR frequency, some studies have managed to improve the efficiency by delivering the nuclease using mRNA nucleofection and favoring HDR by using nonintegrative lentiviral vectors to deliver the donor DNA (Genovese et al., 2014, Nature; Martin et al., 2016, Expert Opin Orphan Drugs, the contents of each of which are herein incorporated by reference in their entireties).

Despite advances in clinical care and medical research in WAS, there remains an unmet need for effective and safe treatments for WAS patients. The present disclosure presents a novel approach to correct the genetic causes of WAS and related disorders. By using this strategy, stable engraftment and physiological expression of WASp in blood cell lineages can be achieved. This approach can create permanent changes to the genome that can address the WAS gene related disorders and ultimately stop the progression of the disease.

### Genome Editing Strategy

### Knock-in Strategy

In one aspect, the present disclosure proposes insertion of a nucleic acid sequence of a WAS gene or functional derivative thereof into a genome of a cell. In embodiments, the WAS gene may encode a wild-type WAS protein. The functional derivative of a WAS gene may include a nucleic acid sequence encoding a functional derivative of the WAS protein that has a substantial activity of the wildtype WAS protein, e.g, at least about 30%, about 40%, about 50%, about 60%, about 70%, about 80%, about 90%, about 95% or about 100% of the activity that the wildtype WAS protein exhibits. In some embodiments, one having ordinary skill in the art can use a number of methods known in the field to test the functionality or activity of a compound, e.g. peptide or protein. Such methods may include, but not limited to, in vitro cell based assays as well as in vitro non-cell based assays such as measuring biding ARP2/3 complex with actin (see, e.g. Higgs H, and Pollard T D, “Regulation of Actin Polymerization by Arp2/3 Complex and Wasp/Scar Proteins,” (1999), The Journal of Biological Chemistry 274, 32531-32534 and Marchand J B et al., “Interaction of WASP/Scar proteins with actin and vertebrate Arp2/3 complex,” (2001). Nat Cell Biol. 3(1):76-82 which are incorporated by reference in entirety). The functional derivative of the WAS protein may also include any fragment of the wildtype WAS protein or fragment of a modified WAS protein that has conservative modification on one or more of amino acid residues in the full length, wildtype WAS protein. Thus, in some embodiments, the functional derivative of a nucleic acid sequence of a WAS gene may have at least about 30%, about 40%, about 50%, about 60%, about 70%, about 80%, about 85%, about 90%, about 95%, about 96%, about 97%, about 98% or about 99% nucleic acid sequence identity to the WAS gene.

In one aspect, the present disclosure proposes the use of CRISPR/Cas9 to genetically introduce (knock-in) the cDNA of the complete WAS-gene. As mutations are often scattered across the entire gene, this approach can cover the majority of patients.

In some embodiments, the genome of a cell can be edited by inserting a nucleic acid sequence of a WAS gene or functional derivative thereof into a genomic sequence of the cell. In some embodiments, the cell subject to the genome-edition has one or more mutation(s) in the genome which results in reduction of the expression of endogenous WAS gene as compared to the expression in a normal that does not have such mutation(s). The normal cell may be a healthy or control cell that is originated (or isolated) from a different subject who does not have WAS gene defects. In some embodiments, the cell subject to the genome-edition may be originated (or isolated) from a subject who is in need of treatment of WAS gene related condition or disorder. Therefore, in some embodiments the expression of endogenous WAS gene in such cell is about 10%, about 20%, about 30%, about 40%, about 50%, about 60%, about 70%, about 80%, about 90% or about 100% reduced as compared to the expression of endogenous WAS gene expression in the normal cell.

In some embodiments, the insertion of a nucleic acid sequence of a WAS gene or functional derivative thereof can be done by providing the following to the cell: a deoxyribonucleic acid (DNA) endonuclease or an oligonucleotide encoding the DNA endonuclease, a targeting oligonucleotide, and a donor template.

In embodiments, the DNA endonuclease is an enzyme selected from the group consisting of any of those in Table 1, Table 2, and variants having at least 70% homology to any of those listed in Table 1 or Table 2. In some embodiments, the DNA endonuclease is Cas 9. In embodiments, the oligonucleotide encoding the DNA endonuclease is codon optimized. In embodiments, the oligonucleotide encoding the DNA endonuclease is a deoxyribonucleic acid (DNA) sequence or a ribonucleic acid (RNA) sequence. In certain embodiments where the oligonucleotide encoding the DNA endonuclease is a RNA sequence, the RNA sequence can be linked to the targeting oligonucleotide via a covalent bond.

In embodiments, the targeting oligonucleotide has a region that is complementary to the genomic sequence at which a WAS gene or derivative thereof is inserted. In some embodiments, the complementary region is a spacer sequence that has at least 15 bases complementary to the genomic sequence targeted for insertion. In embodiments, the targeting oligonucleotide is a guide RNA (gRNA).

In embodiments, the genomic sequence that is targeted by the targeting oligonucleotide such as gRNA is at, within, or near the endogenous WAS gene. In some embodiments, the target site in the genome is in an intergenic region that is upstream of the promoter of the WAS gene in the genome. In certain embodiments, the intergenic region is at least 500 bp upstream of the first exon of the WAS gene. In certain embodiments, the intergenic region is about 500 bp upstream of the first exon of the WAS gene. In certain embodiments, the intergenic region is at least, about or at most 50, 100, 150, 200, 250, 300, 350, 400, 450 or 500 bp upstream of the WAS promoter or the first exon. In some embodiments, the target site is in an intergenic region that is upstream of the WAS gene, for example, at least, about or at most 0.1 kb, about 0.2 kb, about 0.3 kb, about 0.4 kb, about 0.5 kb, about 1 kb, about 1.5 kb, about 2 kb, about 2.5 kb, about 3 kb, about 3.5 kb, about 4 kb, about 4.5 kb or about 5 kb upstream of the WAS promoter or the first exon. In some embodiments, the target site is anywhere within about 0 bp to about 100 bp upstream, about 101 bp to about 200 bp upstream, about 201 bp to about 300 bp upstream, about 301 bp to about 400 bp upstream, about 401 bp to about 500 bp upstream, about 501 bp to about 600 bp upstream, about 601 bp to about 700 bp upstream, about 701 bp to about 800 bp upstream, about 801 bp to about 900 bp upstream, about 901 bp to about 1000 bp upstream, about 1001 bp to about 1500 bp upstream, about 1501 bp to about 2000 bp upstream, about 2001 bp to about 2500 bp upstream, about 2501 bp to about 3000 bp upstream, about 3001 bp to about 3500 bp upstream, about 3501 bp to about 4000 bp upstream, about 4001 bp to about 4500 bp upstream or about 4501 bp to about 5000 bp upstream of the WAS promoter or the first exon.

In some embodiments, the genomic sequence that is targeted by the targeting oligonucleotide such as gRNA is at, within, or near a safe harbor locus or a safe harbor site. In some embodiments, the safe-harbor locus is selected from the group consisting of albumin gene, an AAVS1 gene, an HRPT gene, a CCR5 gene, a globin gene, TTR gene, TF gene, F9 gene, Alb gene. Gys2 gene and PCSK9 gene. In some embodiments, the safe harbor site is selected from the group consisting of the following regions: AAVS1 19q13.4-qter, HRPT 1q31.2, CCR5 3p21.31, Globin 11p15.4, TTR 18q12.1, TF 3q22.1, F9 Xq27.1, Alb 4q13.3, Gys2 12p12.1, and PCSK9 1p32.3.

In some embodiments, the target site in the genome is at, within, or near the AAVS1 gene. In some other embodiments, the target site in the genome is upstream of the AAVS1 gene. In some embodiments, the target site is in an intergenic region that is upstream of the AAVS1 gene, e.g, about 2.5 kb upstream of the first exon of the AAVS1 gene. In some embodiments, the target site is in an intergenic region that is upstream of the AAVS1 gene, e.g, at least 2.5 kb upstream of the first exon of the AAVS1 gene. In some embodiments, the target site is in an intergenic region that is upstream of the AAVS1 gene, e.g, about 2.5 kb to about 5 kb upstream of the first exon of the AAVS1 gene. In some embodiments, the target site is in an intergenic region that is upstream of the AAVS1 gene, for example, about 2.5 kb to about 3 kb, about 2.5 kb to about 3.5 kb, about 2.5 kb to about 4 kb, about 2.5 kb to about 4.5 kb, about 2.5 kb to about 5 kb, about 2.5 kb to about 5.5 kb, about 2.5 kb to about 6 kb, about 2.5 kb to about 6.5 kb, about 2.5 kb to about 7 kb, about 2.5 kb to about 7.5 kb, about 2.5 kb to about 8 kb, about 2.5 kb to about 8.5 kb, about 2.5 kb to about 9 kb, about 2.5 kb to about 9.5 kb or about 2.5 kb to about 10 kb upstream of the first exon of the AAVS1 gene. In some embodiments, the target site is in an intergenic region that is upstream of the AAVS1 gene, for example, at least, about or at most 0.5 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb or about 10 kb upstream of the AAVS1 promoter or the first exon. In some embodiments, the target site is anywhere whtin about 0 bp to about 500 bp upstream, about 501 bp to about 1000 bp upstream, about 1001 bp to about 1500 bp upstream, about 1501 bp to about 2000 bp upstream, about 2001 bp to about 2500 bp upstream, about 2501 bp to about 3000 bp upstream, about 3001 bp to about 3500 bp upstream, about 3501 bp to about 4000 bp upstream, about 4001 bp to about 4500 bp upstream, about 4501 bp to about 5000 bp, about 5001 bp to about 5500 bp, about 5501 bp to about 6000 bp, about 6001 bp to about 6500 bp, about 6501 bp to about 7000 bp, about 7001 bp to about 7500 bp, about 7501 bp to about 8000 bp, about 8001 bp to about 8500 bp, about 8501 bp to about 9000 bp or about 9501 bp to about 10000 bp upstream of the AAVS1 promoter or the first exon.

In some embodiments, the target site in the genome is at, within, or near the AAVS1 gene. In some other embodiments, the target site in the genome is upstream of the AAVS1 gene encompassing nucleotides 55,120,000 to 55,122,500 on chromosome 19.

In some embodiments, the donor template can have a WAS cDNA sequence or a derivative thereof. The derivative of a WAS gene may include a nucleic acid sequence that encodes a functional derivative of the wildtype WAS protein or fragment thereof. In some embodiments, the donor template can have additional element(s) such as one or more regulatory sequences and reporter genes. The regulatory sequences and reporter genes are optional and used when needed in that in some embodiments such optional sequences may not be present in the donor template.

In some embodiments, the donor template can have a promoter sequence for the expression of the introduced WAS gene or derivative thereof. In some embodiments, the promoter can be a WAS proximal promoter, WAS distal promoter or MND synthetic promoter. In alternative embodiments, other eukaryotic promoters can be and such suitable eukaryotic promoters (i.e., promoters functional in a eukaryotic cell) can include, but not limited to, those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-1 promoter.

In some embodiments, the donor template does not have a promoter for the expression of the introduced WAS gene or functional derivative thereof. Therefore, in such embodiments, the transgene expression is controlled by an endogenous promoter that is present at, within, or near the targeted genomic sequence. In some embodiments, the endogenous promoter is the endogenous WAS promoter.

In some embodiments, one or more of any oligonucleotides or nucleic acid sequences that are provided to the cell for genome-edition can be encoded in an Adeno Associated Virus (AAV) vector. Therefore, in some embodiments, a targeting oligonucleotide can be encoded in an AAV vector. In some embodiments, a nucleic acid encoding a DNA endonuclease can be encoded in an AAV vector. In some embodiments, a donor template can be encoded in an AAV vector. In some embodiments, two or more oligonucleotides or nucleic acid sequences can be encoded in a single AAV vector. Thus, in some embodiments, a gRNA sequence and a DNA endonuclease-encoding nucleic acid can be encoded in a single AAV vector.

In some embodiments, any compounds (e.g, a DNA endonuclease or a nucleic acid encoding thereof, gRNA and donor template) that are provided to the cell for genome-edition can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA and donor template is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA and donor template are formulated in a liposome or lipid nanoparticle together.

In some embodiments, any compounds (e.g, a DNA endonuclease or a nucleic acid encoding thereof, gRNA and donor template) that are provided to the cell for genome-edition can be delivered via transfection such as electroporation. In some exemplary embodiments, a DNA endonuclease can be precomplexed with a gRNA, forming a Ribonucleoprotein (RNP) complex, prior to the provision to the cell and the RNP complex can be electroporated. In such embodiments, the donor template can delivered via electroporation.

In some embodiments, the cell subject to the genome-edition may have one or more mutation(s) at, within, or near the endogenous WAS gene in the genome. Therefore, in some embodiments the expression of endogenous WAS gene or activity of WAS gene products in such cell is substantially less as compared to those of a normal, healthy cell, e.g, at least the about 10% to about 100% reduction as compared to the normal cell. Upon successful insertion of the transgene, e.g, a nucleic acid encoding a WAS gene or functional fragment thereof, the expression of the introduced WAS gene or functional derivative thereof in the cell can be at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous WAS gene of the cell. In some embodiments, the activity of introduced WAS gene products including the functional fragment of WAS in the genome-edited cell can be at least about 10%, about 20, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10.000% or more as compared to the expression of endogenous WAS gene of the cell. In some embodiments, the expression of the introduced WAS gene or functional derivative thereof in the cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the cell. Also, in some embodiments, the activity of introduced WAS gene products including the functional fragment of WAS in the genome-edited cell can be comparable to or more than the activity of WAS gene products in a normal, healthy cell.

In some embodiments, the cell subject to the genome-edition is a stem cell. In some embodiments, the stem cell is a CD34+ hematopoietic stem and progenitor cell (HSPC). In some embodiments, the stem cell is an induced pluripotent stem cell (iPSC). In some embodiments, the stem cell is a mesenchymal stem cell.

In some embodiments, two guide RNAs specific for the cleavage site can be delivered together with a cDNA template containing homology sequences. The two guide RNAs will delete the mutated region and the cDNA will be introduced directly downstream of the physiological promotor. The knock-in will be achieved by cellular repair mechanism of HDR. The best position for cleavage will be assessed experimentally in order to obtain translated, non-toxic proteins. The Cas9 nuclease can be delivered as a protein or as nucleic acids (mRNA or DNA), whereas the guide RNAs can be delivered as RNA or co-expressed with the same DNA as the Cas9. The delivery to the cells can be viral or non-viral, e.g, nanoparticles for the protein Cas9 and mRNA together with an AAV for the DNA donor template.

### Alternative Strategies

In some embodiments, alternative strategies such as 1) correcting, by insertions or deletions that arise due to the imprecise NHEJ pathway, one or more mutations at, within, or near the WAS gene or other DNA sequences that encode regulatory elements of the WAS gene or 2) correcting, by HDR, one or more mutations at, within, or near the WAS gene or other DNA sequences that encode regulatory elements of the WAS gene can be employed to alter the expression of WAS in a cell via genome-edition.

In some embodiments, the treatment method may target hematopoietic stem and progenitor cells (HSPCs) for therapeutic gene editing. HSPCs are an important target for gene therapy as they provide a prolonged source of the corrected cells. HSCs give rise to both the myeloid and lymphoid lineages of blood cells. Mature blood cells have a finite life-span and must be continuously replaced throughout life. Blood cells are continually produced by the proliferation and differentiation of a population of pluripotent hematopoietic stem cells (HSCs) that can be replenished by self-renewal. Bone marrow is the major site of hematopoiesis in humans and a good source for HSPCs. HSPCs can be found in small numbers in the peripheral blood. In some indications or treatments their numbers increase. The progenies of HSCs mature through stages, generating multi-potential and lineage-committed progenitor cells including T- and B-cells. Edited cells, such as CD34+ HSPCs would be returned to the patient.

### Ex Vivo Delivery

Ex vivo delivery method can be used for delivery of CRISPR/Cas9 to cells. This method has the steps of 1) isolate CD34+ HSPCs from the patient; 2) editing at, within, or near the WAS gene or other DNA sequences that encode the regulatory elements of the WAS gene of the HSCs from the patient; and 3) reinfuse the HSPCs into the patient. Transplantation requires clearance of bone-marrow niches for the donor HSPCs to engraft. Current methods rely on radiation and/or chemotherapy. Due to the limitations these impose, safer conditioning regimens have been developed. Success of HSPC transplantation depends upon efficient homing to bone marrow, subsequent engraftment, and bone marrow repopulation. In some embodiments, hematopoietic stem cells (HSCs) are an important target for ex vivo gene therapy as they provide a prolonged source of the corrected cells. Treated CD34+ cells would be returned to the patient. The level of engraftment is important, as is the ability of the gene-edited cells to differentiate into all lineages following CD34+ HSPCs infusion in vivo.

In one aspect, the disclosures provide a method of treating a subject for a Wiskott-Aldrich syndrome (WAS) gene related condition or disorder via an ex vivo approach. The method may have providing a genetically modified cell to the subject. The genome of the genetically modified cell may have been edited such that an exogenous nucleic acid sequence of a WAS gene or functional derivative thereof is inserted in the genome.

In some embodiments, the subject who is in need of the treatment method accordance with the disclosures is a patient having symptoms of the Wiskott-Aldrich syndrome (WAS) gene related condition or disorder. Alternatively, the subject can be a human diagnosed with a risk of the Wiskott-Aldrich syndrome (WAS) gene related condition or disorder.

In some embodiments, the genetically modified cell that is used for the treatment is originated from the subject who is in need of the treatment method according to the disclosure. Therefore, in some embodiments, the genetically modified cell originated from a subject has one or more mutation(s) in the genome which results in reduction of the expression of endogenous WAS gene as compared to the expression of endogenous WAS gene in a normal cell that does not have such mutation(s). In some embodiments, such mutation(s) are present at, within, or near the endogenous WAS gene in the genome. Due to such mutation, in some embodiments, the expression of endogenous WAS gene in the subject-originated cell is about 10%, about 20%, about 30%, about 40%, about 50%, about 60%, about 70%, about 80%, about 90% or about 100% reduced as compared to the expression of endogenous WAS gene expression in a normal cell that does not have such mutation(s).

In some embodiments, the genetically modified cell originated from a subject has one or more mutation(s) in the genome which results in reduction of the expression of functional endogenous WAS gene as compared to the expression of functional endogenous WAS gene in a normal cell that does not have such mutation(s). In some embodiments, such mutation(s) are present at, within, or near the endogenous WAS gene in the genome. Due to such mutation, in some embodiments, the expression of functional endogenous WAS gene in the subject-originated cell is about 10%, about 20%, about 30%, about 40%, about 50%, about 60%, about 70%, about 80%, about 90% or about 100% reduced as compared to the expression of functional endogenous WAS gene expression in a normal cell that does not have such mutation(s).

In some embodiments, one or more cells are isolated from a subject who is in need of the treatment according to the disclosure and the cells are modified. The modification may include a genome-edition which is done by inserting a WAS gene or derivative thereof into the genome of the cell. With this modification, the expression of the introduced WAS gene or functional derivative thereof in the genetically modified cell can be at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3.000%, about 5,000%, about 10,000% or more as compared to the expression of endogenous WAS gene of the cell. In some embodiments, the activity of introduced WAS gene products including the functional fragment of WAS in the genome-edited (or genetically modified) cell can be at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous WAS gene of the cell. In some embodiments, the expression of the introduced WAS gene or functional derivative thereof in the genetically modified cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the cell. Also, in some embodiments, the activity of introduced WAS gene products including the functional fragment of WAS in the genome-edited cell can be comparable to or more than the activity of WAS gene products in a normal, healthy cell.

In some embodiments, the cell subject to the genome-edition is a stem cell. In some embodiments, the stem cell is a CD34+ hematopoietic stem and progenitor cell (HSPC). In some embodiments, the stem cell is an induced pluripotent stem cell (iPSC). In some embodiments, the stem cell is a mesenchymal stem cell.

In some embodiments, the treatment method in accordance with the disclosure may also include a process of producing a genetically modified cell. The production process may include isolating a cell from the subject who is in need of the treatment and editing the genome of the cell by inserting the exogenous nucleic acid sequence of a WAS gene or functional derivative thereof into a genomic sequence of the cell. In some embodiments, the isolated cell is a somatic cell and the method further has introducing one or more of pluripotency-associated genes into the somatic cell to induce the somatic cell to become a pluripotent stem cell. In some embodiments, the pluripotency-associated genes are selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some embodiments, the exogenous nucleic acid sequence (or transgene) of a WAS or functional derivative thereof is inserted at, within, or near the endogenous WAS gene or WAS gene regulatory elements in the genome of the cell. In some embodiments, the exogenous nucleic acid sequence or transgene is inserted in an intergenic region that is upstream of the promoter of the WAS gene in the genome. In certain embodiments, the intergenic region is about 500 bp upstream of the first exon of the WAS gene. In certain embodiments, the intergenic region is at least 500 bp upstream of the first exon of the WAS gene.

In some embodiments, the exogenous nucleic acid sequence or transgene is inserted at, within, or near a safe harbor locus or a safe harbor site. In some embodiments, the safe-harbor locus is selected from the group consisting of albumin gene, an AAVS1 gene, an HRPT gene, a CCR5 gene, a globin gene, TTR gene, TF gene, F9 gene, Alb gene, Gys2 gene and PCSK9 gene. In some embodiments, the safe harbor site is selected from the group consisting of the following regions: AAVS119q13.4-qter, HRPT 1q31.2, CCR5 3p21.31, Globin 11p15.4, TTR 18q12.1. TF 3q22.1, F9 Xq27.1, Alb 4q13.3, Gys2 12p12.1 and PCSK9 1p32.3. In some embodiments, the exogenous nucleic acid sequence or transgene is inserted at, within, or near the AAVS1 gene. In some embodiments, the exogenous nucleic acid sequence or transgene is inserted upstream of the AAVS1 gene, in particular an intergenic region that is at least or about 2.5 kb upstream of the first exon of the AAVS1 gene.

### In Vive Delivery

In vivo delivery method can also be used for delivery of CRISPR/Cas9 to cells. This method has the step of editing the WAS gene in a cell of the patient. Although blood cells present an attractive target for ex vivo therapy, increased efficacy in delivery may permit direct in vivo delivery to the HSCs and/or other progenitors such as CD34+ HSPCs. In vivo treatment would eliminate a number of treatment steps and losses associated with ex vivo treatment and engraftment. However, a lower rate of delivery may require higher rates of editing. Unwanted Cas9 mediated cleavage in cells other than the target cells may be prevented by the use of promotors that are cell type-specific or development stage specific. Alternatively, the promotors can be inducible, allowing for temporal control of Cas9 expression if it is delivered as a plasmid. The amount of time that delivered RNA and protein remain in the cell can also be adjusted by modifying the RNA or protein to modulate the half-life.

### Development Plan

In order to minimize off-target cleavage to reduce the detrimental effects of mutations and chromosomal rearrangements, bioinformatics can be used. Studies on CRISPR/Cas9 systems suggested the possibility of high off-target activity due to nonspecific hybridization of the guide strand to DNA sequences with base pair mismatches and/or bulges, particularly at positions distal from the PAM region (Cradick et al., 2013, Nucleic Acids Res: Hsu et al., 2013, Nat. Biotechnol; Fu et al., 2013, Nat. Biotechnol; Lin et al., 2014, Nucleic Acids Res; the contents of each of which are herein incorporated by reference in their entireties). It is therefore important to have a bioinformatics tool that can identify potential off-target sites that have insertions and/or deletions between the RNA guide strand and genomic sequences, in addition to base-pair mismatches. The bioinformatics based tool (e.g. software), COSMID (CRISPR Off-target sites with Mismatches, Insertions and Deletions), autoCOSMID or ccTOP (https://crispr.cos.uni-heidelberg.de/) can be used to search genomes for potential CRISPR off-target-sites (http://crispr.bme.gatech.edu). COSMID output ranked lists of the potential off-target sites based in the number and location of mismatches, allowing more informed choice of target sites and avoiding the use of sites with more likely off-target cleavage. Additional bioinformatics pipelines can be employed that weigh the estimated on- and/or off-target activity of gRNA targeting sites in a region. Other features that may be used to predict activity include information about the cell type in question, DNA accessibility, chromatin state, transcription factor binding sites, transcription factor binding data and other CHIP-seq data. Additional factors are weighed that predict editing efficiency such as relative positions and directions of the gRNA, local sequence features and micro-homologies (Bae et al., 2014, Nat. Methods; Prykhozhij et al., 2015, Plos One; Naito et al., 2015, Bioinformatics; Stemmer et al., 2015, Plos One; the contents of each of which are herein incorporated by reference in their entireties).

The various constructs can be screened for activity and specificity via transfection of tissue cultures (Cradick et al., 2014, Mol Ther Nucleic Acids; the contents of which are herein incorporated by reference in their entireties). Tissue culture cell lines, such as K-562 or 293T are easily transfected and result in high activity. These or other cell lines are evaluated to determine the cell lines that provide the best surrogate. These cells are then used for many early stage tests. Individual gRNAs will be transfected into the cells. Several days later the genomic DNA is harvested and the target site amplified by PCR. The cutting activity can be measured by the rate of insertions, deletions and mutations introduced by NHEJ repair of the free DNA ends. Although this method cannot differentiate correctly repaired sequences from uncleaved DNA, the level of cutting can be gauged by the amount of mis-repair. Off-target activity can be observed by amplifying identified putative off-target sites and using similar methods to detect cleavage. Translocation can also be assayed using primers flanking cut sites, to determine if specific cutting and translocations happen. Un-guided assays have been developed allowing complementary testing of off-target cleavage including guide-seq (Kim et al., 2015, Nat. Methods; Frock et al., 2015, Nat. Biotechnol; Kuscu et al., 2014, Nat. Biotechnol; the contents of which are herein incorporated by reference in their entireties). The gRNA with significant activity can then be followed up in cultured cells to measure the silencing of the WAS gene. The off-target effects can also be followed. Similarly, CD34+ HSPCs can be transfected and the level of gene correction and possible off-target events measured. These experiments allow for the optimization of nuclease and donor design and delivery.

Mouse models can be used in gene therapy studies to measure activity when targeting mutations in the similar mouse WAS gene. These studies are useful for the determination of the levels of correction needed. Animal models can also be used to test the levels of correction expected to result in phenotypic correction, as dose curves of wild-type (WT) cells can also be transferred to the knockout animal. Specificity and safety can be assayed in mouse models, though the differences between the genomes limit these to be surrogate studies only. Culture in human cells allows direct testing on the human target and the background human genome, as described above. Preclinical efficacy and safety evaluations can be observed through engraftment of modified mouse or human CD34+ HPSCs in a WAS− mouse model or similar mice.

The gene editing approach presented in the present disclosure would result in developing B- and T-cells in central lymphoid organs and in the appearance of B- and T-cells in peripheral blood. Serum Ig levels can be measured and compared to the range found in patients without immunodeficiencies. Ig and TCR Vβ gene segment usage in B- and T-cells, respectively, should be comparable to WT controls. Animal models have indicated that even low frequencies of B cells produced WT levels of serum immunoglobulins. T cell function can be assayed through TCR stimulation and cytokine measurement and compared to WT levels. Correction of platelet function can be evaluated through total platelet count, evaluation of platelet phenotype and measurement of clotting times. Clinical testing would include long term following of modified B- and T-cells to determine if there are any resulting growth abnormalities or signs of oncogene activation.

### VI. Other Therapeutic Approaches

Gene editing can be conducted using nucleases engineered to target specific sequences. To date there are four major types of nucleases: meganucleases and their derivatives, zinc finger nucleases (ZFNs), transcription activator like effector nucleases (TALENs), and CRISPR-Cas9 nuclease systems. The nuclease platforms vary in difficulty of design, targeting density and mode of action, particularly as the specificity of ZFNs and TALENs is through protein-DNA interactions, while RNA-DNA interactions primarily guide Cas9. Cas9 cleavage also requires an adjacent motif, the PAM, which differs between different CRISPR systems. Cas9 from Streptococcus pyogenes cleaves using a NGG PAM, CRISPR from Neisseria meningitidis can cleave at sites with PAMs including NNNNGATT, NNNNNGTTT and NNNNGCTT. A number of other Cas9 orthologs target protospacer adjacent to alternative PAMs.

CRISPR endonucleases, such as Cas9, can be used in the methods of the present disclosure. However, the teachings described herein, such as therapeutic target sites, could be applied to other forms of endonucleases, such as ZFNs, TALENs, HEs, or MegaTALs, or using combinations of nulceases. However, in order to apply the teachings of the present disclosure to such endonucleases, one would need to, among other things, engineer proteins directed to the specific target sites.

Additional binding domains can be fused to the Cas9 protein to increase specificity. The target sites of these constructs would map to the identified gRNA specified site, but would require additional binding motifs, such as for a zinc finger domain. In the case of Mega-TAL, a meganuclease can be fused to a TALE DNA-binding domain. The meganuclease domain can increase specificity and provide the cleavage. Similarly, inactivated or dead Cas9 (dCas9) can be fused to a cleavage domain and require the sgRNA/Cas9 target site and adjacent binding site for the fused DNA-binding domain. This likely would require some protein engineering of the dCas9, in addition to the catalytic inactivation, to decrease binding without the additional binding site.

### Zinc Finger Nucleases

Zinc finger nucleases (ZFNs) are modular proteins having an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN typically has 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers, although considerable expertise is required to do this well. In most applications of ZFNs, proteins of 4-6 fingers are used, recognizing 12-18 bp respectively. Hence, a pair of ZFNs will typically recognize a combined target sequence of 24-36 bp, not including the typical 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96(6):2758-63 (1999); Dreier B et al., J Mol Biol. 303(4):489-502 (2000); Liu Q et al., J Biol Chem. 277(6):3850-6 (2002); Dreier et al., J Biol Chem 280(42):35588-97 (2005); and Dreier et al., J Biol Chem. 276(31):29466-78 (2001).

### Transcription Activator-Like Effector Nucleases (TALENs)

TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs have tandem arrays of 33-35 amino acid repeats, with each repeat recognizing a single base pair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science 326(5959): 1509-12 (2009); Mak et al., Science 335(6069):716-9 (2012); and Moscou et al., Science 326(5959): 1501 (2009). The use of TALENs based on the “Golden Gate” platform, or cloning scheme, has been described by multiple groups; see, e.g., Cermak et al., Nucleic Acids Res. 39(12):e82 (2011); Li et al., Nucleic Acids Res. 39(14):6315-25(2011); Weber et al., PLoS One. 6(2):e16765 (2011); Wang et al., J Genet Genomics 41(6):339-47. Epub 2014 May 17 (2014); and Cermak T et al., Methods Mol Biol. 1239:133-59 (2015).

### Homing Endonucleases

Homing endonucleases (HEs) are sequence-specific endonucleases that have long recognition sequences (14-44 base pairs) and cleave DNA with high specificity—often at sites unique in the genome. There are at least six known families of HEs as classified by their structure, including LAGLIDADG (SEQ ID NO: 20,209). GIY-YIG, His-Cis box, H-N-H, PD-(D/E)xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24(8):663-80 (2014); Belfort and Bonocora, Methods Mol Biol. 1123:1-26 (2014); Hafez and Hausner. Genome 55(8):553-69 (2012); and references cited therein.

### MegaTAL/Tev-mTALEN/MegaTev

As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., NAR 42: 2591-2601 (2014); Kleinstiver et al., G3 4:1155-65 (2014); and Boissel and Scharenberg, Methods Mol. Biol. 1239: 171-96 (2015).

In a further variation, the MegaTev architecture is the fusion of a meganuclease (Mega) with the nuclease domain derived from the GIY-YIG homing endonuclease I-TevI (Tev). The two active sites are positioned ˜30 bp apart on a DNA substrate and generate two DSBs with non-compatible cohesive ends; see, e.g., Wolfs et al., NAR 42, 8816-29 (2014). It is anticipated that other combinations of existing nuclease-based approaches will evolve and be useful in achieving the targeted genome modifications described herein.

dCas9-FokI or dCpf1-Fok1 and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 24 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a FokI domain to the deactivated Cas9; see, e.g., Tsai et al., Nature Biotech 32: 569-76 (2014); and Guilinger et al., Nature Biotech. 32: 577-82 (2014). Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### VII. Kits

The present disclosure provides kits for carrying out the methods described herein. A kit can include one or more of a genome-targeting nucleic acid, a polynucleotide encoding a genome-targeting nucleic acid, a site-directed polypeptide, a polynucleotide encoding a site-directed polypeptide, and/or any nucleic acid or proteinaceous molecule necessary to carry out the aspects of the methods described herein, or any combination thereof.

In some embodiments, a kit can include: (1) a vector having a nucleotide sequence encoding a genome-targeting nucleic acid, (2) the site-directed polypeptide or a vector having a nucleotide sequence encoding the site-directed polypeptide, and (3) a reagent for reconstitution and/or dilution of the vector(s) and or polypeptide.

In some embodiments, a kit can have: (1) a vector having (i) a nucleotide sequence encoding a genome-targeting nucleic acid, and (ii) a nucleotide sequence encoding the site-directed polypeptide; and (2) a reagent for reconstitution and/or dilution of the vector.

In some embodiments, a kit can contain composition that includes one or more gRNA that can be used for genome-edition, in particular, insertion of a WAS gene or derivative thereof into a genome of a cell. The gRNA for the kit can be target a genomic site at, within, or near the endogenous WAS gene. Alternatively, the gRNA for the kit can target a safe harbor locus or a safe harbor site. Therefore, in some embodiments, the gRNA can have a spacer sequence complementary to (i) a genomic sequence at, within, or near Wiskott-Aldrich syndrome (WAS) gene or (ii) a genomic sequence at, within, or near a safe harbor locus or a safe harbor site. In some embodiments, the safe harbor locus is selected from the group consisting of albumin gene, an AAVS 1 gene, an HRPT gene, a CCR5 gene, a globin gene, TTR gene, TF gene, F9 gene, Alb gene, Gys2 gene and PCSK9 gene. In some embodiments, the safe harbor site is selected from the group consisting of the following regions: AAVS1 19q13.4-qter. HRPT 1q31.2, CCR5 3p21.31, Globin 1p15.4, TTR 18q12.1, TF 3q22.1, F9 Xq27.1, Alb 4q13.3, Gys2 12p12.1, and PCSK9 1p32.3.

In some embodiments, a gRNA for a kit is a sequence selected from those listed in Table 4 and variants thereof having at least 85% homology to any of those listed in Table 4.

In some embodiments, a gRNA for a kit has a spacer sequence that is complementary to a target site in the genome. In some embodiments, the spacer sequence is 15 bases to 20 bases in length. In some embodiments, a complementarity between the spacer sequence to the genomic sequence is at least 80%, at least 85%, at least 90%, at least 95%, at least 96%, at least 97%, at least 98%, at least 99% or at least 100%.

In some embodiments, the kit can have a deoxyribonucleic acid (DNA) endonuclease or an oligonucleotide encoding said DNA endonuclease and/or a donor template having a nucleic acid sequence of a WAS gene or functional derivative thereof.

In some embodiments, the DNA endonuclease for the kit is an enzyme selected from the group consisting of any of those in Table 1, Table 2, and variants having at least 70% homology to any of those listed in Table 1 or Table 2. In some embodiments, the DNA endonuclease is Cas 9. In some embodiments, the oligonucleotide encoding the DNA endonuclease is codon optimized. In some embodiments, the oligonucleotide encoding the DNA endonuclease is a deoxyribonucleic acid (DNA) sequence. In some embodiments, the oligonucleotide encoding the DNA endonuclease is a ribonucleic acid (RNA) sequence. In some embodiments where RNA is used as an oligonucleotide encoding the DNA endonuclease, the RNA is linked to the gRNA via a covalent bond.

In some embodiments, one or more of any oligonucleotides or nucleic acid sequences for the kit can be encoded in an Adeno Associated Virus (AAV) vector. Therefore, in some embodiments, a gRNA can be encoded in an AAV vector. In some embodiments, a nucleic acid encoding a DNA endonuclease can be encoded in an AAV vector. In some embodiments, a donor template can be encoded in an AAV vector. In some embodiments, two or more oligonucleotides or nucleic acid sequences can be encoded in a single AAV vector. Thus, in some embodiments, a gRNA sequence and a DNA endonuclease-encoding nucleic acid can be encoded in a single AAV vector.

In some embodiments, a kit can have a liposome or a lipid nanoparticle. Therefore, in some embodiments, any compounds (e.g, a DNA endonuclease or a nucleic acid encoding thereof, gRNA and donor template) of the kit can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA and donor template is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA and donor template are formulated in a liposome or lipid nanoparticle together.

In any of the above kits, the kit can have a single-molecule guide genome-targeting nucleic acid. In any of the above kits, the kit can have a double-molecule genome-targeting nucleic acid. In any of the above kits, the kit can have two or more double-molecule guides or single-molecule guides. The kits can have a vector that encodes the nucleic acid targeting nucleic acid.

In any of the above kits, the kit can further have a polynucleotide to be inserted to effect the desired genetic modification.

In embodiments, each of three components, i.e, a DNA endonuclease or an oligonucleotide encoding the DNA endonuclease, one or more gRNA and a donor template having a nucleic acid of a WAS gene or functional derivative thereof can be in separate kits. Alternatively, there are at least two kits and a first kit has a DNA endonuclease or an oligonucleotide encoding the DNA endonuclease and one or more gRNAs and a second kit has the donor template. In some embodiments, all three components are contained in one kit.

Components of a kit can be in separate containers, or combined in a single container.

Any kit described above can further have one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. A kit can also have one or more components that can be used to facilitate or enhance the on-target binding or the cleavage of DNA by the endonuclease, or improve the specificity of targeting.

In addition to the above-mentioned components, a kit can further have instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that has a web address where the instructions can be viewed and/or from which the instructions can be downloaded. As with the instructions, this means for obtaining the instructions can be recorded on a suitable substrate.

### VIII. Definitions

The term “comprising” or “comprises” is used in reference to compositions, methods, and respective component(s) thereof, that are essential to the invention, yet open to the inclusion of unspecified elements, whether essential or not.

The term “consisting essentially of” refers to those elements required for a given aspect. The term permits the presence of additional elements that do not materially affect the basic and novel or functional characteristic(s) of that aspect of the invention.

The term “consisting of” refers to compositions, methods, and respective components thereof as described herein, which are exclusive of any element not recited in that description of the aspect.

The singular forms “a,” “an,” and “the” include plural references, unless the context clearly dictates otherwise.

Certain numerical values presented herein are preceded by the term “about.” The term “about” means numerical values within ±10% of the recited numerical value.

When a range of numerical values is presented herein, it is contemplated that each intervening value between the lower and upper limit of the range, the values that are the upper and lower limits of the range, and all stated values with the range are encompassed within the disclosure. All the possible sub-ranges within the lower and upper limits of the range are also contemplated by the disclosure.

The terms “polypeptide”, “peptide”, and “protein” are used interchangeably herein to designate a linear series of amino acid residues connected one to the other by peptide bonds, which series may include proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be taken to mean that such peptide bond or covalent bond to a carboxyl or hydroxyl end group is not present, as it is conventional in representation of amino acid sequences to omit such.

The term “nucleic acid” is used herein in reference to either DNA or RNA, or molecules which contain deoxy- and/or ribonucleotides. Nucleic acids may be naturally occurring or synthetically made, and as such, include analogs of naturally occurring polynucleotides in which one or more nucleotides are modified over naturally occurring nucleotides.

The terms “derivative” and “variant” refer without limitation to any compound such as nucleic acid or protein that has a structure or sequence derived from the compounds disclosed herein and whose structure or sequence is sufficiently similar to those disclosed herein such that it has the same or similar activities and utilities or, based upon such similarity, would be expected by one skilled in the art to exhibit the same or similar activities and utilities as the referenced compounds, thereby also interchangeably referred to “functionally equivalent” or as “functional equivalents”. Modifications to obtain “derivatives” or “variants” may include, for example, addition, deletion and/or substitution of one or more of the nucleic acids or amino acid residues.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/ | Ser, Cys, Thr, Met |
| Selenium-containing | |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a preferred predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The terms “plasmid”. “vector”, “expression cassette” or “expression vector” refer to a nucleic acid molecule that encodes for one or more genes of interest and/or one or more regulatory elements necessary for the expression of the genes of interest.

The term “isolated” is intended to mean that a compound is separated from all or some of the components that accompany it in nature. “Isolated” also refers to the state of a compound separated from all or some of the components that accompany it during manufacture (e.g., chemical synthesis, recombinant expression, culture medium, and the like). “Isolated,” in the context of isolating a cell, can also mean that one or more cells are separated from a group of cells or from a tissue, organ or subject such as an animal or human.

The term “purified” is intended to mean that a compound of interest is isolated and further enriched.

The term “recombinant” or “engineered” when used with reference, for example, to a cell, a nucleic acid, a protein, or a vector, indicates that the cell, nucleic acid, protein or vector has been modified by or is the result of laboratory methods. Thus, for example, recombinant or engineered proteins include proteins produced by laboratory methods. Recombinant or engineered proteins can include amino acid residues not found within the native (non-recombinant) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Given the large number of gene sequences available for a wide variety of animal, plant and microbial species, it is possible to calculate the relative frequencies of codon usage. Codon usage tables are readily available, for example, at the “Codon Usage Database” available at www.kazusa.or.jp/codon/(visited Mar. 20, 2008). By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

As used herein, “transgene,” “exogenous gene” or “exogenous sequence”, in the context of nucleic acid, refers to a nucleic acid sequence or gene that was not present in the genome of a cell but artificially introduced into the genome, e.g. via genome-edition.

As used herein. “endogenous gene” or “endogenous sequence”, in the context of nucleic acid, refers to a nucleic acid sequence or gene that is naturally present in the genome of a cell, without being introduced via any artificial means.

The term “concentration” used in the context of a molecule such as peptide fragment refers to an amount of molecule, e.g., the number of moles of the molecule, present in a given volume of solution.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### IX. Examples

The disclosure will be more fully understood by reference to the following examples, which provide illustrative non-limiting aspects of the disclosure.

The examples describe the use of the CRISPR system as an illustrative genome editing technique to create defined therapeutic genomic deletions, insertions, or replacements, termed “genomic modifications” herein, in the WAS gene that lead to permanent correction of mutations in the genomic locus, or expression at a heterologous locus, that restore WAS protein activity. Introduction of the defined therapeutic modifications represents a novel therapeutic strategy for the potential amelioration of Wiskott-Aldrich Syndrome (WAS), as described and illustrated herein.

### Example 1—CRISPR/SnCas9 Target Sites

Regions of the WAS gene were scanned for target sites. Each area was scanned for a protospacer adjacent motif (PAM) having the sequence NRG. gRNA 20 bp spacer sequences corresponding to the PAM were then identified. Such gRNA is between 15 and 200 nucleotides in length

Regions of the AAVS1 gene which is one of safe harbor genes are scanned for target sites. Each area was scanned for a protospacer adjacent motif (PAM) having the sequence NRG. gRNA 20 bp spacer sequences corresponding to the PAM were then identified. Such gRNA is between 15 and 200 nucleotides in length

### Example 2—CRISPR/SaCas9 Target Sites

Regions of the WAS gene and the AAVS1 gene were scanned for target sites as described in Example 1. Each area was scanned for a protospacer adjacent motif (PAM) having the sequence NNGRRT. gRNA 24 bp spacer sequences corresponding to the PAM are then identified. Such gRNA is be between 15 and 200 nucleotides in length

### Example 3—CRISPR/StCas9 Target Sites

Regions of the WAS gene and the AAVS1 gene were scanned for target sites as described in Example 1. Each area was scanned for a protospacer adjacent motif (PAM) having the sequence NNAGAAW. gRNA 24 bp spacer sequences corresponding to the PAM are then identified. Such gRNA is between 15 and 200 nucleotides in length

### Example 4—CRISPR/TdCas9 Target Sites

Regions of the WAS gene and the AAVS1 gene were scanned for target sites as described in Example 1. Each area was scanned for a protospacer adjacent motif (PAM) having the sequence NAAAAC. gRNA 24 bp spacer sequences corresponding to the PAM are then identified. Such gRNA is between 15 and 200 nucleotides in length

### Example 5—CRISPR/NmCas9 Target Sites

Regions of the WAS gene and the AAVS1 gene were scanned for target sites as described in Example 1. Each area was scanned for a protospacer adjacent motif (PAM) having the sequence NNNNGATT. gRNA 24 bp spacer sequences corresponding to the PAM are then identified. Such gRNA is between 15 and 200 nucleotides in length

### Example 6—CRISPR/Cpf1 Target Sites

Regions of the WAS gene and the AAVS1 gene were scanned for target sites as described in Example 1. Each area was scanned for a protospacer adjacent motif (PAM) having the sequence was TTN or YTN. gRNA 24 bp spacer sequences corresponding to the PAM are then identified. Such gRNA is between 15 and 200 nucleotides in length

### Example 7—Bioinformatics Analysis of the Guide Strands

Candidate guides are then screened and selected in a single process or multi-step process that involves both theoretical binding and experimentally assessed activity. By way of illustration, candidate guides having sequences that match a particular on-target site, such as a site at, within, or near the WAS gene or a safe harbor site, with adjacent PAM are assessed for their potential to cleave at off-target sites having similar sequences, using one or more of a variety of bioinformatics tools available for assessing off-target binding in order to assess the likelihood of effects at chromosomal positions other than those intended.

Candidates predicted to have relatively lower potential for off-target activity are assessed experimentally to measure their on-target activity, and then off-target activities at various sites. Preferred guides have sufficiently high on-target activity to achieve desired levels of gene editing at the selected locus, and relatively lower off-target activity to reduce the likelihood of alterations at other chromosomal loci. The ratio of on-target to off-target activity is referred to as the “specificity” of a guide.

For initial screening of predicted off-target activities, there are a number of bioinformatics tools known and publicly available that can be used to predict the most likely off-target sites; and since binding to target sites in the CRISPR/Cas9/Cpf1 nuclease system is driven by Watson-Crick base pairing between complementary sequences, the degree of dissimilarity (and therefore reduced potential for off-target binding) is essentially related to primary sequence differences; mismatches and bulges, i.e. bases that are changed to a non-complementary base, and insertions or deletions of bases in the potential off-target site relative to the target site. An exemplary bioinformatics tool called COSMID (CRISPR Off-target Sites with Mismatches. Insertions and Deletions) (available on the web at crispr.bme.gatech.edu) compiles such similarities. Other bioinformatics tools include, but are not limited to, GUIDO, autoCOSMID, and CCtop.

Bioinformatics are used to minimize off-target cleavage in order to reduce the detrimental effects of mutations and chromosomal rearrangements. Studies on CRISPR/Cas9 systems suggested the possibility of high off-target activity due to nonspecific hybridization of the guide strand to DNA sequences with base pair mismatches and/or bulges, particularly at positions distal from the PAM region. Therefore, it is important to have a bioinformatics tool that can identify potential off-target sites that have insertions and/or deletions between the RNA guide strand and genomic sequences, in addition to base-pair mismatches. The bioinformatics-based tool, COSMID (CRISPR Off-target Sites with Mismatches, Insertions and Deletions) was therefore used to search genomes for potential CRISPR off-target sites (available on the web at crispr.bme.gatech.edu). COSMID output ranked lists of the potential off-target sites based on the number and location of mismatches, allowing more informed choice of target sites, and avoiding the use of sites with more likely off-target cleavage.

Additional bioinformatics pipelines are employed that weigh the estimated on- and/or off-target activity of gRNA targeting sites in a region. Other features that are used to predict activity include information about the cell type in question, DNA accessibility, chromatin state, transcription factor binding sites, transcription factor binding data, and other CHIP-seq data. Additional factors are weighed that predict editing efficiency, such as relative positions and directions of pairs of gRNAs, local sequence features and micro-homologies.

### Example 8—Testing of Preferred Guides in Cells for On-Target Activity

The gRNAs predicted to have the lowest off-target activity were tested for on-target activity in a model cell line of stable HEK293T cells that expresses S. pyogenes Cas9, and evaluated for InDel frequency using TIDE or next generation sequencing. TIDE is a web tool to rapidly assess genome editing by CRISPR-Cas9 of a target locus determined by a guide RNA (gRNA or sgRNA). Based on quantitative sequence trace data from two standard capillary sequencing reactions, the TIDE software quantifies the editing efficacy and identifies the predominant types of insertions and deletions (InDels) in the DNA of a targeted cell pool. See Brinkman et al., Nucl. Acids Res. (2014) for a detailed explanation and examples. Next-generation sequencing (NGS), also known as high-throughput sequencing, is the catch-all term used to describe a number of different modern sequencing technologies including: Illumina (Solexa) sequencing, Roche 454 sequencing, Ion torrent: Proton/PGM sequencing, and SOLiD sequencing. These recent technologies allow one to sequence DNA and RNA much more quickly and cheaply than the previously used Sanger sequencing, and as such have revolutionized the study of genomics and molecular biology.

Transfection of tissue culture cells is used for screening of different constructs and a robust means of testing activity and specificity. Tissue culture cell lines, such as HEK293T, are easily transfected and result in high activity. These or other cell lines are evaluated to determine the cell lines that match with HEK293T and provide the best surrogate. These cells are then be used for many early stage tests. In one test, individual gRNAs for S. pyogenes Cas9 are transfected into the cells via lipofection. Several days later, the genomic DNA is harvested and the target site amplified by PCR The cutting activity is measured by the rate of insertions, deletions and mutations introduced by NHEJ repair of the free DNA ends. Although this method cannot differentiate correctly repaired sequences from uncleaved DNA, the level of cutting is gauged by the amount of mis-repair. Off-target activity is observed by amplifying identified putative off-target sites and using similar methods to detect cleavage. In another test, translocation is assayed using primers flanking cut sites, to determine if specific cutting and translocations happen. Un-guided assays is also preformed to conduct complementary testing of off-target cleavage including guide-seq. The gRNA or pairs of gRNA with significant activity is followed up in cultured cells to measure correction of the WAS gene mutation. Off-target events is followed again. A variety of cells is transfected and the level of gene correction and possible off-target events are measured. With this series of test, optimization of nuclease and donor design and delivery are achieved.

### Example 9—Testing of Preferred Guides in Cells for Off-Target Activity

The gRNAs having the best on-target activity from the TIDE and next generation sequencing studies in the above example were tested for off-target activity using whole genome sequencing.

Table 4 shows spacer sequences of gRNAs that were selected for further testing. Each of the gRNA sequences was introduced with DNA endonuclease into a cell, cutting at the target site upstream of the endogenous WAS gene and their cutting efficiency at the intended target site was measured.

### Example 10—Testing Different Approaches for HDR Gene Editing

After testing the gRNAs for both on-target activity and off-target activity, mutation correction and knock-in strategies are tested for HDR gene editing.

For the mutation correction approach, donor DNA template is provided as a short single-stranded oligonucleotide, a short double-stranded oligonucleotide (PAM sequence intact/PAM sequence mutated), a long single-stranded DNA molecule (PAM sequence intact/PAM sequence mutated) or a long double-stranded DNA molecule (PAM sequence intact/PAM sequence mutated). In addition, the donor DNA template is delivered by AAV.

For the cDNA knock-in approach, a single-stranded or double-stranded DNA having homologous arms to the WAS gene chromosomal region includes more than 40 nt of the first exon (the first coding exon) of the WAS gene, the complete CDS of the WAS gene and 3′ UTR of the WAS gene, and at least 40 nt of the following intron. The single-stranded or double-stranded DNA having homologous arms to the WAS gene chromosomal region includes more than 80 nt of the first exon of the WAS gene, the complete CDS of the WAS gene and 3′ UTR of the WAS gene, and at least 80 nt of the following intron. The single-stranded or double-stranded DNA having homologous arms to the WAS gene chromosomal region include more than 100 nt of the first exon of the WAS gene, the complete CDS of the WAS gene and 3′ UTR of the WAS gene, and at least 100 nt of the following intron. The single-stranded or double-stranded DNA having homologous arms to the WAS gene chromosomal region includes more than 150 nt of the first exon of the WAS gene, the complete CDS of the WAS gene and 3′ UTR of the WAS gene, and at least 150 nt of the following intron. The single-stranded or double-stranded DNA having homologous arms to the WAS gene chromosomal region includes more than 300 nt of the first exon of the WAS gene, the complete CDS of the WAS gene and 3′ UTR of the WAS gene, and at least 300 nt of the following intron. The single-stranded or double-stranded DNA having homologous arms to the WAS gene chromosomal region includes more than 400 nt of the first exon of the WAS gene, the complete CDS of the WAS gene and 3′ UTR of the WAS gene, and at least 400 nt of the following intron.

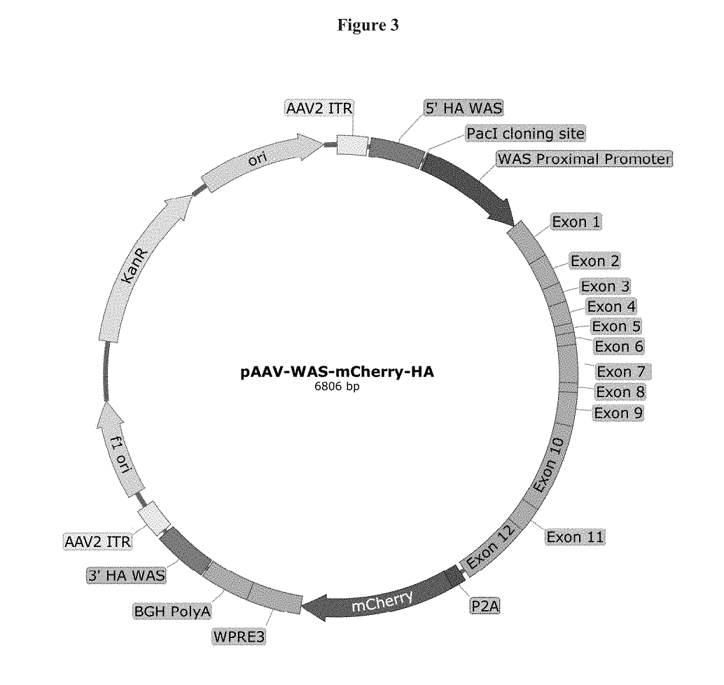

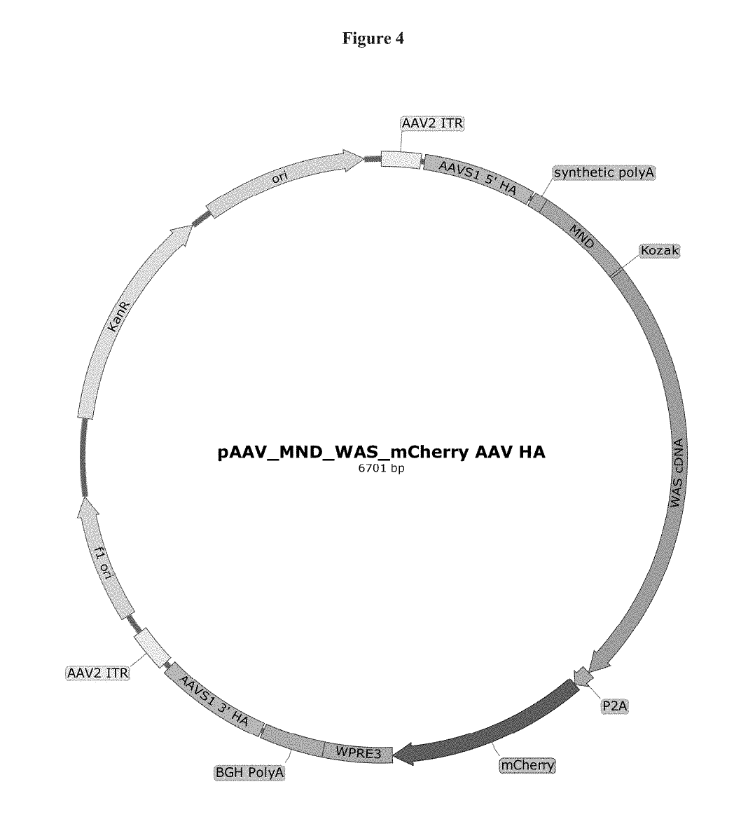

The constructs illustrated in FIGS. 3 and 4 show exemplary AAV vectors that were used to insert a WAS gene or functional derivative thereof into a genome. The construct of FIG. 3 was used for the insertion at, within, or near the WAS gene locus. The construct of FIG. 4 was used for the insertion in a safe harbor locus or site, e.g, at, within, or near the AAVS1 gene locus. The vectors also contained a reporter gene mCherry for in vitro analysis of integration, besides a sequence encoding the WAS gene. The reporter gene is optional for therapeutic purposes.

Alternatively, the DNA template is delivered by a recombinant AAV particle such as those taught herein.

A knock-in of WAS gene cDNA is performed into any selected chromosomal location or in one of the “safe-harbor” locus, i.e., albumin gene, an AAVS1 gene, an HRPT gene, a CCR5 gene, a globin gene. TTR gene, TF gene. F9 gene, Alb gene. Gys2 gene and PCSK9 gene. Assessment of efficiency of HDR mediated knock-in of cDNA into the first exon utilizes cDNA knock-in into “safe harbor” sites such as: single-stranded or double-stranded DNA having homologous arms to one of the following regions, for example: AAVS1 19q13.4-qter. HRPT 1q31.2, CCR5 3p21.31, Globin 11p15.4. TTR 18q12.1, TF 3q22.1, F9 Xq27.1. Alb 4q13.3, Gys2 12p12.1, PCSK9 1p32.3; 5′UTR correspondent to WAS gene or alternative 5′ UTR, complete CDS of WAS gene and 3′ UTR of WAS gene or modified 3′ UTR and at least 80 nt of the first intron, alternatively same DNA template sequence will be delivered by AAV.

### Example 11—Re-Assessment of Lead CRISPR-Cas9/DNA Donor Combinations

After testing the different strategies for HDR gene editing, the lead CRISPR-Cas9/DNA donor combinations were re-assessed in cells for efficiency of deletion, recombination, and off-target specificity. Cas9 mRNA or RNP are formulated into lipid nanoparticles for delivery, sgRNAs are formulated into nanoparticles or delivered as a recombinant AAV particle, and donor DNA are formulated into nanoparticles or delivered as recombinant AAV particle.

### Example 12—In Vivo Testing in Relevant Animal Model

After the CRISPR-Cas9/DNA donor combinations have been re-assessed, the lead formulations are tested in vivo in a FGR mouse model with the livers repopulated with human hepatocytes (normal or WAS gene-deficient).

### Example 13—In Vivo Testing in Relevant Animal Model

After the CRISPR-Cas9/DNA donor combinations have been assessed, the expression of WAS or functional derivative thereof is tested in vivo in the WAS knock-out (Was^{-/-}) or WAS-deficient mouse model that is constructed as described in Shimizua. M, et al., “Development of IgA nephropathy-like glomerulonephritis associated with Wiskott-Aldrich syndrome protein deficiency,” 2011, Clin. Immunol., vol. 142, issue 2, pp.: 160-166, which is incorporated herein by reference in entirety.

### Example 14—In Vivo Testing in Relevant Animal Model

After the CRISPR-Cas9/DNA donor combinations have been assessed, the expression of WAS or functional derivative thereof is tested in vivo in the WAS knock-out (Was^{-/-}) or WAS-deficient mouse model that is constructed as described in Snapper. S, et al., “Wiskott-Aldrich Syndrome Protein-Deficient Mice Reveal a Role for WASP in T but Not B Cell Activation,” 1998, Immunity, vol. 9, issue 1, pp.: 81-91, which is incorporated herein by reference in entirety.

### Example 15-Functional Analysis of WAS Expression

WAS-deficient T and B cell lines were generated by introducing single base insertions in exon 7 of the WAS gene by introducing CRISPR/Cas9 and site specific gRNA (CCCTGGGGCTGGCGACAGTGG) through nucleofection. WAS-deficient T and B cell clones were expanded and genomic sequencing confirmed interruption of WAS gene. Absence of WAS protein was confirmed using standard protein analysis techniques. Rescue of WAS expression was achieved by insertion of full length WAS cDNA and 500 bp upstream sequence for proximal promoter at endogenous WAS locus or MND with full length WAS cDNA at AAVS1 locus in WAS-deficient cell lines.

WAS-deficient T and B cell lines were analyzed for ability to migrate toward a chemoattractant, such as SDF1-alpha, through a transwell membrane. WAS-deficient T and B cell lines have impaired migration toward chemo attractants in comparison to WAS-expressing T and B cell lines as determined by quantification of cells that have migrated through the membrane. WAS-deficient T and B cell lines were analysed for ability to proliferate in response to activation by a stimulator, such as phorbol myristate acetate (PMA) or lipopolysaccharide (LPS). Briefly, cells were labeled with a fluorophore, such as carboxyfluorescein succinimidyl ester to allow tracking of number of cell divisions based on fluorescent intensity of labeled cells after 3 or more days in culture. Fluorescent intensity was measured on individual cells flow cytometrically and numbers of cells which had completed 1, 2, 3, 4, 5 or more cell divisions were quantified. WAS-deficient T and B cell lines had impaired proliferation in response to stimulation in comparison to WAS-expressing T and B cell lines.

### Example 16—Screening of gRNAs

To identify a large spectrum of pairs of gRNAs able to edit the WAS DNA target region, an in vitro transcribed (IVT) gRNA screen was conducted. WAS genomic sequence was analyzed using a gRNA design software as described herein. The resulting list of gRNAs was narrowed to a list of 188 gRNAs (see Table 4). This set of gRNAs was in vitro transcribed, and transfected together with Cas9 protein in a test cell using electroporation (Lonza 4D with 96 well shuttle). Cells were harvested 48 hours post transfection, the genomic DNA was isolated, and cutting efficiency was evaluated using TIDE analysis.

### X. Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

What is claimed is:

1. A method of editing a genome in a cell, the method comprising:
inserting a nucleic acid sequence of a Wiskott-Aldrich syndrome gene (WAS gene) or functional derivative thereof into a genomic sequence of the cell,
wherein said cell has one or more mutation(s) in the genome which results in reduction of the expression of endogenous WAS gene as compared to said expression in a normal cell that does not have such mutation(s).

2. The method of claim 1 further comprising providing the following to the cell:
(a) a deoxyribonucleic acid (DNA) endonuclease or an oligonucleotide encoding said DNA endonuclease; and
(b) a targeting oligonucleotide comprising a first region of at least 15 bases complementary to the genomic sequence; wherein the WAS gene or functional derivative thereof is inserted using a donor template comprising the nucleic acid sequence of the WAS gene or functional derivative thereof.

3. The method of claim 2, wherein said DNA endonuclease is an enzyme selected from the group consisting of any of those in Table 1, Table 2, and variants having at least 70% homology to any of those listed in Table 1 or Table 2.

4. The method of claim 2, wherein said DNA endonuclease is Cas 9.

5. The method of claim 2, wherein the oligonucleotide encoding said DNA endonuclease is codon optimized.

6. The method of claim 2, wherein the oligonucleotide encoding said DNA endonuclease is a deoxyribonucleic acid (DNA) sequence.

7. The method of claim 2, wherein the oligonucleotide encoding said DNA endonuclease is a ribonucleic acid (RNA) sequence.

8. The method of claim 7, wherein the RNA sequence encoding said DNA endonuclease is linked to the targeting oligonucleotide via a covalent bond.

9. The method of claim 2, wherein said targeting oligonucleotide is a guide RNA (gRNA).

10. The method of claim 9, wherein the first region of said gRNA is selected from those listed in Table 4 and variants thereof having at least 85% homology to any of those listed in Table 4.

11. The method of claim 1, wherein said genomic sequence is at, within, or near the WAS gene or WAS gene regulatory elements.

12. The method of claim 11, wherein said genomic sequence is in an intergenic region that is upstream of the promoter of the endogenous WAS gene in the genome.

13. The method of claim 11, wherein said intergenic region is at least 500 bp upstream of the first exon of the endogenous WAS gene in the genome.

14. The method claim 1, wherein said inserting is at, within, or near a safe harbor locus or a safe harbor site.

15. The method of claim 14, wherein said safe-harbor locus is selected from the group consisting of albumin gene, AAVS1 gene, HRPT gene, CCR5 gene, globin gene, TTR gene, TF gene, F9 gene, Alb gene, Gys2 gene and PCSK9 gene.

16. The method of claim 14, wherein said safe harbor site is selected from the group consisting of the following regions: AAVS119q13.4-qter, HRPT 1q31.2, CCR5 3p21.31, Globin 11p15.4, TTR 18q12.1, TF 3q22.1, F9 Xq27.1, Alb 4q113.3, Gys2 12p12.1, and PCSK9 1p32.3.

17. The method of claim 15, wherein said genomic sequence is at, within, or near the AAVS1 gene.

18. The method of claim 17, wherein said genomic sequence is in an intergenic region that is upstream of the promoter of the AAVS11 gene in the genome.

19. The method of claim 17, wherein said intergenic region is at least 2.5 kb upstream of the first exon of the AAVS1 gene in the genome.

20. The method of claim 17, wherein said intergenic region is about 2.5 kb to about 5 kb upstream of the first exon of the AAVS1 gene in the genome.

21. The method of claim 2, wherein one or more of said oligonucleotides are encoded in an Adeno Associated Virus (AAV) vector.

22. The method of claim 2, wherein said DNA endonuclease and/or one or more of said oligonucleotide are formulated in a liposome or lipid nanoparticle.

23. The method of claim 22, wherein said DNA endonuclease is formulated in a liposome or lipid nanoparticle.

24. The method of claim 23, wherein said liposome or lipid nanoparticle further comprises the targeting oligonucleotide.

25. The method of claim 2, wherein said one or more of (a), (b) and (c) are provided to the cell via electroporation.

26. The method of claim 2, wherein said one or more of (a), (b) and (c) are provided to the cell via chemical transfection.

27. The method of claim 2, wherein said DNA endonuclease is precomplexed with the targeting oligonucleotide, forming a Ribonucleoprotein (RNP) complex, prior to the provision to the cell.

28. The method of claim 27, wherein said RNP is provided to the cell via electroporation.

29. The method of claim 1, wherein said one or more mutation(s) are present at, within, or near the endogenous WAS gene in the genome.

30. The method of claim 1, the expression of endogenous WAS gene in said cell is about 10%, about 20%, about 30%, about 40%, about 50%, about 60%, about 70%, about 80%, about 90% or about 100% reduced as compared to the expression of endogenous WAS gene expression in the normal cell.

31. The method of claim 1, wherein the expression of the introduced WAS gene or functional derivative thereof in the cell is at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%6, about 3,000%, about 5,000%, about 10,000% or more as compared to the expression of endogenous WAS gene of the cell.

32. The method of claim 1, wherein the expression of the introduced WAS gene or functional derivative thereof in the cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the cell

33. The method of claim 1, wherein said cell is a stem cell.

34. The method of claim 33, wherein said stem cell is a CD34⁺ hematopoietic stem and progenitor cell (HSPC).

35. A method of treating a subject for a Wiskott-Aldrich syndrome (WAS) gene related condition or disorder comprising:
providing a genetically modified cell to the subject,
wherein a genome of the genetically modified cell is edited such that an exogenous nucleic acid sequence of a WAS gene or functional derivative thereof is inserted in the genome.

36. The method of claim 35, wherein said subject is a patient having or is suspected of having Wiskott-Aldrich syndrome (WAS).

37. The method of claim 35, wherein said subject is diagnosed with a risk of the Wiskott-Aldrich syndrome (WAS) gene related condition or disorder.

38. The method of claim 35, wherein said genetically modified cell is autologous.

39. The method of claim 38, wherein said autologous cell has one or more mutation(s) in the genome which results in reduction of the expression of endogenous WAS gene as compared to the expression of endogenous WAS gene in a normal cell that does not have such mutation(s).

40. The method of claim 39, wherein said one or more mutation(s) are present at, within, or near the endogenous WAS gene in the genome.

41. The method of claim 39, the expression of endogenous WAS gene in the genetically modified cell is about 10%, about 20%, about 30%, about 40%, about 50%, about 60%, about 70%, about 80%, about 90%6 or about 100% reduced as compared to the expression of endogenous WAS gene expression in a normal cell that does not have such mutation(s).

42. The method of claim 39, wherein the expression of the introduced WAS gene or functional derivative thereof in the genetically modified cell is at least about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, about 1,000%, about 2,000%, about 3,000%, about 5,000%, about 10,000% or more as compared to the expression of endogenous WAS gene of the genetically modified cell.

43. The method of claim 39, wherein the expression of the introduced WAS gene or functional derivative thereof in the genetically modified cell is at least about 2 folds, about 3 folds, about 4 folds, about 5 folds, about 6 folds, about 7 folds, about 8 folds, about 9 folds, about 10 folds, about 15 folds, about 20 folds, about 30 folds, about 50 folds, about 100 folds or more of the expression of endogenous WAS gene of the genetically modified cell.

44. The method of claim 35, wherein said cell is a stem cell.

45. The method of claim 44, wherein said stem cell is a CD34⁺ hematopoietic stem and progenitor cell (HSPC).

46. The method of claim 35 further comprising:
obtaining a biological sample from the subject wherein the biological sample comprises a CD34⁺ cell; and
editing the genome of at least one cell by inserting the exogenous nucleic acid sequence of a WAS gene or functional derivative thereof into a genomic sequence of the cell, thereby producing the genetically modified cell.

47. The method of claim 35, wherein the exogenous nucleic acid sequence is inserted at, within, or near the WAS gene or WAS gene regulatory elements.

48. The method of claim 35, wherein said genomic sequence is in an intergenic region that is upstream of the promoter of the endogenous WAS gene in the genome.

49. The method of claim 35, wherein said intergenic region is at least 500 bp upstream of the first exon of the endogenous WAS gene in the genome.

50. The method of claim 35, wherein the exogenous nucleic acid sequence is inserted at, within, or near a safe harbor locus or a safe harbor site.

51. The method of claim 50, wherein said safe-harbor locus is selected from the group consisting of albumin gene, AAVS1 gene, HRPT gene, CCR5 gene, globin gene, TTR gene, TF gene, F9 gene, Alb gene, Gys2 gene and PCSK9 gene.

52. The method of claim 50, wherein said safe harbor site is selected from the group consisting of the following regions: AAVS1 19q113.4-qter, HRPT 1q31.2, CCR5 3p21.31, Globin 11p15.4, TTR 18q12.1, TF 3q22.1, F9 Xq27.1, Alb 4q13.3, Gys2 12p12.1, and PCSK9 1p32.3.

53. The method of claim 51, wherein the exogenous nucleic acid sequence is inserted at, within, or near the AAVS1 gene.

54. The method of claim 53, wherein said genomic sequence is in an intergenic region that is upstream of the promoter of the AAVS1 gene in the genome.

55. The method of claim 53, wherein said intergenic region is at least 2.5 kb upstream of the first exon of the AAVS1 gene in the genome.

56. The method of claim 53, wherein said intergenic region is about 2.5 kb to about 5 kb upstream of the first exon of the AAVS1 gene in the genome.

57. A composition comprising a guide RNA (gRNA) sequence comprising a sequence selected from those listed in Table 4 and/or variants thereof having at least 85% homology to any of those listed in Table 4.

58. The composition of claim 57 further comprising a DNA endonuclease or an oligonucleotide encoding said DNA endonuclease.

59. The composition of claim 58 further comprising a donor template comprising a nucleic acid sequence of a WAS gene or functional derivative thereof.

60. The composition of claim 59, wherein said DNA endonuclease is an enzyme selected from the group consisting of any of those in Table 1, Table 2, and variants having at least 70% homology to any of those listed in Table 1 or Table 2.

60. The composition of claim 59, wherein said DNA endonuclease is Cas 9.

61. The composition of claim 58, wherein the oligonucleotide encoding said DNA endonuclease is codon optimized.

62. The composition of claim 58, wherein the oligonucleotide encoding said DNA endonuclease is a deoxyribonucleic acid (DNA) sequence.

63. The composition of claim 58, wherein the oligonucleotide encoding said DNA endonuclease is a ribonucleic acid (RNA) sequence.

64. The composition of claim 63, wherein the RNA sequence encoding said DNA endonuclease is linked to the gRNA via a covalent bond.

65. The composition of claim 58 further comprising a liposome or lipid nanoparticle.

66. The composition of claim 58, wherein said DNA endonuclease is precomplexed with the gRNA, forming a Ribonucleoprotein (RNP) complex.

67. A composition comprising a guide RNA (gRNA) sequence that has a spacer sequence complementary to (i) a genomic sequence at, within, or near Wiskott-Aldrich syndrome (WAS) gene or (ii) a genomic sequence at, within, or near a safe harbor locus or a safe harbor site.

68. The composition of claim 67, wherein said safe harbor locus is selected from the group consisting of albumin gene, AAVS1 gene, HRPT gene, CCR5 gene, globin gene, TTR gene, TF gene, F9 gene, Alb gene, Gys2 gene and PCSK9 gene.

69. The composition of claim 67, wherein said safe harbor site is selected from the group consisting of the following regions: AAVS1 19q13.4-qter, HRPT 1q31.2, CCR5 3p21.31, Globin 1p15.4, TTR 18q12.1, TF 3q22.1, F9 Xq27.1, Alb 4q13.3, Gys2 12p12.1, and PCSK9 1p32.3.

70. The composition of claim 67, wherein said spacer sequence is 15 bases to 20 bases in length.

71. The composition of claim 67, wherein a complementarity between the spacer sequence to the genomic sequence is at least 80%, at least 85%, at least 90%, at least 950%, at least 96%, at least 97%, at least 98%, at least 99% or at least 100%.

72. The composition of claim 67 further comprising one or more of the following:
a deoxyribonucleic acid (DNA) endonuclease or an oligonucleotide encoding said DNA endonuclease; and
a donor template comprising a nucleic acid sequence of a WAS gene or functional derivative thereof.

73. The composition of claim 72, wherein said DNA endonuclease is an enzyme selected from the group consisting of any of those in Table 1, Table 2, and variants having at least 70% homology to any of those listed in Table 1 or Table 2.

74. The composition of claim 72, wherein said DNA endonuclease is Cas 9.

75. The composition of claim 72, wherein the oligonucleotide encoding said DNA endonuclease is codon optimized.

76. The composition of claim 72, wherein the oligonucleotide encoding said DNA endonuclease is a ribonucleic acid (RNA) sequence.

77. The composition of claim 77, wherein the RNA sequence encoding said DNA endonuclease is linked to the gRNA via a covalent bond.

78. The composition of claim 67 further comprising a liposome or lipid nanoparticle.

79. The composition of claim 72, wherein said DNA endonuclease is precomplexed with the gRNA, forming a Ribonucleoprotein (RNP) complex.

80. A kit comprising the composition of claim 59 further comprising instructions for use.
